(19) United States
(12) Patent Application Publication
(10) Pub. No.: US 2012/0005770 A1
(43) Pub. Date: Jan. 5, 2012

(54) VEGETABILE MATERIAL, PLANTS AND A METHOD OF PRODUCING A PLANT HAVING ALTERED LIGNIN PROPERTIES

(75) Inventors: Magnus Hertzberg, Umea (SE); Björn Sundberg, Umea (SE); Göran Sandberg, Umea (SE); Jarmo Schrader, Bad Salzdetfurth (DE); Tuula Teeri, Taby (SE); Henrik Aspeborg, Segeltorp (SE); Lars Wallbäcks, Umea (SE); Rishikeshi Bhalerao, Umea (SE); Johan Trygg, Umea (SE); Karin Johansson, Roeback (SE); Ann Karlsson, Umea (SE); Pär Jonsson, Umea (SE)

(73) Assignee: Swetree Technologies AB, Umea (SE)

(21) Appl. No.: 13/127,457

(22) PCT Filed: Nov. 3, 2009
(86) PCT No.: PCT/SE09/51239

§ 371 Date: Sep. 16, 2011

Related U.S. Application Data
(60) Provisional application No. 61/110,614, filed on Nov. 3, 2008.

(30) Foreign Application Priority Data
Nov. 3, 2008 (SE) 0850065-4

(51) Int. Cl. A01H 1/02; C12N 15/82; C12N 5/10; A01H 1/00; A01H 5/00; C12N 5/04; C12N 15/11; A01H 1/06

(52) U.S. Cl. 800/260; 536/23.1; 435/320.1; 800/276; 800/278; 800/295; 800/298; 435/419; 435/410

(57) ABSTRACT

The present invention is related to a set of genes, which when modified in plants gives altered lignin properties. The invention provides DNA construct such as a vector useful in the method of the invention. Further, the invention relates to a plant cell or plant progeny of the plants and wood produced by the plants according to the invention Lower lignin levels will result in improved saccharification for bio-refining and ethanol production and improved pulp and paper. Increased lignin levels will utilise lignin properties for energy production. The genes and DNA constructs may be used for the identification of plants having altered lignin characteristics as compared to the wild-type. According to the invention genes and DNA constructs may also be used as candidate genes in marker assisted breeding.

### TECHNICAL FIELD

The present invention is related to a set of genes, which when modified in plants gives altered lignin properties. More specifically, the invention relates to methods for phenotypically modifying plants and transgenic plants and plants obtained by a specific crossing method having altered expression of a gene resulting in a modified lignin expression. The invention also provides DNA construct such as a vector useful in the method of the invention. Further, the invention relates to a plant cell or plant progeny of the plants and wood produced by the plants according to the invention.

Lower lignin levels will result in improved saccharification for bio-refining and ethanol production and improved pulp and paper. Increased lignin levels will utilise lignin properties for energy production.

The genes and DNA constructs may be used for the identification of plants having altered lignin characteristics as compared to the wild-type.

According to the invention genes and DNA constructs may also be used as candidate genes in marker assisted breeding.

### BACKGROUND

Wood from forest trees provides the biopolymers cellulose, hemicellulose and lignin for industrial uses. Major products produced from wood include pulp and paper, energy and building materials. However, we now see a shift where industry looks for novel production possibilities as well as for improved bio-refining of the wood raw material to more valuable products. This is driven by the aim to replace the use of fossil materials with renewable resources. Wood is such a resource of major importance, and it is therefore important to supply the industry with large amount of wood with designed properties for variable uses.

Lignin is a major wood biopolymer that is highly important for industrial processing of most wood based materials. Lignin biosynthesis has been well described by biochemical and genetic research tools (Baucher, Critical Reviews in Biochemistry and Molecular Biology, 38:305-350, 2003). All major enzymes in the biosynthetic pathway have been identified, and reverse genetics has been helpful in highlighting the major route of the pathway(s). Lignin is a highly complex biopolymer. It is composed of p-hydroxycinnamyl alcohol monomers and related compounds. This are polymerized oxidised and coupled in a chemical process, and the lignin is encrusting the cellulose and hemicellulose matrix in the wood cell walls. It links in complex, and hitherto, poorly described ways to the hemicellulose network with so-called “lignin carbon complexes”. It has recently also been obvious that the lignin structure is very plastic and variable. Both lignin content and its composition are naturally variable between organs and cell types, and this variability can be mimicked and extended by genetic engineering (Vanholme et al. Current Opinion in Plant Biology 2008, 11:1-8). Not surprisingly in transgenic trees with modified lignin, pulping properties such as yield and kappa number has been modified (Bauhcer, Critical Reviews in Biochemistry and Molecular Biology, 38:305-350, 2003). More recent research has also demonstrated that saccharification needed for wood bio-refining and for example ethanol production is also modified when lignin is modified (Chen and Dixon. Nature Biotechnology Vol. 25 no 7: pp 759-761, 2007). This paper shows that lower lignin levels results in improved saccharification. Saccharification is a process were the poly carbohydrates are hydrolysed into sugar monomers such as glucose. This process is expensive and advanced for lignocellulosic raw materials (Breaking the Biological Barriers to Cellulosic Ethanol: A Joint Research Agenda. A Research Roadmap Resulting from the Biomass to Biofuels Workshop, Dec. 7-9, 2005, Rockville, Md.).

To access the cellulose and hemicellulose for the saccharification, the cell wall structure has to be loosened, thereby enabling it for enzymatic degradation or hydrolysis.

Furthermore, this pre-treatment may increase the surface area of the cellulose thereby enhancing its reactivity with the enzyme and the transformation to monosaccharides.

Despite the rather well researched lignin biosynthetic pathway recent observations point to the fact that lignin content can also be modified through perturbing genes related to other metabolic and cell wall biopolymer pathways in a rather unexplored, and not yet understood, fashion This is an unexplored potential to modify wood properties and lignin chemistry in trees.

The commercial value of engineering lignin content in wood is obvious, but the usefulness of more or less lignin depends upon the utilization of wood and the industrial process used. For energy production through burning or other combustion technology high lignin content is valuable because lignin has a high-energy value compared to other wood biopolymers.

Also, in chemical pulping, forage digestibility and processing of plant biomass to bio fuels lignin represent a major obstacle. This is because lignin is covalently bound to the carbon matrix, and energy is required to isolate or degrade the lignin. In pulp and paper processing, for example, it becomes a rest product ending up in the rest liquor, and separation of lignin from the cellulose is an energy consuming process. Therefore low lignin content has been viewed as a valuable property.

One example of genes involved in the biosynthetic pathway of lignin is the genes involved in the monolignol synthesis. Forster and colleagues describe in the U.S. Pat. No. 7,402,428 that they can reduce or modulate the lignin content in plants by RNAi knockout of a gene in the monolignol biosynthetic pathway. This is one gene of many genes involved in the lignin synthesis pathway, due to different growth and environment condition very little is know how the this gene construct will function in field growth conditions and which genes will be best for different applications. Thus there is still a need for additional genes that can be used for modulation of lignin content in plants.

But ongoing research trying to establish wood bio refining, where all wood components are processed may change this view. The use of lignin as a dispergent, emulsion, binding agent or stabilizer has already been developed. If novel lignin based products are becoming more attractive, the lignin content of the wood may be looked at in different views. It is therefore of value to be able to modify lignin content in wood in a desirable way, e.g. up or down depending on the intended use.

Problems remaining are how to identify the potentially most important genes involved in regulation of lignin and expression of lignin related genes, in order to utilise lignin properties for energy production or remove unwanted lignin properties in paper production. In this present invention we examined a number of genes affecting the lignin levels in living trees.

### BRIEF DESCRIPTION OF THE INVENTION

The present invention pertains to genes, and DNA constructs SEQ ID No: 23 to 115 that can be used to modify lignin level in plants. This is done by modification of the expression level of these genes.

The invention relates to methods for phenotypically modifying plants and transgenic plants and plants obtained by a specific crossing method having altered expression of a gene resulting in a modified lignin expression. The invention also provides DNA constructs useful in the method of the invention. Further, the invention relates to a plant cell, plant progeny or any vegetable material of the plants and wood produced by the plants according to the invention and uses thereof in applications where increased or decreased lignin content is an advantage.

The inventors have found that the down regulation and/or over expression of certain genes, gives changed wood chemistry in the produced transgenic trees. Especially it was realized that certain genes can be used to modify lignin levels. These genes have been identified from a program where gene have been transferred to trees and been down regulated by the use of RNAi and over expressed by high level expression promoters.

These genes were indentified to affect the lignin level after a two step analysis, firstly the wood was analysed with FT-IR analysis (general reference Griffiths, P. R. and De Haseth, J. A. Fourier Transform Infrared Spectrometry. New York: Wiley, 1986 and the text below) and secondly the wood was analysed with the Klason-lignin analysis (see below in text.).

The genes (SEQ ID No: 23 to 115, 157-195) have changed chemical wood properties, especially changed lignin levels.

The genes (SEQ ID 23 to 115, 157-195) can be expressed in plants for further use as energy crops, for example if the lignin content is increased a high-energy crop will be generated.

Furthermore, these genes (SEQ ID 23 to 115, 157-195) can when expressed in plants further be used to increase and improve saccharification potentials and thereby enhance the economics in producing fuels and chemicals from ligno-cellulose.

### DETAILED DESCRIPTION OF THE INVENTION

### Definitions

Prior to discussing the present invention in further details, the following terms and conventions will first be defined:

Definition of “saccharification” is the process of converting a complex carbohydrate, such as starch, cellulose or hemicellulose, into sugars such as fermentable sugars. It is essentially a hydrolysis. The process may for example be accomplished by the use of enzymes or acids or other chemicals.

The term “transgenic plant” refers to a plant that contains genetic material, not found in a wild type plant of the same species, variety or cultivar.

“Over expression” refers to the increased expression of an mRNA or polypeptide compared to control plants, wherein said mRNA or polypeptide or protein is either not normally present in the host cell, or wherein said mRNA or polypeptide or protein is present in said host cell at a higher level than that normally expressed.

Over expression of the proteins of the instant invention may be accomplished by first constructing a chimeric gene in which the coding region is operably linked to a promoter capable of directing expression of a gene in the desired tissues at the desired stage of development. The chimeric gene may comprise promoter sequences and translation leader sequences derived from the same genes. 3′ Non-coding sequences encoding transcription termination signals may also be provided. The instant chimeric gene may also comprise one or more introns in order to facilitate gene expression. A suitable promoter may be the CaMV 35 S promoter which may be used with Agrobacterium as a vector.

The term “RNA interference” or “RNAi” may refer to a process in which a double-stranded RNA molecule or a short hairpin RNA changes the expression of a nucleic acid sequence with which they share substantial or total homology.

The term “RNAi down-regulation” refers to the reduction in the expression of a nucleic acid sequence which may be mediated by one or more RNAi species. The term “RNAi species” refers to a distinct RNA Sequence that elicits RNAi.

The term “RNAi construction group” refers to different transgenic trees emanating from one RNAi construct.

The term “photoperiod” refers to the daily cycle of light and darkness.

The term “recombinant” when used with reference, e.g., to a cell, nucleotide, vector, protein, or polypeptide typically indicates that the cell, nucleotide, or vector has been modified by the introduction of a heterologous (or foreign) nucleic acid or the alteration of a native nucleic acid, or that the protein or polypeptide has been modified by the introduction of a heterologous amino acid, or that the cell is derived from a cell so modified. Recombinant cells express nucleic acid sequences (e.g., genes) that are not found in the native (non-recombinant) form of the cell or express native nucleic acid sequences (e.g. genes) that would be abnormally expressed under-expressed, or not expressed at all. The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

In the context of the present invention “complementary” refers to the capacity for precise pairing between two nucleotides sequences with one another. For example, if a nucleotide at a certain position of an oligonucleotide is capable of hydrogen bonding with a nucleotide at the corresponding position of a DNA or RNA molecule, then the oligonucleotide and the DNA or RNA are considered to be complementary to each other at that position. The DNA or RNA strand are considered complementary to each other when a sufficient number of nucleotides in the oligonucleotide can form hydrogen bonds with corresponding nucleotides in the target DNA or RNA to enable the formation of a stable complex.

In the present context the expressions “complementary sequence” or “complement” therefore also refer to nucleotide sequences which will anneal to a nucleic acid molecule of the invention under stringent conditions.

The term “stringent conditions” refers to general conditions of high, weak or low stringency.

The term “stringency” is well known in the art and is used in reference to the conditions (temperature, ionic strength and the presence of other compounds such as organic solvents) under which nucleic acid hybridisations are conducted.

Hybridisation means matching one nucleic acid with another e.g. base pairing between single-stranded nucleic acid molecules with each other to form a duplex.

A “sub sequence” or a “fragment” is any portion of an entire sequence. Thus, a fragment or sub sequence refers to a sequence of amino acids or nucleic acids that comprises a part of a longer sequence of amino acids (e.g. polypeptide) or nucleic acids (e.g. polynucleotide), respectively.

In the present context, the term “homology” indicates similarities between two amino acid sequences or between two nucleotide sequences is described by the parameter “sequence identity”.

The term “sequence identity” indicates a quantitative measure of the degree of homology between two amino acid sequences or between two nucleic acid sequences of equal length. If the two sequences to be compared are not of equal length, they must be aligned to give the best possible fit, allowing the insertion of gaps or, alternatively, truncation at the ends of the polypeptide Sequences or nucleotide Sequences. The sequence identity can be calculated as

$$\frac{( {N}_{ref} - {N}_{dif} ) 100}{{N}_{ref}} ,$$

wherein N_{dif} is the total number of non-identical residues in the two Sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8). A gap is counted as non-identity of the specific residue(s), i.e. the DNA sequence AGTGTC will have a sequence identity of 75% with the DNA sequence AGTCAGTC (N_{dif}=2 and N_{ref}=8).

With respect to all embodiments of the invention relating to nucleotide sequences, the percentage of sequence identity between one or more sequences may also be based on alignments using the clustalW software (http:/www.ebi.ac.uk/clustalW/index.html) with default settings. For nucleotide Sequence alignments these settings are: Alignment=3Dfull, Gap Open 10.00, Gap Ext. 0.20, Gap separation Dist. 4, DNA weight matrix: identity (IUB). Alternatively, the sequences may be analysed using the program DNASIS Max and the comparison of the sequences may be done at http://www.paralign.org/. This service is based on the two comparison algorithms called Smith-Waterman (SW) and ParAlign. The first algorithm was published by Smith and Waterman (1981) and is a well established method that finds the optimal local alignment of two sequences The other algorithm, ParAlign, is a heuristic method for sequence alignment; details on the method is published in Rognes (2001). Default settings for score matrix and Gap penalties as well as E-values were used.

The phrase “substantially identical” or “substantial identity” in the context of two nucleic acids or polypeptides, may refer to two or more sequences or sub-sequences that have at least about 60%, 65%, 70%, 75%, preferably 80% or 85%, more preferably 90%, 91%, 92%, 93%, 94%, 94.5%, 95%, 95.5%, 96%, 96.5%, 97%, 97.5%, 98%, 98.5%, 99%, 99.5%, or greater nucleotide or amino acid residue percent identity, respectively, when compared and aligned for maximum correspondence, as measured using a sequence comparison algorithm or by visual inspection. Thus, the percent identity may be 99.6%, 99.7%, 99.8%, 99.9%, The substantially identity may refer to one gene, two or a group of genes separately and not to the other genes in the present invention, for example one gene might be 75% homologous and another might be 60% homologous and further another might be 93% homologous to the gene of interests. In certain aspects, the substantial identity exists over a region of amino acid sequences of at least about 50 residues in length, such as, at least about 100, 110, 120, 125, 130, 135, 140, 145, 150, 155, 160, or 165 amino acid residues. In certain aspects, substantial identity exists over a region of nucleic acid sequences of at least about 150 nucleic acid residues, such as at least about 200, 250, 300, 330, 360, 375, 400, 425, 450, 460, 480, 500, 600, 700, 800 such as at least about 900 nucleotides or such as at least about 1 kb, 2 kb, or such as at least about 3 kb. In some aspects, the amino acid or nucleic acid sequences are substantially identical over the entire length of the polypeptide sequence or the corresponding coding region. All definitions regarding to “substantially identical” or “substantial identity” relates mutatis mutandis to all SEQ ID numbers mentioned herein and to all aspects of the invention, wherein these SEQ ID numbers are involved.

Examples of “conservative substitutions” are within the group of basic amino acids (arginine, lysine and histidine), acidic amino acids (glutamic acid and aspartic acid), polar amino acids (glutamine and asparagine), hydrophobic amino acids (leucine, isoleucine, valine and methionine), aromatic amino acids (phenylalanine, tryptophan and tyrosine), and small amino acids (glycine, alanine, serine and threonine). Amino acid substitutions which do not generally alter the specific activity are known in the art and are described, for example, by Neurath and Hill, 1979. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

The term “conservatively substituted variant” as used herein refers to a variant of a nucleotide sequence comprising one or more conservative substitutions.

Generally and in the present context, the term “silent substitution” refers to a base substitution which does not affect the sense of a codon and thus has no effect on polypeptide structure. As the skilled person will know silent substitutions are possible because of the degeneracy of the genetic code.

The term “conserved domain” may be a sequence of amino acids in a polypeptide or a sequence of nucleotides in DNA or RNA that is similar across multiple species. A known set of conserved sequences is represented by a consensus sequence. Amino acid motifs are often composed of conserved sequences. Additionally, the term “conserved sequence” refers to a base sequence in a nucleic acid sequence molecule or an amino acid sequence in a protein that has remained essentially unchanged throughout evolution. A “consensus sequence” may be defined in terms of an idealized sequence that represents the base most often present at each position in a nucleic acid sequence or the amino acid most often present at each position in a protein. A “consensus sequence” is identified by aligning all known examples of a nucleic acid Sequence or a protein so as to maximise their sequence identity. For a sequence to be accepted as a consensus sequence each particular base or amino acid must be reasonably predominant at its position and most of the sequences must be related to the consensus by only few substitutions, such as 1 or 2.

A homologue may also be in the form of an “insertional variant” of a protein, i.e. where one or more amino acid residues are introduced into a predetermined site in a protein. Insertions may comprise N-terminal and/or C-terminal fusions as well as intra-sequence insertions of single or multiple amino acids.

Homologues in the form of “deletion variants” of a protein are characterised by the removal of one or more amino acids from a protein.

Homologues in the form of “addition variants” of a protein are characterised by the addition of one or more amino acids from a protein, whereby the addition may be at the end of the sequence.

Substitution is another variant of a protein wherein one or more amino acids have been changed for another (other) amino acid(s).

The terms “Orthologs” and “Paralogs”-sequences are also a type of homologous sequences as described above. Several different methods are known by those of skill in the art for identifying and defining these functionally homologous sequences. Three general methods for defining orthologs and paralogs are described; an ortholog, paralog or homolog may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species that are derived by a duplication event.

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same Glade (a group of similar genes) when a gene family phylogeny is analyzed using programs such as CLUSTAL (Thompson et al.; Higgins et al.) Groups of similar genes can also be identified with pair-wise BLAST analysis (Feng and Doolittle). For example, a Glade of very similar MADS domain transcription factors from Arabidopsis all share a common function in flowering time (Ratcliffe et al.), and a group of very similar AP2 domain transcription factors from Arabidopsis are involved in tolerance of plants to freezing (Gilmour et al.). Analysis of groups of similar genes with similar function that fall within one Glade can yield sub-Sequences that are particular to the Glade. These sub-sequences, known as consensus sequences, can not only be used to define the sequences within each Glade, but define the functions of these genes; genes within a Glade may contain paralogous sequences, or orthologous sequences that share the same function (see also, for example, Mount (2001), in Bioinformatics: sequence and Genome Analysis Cold Spring Harbor Laboratory Press, Cold Spring Harbor, N.Y., page 543.)

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Orthologous genes from different organisms may have highly conserved functions, and very often essentially identical functions (Lee et al. and Remm et al.). Paralogous genes, which have diverged through gene duplication, may retain similar functions of the encoded proteins. In such cases, paralogs can be used interchangeably with respect to certain embodiments of the instant invention (for example, transgenic expression of a coding sequence). An example of such highly related paralogs is the CBF family, with three well-defined members in Arabidopsis and at least one ortholog in Brassica napus, all of which control pathways involved in both freezing and drought stress (Gilmour et al. and Jaglo et al.)

Hybrid aspen is a hybrid of the two species Populus tremuloides and Populus tremula.

The following references represent a small sampling of the many studies that demonstrate that orthologous transcription factor genes from diverse species are likely to function similarly (i.e., regulate similar target sequences and control the same traits), and that transcription factors may be transformed into diverse species to confer or improve traits.

(1) The Arabidopsis NPR1 gene regulates systemic acquired resistance (SAR); over-expression of NPR1 leads to enhanced resistance in Arabidopsis. When either Arabidopsis NPR1 or the rice NPR1 ortholog was overexpressed in rice (which, as a monocot, is diverse from Arabidopsis), challenge with the rice bacterial blight pathogen Xanthomonas oryzae pv. Oryzae, the transgenic plants displayed enhanced resistance (Chem et al.). NPR1 acts through activation of expression of transcription factor genes, such as TGA2 (Fan and Dong).

(2) E2F genes are involved in transcription of plant genes for proliferating cell nuclear antigen (PCNA). Plant E2Fs share a high degree of similarity in amino acid Sequence between monocots and dicots, and are even similar to the conserved domains of the animal E2Fs. Such conservation indicates a functional similarity between plant and animal E2Fs. E2F transcription factors that regulate meristem development act through common cis-elements, and regulate related (PCNA) genes (Kosugi and Ohashi).

The term “closely related” genes is used for genes that are orthologous or paralogous.

The term “promoter,” as used herein, refers to a region of sequence determinants located upstream from the start of transcription of a gene and which are involved in recognition and binding of RNA polymerase and other proteins to initiate and modulate transcription. Promoters useful in plants need not be of plant origin. A “basal promoter” is the minimal sequence necessary for assembly of a transcription complex required for transcription initiation. Basal promoters frequently include a TATA box” element usually located between 15 and 35 nucleotides upstream from the site of initiation of transcription. Basal promoters also sometimes include a CCAAT box” element (typically a Sequence CCAAT) and/or a GGGCG Sequence, usually located between 40 and 200 nucleotides, preferably 60 to 120 nucleotides, upstream from the start site of transcription.

Promoters referred to herein as “constitutive promoters” may actively promote transcription under most, but not necessarily all, environmental conditions and states of development or cell differentiation. Examples of constitutive promoters include the cauliflower mosaic virus (CaMV) 35S transcript initiation region and the 1′ or 2′ promoter derived from T-DNA of Agrobacterium tumefaciens, and other transcription initiation regions from various plant genes, such as the maize ubiquitin-1 promoter, known to those of skill. Organ-specific promoters may be, for example, a promoter from storage sink tissues such as seeds, potato tubers, and fruits, or from metabolic sink tissues such as meristems. Furthermore, the promoter may be a leaf specific promoter such as the rbcs promoter from rice or tomato.

An “inducible promoter” in the context of the present invention refers to a promoter which is regulated under certain conditions, such as light, chemical concentration, protein concentration, conditions in an organism, cell, or organelle, etc. An example of an inducible promoter is the HSP promoter and the PARSK1, the promoter from the Arabidopsis gene encoding a serine-threonine kinase enzyme and which is induced by dehydration, abscissic acid and sodium chloride. In essence, expression under the control of an inducible promoter is “switched on” or increased in response to an applied stimulus. The nature of the stimulus varies between promoters and may include the above environmental factors. Whatever the level of expression is in the absence of the stimulus, expression from any inducible promoter is increased in the presence of the correct stimulus.

As used herein, the term “tissue specific” refers to a characteristic of a particular tissue that is not generally found in all tissues, or may be exclusive found in a tissue of interest. In the present application, “tissue specific” is used in reference to a gene regulatory element (promoter or promoter plus enhancer and/or silencer), the gene it encodes, or the polypeptide product of such a gene. In the context of a gene regulatory element or a “tissue specific promoter”, the term means that the promoter (and also other regulatory elements such as enhancer and/or silencer elements) directs the transcription of a linked sequence in a cell of a particular lineage, tissue, or cell type, but is substantially inactive in cells or tissues not of that lineage, tissue, or cell type. A tissue specific promoter useful according to the invention is at least 5-fold, 10-fold, 25-fold, 50-fold, 100-fold, 500-fold or even 1,000 times more active in terms of transcript production in the particular tissue than it is in cells of other tissues or in transformed or malignant cells of the same lineage. In the context of a gene or the polypeptide product of a gene, the term tissue specific means that the polypeptide product of the gene is detectable in cells of that particular tissue or cell type, but not substantially detectable in certain other cell types. Particularly relevant tissue specific promoters include promoter sequences specifically expressed or active in the xylem forming tissue in a plant. Examples of such promoters are the Lmp1, Lmx2, Lmx3, Lmx4 and Lmx5 promoters, described in WO2004097024.

A “terminator sequence” refers to a section of genetic sequence that marks the end of gene or operon on genomic DNA for transcription. Terminator sequences are recognized by protein factors that co-transcriptionally cleave the nascent RNA at a polyadenylation signal, halting further elongation of the transcript by RNA polymerase. A nucleic acid is “operably linked” when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

In the context of the present invention the terms “transformation” and “transforming” are used interchangeably and as synonyms to “transfecting” and “transfection”, respectively, to refer to the process of introducing DNA into a cell. The DNA constructs, including at least a portion of the gene or promoter of interest, can be introduced into host cells, which as stated previously, can be individual cells, cells in culture, cells as part of a host organism, a fertilized oocyte orgametophyte or an embryonic cell. By the term “introduction” when used in reference to a host cell is meant to refer to standard procedures known in the art for introducing recombinant vector DNA into the target host cell. Such procedures include, but are not limited to, transfection, infection, transformation, natural uptake, electroporation, biolistics and Agrobacterium mediated.

By “regenerable cell” is meant a plant cell from which a whole plant can be regenerated. It will be understood that the regenerable cell is a cell that has maintained its genetic potential, also known in the art as “totipotency”. It will further be understood that the regenerable cells, when grown in culture, may need the appropriate stimuli to express the total genetic potential of the parent plant.

A number of genes analyzed using the analytical platform show interesting and most often unexpected commercial features.

One aspect of the present invention with intentionally changed (increased or decreased expression) levels of one gene's SEQ ID No: 23 to 115 provide a method of producing a plant having modified lignin levels.

### Method of Producing a Plant Having Altered Lignin Properties

In specific embodiments of the invention advantageous plant phenotypes are generated by modifying, relative to the corresponding wild-type plant, the expression level of candidate genes that have been evaluated and selected according to the above criteria to modify lignin quantity, content and/or quality. According to these aspects a method is provided which comprises altering in the plant the level of a gene product of at least one gene comprising a nucleotide sequence selected from the group consisting of
a) a nucleotide sequence from SEQ ID NO 23-115, 157-195 or
b) a nucleotide sequence being at least 60% identical to a nucleotide sequence from SEQ ID NO 24-115, 157-195 or being at least 99.6% identical to the nucleotide sequence SEQ ID NO 23 or
c) a sub sequence or fragment of a nucleotide sequence of a) or b).

Higher lignin content may be obtained by over expression of at least one of the genes with SEQ ID NO: 113, 114, 115, 178 and 179 and/or down regulating at least one of the genes with SEQ ID NO: 24, 25, 26, 30, 32, 46, 47, 53, 54, 58, 157, 159, 160 and 162 in the groups of a), b) or c).

Thus at least one, at least two, at least three, lat least four or all of SEQ ID NO: 113, 114, 115, 178 and 179 may be over expressed. Further, at least one, at least two, at least three, at least four, at least five, at least six, at least seven, at least eight, at least nine, at least then, at least 11, at least 12, at least 13, or all of SEQ ID NO: 24, 25, 26, 30, 32, 46, 47, 53, 54, 58, 157, 159, 160 and 162 may be down regulated.

Lower lignin content, may be obtained by over expression of at least one of the genes with SEQ ID NO: 176, 177 and 180, 181, 182, 183, 184, 185, 186 and 187 and/or down regulating at least one of the genes with SEQ ID NO: 23, 27, 28, 29, 31, 33, 34, 35, 36, 37, 38, 39, 40, 41, 42, 43, 44, 48, 49, 50, 51, 52, 55, 56, 57, 158, 161 and 163 in the groups of a), b) or c).

Thus, at least one, at least two, at least three, lat least four, at least five, at least six, at least seven, at least eight, at least nine or all of the genes with SEQ ID NO: 176, 177 and 180, 181, 182, 183, 184, 185, 186 and 187 may be over expressed.

Further, at least one, at least two, at least three, lat least four, at least five, at least six, at least seven, at least eight, at least nine, at least then, at least 11, 12, 13, 14, 15, 16, 17, 18, 19, 20, 21, 22, 23, 24, 25, 26, 27 or all of SEQ ID NO: 23, 27, 28, 29, 31, 33, 34, 35, 36, 37, 38, 39, 40, 41, 42, 43, 44, 48, 49, 50, 51, 52, 55, 56, 57, 158, 161 and 163 may be down regulated.

All these definitions regarding SEQ ID numbers mentioned above relating to the higher and lower lignin content are also combined with the above made definition of the homology language “substantially identical” or “substantial identity”.

Plant phenotypes may be obtained by following technically modified crossing method comprising
i) selecting plant species expressing at least one of the nucleotide Sequences sequence selected from the group consisting of
   a) a nucleotide sequence from SEQ ID NO 23-115, 157-195 or
   b) a nucleotide sequence being at least 60% identical to a nucleotide sequence from SEQ ID NO 24-115, 157-195 or being at least 99.6% identical to the nucleotide sequence SEQ ID NO 23 or
   c) a sub sequence or fragment of a nucleotide sequence of a) or b).
ii) crossing a plant species selected in i) with the same or another plant species selected in i),
iii) selecting plants with modulated expression of at least one of the nucleotide sequences selected from the group consisting of
   a) a nucleotide sequence from SEQ ID NO 23-115, 157-195 or
   b) a nucleotide sequence being at least 60% identical to a nucleotide sequence from SEQ ID NO 24-115, 157-195 or being at least 99.6% identical to the nucleotide sequence SEQ ID NO 23 or
   c) a sub sequence or fragment of a nucleotide sequence of a) or b).
   compared to the plant species selected under i)
iv) optionally backcrossing one or more times the plants obtained in iii) and selecting plants with modulated expression of at least one of the nucleotide sequences selected from the group consisting of
   a) a nucleotide sequence from SEQ ID NO 23-115, 157-195 or
   b) a nucleotide sequence being at least 60% identical to a nucleotide sequence from SEQ ID NO 24-115, 157-195 or being at least 99.6% identical to the nucleotide sequence SEQ ID NO 23 or
   c) a sub sequence or fragment of a nucleotide sequence of a) or b).
   compared to any of the plant species used in i) and/or plants obtained in iii).

According to one aspect of the invention a method is provided comprising the following steps:
(i) providing an expression vector comprising a nucleotide sequence selected from the group consisting of
   a) a nucleotide sequence from SEQ ID NO 23-115, 157-195 or
   b) a nucleotide sequence being at least 60% identical to a nucleotide sequence from SEQ ID NO 24-115, 157-195 or being at least 99.6% identical to the nucleotide sequence SEQ ID NO 23 or
   c) a sub sequence or fragment of a nucleotide sequence of a) or b). and
   d) at least one regulatory element operable linked to the polynucleotide sequence, wherein said at least one regulatory element controls expression of the polynucleotide sequence in a target plant;
(ii) introducing the expression vector into at least one plant; and
(iii) selecting at least one transgenic plant that has a modulated lignin quantity compared to its wild type.

The sequences specified by Sequence ID numbers 59 to 93, 164-169, 170 and 188-195 represent sequences of the candidate genes as predicted from Populus trichocarpa, the sequences specified by sequence ID numbers 94 to 112, 157, 171-175, represent sequences of the candidate genes from hybrid aspen and SEQ ID numbers 23 to 58 113 to 115 and 157-163, 176-187 as cloned from hybrid aspen. Additional sequence from these genes 5′ as well as 3′ to the sequence described in SEQ ID No: 23 to 115, and 157-195 is readily achievable using conventional cloning techniques, as the skilled person will understand, such as those described in Sambrook et al.

In further embodiments of the invention the nucleic acid sequences in a)-d) above are substantially identical to SEQ ID NO 23 to 115 and 157-195 as defined herein.

The modulated expression may be effected by introducing a genetic modification preferably in the locus of a gene comprising SEQ ID NO 23-115 and 157-195 encoding a polypeptide or a homologue of such polypeptide

The modification may also for example be effected by one of: T-DNA activation, TILLING, homologous recombination, site-directed mutagenesis or directed breeding using one or more of SEQ ID NO 23 to 115 and 157-195 or a fragment thereof as markers in any step of the process of producing the desirable plants.

According to one aspect of the invention the modulation is decreased yield in lignin.

According to one other aspect of the invention the modulation is increased yield in lignin.

According to more particular embodiments of the invention, the method comprises the step of providing a nucleic acid construct, such as a recombinant DNA construct, comprising a nucleotide sequence selected from the group consisting of:
a) a nucleotide sequence comprising a sequence selected from SEQ ID No: 23 to 58, 113 to 115 and 157-163, 176-187;
b) a complementary nucleotide sequence of a nucleotide sequence of a);
c) a sub-sequence or fragment of a nucleotide sequence of a) or b);
d) a nucleic acid sequence being at least 60% identical to any one of the sequences in a), b) and c); and but not SEQ ID No 23 where the identity is 99.6%
e) a nucleotide sequence which hybridizes under stringent conditions to a nucleotide sequence of a), b) or c).

In further embodiments of the invention the nucleic acid sequence in d) is substantially identical as herein defined to any one of the sequences in a), b), or c),

In preferred embodiments of this aspect of the invention the nucleotide sequence of a) is selected from the group consisting of SEQ ID NOs: 23 to 58.

A variety of methods exist in the art for producing the nucleic acid sequences and nucleic acid/DNA constructs of the invention. Procedures for identifying and isolating DNA clones are well known to those of skill in the art, and are described in, e.g. Sambrook et al., Molecular Cloning-A Laboratory Manual (3rd Ed.), Vol. 1-3, Cold Spring Harbor Laboratory, Cold Spring Harbor, N.Y., 2001.

Nucleic acid sequences of the present invention can be produced by a variety of in vitro amplification methods adapted to the present invention by appropriate selection of specific or degenerate primers. Examples of protocols sufficient to direct persons of skill through in vitro amplification methods, including the polymerase chain reaction (PCR) the ligase chain reaction (LCR), Qbeta-replicase amplification and other RNA polymerase mediated techniques (e.g., NASBA), e.g., for the production of the homologous nucleic acids of the invention are found in Sambrook, supra.

Alternatively, nucleic acid constructs of the present invention can be assembled from fragments made by solid-phase synthesis methods. Typically, fragments of up to approximately 100 bases are individually synthesized and then enzymatically or chemically ligated to produce a desired sequence, e.g., a polynucletotide encoding all or part of a transcription factor. For example, chemical synthesis using the phosphoramidite method is well known to the skilled person. According to such methods, oligonucleotides are synthesized, purified, annealed to their complementary strand, ligated and then optionally cloned into suitable vectors.

As mentioned, the above described sequences are from hybrid aspen and Populus trichocarpa. As the skilled person will understand, homologues of the described Sequences may be isolated from other species, non-limiting examples of which include acacia, eucalyptus, hornbeam, beech, mahogany, walnut, oak, ash, hickory, birch, chestnut, alder, maple, sycamore, ginkgo, palm tree, sweet gum, cypress, Douglas fir, fir, sequoia, hemlock, cedar, juniper, larch, pine, redwood, spruce and yew, apple, plum, pear, banana, orange, kiwi, lemon, cherry, grapevine, fig, cotton, bamboo, switch grass, red canary grass, Miscantus species and rubber plants. Useful homologues of the described sequences may also be isolated from hardwood plants from the Salicaceae family, e.g. from the Salix and Populus genus. Members of this genus are known by their common names: willow, poplar and aspen.

In particular, the method according to the present invention may comprise a step of providing a nucleic acid construct, such as a recombinant DNA construct, comprising a nucleotide sequence which relative to the particular sequences described, comprises conservative variations altering only one, or a few amino acids in the encoded polypeptide may also be provided and used according to the present invention. Accordingly, it is within the scope of the invention to provide and use a recombinant DNA construct comprising a nucleotide sequence which encodes a polypeptide comprising a conservatively substituted variant of a polypeptide encoded from a nucleotide sequence from SEQ ID NO 23 to 115 and 157-195.

Sequence alterations that do not change the amino acid sequence encoded by the polynucleotide are termed “silent” substitutions. With the exception of the codons ATG and TGG, encoding methionine and tryptophan, respectively, any of the possible codons for the same amino acid can be substituted by a variety of techniques, e.g., site-directed mutagenesis, available in the art. Accordingly, the present invention may also provide a recombinant nucleic acid construct, wherein the nucleotide sequence comprises a silent substitution in a nucleotide sequence.

In certain further embodiments of the invention, the sub-sequences or fragments of c) or d) have substantial sequence identity as herein defined to a conserved domain of a nucleotide sequence as described above under item a) or b).

Thus, there are methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a Sequence.

### DNA Construct

According to a second main aspect of the invention a DNA construct, such as a recombinant DNA construct, is provided comprising at least one sequence as described above. The DNA construct may be a vector. In particular, the recombinant DNA construct may comprise a nucleotide sequence selected from the group consisting of:
a) a nucleotide sequence comprising a sequence selected from SEQ ID NO: 23-115, 157-195 or;
b) a complementary nucleotide sequence of a nucleotide sequence of a) or;
c) a sub-sequence or fragment of a nucleotide sequence of a) or b) or;
d) a nucleic acid sequence being at least 60% identical to any one of the sequences in a), b) and c); but not SEQ ID NO 23, where the identity is 99.6% or;
e) a nucleotide sequence which hybridizes under stringent conditions to a nucleotide sequence of a), b) or c).

In selected embodiments of the invention the nucleic acid sequence in d) is substantially identical as herein defined to any one of the sequences in a), b) and c).

Variants of DNA constructs of a), b), c) and d) above that encode polypeptides having additions, deletions, substitutions or insertions of one or more amino acids are also within the scope of the invention as long as lignin properties are affected.

Also, in accordance with the discussion above, the nucleotide Sequence encodes a polypeptide comprising a conservatively substituted variant of a polypeptide of (a). Further, the nucleotide sequence comprises a silent substitution in a nucleotide sequence.

In additional embodiments of the pertaining to this aspect of the invention, the sub-sequences or fragments have substantial sequence identity as herein defined to a conserved domain of a nucleotide sequence as described above under item a).

In further embodiments and in accordance with the description above, the recombinant DNA construct further comprising a promoter, that may be constitutive, inducible, or tissue specific, operably linked to said nucleotide sequence. In particular, the recombinant DNA construct may further comprise a strong constitutive promoter in front of a transcribed cassette consisting of the full open reading frame of the gene followed by a terminator sequence.

In a particular embodiment of the present invention the nucleic acid construct, or recombinant DNA construct, further comprises a strong constitutive promoter in front of a transcribed cassette consisting of part of the target gene followed by a plant functional intron followed by the same part of the target gene in reverse orientation, the transcribed cassette is followed by an terminator sequence. The preferred vector is of such type with one of the nucleotide sequence of the invention is inserted in inverted repeat orientation.

Thus, according to one embodiment the invention relates to the method according to any one of claims 1-13, wherein the recombinant DNA construct further comprises a strong constitutive promoter in front of a transcribed cassette consisting comprising a nucleotide sequence as defined in claim 1 followed by a plant functional intron followed by the nucleotide sequence as defined in claim 1 in reverse orientation

The recombinant DNA construct may be used for transforming regenerable cells of a plant and regenerating a transgenic plant from said transformed cell.

In the presently exemplified embodiments of the invention the recombinant DNA construct comprises the sequence of SEQ ID NO: 23-58 and 112-115.

### Construction of Vectors

In general, those skilled in the art are well able to construct vectors of the present invention and design protocols for recombinant gene expression. For further details on general protocols for preparation of vectors reference is made to: Molecular Cloning: a Laboratory Manual: 3rd edition, Sambrook et al, 2001, Cold Spring Harbor Laboratory Press. Further information about plant vector and cloning in plants can be found in Gelvin S B and Schilperoort R A (eds) (2000) Plant Molecular Biology Manual. Dordrecht: Kluwer Academic Publishers. The promoter used for the gene may influence the level, timing, tissue, specificity, or inducibility of the over expression.

Generally, over expression of a gene can be achieved using a recombinant DNA construct having a promoter operably linked to a DNA element comprising a sense element of a segment of genomic DNA or cDNA of the gene, e.g., the segment should contain enough of the open reading frame to produce a functional protein and preferably the full open reading frame.

In pertinent embodiments of the invention the nucleic acid construct, or recombinant DNA construct, further comprising a constitutive, inducible, or tissue specific promoter operably linked to said nucleotide sequence.

In a presently preferred embodiment of the invention, the nucleic acid construct, or recombinant DNA construct, used for RNAi comprises the sequence of SEQ ID NO: 116 the vector named pK7GWIWG2(I), Karimi et al 2002.

In another preferred embodiment of the invention, the nucleic acid construct, or recombinant DNA construct, used for over expression comprises the sequence of SEQ ID NO: 117 the vector named pK2GW7, Karimi et al 2002.

### Approaches to Obtaining Altering the Level of a Gene Product

Description of each gene and what is known about them and how it might be related to lignin is found in the experimental section below.

One aspect of the invention is to increase the expression of certain genes, non limiting examples how this can be done are presented here. Up regulation or over expression of a gene can for example be achieved by placing the full open reading frame of the gene behind a suitable promoter, which are described elsewhere, and usually placing terminator and poly-adenylation signal sequence 3′ of the gene to be over expressed.

One other aspect of the invention is to decrease the expression of certain genes, non limiting examples how this can be done are presented here. Suppression of gene expression can for instance be achieved using a ribozyme or an antisense sequence, or down-regulated by an RNAi species. Several extensive descriptions on how genes can be down-regulated can be found in the literature and in patent literature as well, one example is the WO 2008/067841, which is hereby incorporated as reference.

One of the genes SEQ ID No: 23-115 and 157-195 could be used as targets for marker assisted breeding because changes in the gene regulatory sequences can give changes in the expression patterns and changes in the coding sequences can give changes in the gene function, and we have shown that manipulating these genes gives changes in the desired traits. This is usually referred to that the genes SEQ ID No: 23-115 and 157-195 can be used as candidate genes in marker assisted breeding Brady and Provart 2007, Varshney et al 2005, and Burke et al. 2007. This can be used to achieve both up and down regulation of gene activity.

One particular way to use this invention is to measure the expression of one or more of the genes SEQ ID No 23 to 115 and 157-195 using for example quantitative RT-PCR in natural populations and select for unusual high or low expression of the measured gene and use such plants as parents in a breeding program, this could be repeated for each breeding cycle. Many methods to quantify gene expression are known to the art, an efficient method is real time PCR, described in Bustin 2000.

In addition, the nucleic acid construct or recombinant DNA construct according to the invention may be used for the purpose of gene replacement in order to modify the plant lignin properties phenotype.

Suppression of endogenous gene expression can for instance be achieved using a ribozyme. Ribozymes are RNA molecules that possess highly specific endoribonuclease activity. The production and use of ribozymes are disclosed in U.S. Pat. No. 4,987,071 and U.S. Pat. No. 5,543,508. While antisense techniques are discussed below, it should be mentioned that synthetic ribozyme sequences including antisense RNAs can be used to confer RNA cleaving activity on the antisense RNA, such that endogenous mRNA molecules that hybridize to the antisense RNA are cleaved, which in turn leads to an enhanced antisense inhibition of endogenous gene expression.

Vectors in which RNA encoded by a relevant gene homologue is over-expressed can also be used to obtain co-suppression of a corresponding endogenous gene, e.g., in the manner described in U.S. Pat. No. 5,231,020 to Jorgensen. Such co-suppression (also termed sense suppression) does not require that the entire gene sequence be introduced into the plant cells, nor does it require that the introduced sequence be exactly identical to the endogenous sequence of interest. However, the suppressive efficiency will be enhanced as specificity of hybridization is increased, e.g., as the introduced sequence is lengthened, and/or as the sequence similarity between the introduced sequence and the endogenous transcription factor gene is increased.

Vectors expressing an untranslatable form of gene, e.g., sequences comprising one or more stop codons, or nonsense mutation, can also be used to suppress expression of an endogenous gene, thereby reducing or eliminating its activity and modifying one or more traits. Methods for producing such constructs are described in U.S. Pat. No. 5,583,021. In particular, such constructs can be made by introducing a premature stop codon into the gene.

One way of performing targeted DNA insertion is by use of the retrovirus DNA integration machinery as described in WO2006/078431. This technology is based on the possibility of altering the integration site specificity of retroviruses and retrotransposons integrase by operatively coupling the integrase to a DNA-binding protein (tethering protein). Engineering of the integrase is preferably carried out on the nucleic acid level, via modification of the wild type coding sequence of the integrase by PCR. The integrase complex may thus be directed to a desired portion or be directed away from an undesired portion of genomic DNA thereby producing a desired integration site characteristic.

Another technology that can be used to alter gene expression and gene activity is the TILLING, “Targeting Induced Local Lesions in Genomes”, which is a non-transgenic way to alter gene function in a targeted way. This approach involves mutating a plant with for example ethyl methanesulfonate (EMS) and later locating the individuals in which a particular desired gene has been modified. The technology is described for instance in Slade and Knauf, 2005 and Henikoff, et al.

A method for abolishing the expression of a gene is by insertion mutagenesis using the T-DNA of Agrobacterium tumefaciens. After generating the insertion mutants, the mutants can be screened to identify those containing the insertion in an appropriate gene. Plants containing a single transgene insertion event at the desired gene can be crossed to generate homozygous plants for the mutation.

The polynucleotides and polypeptides of this invention can also be expressed in a plant in the absence of an expression cassette by manipulating the activity or expression level of the endogenous gene by other means, for example, by ectopically expressing a gene by T-DNA activation tagging, Ichikawa et al. (1997); Kakimoto et al. (1996). This method entails transforming a plant with a gene tag containing multiple transcriptional enhancers and once the tag has inserted into the genome, expression of a flanking gene coding sequence becomes deregulated, Ichikawa et al. (1997); Kakimoto et al. (1996). In another example, the transcriptional machinery in a plant can be modified so as to increase transcription levels of a polynucleotide of the invention (See, e.g., PCT Publications WO 96/06166 and WO 98/53057 which describe the modification of the DNA binding specificity of zinc finger proteins by changing particular amino acids in the DNA binding motif).

However, the recombinant DNA construct, comprising a nucleotide sequence as described above is particularly useful for sense and anti-sense suppression of expression, e.g., to down-regulate expression of a particular gene, in order to obtain a plant phenotype with altered lignin properties. That is, the nucleotide sequence of the invention, or sub-sequences or anti-sense sequences thereof, can be used to block expression of naturally occurring homologous nucleic acids. Varieties of traditional sense and antisense technologies are known in the art, e.g., as set forth in Lichtenstein and Nellen (1997). The objective of the antisense approach is to use a sequence complementary to the target gene to block its expression and create a mutant cell line or organism in which the level of a single chosen protein is selectively reduced or abolished.

For more elaborate descriptions of anti-sense regulation of gene expression as applied in plant cells reference is made to U.S. Pat. No. 5,107,065, the content of which is incorporated herein in its entirety.

Gene silencing that is induced by double-stranded RNA is commonly called RNA interference or RNAi. RNA interference is a molecular mechanism in which fragments of double-stranded ribonucleic acid (dsRNA) interfere with the expression of a particular gene that shares a homologous sequence with the dsRNA. The process that is mediated by the same cellular machinery that processes microRNA, known as the RNA-induced silencing complex (RISC). The process is initiated by the ribonuclease protein Dicer, which binds and cleaves exogenous double-stranded RNA molecules to produce double-stranded fragments of 20-25 base pairs with a few unpaired overhang bases on each end. The short double-stranded fragments produced by Dicer, called small interfering RNAs (siRNAs), are separated and integrated into the active RISC complex.

If one part of an RNA transcript is targeted by an RNAi molecule or construct, the whole transcript is down-regulated.

For more elaborate descriptions of RNAi gene suppression in plants by transcription of a dsRNA reference is made to U.S. Pat. No. 6,506,559, US 2002/0168707, and WO 98/53083, WO 99/53050 and WO 99/61631, all of which are incorporated herein by reference in their entirety.

The presently preferred nucleic acid construct for RNAi is the pDONR vector (Invitrogen USA) and a destination vector pK2GW7 using Gateway technology (Invitrogen USA).

### Transformation of Plant Cells

In accordance with the present invention, the method comprise the further step of transforming regenerable cells of a plant with said nucleic acid construct or recombinant DNA construct and regenerating a transgenic plant from said transformed cell. When introducing the above DNA construct or vector into a plant cell, certain considerations must be taken into account, well known to those skilled in the art. The nucleic acid to be inserted should be assembled within a construct that contains effective regulatory elements that will drive transcription, as described above. There must be available a method of transporting the construct into the cell. Once the construct is within the cell, integration into the endogenous chromosomal material either will or will not occur.

Transformation techniques, well known to those skilled in the art, may be used to introduce the DNA constructs and vectors into plant cells to produce transgenic plants, in particular transgenic trees, with altered lignin properties.

A person of skills in the art will realise that a wide variety of host cells may be employed as recipients for the DNA constructs and vectors according to the invention. Non-limiting examples of host cells include cells in embryonic tissue, callus tissue type I, II, and III, hypocotyls, meristem, root tissue, tissues for expression in phloem, leaf discs, petioles and stem internodes.

As listed above, Agrobacterium transformation is one method widely used by those skilled in the art to transform tree species, in particular hardwood species such as poplar. Production of stable, fertile transgenic plants is now a routine in the art. Other methods, such as microprojectile or particle bombardment, electroporation, microinjection, direct DNA uptake, liposome mediated DNA uptake, or the vortexing method may be used where Agrobacterium transformation is inefficient or ineffective, for example in some gymnosperm species.

Alternatively, a combination of different techniques may be employed to enhance the efficiency of the transformation process, e.g. bombardment with Agrobacterium coated microparticles or microprojectile bombardment to induce wounding followed by co-cultivation with Agrobacterium.

It will be understood, that the particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration.

Following transformation, transgenic plants are preferably selected using a dominant selectable marker incorporated into the transformation vector. Typically, such a marker will confer antibiotic or herbicide resistance on the transformed plants and selection of transformants can be accomplished by exposing the plants to appropriate concentrations of the antibiotic or herbicide. A novel selection marker using the D-form of amino acids and based on the fact that plants can only tolerate the L-form offers a fast, efficient and environmentally friendly selection system. An interesting feature of this selection system is that it enables both selection and counter-selection.

Subsequently, a plant may be regenerated, e.g. from single cells, callus tissue or leaf discs, as is standard in the art. Almost any plant can be entirely regenerated from cells, tissues and organs of the plant, Gelvin S B and Schilperoort R A (eds) (2000) Plant Molecular Biology Manual. Dordrecht: Kluwer Academic Publishers. After transformed plants are selected and grown to maturity, those plants showing an altered lignin properties phenotype are identified. Additionally, to confirm that the phenotype is due to changes in expression levels or activity of the polypeptide or polynucleotide disclosed herein can be determined by analyzing mRNA expression using Northern blots, RT-PCR or microarrays, or protein expression using immunoblots or Western blots or gel shift assays.

### Plants

According to another aspect the invention provides a plant having altered lignin properties compared to its wild type. The plant may be transgenic

A transgenic plant may comprise a recombinant polynucleotide (DNA construct) comprising a nucleotide sequence capable of altering in the plant the level of a gene product of at least one of the genes SEQ ID 23-115 giving altered lignin content when comparing said group of transgenic plants grown for 8-9 weeks in a greenhouse under a photoperiod of 18 hours, a temperature of 22° C./15° C. (day/night) and a weekly were fertilized weekly Weibulls Rika S NPK 7-1-5 diluted 1 to 100 (final concentrations NO₃, 55 g/l; NH₄, 29 g/l; P, 12 g/l; K, 56 g/l; Mg 7.2 g/l; S, 7.2 g/l; B, 0.18 g/l; Cu, 0.02 g/l; Fe, 0.84 g/l; Mn, 0.42 g/l; Mo, 0.03 g/l; Zn, 0.13 g/l) with a group of wild-type plants grown under identical conditions. Growth conditions must be adapted to the particular species transform, since they have different growth optima. The alteration may vary between a decrease in lignin content from −6% to −20% and an increase in lignin content from +5% to +16%.

According to particular embodiments of the invention the level of a gene product of at least one gene comprising a nucleotide sequence selected from the group consisting of
a) a nucleotide sequence from SEQ ID NO 23-115, 157-195 or;
b) a nucleotide sequence being at least 60% identical to a nucleotide sequence from SEQ ID NO 24-115, 157-195 or being at least 99.6% identical to the nucleotide sequence SEQ ID NO 23 or
c) a sub sequence or fragment of a nucleotide sequence of a) or b)
has been altered relative to the level found in the respective corresponding wild-type plant.

The invention especially relates to plants having higher lignin content, and to plants having lower lignin content, comprising the genes as mentioned under the heading “Method of producing a plant having altered lignin properties”.

According to yet another embodiment of the invention, the transgenic plant comprises a recombinant polynucleotide (DNA construct) comprising a nucleotide sequence selected from the group consisting of:
a) a nucleotide sequence comprising a sequence selected from SEQ ID NO: 23-115, 157-195 or
b) a complementary nucleotide sequence of a nucleotide sequence of a) or
c) a sub-sequence or fragment of a nucleotide sequence of a) or b) or
d) a nucleic acid sequence being at least 60% identical to any one of the sequences in a), b) and c); but not SEQ ID no 23, where the identity is 99.6% or
e) a nucleotide sequence which hybridizes under stringent conditions to a nucleotide sequence of a), b) or c).

In further embodiments of this aspect of the invention the nucleic acid sequence in d) is substantially identical as herein defined to any one of the sequences in a), b) or c).

As mentioned above the skilled person will realize that a variety of methods exist in the art for producing the nucleic acid sequences and polynucleotide constructs of the invention, e.g. by cloning techniques, assembly of fragments generated by solid phase synthesis.

In particular, the transgenic plant according to the present invention may comprise a recombinant DNA construct comprising a nucleotide sequence which relative to the particular sequences described, comprises conservative variations altering only one, or a few amino acids in the encoded polypeptide may also be provided and used according to the present invention. Accordingly, it is within the scope of the invention to provide a transgenic plant comprising a recombinant DNA construct comprising a nucleotide sequence which encodes a polypeptide comprising a conservatively substituted variant of a polypeptide of a) or d).

Accordingly, the present invention may also provide a recombinant DNA construct, wherein the nucleotide sequence comprises a silent substitution in a nucleotide sequence, that is, the recombinant DNA construct may comprise a sequence alteration that does not change the amino acid sequence encoded by the polynucleotide.

In certain further embodiments of the invention, the sub-sequences or fragments substantial sequence identity to a conserved domain of a nucleotide sequence as described above under item a) or d).

In further embodiments the transgenic plant provided according to the invention comprises a recombinant polynucleotide construct which further comprises a constitutive, inducible, or tissue specific promoter operably linked to said nucleotide sequence.

In still further embodiments the recombinant polynucleotide construct further comprises a strong constitutive promoter in front of a transcribed cassette.

In a presently preferred embodiment of the invention, the transgenic plant according to the invention comprises a recombinant DNA construct comprising the sequence of one or more SEQ ID NO: 23 to 115, 157-195.

Another aspect of the invention provides a plant cell, plant progeny or any vegetable material of a transgenic plant or a plant with intentionally changed (increased or decreased expression) levels of one or more gene's SEQ ID No: 23 to 115, 157-195 according to the invention or the above mentioned variants thereof and comprising a recombinant polynucleotide.

A further aspect of the invention provides a plant cell, plant progeny or any vegetable material of a transgenic plant or a plant with intentionally changed (increased or decreased expression) levels of one or more gene's SEQ ID: 23 to 115, 157-195 according to the invention and comprising a recombinant polynucleotide, where the lignin content measured by the Klason method is modified.

The invention also relates to wood produced by a plant, which may be transgenic as defined above. It may comprise the nucleic acids of one or more of SEQ ID: 23 to 115, 157-195 or the variants thereof defined above.

Selections of plants may be done by measuring the lignin level according to the Klason method.

### Plant Species

In accordance with the present invention, the transgenic plant may be a perennial plant which preferable is a woody plant or a woody species. In a useful embodiment, the woody plant is a hardwood plant which may be selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum. Hardwood plants from the Salicaceae family, such as willow, poplar and aspen including variants thereof, are of particular interest, as these two groups include fast-growing species of tree or woody shrub which are grown specifically to provide timber and bio-fuel.

In further embodiments, the woody plant is a conifer which may be selected from the group consisting of cypress, Douglas fir, fir, sequoia, hemlock, cedar, juniper, larch, pine, redwood, spruce and yew.

In useful embodiments, the woody plant is a fruit bearing plant which may be selected from the group consisting of apple, plum, pear, banana, orange, kiwi, lemon, cherry, grapevine and fig.

Other woody plants which may be useful in the present method may also be selected from the group consisting of cotton, bamboo and rubber plants.

Other plants which may be useful is grasses grown for biomass production, for example Miscanthus and Switchgrass.

The present invention extends to any plant cell of the above transgenic plants obtained by the methods described herein, and to all plant parts, including harvestable parts of a plant, seeds and propagules thereof, and plant explant or plant tissue. The present invention also encompasses a plant, a part thereof, a plant cell, a plant progeny and any vegetable material comprising a DNA construct according to the invention. The vegetable material may be comprised in any technically useful material. Examples of technically useful material comprising such vegetable material are pellets and pulp. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention.

### Improved Lignin Quality and Amount

In addition, the nucleic acid construct or recombinant DNA construct according to the invention may be used for the purpose of gene replacement in order to modify the lignin level in the plant.

This invention relates to the problem of changing wood chemistry especially for changing lignin quality and amounts.

Such trees can be used in many applications. For example for these usages
1. Changing surface chemistry for e.g. composite material usage.
2. Saccharification potential with emphasis of changing lignin levels and/or composition and/or lignin binding to the carbohydrates of the cell wall.
3. Changing lignin levels for energy content.
4. Changing lignin levels for improved pulping properties.

The inventors have found that the down regulation and/or over expression of certain genes, gives changed wood chemistry in the produced transgenic trees. Especially we have found that certain genes can be used to modify lignin levels. These genes have been found within an RNAi gene mining program, with a gene selection a basic set up identical to the one described in the patent applications WO 2008/067840 and WO 2008/067841, but where the first level of analysis were done with FT-IR analysis of wood and the second level of analysis were done with Klason Lignin analysis.

Thus, according to one aspect the invention regards the use of a nucleotide sequence selected from the group consisting of
a) at least one over expressed gene chosen from genes with SEQ ID NO: 176, 177 and 180-187 and/or one down regulated gene chosen from of the genes with SEQ ID NO 23, 27-29, 31, 33-44, 48-52, 55-57, 158, 161 and 163 or
b) a nucleotide sequence being at least 60% identical to a nucleotide sequence from SEQ ID NO 27-29, 31, 33-44, 48-52, 55-57, 158, 161, 163 176, 177 and 180-187 or at least 99.6% identical to SEQ ID NO 23 or
c) a sub sequence or fragment of a nucleotide sequence of a) or b), in a plant for lowering lignin levels improving saccharification for bio-refining and ethanol production and for lowering lignin levels in pulp and paper processing.

The preferred nucleotide sequences are selected from the group consisting of 23, 28, 29, 31, 33-38, 40, 42, 55, 57, 158, 161 and 163 in a plant for lowering lignin levels improving saccharification for bio-refining and ethanol production and for lowering lignin levels in pulp and paper processing. See table 1 for lignin levels.

According to another aspect the invention relates to the use of a nucleotide sequence selected from the group consisting of
a) at least one over expressed gene chosen from genes with SEQ ID NO: 113, 114, 115, 178 and 179 and/or at least one down regulated gene chosen from genes with SEQ ID NO: 24-26, 30, 32, 46, 47, 53, 54, 58, 157, 159, 160 and 162 or
b) a nucleotide sequence being at least 60% identical to a nucleotide sequence from SEQ ID NO 24-26, 30, 32, 46, 47, 53, 54, 58, 113-115, 157, 159, 160, 162, 178 and 179 or
c) a sub sequence or fragment of a nucleotide sequence of a) or b)
in a plant for increasing lignin levels for energy production.

The preferred nucleotide sequences are selected from the group consisting of 26, 30, 32, 46, or 113-115, 178-179 in a plant for increasing lignin levels for energy production

Especially the invention regards the use of a nucleotide sequence selected from the group consisting of
a) at least one over expressed gene chosen from genes with SEQ ID NO: 113, 114 and 115 and/or at least one down regulated gene chosen from genes with SEQ ID NO: 24-26, 30, 32, 46, 47, 53, 54, 58, 157, 159, 160 and 162 or
b) a nucleotide sequence being at least 60% identical to a nucleotide sequence from SEQ ID NO 24-26, 30, 32, 46, 47, 53, 54, 58, 113-115, 157, 159, 160, 162, 178 and 179 or
c) a sub sequence or fragment of a nucleotide sequence of a) or b)
in a plant for increasing lignin levels for energy production.

The invention also relates to the use of a nucleotide sequence selected from the group consisting of
a) at least one down regulated gene chosen from of the genes with SEQ ID NO a nucleotide sequence from genes with SEQ ID NO 23, 27-29, 31, 33-44, 48-52, 55-57, 158, 161 and 163 or
b) a nucleotide sequence being at least 60% identical to a nucleotide sequence from SEQ ID NO, 27-29, 31, 33-44, 48-52, 55-57, 158, 161, 163, 176, 177, 180 and 187 or being at least 99.6% identical to SEQ ID NO 23 or
c) a sub sequence or fragment of a nucleotide sequence of a) or b)
in a plant for lowering lignin levels improving saccharification for bio-refining and ethanol production and for lowering lignin levels in pulp and paper processing.

The effect of these preferred genes are shown in the table 1a, b, c and d below.

TABLE 1a
Over expressed genes with higher lignin content vs wildtype

| Gene | Construct | T-test | Anova | <T int | >T Int | <Min | >Max | Ratio | Modulation | SEQ ID | +/−vs wt T89 |
|---|---|---|---|---|---|---|---|---|---|---|---|
| STT 14 | 35s005 | Sign | Sign | 0 | 0 | 0 | 4 | 1.08 | Over expression | Seq ID No: 113 | 13% |
| STT 17 | 35s006 | | Sign | 0 | 1 | 0 | 2 | 1.1 | Over expression | Seq ID No: 114 | 16% |
| STT 57 | 35s008 | | | 0 | 1 | 0 | 1 | 1.02 | Over expression | Seq ID No: 115 | 15% |
| STT 167 | LMP1-002 | | | 0 | 1 | 0 | 1 | 1.02 | Over expression | Seq ID No: 178 | 9% |
| STT 551 | TF0003 | Sign | Sign | 0 | 2 | 0 | 2 | 1.04 | Over expression | Seq ID No: 179 | 7% |

TABLE 1b
Over expressed genes with lower lignin content vs wildtype

| Gene | Construct | T-test | Anova | <T int | >T Int | <Min | >Max | Ratio | Modulation | SEQ ID | +/−vs wt T89 |
|---|---|---|---|---|---|---|---|---|---|---|---|
| STT 97 | 35S029 | | | 1 | 0 | 3 | 0 | 0.97 | Over expression | Seq ID No: 176 | −8% |
| STT 97 | 35s029 | | | | | | | | Over expression | Seq ID No: 177 | |
| STT 577 | TF0050 | Sign | Sign | 2 | 0 | 2 | 0 | 0.96 | Over expression | Seq ID No: 180 | −8% |
| STT 577 | TF0050 | | | | | | | | Over expression | Seq ID No: 181 | |
| STT 584 | TF0061 | | | 1 | 0 | 1 | 2 | 1.02 | Over expression | Seq ID No: 182 | −8% |
| STT 606 | TF0094.2nd | Sign | Sign | 0 | 0 | 2 | 0 | 0.96 | Over expression | Seq ID No: 183 | −8% |
| STT 606 | TF0094.2nd | | | | | | | | Over expression | Seq ID No: 184 | |
| STT 633 | TF0138 | | | 1 | 0 | 1 | 0 | 0.99 | Over expression | Seq ID No: 185 | −8% |
| STT 658 | TFSTT021 | | | 2 | 1 | 2 | 1 | 0.98 | Over expression | Seq ID No: 186 | −7% |
| STT 658 | TFSTT021 | | | | | | | | Over expression | Seq ID No: 187 | |

TABLE 1 c
Down regulated genes with higher lignin content vs wildtype

| Gene | Construct | T-test | Anova | <T int | >T Int | <Min | >Max | Ratio | Modulation | SEQ ID | +/−vs wt T89 |
|---|---|---|---|---|---|---|---|---|---|---|---|
| STT 11 | KR008B | | | 0 | 0 | 0 | 1 | 1.01 | Down regulated | Seq ID No: 24 | 7% |
| | | | | | | | | | RNAi | | |
| STT 13 | KR010B | | | 0 | 1 | 0 | 1 | 1.01 | Down regulated | Seq ID No: 25 | 8% |
| | | | | | | | | | RNAi | | |
| STT 14 | KR012B | | | 0 | 2 | 0 | 3 | 1.04 | Down regulated | Seq ID No: 26 | 11% |
| | | | | | | | | | RNAi | | |
| STT 27 | KR100B | | | 0 | 2 | 0 | 2 | 1.04 | Down regulated | Seq ID No: 30 | 13% |
| | | | | | | | | | RNAi | | |
| STT 32 | KR112 | | | 0 | 1 | 0 | 2 | 1.03 | Down regulated | Seq ID No: 32 | 12% |
| | | | | | | | | | RNAi | | |
| STT 95 | KR213 | | | 0 | 1 | 0 | 2 | 1.05 | Down regulated | Seq ID No: 46 | 12% |
| | | | | | | | | | RNAi | | |
| STT 97 | KR215 | | | 0 | 0 | 0 | 2 | 1 | Down regulated | Seq ID No: 47 | 5% |
| | | | | | | | | | RNAi | | |
| STT 167 | KR472 | | | 0 | 0 | 0 | 1 | 1.02 | Down regulated | Seq ID No: 53 | 5% |
| | | | | | | | | | RNAi | | |
| STT 170 | KR475 | | | 0 | 0 | 0 | 1 | 1.01 | Down regulated | Seq ID No: 54 | 5% |
| | | | | | | | | | RNAi | | |
| STT 317 | KR605 | | | 0 | 0 | 0 | 1 | 1.03 | Down regulated | Seq ID No: 58 | 7% |
| | | | | | | | | | RNAi | | |
| STT 187 | KR489 | Sign | Sign | 0 | 0 | 0 | 1 | 1.04 | Down regulated | Seq ID No: 157 | 8% |
| | | | | | | | | | RNAi | | |
| STT 315 | KR603 | | | 0 | 1 | 0 | 3 | 1.05 | Down regulated | Seq ID No: 159 | 11% |
| | | | | | | | | | RNAi | | |
| STT 392 | KR680 | | | 0 | 1 | 0 | 2 | 1.02 | Down regulated | Seq ID No: 160 | 7% |
| | | | | | | | | | RNAi | | |
| STT 442 | KR730 | | | 0 | 1 | 0 | 1 | 1 | Down regulated | Seq ID No: 162 | 7% |
| | | | | | | | | | RNAi | | |

TABLE 1d
Down regulated genes with lower lignin content vs wildtype

| Gene | Construct | T-test | Anova | <T int | >T Int | <Min | >Max | Ratio | Modulation | SEQ ID | +/−vs wt T89 |
|---|---|---|---|---|---|---|---|---|---|---|---|
| STT 5 | KR002B | | | 1 | 0 | 1 | 0 | 0.96 | Down regulated | Seq ID No: 23 | −10% |
| | | | | | | | | | RNAi | | |
| STT 16 | KR014B | | | 0 | 0 | 0 | 1 | 1.02 | Down regulated | Seq ID No: 27 | −6% |
| | | | | | | | | | RNAi | | |
| STT 17 | KR015B | | | 2 | 0 | 2 | 0 | 0.97 | Down regulated | Seq ID No: 28 | −12% |
| | | | | | | | | | RNAi | | |
| STT 23 | KR080B | | | 1 | 0 | 1 | 0 | 0.97 | Down regulated | Seq ID No: 29 | −10% |
| | | | | | | | | | RNAi | | |
| STT 31 | KR111 | | | 2 | 0 | 2 | 0 | 0.95 | Down regulated | Seq ID No: 31 | −19% |
| | | | | | | | | | RNAi | | |
| STT 40 | KR126 | | | 1 | 0 | 1 | 0 | 0.96 | Down regulated | Seq ID No: 33 | −16% |
| | | | | | | | | | RNAi | | |
| STT 49 | KR140 | Sign | Sign | 1 | 0 | 2 | 0 | 0.93 | Down regulated | Seq ID No: 34 | −12% |
| | | | | | | | | | RNAi | | |
| STT 54 | KR151 | | | 0 | 0 | 2 | 0 | 0.95 | Down regulated | Seq ID No: 35 | −10% |
| | | | | | | | | | RNAi | | |
| STT 57 | KR154 | Sign | Sign | 1 | 0 | 3 | 0 | 0.92 | Down regulated | Seq ID No: 36 | −11% |
| | | | | | | | | | RNAi | | |
| STT 59 | KR159 | | Sign | 1 | 0 | 3 | 0 | 0.93 | Down regulated | Seq ID No: 37 | −12% |
| | | | | | | | | | RNAi | | |
| STT 64 | KR165 | | | 1 | 1 | 2 | 1 | 0.98 | Down regulated | Seq ID No: 38 | −15% |
| | | | | | | | | | RNAi | | |
| STT 70 | KR175 | Sign | Sign | 0 | 0 | 2 | 0 | 0.94 | Down regulated | Seq ID No: 39 | −9% |
| | | | | | | | | | RNAi | | |
| STT 74 | KR179 | Sign | Sign | 0 | 0 | 0 | 0 | 0.93 | Down regulated | Seq ID No: 40 | −10% |
| | | | | | | | | | RNAi | | |
| STT 75 | KR182 | Sign | | 0 | 0 | 0 | 0 | 0.94 | Down regulated | Seq ID No: 41 | −8% |
| | | | | | | | | | RNAi | | |
| STT 76 | KR183 | | Sign | 3 | 0 | 3 | 0 | 0.92 | Down regulated | Seq ID No: 42 | −20% |
| | | | | | | | | | RNAi | | |
| STT 82 | KR192 | Sign | | 0 | 0 | 0 | 0 | 0.97 | Down regulated | Seq ID No: 43 | −5% |
| | | | | | | | | | RNAi | | |
| STT 83 | KR198 | | | 1 | 0 | 1 | 0 | 0.99 | Down regulated | Seq ID No: 44 | −7% |
| | | | | | | | | | RNAi | | |
| STT 101 | KR223 | | | 1 | 0 | 1 | 0 | 1 | Down regulated | Seq ID No: 48 | −6% |
| | | | | | | | | | RNAi | | |
| STT 116 | KR242 | Sign | Sign | 0 | 0 | 1 | 0 | 0.96 | Down regulated | Seq ID No: 49 | −9% |
| | | | | | | | | | RNAi | | |
| STT 124 | KR287 | | | 1 | 0 | 1 | 0 | 0.97 | Down regulated | Seq ID No: 50 | −8% |
| | | | | | | | | | RNAi | | |
| STT 128 | KR318 | | | 0 | 0 | 1 | 0 | 0.98 | Down regulated | Seq ID No: 51 | −7% |
| | | | | | | | | | RNAi | | |
| STT 165 | KR470 | | | 1 | 0 | 2 | 0 | 0.97 | Down regulated | Seq ID No: 52 | −7% |
| | | | | | | | | | RNAi | | |
| STT 173 | KR478 | | Sign | 2 | 0 | 3 | 0 | 0.96 | Down regulated | Seq ID No: 55 | −10% |
| | | | | | | | | | RNAi | | |
| STT 178 | KR484 | | | 1 | 1 | 1 | 1 | 0.97 | Down regulated | Seq ID No: 56 | −8% |
| | | | | | | | | | RNAi | | |
| STT 307 | KR595 | | | 0 | 0 | 2 | 0 | 0.98 | Down regulated | Seq ID No: 57 | −10% |
| | | | | | | | | | RNAi | | |
| STT 311 | KR599 | | | 1 | 0 | 2 | 2 | 0.99 | Down regulated | Seq ID No: 158 | −9% |
| | | | | | | | | | RNAi | | |
| STT 398 | KR686 | Sign | Sign | 3 | 0 | 4 | 0 | 0.94 | Down regulated | Seq ID No: 161 | −8% |
| | | | | | | | | | RNAi | | |
| STT 476 | KR764 | | | 1 | 0 | 1 | 2 | 0.99 | Down regulated | Seq ID No: 163 | −8% |
| | | | | | | | | | RNAi | | |

### Further Uses of the Invention

Moreover, the invention relates to the use of a nucleotide sequence selected from the group consisting of
a) a nucleotide sequence from SEQ ID NO 23-115, 157-195 or
b) a nucleotide sequence being at least 60% identical to a nucleotide sequence from SEQ ID NO 24-115, 1557-195, or being at least 99.6% identical to SEQ ID NO 23 or
c) a sub sequence or fragment of a nucleotide sequence of a) or b) for the identification of plants having altered lignin characteristics as compared to the wild-type.

According to the invention these sequences may also be used as candidate genes in marker assisted breeding.

It should be noted that embodiments and features described in the context of one of the aspects of the present invention also apply mutatis mutandis to the all other aspects of the invention. For example the variant and homology language for defining genes and expressed polypeptides regards all aspects of the inventions such as DNA constructs, expressions products of the genes, vectors, plant cells, plant progeny, vegetable material wood and the methods for producing and identification of plants according to the invention.

All patent and non-patent references cited in the present application, are hereby incorporated by reference in their entirety.

The invention will now be described in further details in the following non-limiting examples.

### EXAMPLES

In order to find and elucidate the function of genes involved in wood formation and wood growth, an extensive gene mining program was performed, resulting in the identification of genes useful in wood industrial applications.

### Gene Selection

The genes indentified for changing wood chemistry especially for changing lignin quality and amounts was done in a gene mining program, which is described in detail in the patent applications WO 2008/067840, WO 2008/067841 and WO 2009/084999, which are hereby incorporated as references.

### Cloning of the Selected Genes

Selected genes were subsequently cloned into a RNAi vector under the control of the CaMV 35S promoter (RNA interference vector, pK7GWIWG2(I)) using Gateway technology (Invitrogen USA), SEQ ID NO: 116. Two principal sets of cloning primers were used, one set was a universal primer pair binding to the vector and the poly-A tail, and the other set were gene-specific primers. The PCR product was first transferred into the pDONR vector and subsequently transferred into the destination vector pK7GWIWG2 according to manufacturers' recommendations (Invitrogen USA). The sequences of the genes and the corresponding construct, and cloning primer are listed in Table 2 and 3.

Full Sequence from Populus trichocarpa means sequences of the candidate genes as predicted from Populus trichocarpa genome sequence at JGI (G. A. Tuskan et al. Science 15 Sep. 2006: Vol. 313. no. 5793, pp. 1596-1604). The predicted sequence corresponds to the sequences that best maps the actual transcript sequence from hybrid aspen or the corresponding gene model at JGI. Full Sequence from Populus tremula×tremuloides means the best sequence of the candidate genes resulting from resequencing of hybrid aspen EST's, sequencing of fragments cloned in the RNAi construct and EST sequences present in the Pop-db, sequence used for RNAi construct means the sequence of the actually cloned fragment in the RNAi constructs.

TABLE 2
Summary of the cloning primers used.

| Gene | Construct | Cloning forward primer | Cloning reverse primer |
|---|---|---|---|
| STT5 | KR002B | SEQ ID No: 01 | SEQ ID No: 09 |
| STT11 | KR008B | | |
| STT13 | KR010B | | |
| STT14 | KR012B | | |
| STT16 | KR014B | | |
| STT17 | KR015B | | |
| STT23 | KR080B | | |
| STT27 | KR100B | | |
| STT31 | KR111 | | |
| STT32 | KR112 | | |
| STT40 | KR126 | | |
| STT49 | KR140 | | |
| STT54 | KR151 | | |
| STT57 | KR154 | | |
| STT59 | KR159 | | |
| STT64 | KR165 | | |
| STT70 | KR175 | | |
| STT74 | KR179 | | |
| STT75 | KR182 | | |
| STT76 | KR183 | | |
| STT82 | KR192 | | |
| STT83 | KR198 | | |
| STT95 | KR213 | | |
| STT97 | KR215 | | |
| STT101 | KR223 | | |
| STT116 | KR242 | | |
| STT124 | KR287 | | |
| STT128 | KR318 | | |
| STT165 | KR470 | SEQ ID No: 02 | SEQ ID No: 10 |
| STT167 | KR472 | SEQ ID No: 03 | SEQ ID No: 11 |
| STT170 | KR475 | SEQ ID No: 04 | SEQ ID No: 12 |
| STT173 | KR478 | SEQ ID No: 05 | SEQ ID No: 13 |
| STT178 | KR484 | SEQ ID No: 06 | SEQ ID No: 14 |
| STT307 | KR595 | SEQ ID No: 07 | SEQ ID No: 15 |
| STT317 | KR605 | SEQ ID No: 08 | SEQ ID No: 16 |
| STT187 | KR489 | SEQ ID No: 127 | SEQ ID No: 134 |
| STT311 | KR599 | SEQ ID No: 128 | SEQ ID No: 135 |
| STT315 | KR603 | SEQ ID No: 129 | SEQ ID No: 136 |
| STT392 | KR680 | SEQ ID No: 130 | SEQ ID No: 137 |
| STT398 | KR686 | SEQ ID No: 131 | SEQ ID No: 138 |
| STT442 | KR730 | SEQ ID No: 132 | SEQ ID No: 139 |
| STT476 | KR764 | SEQ ID No: 133 | SEQ ID No: 140 |

TABLE 3
Genes and the corresponding RNAi construct sequences

| | | Sequence used for | Full Sequence from | Full Sequence from Populus |
|---|---|---|---|---|
| Gene | Construct | RNAi construct | Populus trichocarpa | tremula × tremuloides |
| STT5 | KR002B | SEQ ID No: 23 | SEQ ID No: 59 | SEQ ID No: 94 |
| STT11 | KR008B | SEQ ID No: 24 | SEQ ID No: 60 | SEQ ID No: 24 |
| STT13 | KR010B | SEQ ID No: 25 | SEQ ID No: 61 | SEQ ID No: 25 |
| STT14 | KR012B | SEQ ID No: 26 | SEQ ID No: 62 | SEQ ID No: 26 |
| STT16 | KR014B | SEQ ID No: 27 | SEQ ID No: 63 | SEQ ID No: 95 |
| STT17 | KR015B | SEQ ID No: 28 | SEQ ID No: 64 | SEQ ID No: 96 |
| STT23 | KR080B | SEQ ID No: 29 | SEQ ID No: 65 | SEQ ID No: 29 |
| STT27 | KR100B | SEQ ID No: 30 | SEQ ID No: 66 | SEQ ID No: 30 |
| STT31 | KR111 | SEQ ID No: 31 | SEQ ID No: 67 | SEQ ID No: 31 |
| STT32 | KR112 | SEQ ID No: 32 | SEQ ID No: 68 | SEQ ID No: 97 |
| STT40 | KR126 | SEQ ID No: 33 | SEQ ID No: 69 | SEQ ID No: 33 |
| STT49 | KR140 | SEQ ID No: 34 | SEQ ID No: 70 | SEQ ID No: 34 |
| STT54 | KR151 | SEQ ID No: 35 | SEQ ID No: 71 | SEQ ID No: 35 |
| STT57 | KR154 | SEQ ID No: 36 | SEQ ID No: 72 | SEQ ID No: 36 |
| STT59 | KR159 | SEQ ID No: 37 | SEQ ID No: 73 | SEQ ID No: 98 |
| STT64 | KR165 | SEQ ID No: 38 | SEQ ID No: 74 | SEQ ID No: 38 |
| STT70 | KR175 | SEQ ID No: 39 | SEQ ID No: 75 | SEQ ID No: 39 |
| STT74 | KR179 | SEQ ID No: 40 | SEQ ID No: 76 | SEQ ID No: 40 |
| STT75 | KR182 | SEQ ID No: 41 | SEQ ID No: 77 | SEQ ID No: 99 |
| STT76 | KR183 | SEQ ID No: 42 | SEQ ID No: 78 | SEQ ID No: 42 |
| STT82 | KR192 | SEQ ID No: 43 | SEQ ID No: 79 | SEQ ID No: 43 |
| STT83 | KR198 | SEQ ID No: 44 | SEQ ID No: 80 | SEQ ID No: 44 |
| | | SEQ ID No: 45 | | SEQ ID No: 45 |
| STT95 | KR213 | SEQ ID No: 46 | SEQ ID No: 81 | SEQ ID No: 46 |
| STT97 | KR215 | SEQ ID No: 47 | SEQ ID No: 82 | SEQ ID No: 100 |
| | | | | SEQ ID No: 101 |
| STT101 | KR223 | SEQ ID No:48 | SEQ ID No: 83 | SEQ ID No: 102 |
| STT116 | KR242 | SEQ ID No:49 | SEQ ID No: 84 | SEQ ID No: 49 |
| STT124 | KR287 | SEQ ID No: 50 | SEQ ID No: 85 | SEQ ID No: 50 |
| STT128 | KR318 | SEQ ID No: 51 | SEQ ID No: 86 | SEQ ID No: 51 |
| STT165 | KR470 | SEQ ID No: 52 | SEQ ID No: 87 | SEQ ID No: 103 |
| | | | | SEQ ID No: 104 |
| STT167 | KR472 | SEQ ID No: 53 | SEQ ID No: 88 | SEQ ID No: 105 |
| STT170 | KR475 | SEQ ID No: 54 | SEQ ID No: 89 | SEQ ID No: 106 |
| STT173 | KR478 | SEQ ID No: 55 | SEQ ID No: 90 | SEQ ID No: 107 |
| STT178 | KR484 | SEQ ID No: 56 | SEQ ID No: 91 | SEQ ID No: 108 |
| | | | | SEQ ID No: 109 |
| STT307 | KR595 | SEQ ID No: 57 | SEQ ID No: 92 | SEQ ID No: 110 |
| | | | | SEQ ID No: 111 |
| STT317 | KR605 | SEQ ID No: 58 | SEQ ID No: 93 | SEQ ID No: 112 |
| STT187 | KR489 | SEQ ID No: 157 | SEQ ID No: 164 | SEQ ID No: 157 |
| STT311 | KR599 | SEQ ID No: 158 | SEQ ID No: 165 | SEQ ID No: 171 |
| STT315 | KR603 | SEQ ID No: 159 | SEQ ID No: 166 | SEQ ID No: 159 |
| STT392 | KR680 | SEQ ID No: 160 | SEQ ID No: 167 | SEQ ID No: 172 |
| STT398 | KR686 | SEQ ID No: 161 | SEQ ID No: 168 | SEQ ID No: 173 |
| STT442 | KR730 | SEQ ID No: 162 | SEQ ID No: 169 | SEQ ID No: 174 |
| STT476 | KR764 | SEQ ID No: 163 | SEQ ID No: 170 | SEQ ID No: 175 |

### Plant Transformation

CaMV 35S: Inverted repeat DNA constructs were transformed into Agrobacterium and subSEQuent into Hybrid aspen, Populus tremula L.×P. tremuloides Minch. Clone T89, hereafter called “poplar”, was transformed and regenerated essentially as described in Nilsson et al. (1992). Approximately 3-8 independent lines were generated for each construct. One such group of transgenic trees produced using one construct is hereafter called a “construction group”, e.g. different transgenic trees emanating from one construct. Each transgenic line within each construction group, e.g. KR555-2B KR555-3A, KR555-2B and so on, are different transformation events and therefore most probably have the recombinant DNA inserted into different locations in the plant genome. This makes the different lines within one construction group partly different. For example it is known that different transformation events will produce plants with different levels of gene down-regulation when using RNAi constructs of the type used here.

### Plant Growth

The transgenic poplar lines were grown together with their wild type control (wt) trees, in a greenhouse under a photoperiod of 18 h and a temperature of 22° C./15° C. (day/night). The plants were fertilized weekly Weibulls Rika S NPK 7-1-5 diluted 1 to 100 (final concentrations NO₃, 55 g/l; NH₄, 29 g/l; P, 12 g/l; K, 56 g/l; Mg 7.2 g/l; S, 7.2 g/l; B, 0.18 g/l; Cu, 0.02 g/l; Fe, 0.84 g/l; Mn, 0.42 g/l; Mo, 0.03 g/l; Zn, 0.13 g/l). The plants were grown for 8-9 weeks before harvest. During this time their height and diameter was measured 1 to 2 times per week. A number of wild type trees (typically 15-25 trees) and a number of transgenic trees comprising several construction groups (typically 6-20 construction groups) were grown in parallel in the greenhouse under the same above conditions. All comparisons between the wild type trees and construction groups are made within each growth group.

### Sampling

Two principal types of harvest and sampling were performed. One general type was for example for chemical analysis, wood morphological analysis, gene expression analysis, wood density analysis and metabolomics analysis. And another type for dry weight measurements of bark, wood, leafs and roots.

### Selection of Construction Groups

In the first round of growth for each group of trees with a specific gene down regulated using RNAi, i.e. a construction group, a number of the following analyses was performed: FT-IR analysis. These data were analysed in order to single out the Construction Groups that showed a phenotypic variation compared to wild type control trees.

### Selection of Construction Groups Using FT-IR and Multivariate Analysis

### FT-IR Analysis

All samples were freeze dried and milled to a fine powder using a ball mill (Retch NM 301) prior to FT-IR analysis. The measuring procedure has been shifted over time; samples have been measured as pure samples or as 2.5% sample in potassium bromide (KBr). Different instruments (Bruker IFS 66v and Bruker 55 Equinox, Bruker) have been used hence the wavelength range changed from 900-1800 nm to 400-5200 nm in the latter case only the fingerprint region 800-1860 nm have been used for multivariate analysis.

### Baseline Correction and Normalization

Two different approaches have been used to normalize the samples prior to multivariate analysis in order to reduce effects such as baseline and concentration variations.

When the samples are measured using the instrument with the larger wavelength range a baseline is fitted for each sample using one point on each side of the fingerprint region (˜800 nm and ˜1860 nm) the baseline is subtracted and the area under the spectrum normalized to a constant sum. [Orthogonal Projections to Latent Structures Discriminant Analysis Modeling on in Situ FT-IR Spectral Imaging of Liver Tissue for Identifying Sources of Variability Stenlund, Hans, Gorzsás, Andras, Persson, Per, Sundberg, Bjorn, and Trygg, Johan Anal. Chem., 2008, 10.1021/ac8005318 Web Release Date: Aug. 20, 2008]

When samples are measured using the other instrument that only measures between 900-1800 nm the first two principal components has been removed from data since they were considered to only contain variation related to baseline and concentration variations.

### Multivariate Analysis

Baseline corrected and normalized FT-IR data has been subjected to multivariate projection methods, such as PCA (Wold S, Esbensen K, Geladi P. Principal component analysis. Chemometrics and Intelligent Laboratory Systems 1987, 2:37-52), PLS (Geladi P, Kowalski B R. Partial least-squares regression—a Tutorial. Analytica Chimica Acta 1986, 185:1-17) and OPLS (Trygg J, Wold S. Orthogonal projections to latent structures (OPLS). Journal of Chemometrics 2002, 16:119-128), in order to detect constructions groups (genes) that in some mean chemically deviate from the wild type population. A construction group (gene) that has at least 2 samples that deviates from the wild type population in similar way has been scored as a changed phenotype and then been further analyzed by other methods.

A construction group that scored positive in the FT-IR analysis was in most cases analyzed for lignin content.

### Lignin Measurements

Lignin content was measured at MoRe Research, Örnsköldsvik, using their internal method MoRe KA 10.219, which is based upon the Tappi method T 222 om-88 (http://www.tappi.org) with some modifications. The method is known as Klason-lignin and uses the fact that lignin is insoluble in acids.

The dried milled wood samples are pre hydrolyzed in 72% sulfuric acid which then is diluted to 2% and further hydrolyzed during refluxing for 9 hr. The dispersed lignin is filtered of and then washed and dried. The weight of the dried lignin is measured and compared with the original weight to decide the lignin content.

### Statistical Evaluation

Each construction is subjected to 4 different tests;

1) T-test Blom ISBN 91-44-05592-7; the mean value for the samples belonging to each construction is compared with the mean value for the corresponding T89 samples, using a 2-tailed T-test assuming unequal variance. If the probability that the mean values are equal is less that 5% the construction is considered to have changed lignin content.

2) Anova; the between and within group (construction and wild type) variation is compared using an F-test Blom ISBN 91-44-05592-7. If the probability that the between and the within group variation is equal is lower than 5% the construction is considered to have changed lignin content. This is equivalent to a 2-tailed T-test assuming equal variance.

3) A 95% confidence interval is calculated around the mean value for the wild type samples using T statistics. One or more lines of a construction have lignin content outside this confidence interval the construction is considered to affect the lignin content.

4) A non statistical measure, were the samples for each construction is compared with the lowest and highest value for the corresponding T89 samples. If one or more samples from one construction have higher lignin content than the highest or lower than the lowest lignin content for a T89 sample, it the construction is assumed to affect the lignin content.

Construction groups meeting one or more of these criteria were selected.

A number of the construction groups that showed differences in lignin levels were tested with RT-PCR in order to verify the down regulation of the targeted gene.

Real-time RT-PCR (Bustin 2000) was used to compare construct gene expression levels of the construction group with corresponding wild type group. The expression level of 26S ribosome regulatory subunit S2 was used as a reference to which construct gene expression was normalized. The comparative CT method was used for calculation of relative construct gene expression levels, where the ratio between construction and reference gene expression levels is described by (1+E_{target})^{-CTtarget}/(1+E_{reference})^{-CTreference} where E_{target} and E_{reference} are the efficiencies of construct and reference gene PCR amplification respectively and CT_{target} and CT_{reference} are the threshold cycles as calculated for construct and reference gene amplification respectively.

For total RNA extraction, stem samples (approx. 50 mg) were harvested from greenhouse grown plants and flash frozen in liquid nitrogen. Frozen samples were ground in a bead mill. Total RNA was extracted using Aurum Total RNA Mini kit according to manufacturers' recommendations (Bio-Rad). cDNA synthesis was performed using iScript cDNA synthesis kit according to manufacturers recommendations (Bio-Rad).

For real-time PCR, cDNA template was mixed with SYBR Green Supermix according to manufacturers recommendations (Bio-Rad) and corresponding construct gene specific primers or internal reference gene specific primers, forward PCR primer SEQ ID NO: 123 and reverse PCR primer sequence SEQ ID NO: 124. Real-time PCR was run on a MyiQ PCR thermocycler (Bio-Rad) and analysed using included software iQS. For each sample, reactions were set up in three to six replicates, with an equal number of replicates using construct gene specific primers and reference gene specific primers, and the average threshold cycle for the reaction replicates was subsequently used for calculation of relative construct gene expression levels.

The 96 well plate was covered with microfilm and set in the thermocycler to start the reaction cycle. By way of illustration, the reaction cycle may include the following steps: Initial denaturation at 95° C. for 3 minutes 30 seconds followed by 40 rounds of amplification comprising the following steps 95° C. for 10 seconds, 55° C. for 30 seconds and 72° C. for 40 seconds.

Selected genes were in a number of cases tested in over expression experiments were the full length open reading frame (ORF) for the gene corresponding to the RNAi construct were cloned and put under the CaMV 35S promoter and tested in transgenic trees. The RNAi constructs were from Hybrid aspen i.e. a hybrid of the two species Populus tremuloides and Populus tremula.

These plants were in many cases tested for lignin content and a number of these gene constructs also modified the cell wall chemistry.

### Cloning of Full Length Genes for Over Expression Analysis Using the 35S Promoter

For over expression analysis of a corresponding full length gene, previously down regulated using RNAi, the gene coding sequence (CDS) was predicted from the genome sequence of Populus trichocarpa (Tuskan et al., 2006). The gene models were compared to, and in some instances corrected based on, information published for homologous genes in Arabidopsis thaliana and other plant species. This was done using databases such as http://www.ncbi.nlm.nih.gov/ and http://www.arabidopsis.org/. Selected genes were subsequently cloned into an over-expression vector under the control of the CaMV 35S promoter. Cloning primers were designed to be gene specific and to include the full CDS and in some instances parts of the untranslated transcript region (UTR). The full length gene was amplified from hybrid aspen cDNA using Phusion high fidelity DNA polymerase (Finnzymes) and first transferred into the pDONR vector (Invitrogen USA) and subsequently transferred into the destination vector pK2GW7 (Karimi et al., 2002) using Gateway technology (Invitrogen USA). Constructs thus cloned have construct names starting with 35s. The sequences of the selected full length genes, PCR primers etc. are listed in Table 4.

### Cloning of Full Length Genes for Over Expression Analysis Using the LMP1 Promoter

For over expression analysis of a corresponding full length gene, previously down regulated using RNAi, the gene coding sequence (CDS) was predicted from the genome sequence of Populus trichocarpa (Tuskan et al., 2006). The gene models were compared to, and in some instances corrected based on, information published for homologous genes in Arabidopsis thaliana and other plant species. This was done using databases such as http://www.ncbi.nlm.nih.gov/ and http://www.arabidopsis.org/. Selected genes were subsequently cloned into an over-expression vector under the control of the LMP1 promoter, described in patent application WO 2004/097024. Cloning primers were designed to be gene specific and to include the full CDS and in some instances parts of the untranslated transcript region (UTR). The full length gene was amplified from hybrid aspen cDNA using Phusion high fidelity DNA polymerase (Finnzymes) and first transferred into the pDONR vector (Invitrogen USA) and subsequently transferred into the destination vector pPCV812-LMP1-Gateway using Gateway technology (Invitrogen USA). Constructs thus cloned have construct names starting with LMP1. The pPCV812 plant binary vector is described in Koncz et al., 1994. The pPCV812 vector has been further modified to create the pPCV812-LMP1-Gateway vector by insertion of the LMP1 promoter between the BglII and SalI sites and by replacing the GUS reporter gene uidA with a Gateway recombination cassette (attR1-ccdB-attR2, Invitrogen Life Technologies) using BamHI and SacI sites. The sequences of the selected full length genes, PCR primers etc. are listed in Table 4.

### Cloning of Full Length Transcription Factor Genes for Over Expression Analysis Using the 35S Promoter

The corresponding gene models for the selected transcription factor genes were extracted from data derived from the genome sequencing of Populus trichocarpa (Tuskan et al., 2006) using BLAST analysis. The gene models were compared to, and in some instances corrected based on, information published for homologous genes in Arabidopsis thaliana and other plant species. This was done using databases such as http://www.ncbi.nlm.nih.gov/ and http://www.arabidopsis.org/. Selected genes were subsequently cloned into an over-expression vector under the control of the CaMV 35S promoter. For isolation of cDNA, total RNA was isolated from stem, leaf and bark tissue sampled from hybrid aspen clone T89 plants and reverse transcribed to cDNA using Superscript III First Strand Synthesis System (Invitrogen). cDNA were then amplified by PCR with gene specific forward and reverse primers using Phusion high fidelity DNA polymerase (Finnzymes). PCR primers were selected as follows, the 5′-primer was placed at the start codon and the 3′ reverse primer was placed 3′ of the translational stop site. Forward primers were modified by the introduction of a Kozak sequence (5″-AGAACC-3″) upstream and next to the start codon of each target gene. The amplified cDNAs were inserted into a Gateway entry vector pENTR/D-TOPO (Invitrogen), followed by transfer of the genes into the expression vector pK2GW7 (Karimi et al., 2002) using the Gateway LR recombination reaction (Invitrogen). The cloned genes were control sequenced and compared to the selected genes using standard techniques before sub cloning into the plant vector pK2GW7.

TABLE 4
PCR primer sequences (a) and sequences used for 35S over expression constructs (b)
a

| Gene | Construct | Cloning Forward primer | Cloning Reverse primer |
|---|---|---|---|
| STT14 | 35s005 | SEQ ID No: 17 | SEQ ID No: 20 |
| STT17 | 35s006 | SEQ ID No: 18 | SEQ ID No: 21 |
| STT57 | 35s008 | SEQ ID No: 19 | SEQ ID No: 22 |
| STT97 | 35s029 | SEQ ID No: 141 | SEQ ID No: 149 |
| STT167 | LMP1-002 | SEQ ID No: 142 | SEQ ID No: 150 |
| STT551 | TF0003 | SEQ ID No: 143 | SEQ ID No: 151 |
| STT577 | TF0050 | SEQ ID No: 144 | SEQ ID No: 152 |
| STT584 | TF0061 | SEQ ID No: 145 | SEQ ID No: 153 |
| STT606 | TF0094.2nd | SEQ ID No: 146 | SEQ ID No: 154 |
| STT633 | TF0138 | SEQ ID No: 147 | SEQ ID No: 155 |
| STT658 | TFSTT021 | SEQ ID No: 148 | SEQ ID No: 156 |

b

| | | Sequence used for over | Full Sequence from | Full Sequence from Populus |
|---|---|---|---|---|
| Gene | Construct | expression construct | Populus trichocarpa | tremula × tremuloides |
| STT14 | 35s005 | SEQ ID No: 113 | SEQ ID No: 62 | SEQ ID No: 26 |
| STT17 | 35s006 | SEQ ID No: 114 | SEQ ID No: 64 | SEQ ID No: 96 |
| STT57 | 35s008 | SEQ ID No: 115 | SEQ ID No: 72 | SEQ ID No: 36 |
| STT97 | 35s029 | SEQ ID No: 176 | SEQ ID No: 188 | SEQ ID No: 176 |
| | | SEQ ID No: 177 | | SEQ ID No: 177 |
| STT167 | LMP1-002 | SEQ ID No: 178 | SEQ ID No: 189 | SEQ ID No: 105 |
| STT551 | TF0003 | SEQ ID No: 179 | SEQ ID No: 190 | SEQ ID No: 179 |
| STT577 | TF0050 | SEQ ID No: 180 | SEQ ID No: 191 | SEQ ID No: 180 |
| | | SEQ ID No: 181 | | SEQ ID No: 181 |
| STT584 | TF0061 | SEQ ID No: 182 | SEQ ID No: 192 | SEQ ID No: 182 |
| STT606 | TF0094.2nd | SEQ ID No: 183 | SEQ ID No: 193 | SEQ ID No: 183 |
| | | SEQ ID No: 184 | | SEQ ID No: 184 |
| STT633 | TF0138 | SEQ ID No: 185 | SEQ ID No: 194 | SEQ ID No: 185 |
| STT658 | TFSTT021 | SEQ ID No: 186 | SEQ ID No: 195 | SEQ ID No: 186 |
| | | SEQ ID No: 187 | | SEQ ID No: 187 |

Full Sequence from Populus tremula × tremuloides means the best Sequence of the candidate genes resulting from resequencing of hybrid aspen EST's, sequencing of cloned fragments etc. Sequence used for 35S over expression construct means the sequence of the actually cloned fragment in the over expression constructs.

### Results

The isolated genes and the corresponding constructions that has been used is shown below, which passed the selection criteria according to the invention, table 5.

TABLE 5
Summary of modulated lignin results

| Gene | Construct | T-test | Anova | <T int | >T Int | <Min | >Max | Ratio | Modulation | SEQ ID | +/−vs wt T89 |
|---|---|---|---|---|---|---|---|---|---|---|---|
| STT 57 | KR154 | Sign | Sign | 1 | 0 | 3 | 0 | 0.92 | Down reg. RNAi | No: 36 | −11% |
| STT 57 | 35s008 | | | 0 | 1 | 0 | 1 | 1.02 | Over expression | No: 115 | 15% |
| STT 14 | KR012B | | | 0 | 2 | 0 | 3 | 1.04 | Down reg. RNAi | No: 26 | 11% |
| STT 14 | 35s005 | Sign | Sign | 0 | 0 | 0 | 4 | 1.08 | Over expression | No: 113 | 13% |
| STT 17 | KR015B | | | 2 | 0 | 2 | 0 | 0.97 | Down reg. RNAi | No: 28 | −12% |
| STT 17 | 35s006 | | Sign | 0 | 1 | 0 | 2 | 1.1 | Down reg. RNAi | No: 114 | 16% |
| STT 49 | KR140 | Sign | Sign | 1 | 0 | 2 | 0 | 0.93 | Down reg. RNAi | No: 34 | −12% |
| STT 70 | KR175 | Sign | Sign | 0 | 0 | 2 | 0 | 0.94 | Down reg. RNAi | No: 39 | −9% |
| STT 116 | KR242 | Sign | Sign | 0 | 0 | 1 | 0 | 0.96 | Down reg. RNAi | No: 49 | −9% |
| STT 74 | KR179 | Sign | Sign | 0 | 0 | 0 | 0 | 0.93 | Down reg. RNAi | No: 40 | −10% |
| STT 75 | KR182 | Sign | | 0 | 0 | 0 | 0 | 0.94 | Down reg. RNAi | No: 41 | −8% |
| STT 82 | KR192 | Sign | | 0 | 0 | 0 | 0 | 0.97 | Down reg. RNAi | No: 43 | −5% |
| STT 76 | KR183 | | Sign | 3 | 0 | 3 | 0 | 0.92 | Down reg. RNAi | No: 42 | −20% |
| STT 173 | KR478 | | Sign | 2 | 0 | 3 | 0 | 0.96 | Down reg. RNAi | No: 55 | −10% |
| STT 59 | KR159 | | Sign | 1 | 0 | 3 | 0 | 0.93 | Down reg. RNAi | No: 37 | −12% |
| STT 27 | KR100B | | | 0 | 2 | 0 | 2 | 1.04 | Down reg. RNAi | No: 30 | 13% |
| STT 31 | KR111 | | | 2 | 0 | 2 | 0 | 0.95 | Down reg. RNAi | No: 31 | −19% |
| STT 32 | KR112 | | | 0 | 1 | 0 | 2 | 1.03 | Down reg. RNAi | No: 32 | 12% |
| STT 64 | KR165 | | | 1 | 1 | 2 | 1 | 0.98 | Down reg. RNAi | No: 38 | −15% |
| STT 95 | KR213 | | | 0 | 1 | 0 | 2 | 1.05 | Down reg. RNAi | No: 46 | 12% |
| STT 165 | KR470 | | | 1 | 0 | 2 | 0 | 0.97 | Down reg. RNAi | No: 52 | −7% |
| STT 5 | KR002B | | | 1 | 0 | 1 | 0 | 0.96 | Down reg. RNAi | No: 23 | −10% |
| STT 13 | KR010B | | | 0 | 1 | 0 | 1 | 1.01 | Down reg. RNAi | No: 25 | 8% |
| STT 23 | KR080B | | | 1 | 0 | 1 | 0 | 0.97 | Down reg. RNAi | No: 29 | −10% |
| STT 40 | KR126 | | | 1 | 0 | 1 | 0 | 0.96 | Down reg. RNAi | No: 33 | −16% |
| STT 83 | KR198 | | | 1 | 0 | 1 | 0 | 0.99 | Down reg. RNAi | No: 44* | −7% |
| STT 83 | KR198 | | | | | | | | Down reg. RNAi | No: 45** | |
| STT 101 | KR223 | | | 1 | 0 | 1 | 0 | 1 | Down reg. RNAi | No: 48 | −6% |
| STT 124 | KR287 | | | 1 | 0 | 1 | 0 | 0.97 | Down reg. RNAi | No: 50 | −8% |
| STT 178 | KR484 | | | 1 | 1 | 1 | 1 | 0.97 | Down reg. RNAi | No: 56 | −8% |
| STT 54 | KR151 | | | 0 | 0 | 2 | 0 | 0.95 | Down reg. RNAi | No: 35 | −10% |
| STT 97 | K215 | | | 0 | 0 | 0 | 2 | 1 | Down reg. RNAi | No: 47 | 5% |
| STT 307 | KR595 | | | 0 | 0 | 2 | 0 | 0.98 | Down reg. RNAi | No: 57 | −10% |
| STT 11 | KR008B | | | 0 | 0 | 0 | 1 | 1.01 | Down reg. RNAi | No: 24 | 7% |
| STT 16 | KR014B | | | 0 | 0 | 0 | 1 | 1.02 | Down reg. RNAi | No: 27 | −6% |
| STT 128 | KR318 | | | 0 | 0 | 1 | 0 | 0.98 | Down reg. RNAi | No: 51 | −7% |
| STT 167 | KR472 | | | 0 | 0 | 0 | 1 | 1.02 | Down reg. RNAi | No: 53 | 5% |
| STT 170 | KR475 | | | 0 | 0 | 0 | 1 | 1.01 | Down reg. RNAi | No: 54 | 5% |
| STT 317 | KR605 | | | 0 | 0 | 0 | 1 | 1.03 | Down reg. RNAi | No: 58 | 7% |
| STT 187 | KR489 | Sign | Sign | 0 | 0 | 0 | 1 | 1.04 | Down reg. RNAi | No: 157 | 8% |
| STT 311 | KR599 | | | 1 | 0 | 2 | 2 | 0.99 | Down reg. RNAi | No: 158 | −9% |
| STT 315 | KR603 | | | 0 | 1 | 0 | 3 | 1.05 | Down reg. RNAi | No: 159 | 11% |
| STT 392 | KR680 | | | 0 | 1 | 0 | 2 | 1.02 | Down reg. RNAi | No: 160 | 7% |
| STT 398 | KR686 | Sign | Sign | 3 | 0 | 4 | 0 | 0.94 | Down reg. RNAi | No: 161 | −8% |
| STT 442 | KR730 | | | 0 | 1 | 0 | 1 | 1 | Down reg. RNAi | No: 162 | 7% |
| STT 476 | KR764 | | | 1 | 0 | 1 | 2 | 0.99 | Down reg. RNAi | No: 163 | −8% |
| STT 97 | 35s029 | | | 1 | 0 | 3 | 0 | 0.97 | Over expression | No: 176* | −8% |
| STT 97 | 35s029 | | | | | | | | Over expression | No: 177** | |
| STT 167 | LMP1-002 | | | 0 | 1 | 0 | 1 | 1.02 | Over expression | No: 178 | 9% |
| STT 551 | TF0003 | Sign | Sign | 0 | 2 | 0 | 2 | 1.04 | Over expression | No: 179 | 7% |
| STT 577 | TF0050 | Sign | Sign | 2 | 0 | 2 | 0 | 0.96 | Over expression | No: 180 | −8% |
| STT 577 | TF0050 | | | | | | | | Over expression | No: 181 | |
| STT 584 | TF0061 | | | 1 | 0 | 1 | 2 | 1.02 | Over expression | No: 182 | −8% |
| STT 606 | TF0094.2nd | Sign | Sign | 0 | 0 | 2 | 0 | 0.96 | Over expression | No: 183* | −8% |
| STT 606 | TF0094.2nd | | | | | | | | Over expression | No: 184** | |
| STT 633 | TF0138 | | | 1 | 0 | 1 | 0 | 0.99 | Over expression | No: 185 | −8% |
| STT 658 | TFSTT021 | | | 2 | 1 | 2 | 1 | 0.98 | Over expression | No: 186* | −7% |
| STT 658 | TFSTT021 | | | | | | | | Over expression | No: 187** | |

The first column “Gene” denotes the gene number of the construction, which was used in transformation. The following two columns contain the result of a paired t-test and ANOVA respectively. “<T inv” and “>T inv” denotes the number of samples outside the T (95%) distribution for T89 on the lower and upper side respectively. “>Max” and “<Min” denotes the number of samples higher the highest T89 and lower than the lowest T89 sample respectively. Ratio is the ratio between construction average and T89 average.
*Corresponds to the 5′-end of the gene
**Corresponds to the 3′-end of the gene

### Gene STT 5

The gene ST5 is a glycosyltransferase homologous to GT47, (Zhou et al. Plant and Cell Physiology (2006), 47(9), 1229-1240.) This gene is suggested to be involved in xylan biosynthesis, we here show that this gene can be used to modify lignin content in trees, table 6. Construction group KR002B shows a maximum decrease of lignin of 10% compared to the wild type average.

TABLE 6

| Individual Name | Lignin Content (%) |
|---|---|
| T89-103 | 18.9 |
| T89-104 | 19 |
| T89-106 | 17.8 |
| T89-107 | 19.3 |
| T89-108 | 19.7 |
| T89-109 | 19 |
| T89-110 | 19.7 |
| T89-111 | 18.6 |
| T89-112 | 19.8 |
| T89-113 | 19.2 |
| T89-114 | 20.1 |
| T89-115 | 18.6 |
| KR002B-1B | 19.1 |
| KR002B-2A | 17.8 |
| KR002B-3B | 17.2 |
| KR002B-4A | 19.4 |
| KR002B-5B | 18.7 |

| | |
|---|---|
| Number (KR002B) | 5 |
| Average (KR002B) | 18.44 |
| Standard deviation (KR002B) | 0.92 |
| Number (T89) | 12 |
| Average (T89) | 19.14 |
| Standard deviation (T89) | 0.64 |
| Max (T89) | 20.10 |
| Min (T89) | 17.80 |
| Confidence Interval (T89) (95%) | 19.14 +/− 1.41 |
| Ratio (Number (KR002B)/Average (T89)) | 0.96 |
| Ratio (Max(KR002B)/Average(T89)) | 1.01 |
| Ratio (Min(KR002B)/Average(T89)) | 0.90 |
| T-test | 0.1727 |
| Anova | 0.0886 |
| Num KR002B > Confidence Interval (T89) (95%) | 0 |
| (20.55) | |
| Num KR002B < Confidence Interval (T89) (95%) | 1 |
| (17.74) | |
| Num KR002B > Max (T89) (20.1) | 0 |
| Num KR002B < Min (T89) (17.8) | 1 |

### Gene STT 11

The RNAi construction group KR008B shows a maximum increase of lignin of 7% compared to the wild type average, see Table 7 below.

TABLE 7

| Individual Name | Lignin Content (%) |
|---|---|
| T89-103 | 18.9 |
| T89-104 | 19 |
| T89-106 | 17.8 |
| T89-107 | 19.3 |
| T89-108 | 19.7 |
| T89-109 | 19 |
| T89-110 | 19.7 |
| T89-111 | 18.6 |
| T89-112 | 19.8 |
| T89-113 | 19.2 |
| T89-114 | 20.1 |
| T89-115 | 18.6 |
| KR008B-1A-A | 20.5 |
| KR008B-1A-B | 18.7 |
| KR008B-3B | 18.8 |
| KR008B-5A | 19.4 |
| KR008B-5B | 18.8 |

| | |
|---|---|
| Number (KR008B) | 5 |
| Average (KR008B) | 19.24 |
| Standard deviation (KR008B) | 0.76 |
| Number (T89) | 12 |
| Average (T89) | 19.14 |
| Standard deviation (T89) | 0.64 |
| Max (T89) | 20.10 |
| Min (T89) | 17.80 |
| Confidence Interval (T89) (95%) | 19.14 +/− 1.41 |
| Ratio (Number (KR008B)/Average (T89)) | 1.01 |
| Ratio (Max(KR008B)/Average(T89)) | 1.07 |
| Ratio (Min(KR008B)/Average(T89)) | 0.98 |
| T-test | 0.8065 |
| Anova | 0.7873 |
| Num KR008B > Confidence Interval (T89) (95%) | 0 |
| (20.55) | |
| Num KR008B < Confidence Interval (T89) (95%) | 0 |
| (17.74) | |
| Num KR008B > Max (T89) (20.1) | 1 |
| Num KR008B < Min (T89) (17.8) | 0 |

### Gene STT 13

The RNAi construction group KR010B shows a maximum increase of lignin of 8% compared to the wild type average, see Table 8 below.

TABLE 8

| Individual Name | Lignin Content (%) |
|---|---|
| T89-103 | 18.9 |
| T89-104 | 19 |
| T89-106 | 17.8 |
| T89-107 | 19.3 |
| T89-108 | 19.7 |
| T89-109 | 19 |
| T89-110 | 19.7 |
| T89-111 | 18.6 |
| T89-112 | 19.8 |
| T89-113 | 19.2 |
| T89-114 | 20.1 |
| T89-115 | 18.6 |
| KR010B-1A-A | 17.8 |
| KR010B-2B | 20.6 |
| KR010B-3A | 19.3 |
| KR010B-3B | 19.9 |
| KR010B-4A | 19 |

| | |
|---|---|
| Number (KR010B) | 5 |
| Average (KR010B) | 19.32 |
| Standard deviation (KR010B) | 1.05 |
| Number (T89) | 12 |
| Average (T89) | 19.14 |
| Standard deviation (T89) | 0.64 |
| Max (T89) | 20.10 |
| Min (T89) | 17.80 |
| Confidence Interval (T89) (95%) | 19.14 +/− 1.41 |
| Ratio (Number (KR010B)/Average (T89)) | 1.01 |
| Ratio (Max(KR010B)/Average(T89)) | 1.08 |
| Ratio (Min(KR010B)/Average(T89)) | 0.93 |
| T-test | 0.7368 |
| Anova | 0.6694 |
| Num KR010B > Confidence Interval (T89) (95%) | 1 |
| (20.55) | |
| Num KR010B < Confidence Interval (T89) (95%) | 0 |
| (17.74) | |
| Num KR010B > Max (T89) (20.1) | 1 |
| Num KR010B < Min (T89) (17.8) | 0 |

### Gene STT 14

The gene STT 14 is suggested to be a microtubuli associated protein. The RNAi construction group KR012B gives a maximum increase of lignin of 11% compared to the wild type average, see Table 9 below.

TABLE 9

| Individual Name | Lignin Content (%) |
|---|---|
| T89-103 | 18.9 |
| T89-104 | 19 |
| T89-106 | 17.8 |
| T89-107 | 19.3 |
| T89-108 | 19.7 |
| T89-109 | 19 |
| T89-110 | 19.7 |
| T89-111 | 18.6 |
| T89-112 | 19.8 |
| T89-113 | 19.2 |
| T89-114 | 20.1 |
| T89-115 | 18.6 |
| KR012B-1B | 19.3 |
| KR012B-2A | 21.3 |
| KR012B-3B-A | 20.9 |
| KR012B-3B-B | 18.3 |
| KR012B-4A | 20.2 |

| | |
|---|---|
| Number (KR012B) | 5 |
| Average (KR012B) | 20.00 |
| Standard deviation (KR012B) | 1.22 |
| Number (T89) | 12 |
| Average (T89) | 19.14 |
| Standard deviation (T89) | 0.64 |
| Max (T89) | 20.10 |
| Min (T89) | 17.80 |
| Confidence Interval (T89) (95%) | 19.14 +/− 1.41 |
| Ratio (Number (KR012B)/Average (T89)) | 1.04 |
| Ratio (Max(KR012B)/Average(T89)) | 1.11 |
| Ratio (Min(KR012B)/Average(T89)) | 0.96 |
| T-test | 0.1960 |
| Anova | 0.0720 |
| Num KR012B > Confidence Interval (T89) (95%) | 2 |
| (20.55) | |
| Num KR012B < Confidence Interval (T89) (95%) | 0 |
| (17.74) | |
| Num KR012B > Max (T89) (20.1) | 3 |
| Num KR012B < Min (T89) (17.8) | 0 |

The full length gene corresponding to STT 14 was further over expressed with the construct 35S005. Construct 35S005 showed a maximum increase of lignin of 13% compared to the wild type average, see Table 10 below.

TABLE 10

| Individual Name | Lignin Content (%) |
|---|---|
| T89-12 | 19 |
| T89-19 | 19.3 |
| T89-29 | 18.7 |
| T89-43 | 19.5 |
| T89-8 | 17.8 |
| T89-9 | 16.9 |
| 35s005-1A | 20.8 |
| 35s005-1B | 19.8 |
| 35s005-2B | 20.4 |
| 35s005-3BA | 18.5 |
| 35s005-4A | 21 |

| | |
|---|---|
| Number (35s005) | 5 |
| Average (35s005) | 20.10 |
| Standard deviation (35s005) | 1.00 |
| Number (T89) | 6 |
| Average (T89) | 18.53 |
| Standard deviation (T89) | 1.00 |
| Max (T89) | 19.50 |
| Min (T89) | 16.90 |
| Confidence Interval (T89) (95%) | 18.53 +/− 2.56 |
| Ratio (Number (35s005)/Average (T89)) | 1.08 |
| Ratio (Max(35s005)/Average(T89)) | 1.13 |
| Ratio (Min(35s005)/Average(T89)) | 1.00 |
| T-test | 0.0306 |
| Anova | 0.0294 |
| Num 35s005 > Confidence Interval (T89) (95%) (21.1) | 0 |
| Num 35s005 < Confidence Interval (T89) (95%) (15.97) | 0 |
| Num 35s005 > Max (T89) (19.5) | 4 |
| Num 35s005 < Min (T89) (16.9) | 0 |

### Gene STT 16

The RNAi construction group KR014B shows a maximum decrease of lignin of 6% compared to the wild type average, see Table 11 below.

TABLE 11

| Individual Name | Lignin Content (%) |
|---|---|
| T89-103 | 18.9 |
| T89-104 | 19 |
| T89-106 | 17.8 |
| T89-107 | 19.3 |
| T89-108 | 19.7 |
| T89-109 | 19 |
| T89-110 | 19.7 |
| T89-111 | 18.6 |
| T89-112 | 19.8 |
| T89-113 | 19.2 |
| T89-114 | 20.1 |
| T89-115 | 18.6 |
| KR014B-1A-A | 19.9 |
| KR014B-1A-B | 20.2 |
| KR014B-3A | 20.1 |
| KR014B-3B-B | 18.6 |
| KR014B-4B-A | 19.1 |

| | |
|---|---|
| Number (KR014B) | 5 |
| Average (KR014B) | 19.58 |
| Standard deviation (KR014B) | 0.70 |
| Number (T89) | 12 |
| Average (T89) | 19.14 |
| Standard deviation (T89) | 0.64 |
| Max (T89) | 20.10 |
| Min (T89) | 17.80 |
| Confidence Interval (T89) (95%) | 19.14 +/− 1.41 |
| Ratio (Number (KR014B)/Average (T89)) | 1.02 |
| Ratio (Max(KR014B)/Average(T89)) | 1.06 |
| Ratio (Min(KR014B)/Average(T89)) | 0.97 |
| T-test | 0.2660 |
| Anova | 0.2280 |
| Num KR014B > Confidence Interval (T89) (95%) | 0 |
| (20.55) | |
| Num KR014B < Confidence Interval (T89) (95%) | 0 |
| (17.74) | |
| Num KR014B > Max (T89) (20.1) | 1 |
| Num KR014B < Min (T89) (17.8) | 0 |

### Gene STT 17

This gene is a glycosyltransferase and is described in Aspeborg et al (Plant Physiology (2005), 137(3), 983-997), we here show for the first time that this gene can be used to modulate lignin levels in trees. In the construction group KR015B down regulation of this gene gives a maximum decrease of lignin 12% compared to wild type average, when the full length gene corresponding to STT 57 was over expressed with the construct 35S006. Construction group 35S006 showed an maximum increase of lignin of 16% compared to the wild type average. Showing that this gene can both increase and decrease the lignin levels when over expressed and down regulated respectively, see Table 12 below.

TABLE 12

| Individual Name | Lignin Content (%) |
|---|---|
| T89-119 | 18.8 |
| T89-120 | 18.6 |
| T89-121 | 18.8 |
| T89-122 | 19 |
| T89-123 | 18.1 |
| T89-124 | 18.7 |
| T89-125 | 19.4 |
| T89-126 | 19.5 |
| T89-127 | 19.7 |
| T89-128 | 19.2 |
| T89-129 | 19.1 |
| T89-130 | 19 |
| T89-131 | 19.6 |
| T89-132 | 18.6 |
| KR015B-1B | 16.8 |
| KR015B-2B | 18.7 |
| KR015B-4A | 18 |
| KR015B-5A | 19.6 |
| KR015B-5B | 19.3 |

| | |
|---|---|
| Number (KR015B) | 5 |
| Average (KR015B) | 18.48 |
| Standard deviation (KR015B) | 1.12 |
| Number (T89) | 14 |
| Average (T89) | 19.01 |
| Standard deviation (T89) | 0.45 |
| Max (T89) | 19.70 |
| Min (T89) | 18.10 |
| Confidence Interval (T89) (95%) | 19.01 +/− 0.97 |
| Ratio (Number (KR015B)/Average (T89)) | 0.97 |
| Ratio (Max(KR015B)/Average(T89)) | 1.03 |
| Ratio (Min(KR015B)/Average(T89)) | 0.88 |
| T-test | 0.3587 |
| Anova | 0.1495 |
| Num KR015B > Confidence Interval (T89) (95%) | 0 |
| (19.98) | |
| Num KR015B < Confidence Interval (T89) (95%) | 2 |
| (18.04) | |
| Num KR015B > Max (T89) (19.7) | 0 |
| Num KR015B < Min (T89) (18.1) | 2 |

The full length gene corresponding to STT 17 was over expressed with the construct 35S006. Construct 35S006 showed a maximum increase of lignin of 16% compared to the wild type average, see Table 13 below.

TABLE 13

| Individual Name | Lignin Content (%) |
|---|---|
| T89-12 | 19 |
| T89-19 | 19.3 |
| T89-29 | 18.7 |
| T89-43 | 19.5 |
| T89-8 | 17.8 |
| T89-9 | 16.9 |
| 35s006-3B-1 | 19.3 |
| 35s006-4A | 20.6 |
| 35s006-5B-1 | 21.5 |

| | |
|---|---|
| Number (35s006) | 3 |
| Average (35s006) | 20.47 |
| Standard deviation (35s006) | 1.11 |
| Number (T89) | 6 |
| Average (T89) | 18.53 |
| Standard deviation (T89) | 1.00 |
| Max (T89) | 19.50 |
| Min (T89) | 16.90 |
| Confidence Interval (T89) (95%) | 18.53 +/− 2.56 |
| Ratio (Number (35s006)/Average (T89)) | 1.10 |
| Ratio (Max(35s006)/Average(T89)) | 1.16 |
| Ratio (Min(35s006)/Average(T89)) | 1.04 |
| T-test | 0.0679 |
| Anova | 0.0327 |
| Num 35s006 > Confidence Interval (T89) (95%) (21.1) | 1 |
| Num 35s006 < Confidence Interval (T89) (95%) (15.97) | 0 |
| Num 35s006 > Max (T89) (19.5) | 2 |
| Num 35s006 < Min (T89) (16.9) | 0 |

### Gene STT 23

This gene is a UTP-glucose-1-phosphate uridylyltransferase family protein. The RNAi construction group KR080 shows a maximum decrease of lignin of 10% compared to the wild type average see Table 14 below.

TABLE 14

| Individual Name | Lignin Content (%) |
|---|---|
| T89-152 | 19.1 |
| T89-153 | 18.1 |
| T89-154 | 19 |
| T89-155 | 18.5 |
| T89-158 | 18.8 |
| T89-159 | 17.4 |
| T89-160 | 17.9 |
| KR080B-1A-A | 16.6 |
| KR080B-1A-B | 17.7 |
| KR080B-1B-A | 17.4 |
| KR080B-1B-B | 18.8 |
| KR080B-2A-A | 18.6 |
| KR080B-2A-B | 18.3 |
| KR080B-2B-A | 17.8 |
| KR080B-2B-B | 17.6 |

The down regulation of the gene STT 23 in the KR080B construction group were assessed with RT-PCR, see RT-PCR Table 15 below for results. These results shows that the lines tested have a weakly down-regulated expression of the STT 23 gene. Construct gene specific primer sequences used were forward SEQ ID NO: 125 and reverse SEQ ID NO: 126.

TABLE 15
RT-PCR Relative expression level. The table shows the relative
expression levels for the different construction group lines
and the control trees for the gene targeted.

| Line | Relative expression |
|---|---|
| KR080 1AA-1 | 1.1 |
| KR080 1AA-2 | 0.7 |
| KR080 1AA-3 | 1.0 |
| KR080 1BA-1 | 0.4 |
| KR080 1BA-2 | 0.6 |
| KR080 1BA-3 | 0.4 |
| KR080 1BB-1 | 0.3 |
| KR080 1BB-2 | 0.5 |
| KR080 1BB-3 | 0.8 |
| KR080 2AA-1 | 0.5 |
| KR080 2AA-2 | 0.7 |
| KR080 2AA-3 | 0.7 |
| KR080 2BA-1 | 1.6 |
| KR080 2BA-2 | 0.4 |
| KR080 2BA-3 | 0.6 |
| KR080 2BB-1 | 0.5 |
| KR080 2BB-2 | 0.8 |
| KR080 2BB-3 | 0.3 |
| KR080 AB-1 | 0.3 |
| KR080 AB-2 | 0.6 |
| T89-1 | 0.7 |
| T89-15 | 0.7 |
| T89-16 | 0.6 |
| T8923 | 1.0 |
| T89-24 | 0.9 |
| T89-30 | 0.9 |
| T89-39 | 1.3 |
| T89-37 | 0.7 |
| T89-33 | 0.7 |
| T89-12 | 1.0 |
| T89-41 | 0.8 |
| T89-4 | 1.0 |
| T89-26 | 0.7 |
| T89-27 | 1.0 |
| T89-45 | 0.7 |

The table shows the relative expression levels for the different construction group lines and the control trees for the gene targeted.

| | |
|---|---|
| Number (KR080B) | 8 |
| Average (KR080B) | 17.85 |
| Standard deviation (KR080B) | 0.71 |
| Number (T89) | 7 |
| Average (T89) | 18.40 |
| Standard deviation (T89) | 0.63 |
| Max (T89) | 19.10 |
| Min (T89) | 17.40 |
| Confidence Interval (T89) (95%) | 18.4 +/− 1.53 |
| Ratio (Number (KR080B)/Average (T89)) | 0.97 |
| Ratio (Max(KR080B)/Average(T89)) | 1.02 |
| Ratio (Min(KR080B)/Average(T89)) | 0.90 |
| T-test | 0.1349 |
| Anova | 0.1381 |
| Num KR080B > Confidence Interval (T89) (95%) | 0 |
| (19.93) | |
| Num KR080B < Confidence Interval (T89) (95%) | 1 |
| (16.87) | |
| Num KR080B > Max (T89) (19.1) | 0 |
| Num KR080B < Min (T89) (17.4) | 1 |

### Gene STT 27

The gene STT 27 is a fructokinase, we here show that this gene is involved in sugar metabolism and can be used to change the lignin levels when the gene product level is modulated. Construction group KR100B shows a maximum increase of lignin of 13% compared to the wild type average, see Table 16 below.

TABLE 16

| Individual Name | Lignin Content (%) |
|---|---|
| T89-119 | 18.8 |
| T89-120 | 18.6 |
| T89-121 | 18.8 |
| T89-122 | 19 |
| T89-123 | 18.1 |
| T89-124 | 18.7 |
| T89-125 | 19.4 |
| T89-126 | 19.5 |
| T89-127 | 19.7 |
| T89-128 | 19.2 |
| T89-129 | 19.1 |
| T89-130 | 19 |
| T89-131 | 19.6 |
| T89-132 | 18.6 |
| KR100B-1B | 19 |
| KR100B-2A | 21.4 |
| KR100B-2B | 18.2 |
| KR100B-3B | 20.1 |
| KR015B-5B | 19.3 |

| | |
|---|---|
| Number (KR100B) | 4 |
| Average (KR100B) | 19.68 |
| Standard deviation (KR100B) | 1.39 |
| Number (T89) | 14 |
| Average (T89) | 19.01 |
| Standard deviation (T89) | 0.45 |
| Max (T89) | 19.70 |
| Min (T89) | 18.10 |
| Confidence Interval (T89) (95%) | 19.01 +/− 0.97 |
| Ratio (Number (KR100B)/Average (T89)) | 1.04 |
| Ratio (Max(KR100B)/Average(T89)) | 1.13 |
| Ratio (Min(KR100B)/Average(T89)) | 0.96 |
| T-test | 0.4096 |
| Anova | 0.1235 |
| Num KR100B > Confidence Interval (T89) (95%) | 2 |
| (19.98) | |
| Num KR100B < Confidence Interval (T89) (95%) | 0 |
| (18.04) | |
| Num KR100B > Max (T89) (19.7) | 2 |
| Num KR100B < Min (T89) (18.1) | 0 |

### Gene STT 31

STT 31 is a gene coding for a protein in the disulfide isomerase protein family involved in protein biogenesis. The construction group KR111 shows a maximum decrease of lignin of 19% compared to wild type average, see Table 17 below.

TABLE 17

| Individual Name | Lignin Content (%) |
|---|---|
| T89-66 | 19.5 |
| T89-67 | 22.6 |
| T89-68 | 21 |
| T89-69 | 21.4 |
| T89-70 | 20.4 |
| T89-71 | 22.1 |
| T89-72 | 20.7 |
| T89-73 | 19.9 |
| KR111-1B | 22.5 |
| KR111-3A-B | 18.3 |
| KR111-3B | 21.6 |
| KR111-6A-A | 16.9 |
| KR111-6A-B | 20.3 |

| | |
|---|---|
| Number (KR111) | 5 |
| Average (KR111) | 19.92 |
| Standard deviation (KR111) | 2.31 |
| Number (T89) | 8 |
| Average (T89) | 20.95 |
| Standard deviation (T89) | 1.06 |
| Max (T89) | 22.60 |
| Min (T89) | 19.50 |
| Confidence Interval (T89) (95%) | 20.95 +/− 2.5 |
| Ratio (Number (KR111)/Average (T89)) | 0.95 |
| Ratio (Max(KR111)/Average(T89)) | 1.07 |
| Ratio (Min(KR111)/Average(T89)) | 0.81 |
| T-test | 0.3912 |
| Anova | 0.2910 |
| Num KR111 > Confidence Interval (T89) (95%) (23.45) | 0 |
| Num KR111 < Confidence Interval (T89) (95%) (18.45) | 2 |
| Num KR111 > Max (T89) (22.6) | 0 |
| Num KR111 < Min (T89) (19.5) | 2 |

### Gene STT 32

This gene is a Sucrose synthase gene, susy. Suzy is well known to be involved in cell wall synthesis. We here show that this gene can be used to modulate lignin levels in trees. Construction group KR112 shows. A maximum increase of lignin of 12% compared to wild type average, see Table 18 below.

TABLE 18

| Individual Name | Lignin Content (%) |
|---|---|
| T89-24 | 20.8 |
| T89-25 | 19.9 |
| T89-26 | 18 |
| T89-27 | 20.4 |
| T89-28 | 19 |
| T89-29 | 20.5 |
| T89-30 | 19.1 |
| T89-31 | 20.6 |
| T89-32 | 19.8 |
| T89-34 | 18.3 |
| T89-38 | 20.3 |
| T89-39 | 19.5 |
| T89-40 | 18.1 |
| T89-41 | 19.5 |
| KR112 1A | 19.2 |
| KR112 2B | 21.9 |
| KR112 3B | 20.4 |
| KR112 4A | 18.2 |
| KR112 4B | 21.5 |

The down regulation of the gene STT 32 in the KR112 construction group were assessed with RT-PCR, see RT-PCR Table 19 for results. These results shows that the four lines tested have a down-regulated expression of the STT 32 gene. The three lines with the highest down-regulation also have the highest lignin levels. Construct gene specific primer sequences used were forward SEQ ID NO: 121 and reverse SEQ ID NO: 122.

TABLE 19
RT-PCR Relative expression level. The table shows the relative
expression levels for the different construction group lines
and the control trees for the gene targeted.

| Line | Relative expression |
|---|---|
| KR112 1A-1 | 20.9 |
| KR112 1A-2 | 38.8 |
| KR112 1A-3 | 11.3 |
| KR112 2B-1 | 2.2 |
| KR112 2B-2 | 1.6 |
| KR112 2B-3 | 0.4 |
| KR112 3B-1 | 3.7 |
| KR112 3B-2 | 0.6 |
| KR112 3B-3 | 1.1 |
| KR112 4B-1 | 0.4 |
| KR112 4B-2 | 0.5 |
| KR112 4B-3 | 0.3 |
| T89-1 | 101.3 |
| T89-15 | 49.5 |
| T89-16 | 71.8 |
| T8923 | 89.0 |
| T89-24 | 41.9 |
| T89-30 | 75.5 |
| T89-39 | 44.4 |
| T89-37 | 59.9 |
| T89-33 | 60.2 |
| T89-12 | 103.2 |
| T89-41 | 48.9 |
| T89-4 | 61.4 |
| T89-26 | 50.5 |
| T89-27 | 64.2 |
| T89-45 | 27.8 |

| | |
|---|---|
| Number (KR112) | 5 |
| Average (KR112) | 20.24 |
| Standard deviation (KR112) | 1.55 |
| Number (T89) | 14 |
| Average (T89) | 19.56 |
| Standard deviation (T89) | 0.95 |
| Max (T89) | 20.80 |
| Min (T89) | 18.00 |
| Confidence Interval (T89) (95%) | 19.56 +/− 2.04 |
| Ratio (Number (KR112)/Average (T89)) | 1.03 |
| Ratio (Max(KR112)/Average(T89)) | 1.12 |
| Ratio (Min(KR112)/Average(T89)) | 0.93 |
| T-test | 0.3964 |
| Anova | 0.2570 |
| Num KR112 > Confidence Interval (T89) (95%) (21.6) | 1 |
| Num KR112 < Confidence Interval (T89) (95%) (17.52) | 0 |
| Num KR112 > Max (T89) (20.8) | 2 |
| Num KR112 < Min (T89) (18) | 0 |

### Gene STT 40

STT 40 is a gene coding for a LIM domain transcription factor. We here show that this gene can be used to modulate lignin levels in trees. Construction group KR126 shows a maximum decrease of lignin of 16% compared to the wild type average, see Table 20 below.

TABLE 20

| Individual Name | Lignin Content (%) |
|---|---|
| T89-24 | 20.8 |
| T89-25 | 19.9 |
| T89-26 | 18 |
| T89-27 | 20.4 |
| T89-28 | 19 |
| T89-29 | 20.5 |
| T89-30 | 19.1 |
| T89-31 | 20.6 |
| T89-32 | 19.8 |
| T89-34 | 18.3 |
| T89-38 | 20.3 |
| T89-39 | 19.5 |
| T89-40 | 18.1 |
| T89-41 | 19.5 |
| KR126 2A | 18.8 |
| KR126 2B | 16.4 |
| KR126 3A | 20.6 |
| KR126 4B | 19.9 |
| KR126 5A | 18.4 |

| | |
|---|---|
| Number (KR126) | 5 |
| Average (KR126) | 18.82 |
| Standard deviation (KR126) | 1.61 |
| Number (T89) | 14 |
| Average (T89) | 19.56 |
| Standard deviation (T89) | 0.95 |
| Max (T89) | 20.80 |
| Min (T89) | 18.00 |
| Confidence Interval (T89) (95%) | 19.56 +/− 2.04 |
| Ratio (Number (KR126)/Average (T89)) | 0.96 |
| Ratio (Max(KR126)/Average(T89)) | 1.05 |
| Ratio (Min(KR126)/Average(T89)) | 0.84 |
| T-test | 0.3782 |
| Anova | 0.2303 |
| Num KR126 > Confidence Interval (T89) (95%) (21.6) | 0 |
| Num KR126 < Confidence Interval (T89) (95%) (17.52) | 1 |
| Num KR126 > Max (T89) (20.8) | 0 |
| Num KR126 < Min (T89) (18) | 1 |

### Gene STT 49

The RNAi construction group KR140 shows an maximum decrease of lignin of 12% compared to wild type average, see Table 21 below. The corresponding gene STT 49 is of unknown function.

TABLE 21

| Individual Name | Lignin Content (%) |
|---|---|
| T89-24 | 20.8 |
| T89-25 | 19.9 |
| T89-26 | 18 |
| T89-27 | 20.4 |
| T89-28 | 19 |
| T89-29 | 20.5 |
| T89-30 | 19.1 |
| T89-31 | 20.6 |
| T89-32 | 19.8 |
| T89-34 | 18.3 |
| T89-38 | 20.3 |
| T89-39 | 19.5 |
| T89-40 | 18.1 |
| T89-41 | 19.5 |
| KR140 1A | 18.6 |
| KR140 2A | 18.7 |
| KR140 4A | 18.5 |
| KR140 5A | 17.3 |
| KR140 5B | 17.7 |

| | |
|---|---|
| Number (KR140) | 5 |
| Average (KR140) | 18.16 |
| Standard deviation (KR140) | 0.62 |
| Number (T89) | 14 |
| Average (T89) | 19.56 |
| Standard deviation (T89) | 0.95 |
| Max(T89) | 20.80 |
| Min(T89) | 18.00 |
| Confidence Interval (T89) (95%) | 19.56 +/− 2.04 |
| Ratio (Average (KR140)/Average (T89)) | 0.93 |
| Ratio (Max(KR140)/Average(T89)) | 0.96 |
| Ratio (Min(KR140)/Average(T89)) | 0.88 |
| T-test | 0.0034 |
| Anova | 0.0073 |
| Num KR140 > Confidence Interval (T89) (95%) (21.6) | 0 |
| Num KR140 < Confidence Interval (T89) (95%) (17.52) | 1 |
| Num KR140 > Max(T89) (20.8) | 0 |
| Num KR140 < Min(T89) (18) | 2 |

### Gene STT 54

This gene is homologous to quinone reductases. The RNAi construction group KR151 shows a maximum decrease of lignin of 10% compared to the wild type average, see Table 22 below.

TABLE 22

| Individual Name | Lignin Content (%) |
|---|---|
| T89-66 | 19.5 |
| T89-67 | 22.6 |
| T89-68 | 21 |
| T89-69 | 21.4 |
| T89-70 | 20.4 |
| T89-71 | 22.1 |
| T89-72 | 20.7 |
| T89-73 | 19.9 |
| KR151-3A | 20 |
| KR151-4B | 21.1 |
| KR151-5A-A | 18.8− |
| KR151-5A-B | 19.1− |
| KR151-5B | 20.3 |

| | |
|---|---|
| Number (KR151) | 5 |
| Average (KR151) | 19.86 |
| Standard deviation (KR151) | 0.93 |
| Number (T89) | 8 |
| Average (T89) | 20.95 |
| Standard deviation (T89) | 1.06 |
| Max (T89) | 22.60 |
| Min (T89) | 19.50 |
| Confidence Interval (T89) (95%) | 20.95 +/− 2.5 |
| Ratio (Number (KR151)/Average (T89)) | 0.95 |
| Ratio (Max(KR151)/Average(T89)) | 1.01 |
| Ratio (Min(KR151)/Average(T89)) | 0.90 |
| T-test | 0.0811 |
| Anova | 0.0856 |
| Num KR151 > Confidence Interval (T89) (95%) (23.45) | 0 |
| Num KR151 < Confidence Interval (T89) (95%) (18.45) | 0 |
| Num KR151 > Max (T89) (22.6) | 0 |
| Num KR151 < Min (T89) (19.5) | 2 |

### Gene STT 57

The gene STT 57 is inserted and down-regulated in the RNAi construct KR154, which shows up to a maximum decrease of lignin of 11% compared to wild type average, see Table 23 below. The gene STT 57 is homologous to genes suggested to be related to the transcription process and low light responses published in WO2008006033.

TABLE 23

| Individual Name | Lignin Content (%) |
|---|---|
| T89-24 | 20.8 |
| T89-25 | 19.9 |
| T89-26 | 18 |
| T89-27 | 20.4 |
| T89-28 | 19 |
| T89-29 | 20.5 |
| T89-30 | 19.1 |
| T89-31 | 20.6 |
| T89-32 | 19.8 |
| T89-34 | 18.3 |
| T89-38 | 20.3 |
| T89-39 | 19.5 |
| T89-40 | 18.1 |
| T89-41 | 19.5 |
| KR154 1B | 18 |
| KR154 2A | 18.1 |
| KR154 2B | 17.9 |
| KR154 3A-A | 18.7 |
| KR154 3A-B | 18.4 |
| KR154 4A | 17.5 |
| KR154 5B-B | 17.9 |

| | |
|---|---|
| Number (KR154) | 7 |
| Average (KR154) | 18.07 |
| Standard deviation (KR154) | 0.39 |
| Number (T89) | 14 |
| Average (T89) | 19.56 |
| Standard deviation (T89) | 0.95 |
| Max(T89) | 20.80 |
| Min(T89) | 18.00 |
| Confidence Interval (T89) (95%) | 19.56 +/− 2.04 |
| Ratio (Average (KR154)/Average (T89)) | 0.92 |
| Ratio (Max(KR154)/Average(T89)) | 0.96 |
| Ratio (Min(KR154)/Average(T89)) | 0.89 |
| T-test | 0.0001 |
| Anova | 0.0008 |
| Num KR154 > Confidence Interval (T89) (95%) (21.6) | 0 |
| Num KR154 < Confidence Interval (T89) (95%) (17.52) | 1 |
| Num KR154 > Max(T89) (20.8) | 0 |
| Num KR154 < Min(T89) (18) | 3 |

The gene STT 57 was further over expressed in the construct 35S008. The construct 35S008 showed a maximum increase of lignin of 15% compared to the wild type average, see Table 24 below.

TABLE 24

| Individual Name | Lignin Content (%) |
|---|---|
| T89-12 | 19 |
| T89-19 | 19.3 |
| T89-29 | 18.7 |
| T89-43 | 19.5 |
| T89-8 | 17.8 |
| T89-9 | 16.9 |
| 35s008-1AA | 21.4 |
| 35s008-2A | 18.9 |
| 35s008-3AA | 17.8 |
| 35s008-3B | 17.7 |

| | |
|---|---|
| Number (35s008) | 4 |
| Average (35s008) | 18.95 |
| Standard deviation (35s008) | 1.72 |
| Number (T89) | 6 |
| Average (T89) | 18.53 |
| Standard deviation (T89) | 1.00 |
| Max (T89) | 19.50 |
| Min (T89) | 16.90 |
| Confidence Interval (T89) (95%) | 18.53 +/− 2.56 |
| Ratio (Number (35s008)/Average (T89)) | 1.02 |
| Ratio (Max(35s008)/Average(T89)) | 1.15 |
| Ratio (Min(35s008)/Average(T89)) | 0.96 |
| T-test | 0.6825 |
| Anova | 0.6371 |
| Num 35s008 > Confidence Interval (T89) (95%) (21.1) | 1 |
| Num 35s008 < Confidence Interval (T89) (95%) (15.97) | 0 |
| Num 35s008 > Max (T89) (19.5) | 1 |
| Num 35s008 < Min (T89) (16.9) | 0 |

### Gene STT 59

The STT 59 gene is a methylenetetrahydrofolate reductase which irreversibly reduces 5,10-methylenetetrahydrofolate to 5-methyltetrahydrofolate, which is used to convert homocysteine to methionine. There are a number of publications about this enzyme. Falco et al and US2005034176 describes the use of this gene in regulating the levels of 5,10-methylenetetrahydrofolate reductase in a transformed host cell, but not the technical use to change lignin levels in the produced xylem. The RNAi construction group KR159 gives as a maximum decrease of lignin of 12% compared to the wild type average, see Table 25 below.

TABLE 25

| Individual Name | Lignin Content (%) |
|---|---|
| T89-133 | 19.1 |
| T89-134 | 18.9 |
| T89-135 | 18.7 |
| T89-136 | 19.1 |
| T89-137 | 19.2 |
| T89-138 | 19.1 |
| T89-139 | 19.2 |
| T89-140 | 19.8 |
| T89-141 | 19.5 |
| T89-142 | 20.3 |
| T89-143 | 19.3 |
| T89-144 | 19.6 |
| T89-146 | 19.9 |
| T89-147 | 20.5 |
| T89-148 | 20.6 |
| KR159-1A | 18.6 |
| KR159-5A-A | 18.4 |
| KR159-5A-B | 17.2 |

The down regulation of the gene STT 59 in the KR159 construction group were assessed with RT-PCR, see RT-PCR Table 26 for results. These results shows that the 3 lines tested have a down-regulated expression of the STT 59 gene. Construct gene specific primer sequences used were forward SEQ ID: 119 and reverse SEQ ID 120.

Table 26 RT-PCR Relative expression level.

The table shows the relative expression levels for the different construction group lines and the control trees for the gene targeted.

TABLE 26
RT-PCR Relative expression level. The table shows the relative
expression levels for the different construction group lines
and the control trees for the gene targeted.

| Line | Relative expression |
|---|---|
| KR159 1A-1 | 3.3 |
| KR159 1A-2 | 7.8 |
| KR159 1A-3 | 12.6 |
| KR159 2A-2 | 0.6 |
| KR159 3B-1 | 1.0 |
| KR159 3B-2 | 0.8 |
| KR159 3B-3 | 0.9 |
| KR159 5A-1 | 2.1 |
| KR159 5A-2 | 2.5 |
| T89-1 | 7.1 |
| T89-15 | 7.6 |
| T89-16 | 4.7 |
| T8923 | 13.4 |
| T89-24 | 7.0 |
| T89-30 | 12.1 |
| T89-39 | 13.0 |
| T89-37 | 10.5 |
| T89-33 | 14.2 |
| T89-12 | 16.0 |
| T89-41 | 12.7 |
| T89-4 | 10.9 |
| T89-26 | 9.9 |
| T89-27 | 22.2 |
| T89-45 | 12.4 |

| | |
|---|---|
| Number (KR159) | 3 |
| Average (KR159) | 18.07 |
| Standard deviation (KR159) | 0.76 |
| Number (T89) | 15 |
| Average (T89) | 19.52 |
| Standard deviation (T89) | 0.59 |
| Max (T89) | 20.60 |
| Min (T89) | 18.70 |
| Confidence Interval (T89) (95%) | 19.52 +/− 1.26 |
| Ratio (Number (KR159)/Average (T89)) | 0.93 |
| Ratio (Max(KR159)/Average(T89)) | 0.95 |
| Ratio (Min(KR159)/Average(T89)) | 0.88 |
| T-test | 0.0658 |
| Anova | 0.0017 |
| Num KR159 > Confidence Interval (T89) (95%) (20.78) | 0 |
| Num KR159 < Confidence Interval (T89) (95%) (18.26) | 1 |
| Num KR159 > Max (T89) (20.6) | 0 |
| Num KR159 < Min (T89) (18.7) | 3 |

### Gene STT 64

The gene STT 64 is a fructokinase, we here show that this gene involved in sugar metabolism can be used to change the lignin levels when the gene product level is modulated. The construction group KR165 shows variable levels of lignin with some lines contains higher and some lines contain lower amounts of lignin. A maximum decrease of lignin of 15% compared to wild type average and a maximum increase of lignin of 10% compared to wild type average in different transgenic lines, for lignin content see Table 27 below. It is obvious that in order to use this gene for manipulating lignin levels one have to produce many lines and measure lignin levels in those and then selecting transgenic lines with the desirable trait.

TABLE 27

| Individual Name | Lignin Content (%) |
|---|---|
| T89-133 | 19.1 |
| T89-134 | 18.9 |
| T89-135 | 18.7 |
| T89-136 | 19.1 |
| T89-137 | 19.2 |
| T89-138 | 19.1 |
| T89-139 | 19.2 |
| T89-140 | 19.8 |
| T89-141 | 19.5 |
| T89-142 | 20.3 |
| T89-143 | 19.3 |
| T89-144 | 19.6 |
| T89-146 | 19.9 |
| T89-147 | 20.5 |
| T89-148 | 20.6 |
| KR165-3A-B | 21.4 |
| KR165-4B-A | 18.4 |
| KR165-4B-B | 16.6 |
| KR165-5 | 20.1 |

| | |
|---|---|
| Number (KR165) | 4 |
| Average (KR165) | 19.13 |
| Standard deviation (KR165) | 2.08 |
| Number (T89) | 15 |
| Average (T89) | 19.52 |
| Standard deviation (T89) | 0.59 |
| Max (T89) | 20.60 |
| Min (T89) | 18.70 |
| Confidence Interval (T89) (95%) | 19.52 +/− 1.26 |
| Ratio (Number (KR165)/Average (T89)) | 0.98 |
| Ratio (Max(KR165)/Average(T89)) | 1.10 |
| Ratio (Min(KR165)/Average(T89)) | 0.85 |
| T-test | 0.7315 |
| Anova | 0.5023 |
| Num KR165 > Confidence Interval (T89) (95%) (20.78) | 1 |
| Num KR165 < Confidence Interval (T89) (95%) (18.26) | 1 |
| Num KR165 > Max (T89) (20.6) | 1 |
| Num KR165 < Min (T89) (18.7) | 2 |

### Gene STT 70

This gene has a weak homology to genes suggested to belong to DBP family of transcription factors. The RNAi construction group KR175 shows a maximum decrease of lignin of 9% compared to the wild type average, when the gene was down-regulated, See Table 28 below.

TABLE 28

| Individual Name | Lignin Content (%) |
|---|---|
| T89-66 | 19.5 |
| T89-67 | 22.6 |
| T89-68 | 21 |
| T89-69 | 21.4 |
| T89-70 | 20.4 |
| T89-71 | 22.1 |
| T89-72 | 20.7 |
| T89-73 | 19.9 |
| KR175-1B | 19.8 |
| KR175-2B | 20 |
| KR175-5A | 19.1 |
| KR175-5B | 19.6 |
| KR175-6A | 19.8 |
| KR175-7A | 19.3 |

| | |
|---|---|
| Number (KR175) | 6 |
| Average (KR175) | 19.60 |
| Standard deviation (KR175) | 0.34 |
| Number (T89) | 8 |
| Average (T89) | 20.95 |
| Standard deviation (T89) | 1.06 |
| Max(T89) | 22.60 |
| Min(T89) | 19.50 |
| Confidence Interval (T89) (95%) | 20.95 +/− 2.5 |
| Ratio (Average (KR175)/Average (T89)) | 0.94 |
| Ratio (Max(KR175)/Average(T89)) | 0.95 |
| Ratio (Min(KR175)/Average(T89)) | 0.91 |
| T-test | 0.0083 |
| Anova | 0.0113 |
| Num KR175 > Confidence Interval (T89) (95%) (23.45) | 0 |
| Num KR175 < Confidence Interval (T89) (95%) (18.45) | 0 |
| Num KR175 > Max(T89) (22.6) | 0 |
| Num KR175 < Min(T89) (19.5) | 2 |

### Gene STT 74

This gene are of unknown function, one gene similar to this gene ATMAMI, is annotated as an mannitol induced protein at the NCBI gene database. The RNAi construction group KR179 has a maximum decrease of lignin of 10% compared to the wild type average, see Table 29 below.

TABLE 29

| Individual Name | Lignin Content (%) |
|---|---|
| T89-100 | 22.1 |
| T89-101 | 19.7 |
| T89-102 | 21 |
| T89-89 | 18.4 |
| T89-92 | 21.6 |
| T89-93 | 22.2 |
| T89-94 | 23.2 |
| T89-95 | 21 |
| T89-96 | 21.9 |
| T89-97 | 21 |
| T89-98 | 22 |
| T89-99 | 21.5 |
| KR179-1A | 19.2 |
| KR179-1B | 20.1 |
| KR179-2A | 19.3 |
| KR179-2B | 20.3 |

| | |
|---|---|
| Number (KR179) | 4 |
| Average (KR179) | 19.73 |
| Standard deviation (KR179) | 0.56 |
| Number (T89) | 12 |
| Average (T89) | 21.30 |
| Standard deviation (T89) | 1.25 |
| Max (T89) | 23.20 |
| Min (T89) | 18.40 |
| Confidence Interval (T89) (95%) | 21.3 +/− 2.76 |
| Ratio (Number (KR179)/Average (T89)) | 0.93 |
| Ratio (Max (KR179)/Average (T89)) | 0.95 |
| Ratio (Min (KR179)/Average (T89)) | 0.90 |
| T-test | 0.0047 |
| Anova | 0.0313 |
| Num KR179 > Confidence Interval (T89) (95%) (24.06) | 0 |
| Num KR179 < Confidence Interval (T89) (95%) (18.54) | 0 |
| Num KR179 > Max (T89) (23.2) | 0 |
| Num KR179 < Min (T89) (18.4) | 0 |

### Gene STT 75

This gene is a GTPase activating protein with a RabGAP/TBC domain. One of the genes homologues to STT 75, OsGAP1, is published to be a positive regulator of OsRab11 which is involved in vesicular trafficking to the plasma membrane. (Heo et al. Plant and Cell Physiology (2005), 46(12), 2005-2018). It is plausible that some of the cargo proteins in this trafficking is part of the mechanism which gives a reduced lignin in the RNAi construction group KR182, in which the STT 75 gene is down-regulated. The KR182 construction group gives a maximum decrease of lignin of 8% compared to wild the type average, see Table 30 below.

TABLE 30

| Individual Name | Lignin Content (%) |
|---|---|
| T89-100 | 22.1 |
| T89-101 | 19.7 |
| T89-102 | 21 |
| T89-89 | 18.4 |
| T89-92 | 21.6 |
| T89-93 | 22.2 |
| T89-94 | 23.2 |
| T89-95 | 21 |
| T89-96 | 21.9 |
| T89-97 | 21 |
| T89-98 | 22 |
| KR182-1B-A | 20.1 |
| KR182-1B-B | 20.6 |
| KR182-2A | 20 |
| KR182-4A-B | 19.7 |
| KR182-5B | 20 |

| | |
|---|---|
| Number (KR182) | 5 |
| Average (KR182) | 20.08 |
| Standard deviation (KR182) | 0.33 |
| Number (T89) | 12 |
| Average (T89) | 21.30 |
| Standard deviation (T89) | 1.25 |
| Max (T89) | 23.20 |
| Min (T89) | 18.40 |
| Confidence Interval (T89) (95%) | 21.3 +/− 2.76 |
| Ratio (Number (KR182)/Average (T89)) | 0.94 |
| Ratio (Max(KR182)/Average (T89)) | 0.97 |
| Ratio (Min (KR182)/Average (T89)) | 0.92 |
| T-test | 0.0075 |
| Anova | 0.0521 |
| Num KR182 > Confidence Interval (T89) (95%) (24.06) | 0 |
| Num KR182 < Confidence Interval (T89) (95%) (18.54) | 0 |
| Num KR182 > Max (T89) (23.2) | 0 |
| Num KR182 < Min (T89) (18.4) | 0 |

### Gene STT 76

STT 76 is a gene of unknown function, the RNAi construct KR183 gives a maximum decrease of lignin of 20% compared to the wild type average, see Table 31 below.

TABLE 31

| Individual Name | Lignin Content (%) |
|---|---|
| T89-103 | 18.9 |
| T89-104 | 19 |
| T89-106 | 17.8 |
| T89-107 | 19.3 |
| T89-108 | 19.7 |
| T89-109 | 19 |
| T89-110 | 19.7 |
| T89-111 | 18.6 |
| T89-112 | 19.8 |
| T89-113 | 19.2 |
| T89-114 | 20.1 |
| T89-115 | 18.6 |
| KR183-1A | 16.8 |
| KR183-2 | 19.2 |
| KR183-3A | 17.6 |
| KR183-3B | 19 |
| KR183-6B-B | 15.4 |

| | |
|---|---|
| Number (KR183) | 5 |
| Average (KR183) | 17.60 |
| Standard deviation (KR183) | 1.58 |
| Number (T89) | 12 |
| Average (T89) | 19.14 |
| Standard deviation (T89) | 0.64 |
| Max (T89) | 20.10 |
| Min (T89) | 17.80 |
| Confidence Interval (T89) (95%) | 19.14 +/− 1.41 |
| Ratio (Number (KR183)/Average (T89)) | 0.92 |
| Ratio (Max (KR192)/Average (T89)) | 1.00 |
| Ratio (Min (KR192)/Average (T89)) | 0.80 |
| T-test | 0.0941 |
| Anova | 0.0100 |
| Num KR183 > Confidence Interval (T89) (95%) (20.55) | 0 |
| Num KR183 < Confidence Interval (T89) (95%) (17.74) | 3 |
| Num KR183 > Max (T89) (20.1) | 0 |
| Num KR183 < Min (T89) (17.8) | 3 |

### Gene STT 82

The RNAi construction group KR192 giva a maximum decrease of lignin of 5% compared to the wild type average, see Table 32 below.

TABLE 32

| Individual Name | Lignin Content (%) |
|---|---|
| T89-103 | 18.9 |
| T89-104 | 19 |
| T89-106 | 17.8 |
| T89-107 | 19.3 |
| T89-108 | 19.7 |
| T89-109 | 19 |
| T89-110 | 19.7 |
| T89-111 | 18.6 |
| T89-112 | 19.8 |
| T89-113 | 19.2 |
| T89-114 | 20.1 |
| T89-115 | 18.6 |
| KR192-1A | 18.6 |
| KR192-2A | 19.1 |
| KR192-3A | 18.4 |
| KR192-3B | 18.3 |
| KR192-4 | 18.2 |

| | |
|---|---|
| Number (KR192) | 5 |
| Average (KR192) | 18.52 |
| Standard deviation (KR192) | 0.36 |
| Number (T89) | 12 |
| Average (T89) | 19.14 |
| Standard deviation (T89) | 0.64 |
| Max (T89) | 20.10 |
| Min (T89) | 17.80 |
| Confidence Interval (T89) (95%) | 19.14 +/− 1.41 |
| Ratio (Number (KR192)/Average (T89)) | 0.97 |
| Ratio (Max (KR192)/Average (T89)) | 1.00 |
| Ratio (Min (KR192)/Average (T89)) | 0.95 |
| T-test | 0.0239 |
| Anova | 0.0612 |
| Num KR192 > Confidence Interval (T89) (95%) (20.55) | 0 |
| Num KR192 < Confidence Interval (T89) (95%) (17.74) | 0 |
| Num KR192 > Max (T89) (20.1) | 0 |
| Num KR192 < Min (T89) (17.8) | 0 |

### Gene STT 83

The RNAi construction group KR198 shows a maximum decrease of lignin of 7% compared to the wild type average, see Table 33 below.

TABLE 33

| Individual Name | Lignin Content (%) |
|---|---|
| T89-133 | 19.1 |
| T89-134 | 18.9 |
| T89-135 | 18.7 |
| T89-136 | 19.1 |
| T89-137 | 19.2 |
| T89-138 | 19.1 |
| T89-139 | 19.2 |
| T89-140 | 19.8 |
| T89-141 | 19.5 |
| T89-142 | 20.3 |
| T89-143 | 19.3 |
| T89-144 | 19.6 |
| T89-146 | 19.9 |
| T89-147 | 20.5 |
| T89-148 | 20.6 |
| KR198-5A | 19.8 |
| KR198-5A-A1 | 19.4 |
| KR198-5A-A2 | 18.1 |
| KR198-5B-2 | 19.5 |
| KR198-5B-3 | 20.3 |

| | |
|---|---|
| Number (KR198) | 5 |
| Average (KR198) | 19.42 |
| Standard deviation (KR198) | 0.82 |
| Number (T89) | 15 |
| Average (T89) | 19.52 |
| Standard deviation (T89) | 0.59 |
| Max (T89) | 20.60 |
| Min (T89) | 18.70 |
| Confidence Interval (T89) (95%) | 19.52 +/− 1.26 |
| Ratio (Number (KR198)/Average (T89)) | 0.99 |
| Ratio (Max(KR198)/Average(T89)) | 1.04 |
| Ratio (Min(KR198)/Average(T89)) | 0.93 |
| T-test | 0.8096 |
| Anova | 0.7672 |
| Num KR198 > Confidence Interval (T89) (95%) (20.78) | 0 |
| Num KR198 < Confidence Interval (T89) (95%) (18.26) | 1 |
| Num KR198 > Max (T89) (20.6) | 0 |
| Num KR198 < Min (T89) (18.7) | 1 |

### Gene STT 95

The gene STT 95 codes for a pectate lyase, pectin is a substance in the plant cell wall. We here show that modulating the level of Pecatate lyase genes can be used to modify lignin levels in wood. The RNAi construction group KR213 shows a maximum increase of lignin of 12% compared to the wild type average see Table 34 below.

TABLE 34

| Individual Name | Lignin Content (%) |
|---|---|
| T89-12 | 18.5 |
| T89-17 | 18.4 |
| T89-18 | 19.2 |
| T89-20 | 19.1 |
| T89-21 | 18.3 |
| T89-5 | 17.1 |
| T89-9 | 19.1 |
| KR213-1A-1 | 20.8 |
| KR213-2B-A | 18.3 |
| KR213-2B-B1 | 18.8 |
| KR213-4B-1 | 20.1 |

| | |
|---|---|
| Number (KR213) | 4 |
| Average (KR213) | 19.50 |
| Standard deviation (KR213) | 1.15 |
| Number (T89) | 7 |
| Average (T89) | 18.53 |
| Standard deviation (T89) | 0.73 |
| Max (T89) | 19.20 |
| Min (T89) | 17.10 |
| Confidence Interval (T89) (95%) | 18.53 +/− 1.79 |
| Ratio (Number (KR213)/Average (T89)) | 1.05 |
| Ratio (Max(KR213)/Average(T89)) | 1.12 |
| Ratio (Min(KR213)/Average(T89)) | 0.99 |
| T-test | 0.1963 |
| Anova | 0.1170 |
| Num KR213 > Confidence Interval (T89) (95%) (20.32) | 1 |
| Num KR213 < Confidence Interval (T89) (95%) (16.74) | 0 |
| Num KR213 > Max (T89) (19.2) | 2 |
| Num KR213 < Min (T89) (17.1) | 0 |

### Gene STT 97

The RNAi construction group KR215 shows a maximum increase of lignin of 5% compared to the wild type average, see Table 35 below.

TABLE 35

| Individual Name | Lignin Content (%) |
|---|---|
| T89-12 | 18.5 |
| T89-17 | 18.4 |
| T89-18 | 19.2 |
| T89-20 | 19.1 |
| T89-21 | 18.3 |
| T89-5 | 17.1 |
| T89-9 | 19.1 |
| KR215-1A | 17.7 |
| KR215-2A | 18.1 |
| KR215-3A | 19.3 |
| KR215-3B | 18.7 |
| KR215-4B | 19.5 |
| KR215-6A | 18.3 |
| KR215-8A | 18.7 |

| | |
|---|---|
| Number (KR215) | 7 |
| Average (KR215) | 18.61 |
| Standard deviation (KR215) | 0.64 |
| Number (T89) | 7 |
| Average (T89) | 18.53 |
| Standard deviation (T89) | 0.73 |
| Max (T89) | 19.20 |
| Min (T89) | 17.10 |
| Confidence Interval (T89) (95%) | 18.53 +/− 1.79 |
| Ratio (Number (KR215)/Average (T89)) | 1.00 |
| Ratio (Max(KR215)/Average(T89)) | 1.05 |
| Ratio (Min(KR215)/Average(T89)) | 0.96 |
| T-test | 0.8197 |
| Anova | 0.8197 |
| Num KR215 > Confidence Interval (T89) (95%) (20.32) | 0 |
| Num KR215 < Confidence Interval (T89) (95%) (16.74) | 0 |
| Num KR215 > Max (T89) (19.2) | 2 |
| Num KR215 < Min (T89) (17.1) | 0 |

### Gene STT 101

The RNAi construction group KR223 shows a maximum decrease of lignin of 6% compared to the wild type average, see Table 36 below.

TABLE 36

| Individual Name | Lignin Content (%) |
|---|---|
| T89-119 | 18.8 |
| T89-120 | 18.6 |
| T89-121 | 18.8 |
| T89-122 | 19 |
| T89-123 | 18.1 |
| T89-124 | 18.7 |
| T89-125 | 19.4 |
| T89-126 | 19.5 |
| T89-127 | 19.7 |
| T89-128 | 19.2 |
| T89-129 | 19.1 |
| T89-130 | 19 |
| T89-131 | 19.6 |
| T89-132 | 18.6 |
| KR223-1B | 17.8 |
| KR223-2B | 18.8 |
| KR223-3A | 19.6 |
| KR223-3B | 19.6 |
| KR223-4B | 19.2 |

| | |
|---|---|
| Number (KR223) | 5 |
| Average (KR223) | 19.00 |
| Standard deviation (KR223) | 0.75 |
| Number (T89) | 14 |
| Average (T89) | 19.01 |
| Standard deviation (T89) | 0.45 |
| Max (T89) | 19.70 |
| Min (T89) | 18.10 |
| Confidence Interval (T89) (95%) | 19.01 +/− 0.97 |
| Ratio (Number (KR223)/Average (T89)) | 1.00 |
| Ratio (Max(KR223)/Average(T89)) | 1.03 |
| Ratio (Min(KR223)/Average(T89)) | 0.94 |
| T-test | 0.9847 |
| Anova | 0.9798 |
| Num KR223 > Confidence Interval (T89) (95%) (19.98) | 0 |
| Num KR223 < Confidence Interval (T89) (95%) (18.04) | 1 |
| Num KR223 > Max (T89) (19.7) | 0 |
| Num KR223 < Min (T89) (18.1) | 1 |

### Gene STT 116

This gene is a small GTP-binding protein similar to the Pra2 gene. The Pra gene is a dark induced small G protein that regulates an P450 cytochrome that is involved in brassosteroid synthesis (Kang et al, Cell. 2001 Jun. 1; 105(5):625-36). The RNAi construction group KR242 shows a maximum decrease of lignin of 9% compared to the wild type average see Table 37 below.

TABLE 37

| Individual Name | Lignin Content (%) |
|---|---|
| T89-24 | 20.8 |
| T89-25 | 19.9 |
| T89-26 | 18 |
| T89-27 | 20.4 |
| T89-28 | 19 |
| T89-29 | 20.5 |
| T89-30 | 19.1 |
| T89-31 | 20.6 |
| T89-32 | 19.8 |
| T89-34 | 18.3 |
| T89-38 | 20.3 |
| T89-39 | 19.5 |
| T89-40 | 18.1 |
| T89-41 | 19.5 |
| KR242 1A | 17.7 |
| KR242 1B | 19 |
| KR242 2B | 18.6 |
| KR242 3A | 19 |
| KR242 4A | 19.4 |
| KR242 4B | 18.7 |
| KR242 5A | 18.6 |
| KR242 5B | 18.8 |

| | |
|---|---|
| Number (KR242) | 8 |
| Average (KR242) | 18.73 |
| Standard deviation (KR242) | 0.49 |
| Number (T89) | 14 |
| Average (T89) | 19.56 |
| Standard deviation (T89) | 0.95 |
| Max(T89) | 20.80 |
| Min(T89) | 18.00 |
| Confidence Interval (T89) (95%) | 19.56 +/− 2.04 |
| Ratio (Average (KR242)/Average (T89)) | 0.96 |
| Ratio (Max (KR242)/Average (T89)) | 0.99 |
| Ratio (Min (KR242)/Average (T89)) | 0.91 |
| T-test | 0.0134 |
| Anova | 0.0322 |
| Num KR242 > Confidence Interval (T89) (95%) (21.6) | 0 |
| Num KR242 < Confidence Interval (T89) (95%) (17.52) | 0 |
| Num KR242 > Max(T89) (20.8) | 0 |
| Num KR242 < Min(T89) (18) | 1 |

### Gene STT 124

The RNAi construction group KR287 shows a maximum decrease of lignin of 8% compared to the wild type average see Table 38 below.

TABLE 38

| Individual Name | Lignin Content (%) |
|---|---|
| T89-103 | 18.9 |
| T89-104 | 19 |
| T89-106 | 17.8 |
| T89-107 | 19.3 |
| T89-108 | 19.7 |
| T89-109 | 19 |
| T89-110 | 19.7 |
| T89-111 | 18.6 |
| T89-112 | 19.8 |
| T89-113 | 19.2 |
| T89-114 | 20.1 |
| T89-115 | 18.6 |
| KR287-4A | 18.4 |
| KR287-4B | 19.1 |
| KR287-6A | 17.6 |
| KR287-6B | 18.2 |
| KR287-7B | 19.1 |

| | |
|---|---|
| Number (KR287) | 5 |
| Average (KR287) | 18.48 |
| Standard deviation (KR287) | 0.64 |
| Number (T89) | 12 |
| Average (T89) | 19.14 |
| Standard deviation (T89) | 0.64 |
| Max (T89) | 20.10 |
| Min (T89) | 17.80 |
| Confidence Interval (T89) (95%) | 19.14 +/− 1.41 |
| Ratio (Number (KR287)/Average (T89)) | 0.97 |
| Ratio (Max(KR287)/Average(T89)) | 1.00 |
| Ratio (Min(KR287)/Average(T89)) | 0.92 |
| T-test | 0.0894 |
| Anova | 0.0706 |
| Num KR287 > Confidence Interval (T89) (95%) (20.55) | 0 |
| Num KR287 < Confidence Interval (T89) (95%) (17.74) | 1 |
| Num KR287 > Max (T89) (20.1) | 0 |
| Num KR287 < Min (T89) (17.8) | 1 |

### Gene STT 128

The RNAi construction group KR318 shows a maximum decrease of lignin of 7% compared to the wild type average see Table 39 below.

TABLE 39

| Individual Name | Lignin Content (%) |
|---|---|
| T89-66 | 19.5 |
| T89-67 | 22.6 |
| T89-68 | 21 |
| T89-69 | 21.4 |
| T89-70 | 20.4 |
| T89-71 | 22.1 |
| T89-72 | 20.7 |
| T89-73 | 19.9 |
| KR318-1B | 21.5 |
| KR318-3A-A | 19.4 |
| KR318-3A-B | 20.5 |
| KR318-4A | 20.3 |
| KR318-4B-B | 20.6 |

| | |
|---|---|
| Number (KR318) | 5 |
| Average (KR318) | 20.46 |
| Standard deviation (KR318) | 0.75 |
| Number (T89) | 8 |
| Average (T89) | 20.95 |
| Standard deviation (T89) | 1.06 |
| Max (T89) | 22.60 |
| Min (T89) | 19.50 |
| Confidence Interval (T89) (95%) | 20.95 +/− 2.5 |
| Ratio (Number (KR318)/Average (T89)) | 0.98 |
| Ratio (Max(KR318)/Average(T89)) | 1.03 |
| Ratio (Min(KR318)/Average(T89)) | 0.93 |
| T-test | 0.3508 |
| Anova | 0.3883 |
| Num KR318 > Confidence Interval (T89) (95%) (23.45) | 0 |
| Num KR318 < Confidence Interval (T89) (95%) (18.45) | 0 |
| Num KR318 > Max (T89) (22.6) | 0 |
| Num KR318 < Min (T89) (19.5) | 1 |

### Gene STT 165

The RNAi construction group KR470 shows a maximum decrease of lignin 7% compared to wild type average see Table 40 below.

TABLE 40

| Individual Name | Lignin Content (%) |
|---|---|
| T89-133 | 19.1 |
| T89-134 | 18.9 |
| T89-135 | 18.7 |
| T89-136 | 19.1 |
| T89-137 | 19.2 |
| T89-138 | 19.1 |
| T89-139 | 19.2 |
| T89-140 | 19.8 |
| T89-141 | 19.5 |
| T89-142 | 20.3 |
| T89-143 | 19.3 |
| T89-144 | 19.6 |
| T89-146 | 19.9 |
| T89-147 | 20.5 |
| T89-148 | 20.6 |
| KR470-1A | 18.2 |
| KR470-2A | 18.3 |
| KR470-3B-B | 18.9 |
| KR470-5A-A | 20.2 |
| KR470-5A-B | 19.4 |

| | |
|---|---|
| Number (KR470) | 5 |
| Average (KR470) | 19.00 |
| Standard deviation (KR470) | 0.83 |
| Number (T89) | 15 |
| Average (T89) | 19.52 |
| Standard deviation (T89) | 0.59 |
| Max (T89) | 20.60 |
| Min (T89) | 18.70 |
| Confidence Interval (T89) (95%) | 19.52 +/− 1.26 |
| Ratio (Number (KR470)/Average (T89)) | 0.97 |
| Ratio (Max(KR470)/Average(T89)) | 1.03 |
| Ratio (Min(KR470)/Average(T89)) | 0.93 |
| T-test | 0.2461 |
| Anova | 0.1372 |
| Num KR470 > Confidence Interval (T89) (95%) (20.78) | 0 |
| Num KR470 < Confidence Interval (T89) (95%) (18.26) | 1 |
| Num KR470 > Max (T89) (20.6) | 0 |
| Num KR470 < Min (T89) (18.7) | 2 |

### Gene STT 167

The RNAi construction group KR472 shows a maximum increase of lignin of 5% compared to the wild type average see Table 41 below.

TABLE 41

| Individual Name | Lignin Content (%) |
|---|---|
| T89-12 | 18.5 |
| T89-17 | 18.4 |
| T89-18 | 19.2 |
| T89-20 | 19.1 |
| T89-21 | 18.3 |
| T89-5 | 17.1 |
| T89-9 | 19.1 |
| KR472-1A-1 | 19.4 |
| KR472-4A-2 | 18.5 |
| KR472-5B-2 | 18.6 |

| | |
|---|---|
| Number (KR472) | 3 |
| Average (KR472) | 18.83 |
| Standard deviation (KR472) | 0.49 |
| Number (T89) | 7 |
| Average (T89) | 18.53 |
| Standard deviation (T89) | 0.73 |
| Max (T89) | 19.20 |
| Min (T89) | 17.10 |
| Confidence Interval (T89) (95%) | 18.53 +/− 1.79 |
| Ratio (Number (KR472)/Average (T89)) | 1.02 |
| Ratio (Max(KR472)/Average(T89)) | 1.05 |
| Ratio (Min(KR472)/Average(T89)) | 1.00 |
| T-test | 0.4727 |
| Anova | 0.5343 |
| Num KR472 > Confidence Interval (T89) (95%) (20.32) | 0 |
| Num KR472 < Confidence Interval (T89) (95%) (16.74) | 0 |
| Num KR472 > Max (T89) (19.2) | 1 |
| Num KR472 < Min (T89) (17.1) | 0 |

### Gene STT 170

The RNAi construction group KR475 shows a maximum increase of lignin of 5% compared to the wild type average see Table 42 below.

TABLE 42

| Individual Name | Lignin Content (%) |
|---|---|
| T89-12 | 18.5 |
| T89-17 | 18.4 |
| T89-18 | 19.2 |
| T89-20 | 19.1 |
| T89-21 | 18.3 |
| T89-5 | 17.1 |
| T89-9 | 19.1 |
| KR475-1A | 19.4 |
| KR475-1B | 18.7 |
| KR475-2B | 18.9 |
| KR475-3A | 19.2 |
| KR475-3B | 17.7 |

| | |
|---|---|
| Number (KR475) | 5 |
| Average (KR475) | 18.78 |
| Standard deviation (KR475) | 0.66 |
| Number (T89) | 7 |
| Average (T89) | 18.53 |
| Standard deviation (T89) | 0.73 |
| Max (T89) | 19.20 |
| Min (T89) | 17.10 |
| Confidence Interval (T89) (95%) | 18.53 +/− 1.79 |
| Ratio (Number (KR475)/Average (T89)) | 1.01 |
| Ratio (Max(KR475)/Average(T89)) | 1.05 |
| Ratio (Min(KR475)/Average(T89)) | 0.96 |
| T-test | 0.5495 |
| Anova | 0.5557 |
| Num KR475 > Confidence Interval (T89) (95%) (20.32) | 0 |
| Num KR475 < Confidence Interval (T89) (95%) (16.74) | 0 |
| Num KR475 > Max (T89) (19.2) | 1 |
| Num KR475 < Min (T89) (17.1) | 0 |

### Gene STT 173

EST sequences related to this gene have been found in tissues were some cells are performing controlled cell death, (Flinn et al. U.S. Pat. No. 6,451,604) we find when down-regulating the gene STT 173 in the RNAi construct KR478, that within the construction group a maximum decrease of lignin of 10% compared to the wild type average see Table 43 below.

TABLE 43

| Individual Name | Lignin Content (%) |
|---|---|
| T89-133 | 19.1 |
| T89-134 | 18.9 |
| T89-135 | 18.7 |
| T89-136 | 19.1 |
| T89-137 | 19.2 |
| T89-138 | 19.1 |
| T89-139 | 19.2 |
| T89-140 | 19.8 |
| T89-141 | 19.5 |
| T89-142 | 20.3 |
| T89-143 | 19.3 |
| T89-144 | 19.6 |
| T89-146 | 19.9 |
| T89-147 | 20.5 |
| T89-148 | 20.6 |
| KR478-1B-A | 19.4 |
| KR478-1B-B | 18.3 |
| KR478-2A | 20.2 |
| KR478-3A | 18.8 |
| KR478-4B-A | 18 |
| KR478-4B-B | 17.5 |

| | |
|---|---|
| Number (KR478) | 6 |
| Average (KR478) | 18.70 |
| Standard deviation (KR478) | 0.98 |
| Number (T89) | 15 |
| Average (T89) | 19.52 |
| Standard deviation (T89) | 0.59 |
| Max (T89) | 20.60 |
| Min (T89) | 18.70 |
| Confidence Interval (T89) (95%) | 19.52 +/− 1.26 |
| Ratio (Number (KR478)/Average (T89)) | 0.96 |
| Ratio (Max (KR183)/Average (T89)) | 1.03 |
| Ratio (Min (KR183)/Average (T89)) | 0.90 |
| T-test | 0.1011 |
| Anova | 0.0278 |
| Num KR478 > Confidence Interval (T89) (95%) (20.78) | 0 |
| Num KR478 < Confidence Interval (T89) (95%) (18.26) | 2 |
| Num KR478 > Max (T89) (20.6) | 0 |
| Num KR478 < Min (T89) (18.7) | 3 |

### Gene STT 178

The gene STT 178 is homologous to AtHB8 which is a known regulator of xylem formation, we show here that this gene can be used to alter lignin levels in trees. The RNAi construction group KR484 shows variable levels of lignin with some lines contain higher and some lines containing lower amounts of lignin. A maximum decrease of lignin of 8% compared to wild type average and a maximum increase of lignin of 8% compared to wild type average in different transgenic lines see Table 44 below. It is obvious that in order to use this gene for manipulating lignin levels one have to produce many lines and measure lignin levels in those and then selecting transgenic lines with the desirable trait.

TABLE 44

| Individual Name | Lignin Content (%) |
|---|---|
| T89-74 | 20.8 |
| T89-75 | 20.9 |
| T89-76 | 20 |
| T89-77 | 19.9 |
| T89-78 | 21.2 |
| T89-79 | 19.3 |
| T89-80 | 20.3 |
| T89-83 | 20.5 |
| T89-84 | 20.4 |
| T89-85 | 20.1 |
| T89-87 | 20 |
| T89-88 | 18.9 |
| KR484-1A-A | 20 |
| KR484-2A | 21.8 |
| KR484-4A | 19 |
| KR484-5A-A | 18.9 |
| KR484-5A-B | 18.6 |

| | |
|---|---|
| Number (KR484) | 5 |
| Average (KR484) | 19.66 |
| Standard deviation (KR484) | 1.31 |
| Number (T89) | 12 |
| Average (T89) | 20.19 |
| Standard deviation (T89) | 0.65 |
| Max (T89) | 21.20 |
| Min (T89) | 18.90 |
| Confidence Interval (T89) (95%) | 20.19 +/− 1.43 |
| Ratio (Number (KR484)/Average (T89)) | 0.97 |
| Ratio (Max(KR484)/Average(T89)) | 1.08 |
| Ratio (Min(KR484)/Average(T89)) | 0.92 |
| T-test | 0.4273 |
| Anova | 0.2719 |
| Num KR484 > Confidence Interval (T89) (95%) (21.63) | 1 |
| Num KR484 < Confidence Interval (T89) (95%) (18.76) | 1 |
| Num KR484 > Max (T89) (21.2) | 1 |
| Num KR484 < Min (T89) (18.9) | 1 |

### Gene STT 307

The RNAi construction group KR595 shows a maximum decrease of lignin of 10% compared to the wild type average see Table 45 below.

TABLE 45

| Individual Name | Lignin Content (%) |
|---|---|
| T89-15 | 21.7 |
| T89-16 | 20.2 |
| T89-22 | 21.3 |
| T89-26 | 20 |
| T89-27 | 18.7 |
| T89-28 | 20.1 |
| T89-39 | 21.3 |
| KR595-1A | 18.5 |
| KR595-2A | 20.3 |
| KR595-2B | 21.6 |
| KR595-3A | 20.9 |
| KR595-3B | 18.5 |

| | |
|---|---|
| Number (KR595) | 5 |
| Average (KR595) | 19.96 |
| Standard deviation (KR595) | 1.41 |
| Number (T89) | 7 |
| Average (T89) | 20.47 |
| Standard deviation (T89) | 1.04 |
| Max (T89) | 21.70 |
| Min (T89) | 18.70 |
| Confidence Interval (T89) (95%) | 20.47 +/− 2.54 |
| Ratio (Number (KR595)/Average (T89)) | 0.98 |
| T-test | 0.5131 |
| Anova | 0.4835 |
| Num KR595 > Confidence Interval (T89) (95%) (23.01) | 0 |
| Num KR595 > Confidence Interval (T89) (95%) (17.93) | 0 |
| Num KR595 > Max (T89) (21.7) | 0 |
| Num KR595 < Min (T89) (18.7) | 2 |

### Gene STT 317

The RNAi construction group KR605 shows a maximum increase of lignin of 7% compared to the wild type average see Table 46 below.

TABLE 46

| Individual Name | Lignin Content (%) |
|---|---|
| T89-15 | 21.7 |
| T89-16 | 20.2 |
| T89-22 | 21.3 |
| T89-26 | 20 |
| T89-27 | 18.7 |
| T89-28 | 20.1 |
| T89-39 | 21.3 |
| KR605-3B | 21 |
| KR605-4A | 21.9 |
| KR605-4B | 21.6 |
| KR605-5A | 20.5 |
| KR605-5B | 20 |

| | |
|---|---|
| Number (KR605) | 5 |
| Average (KR605) | 21.00 |
| Standard deviation (KR605) | 0.78 |
| Number (T89) | 7 |
| Average (T89) | 20.47 |
| Standard deviation (T89) | 1.04 |
| Max (T89) | 21.70 |
| Min (T89) | 18.70 |
| Confidence Interval (T89) (95%) | 20.47 +/− 2.54 |
| Ratio (Number (KR605)/Average (T89)) | 1.03 |
| Ratio (Max(KR605)/Average(T89)) | 1.07 |
| Ratio (Min(KR605)/Average(T89)) | 0.98 |
| T-test | 0.3371 |
| Anova | 0.3605 |
| Num KR605 > Confidence Interval (T89) (95%) (23.01) | 0 |
| Num KR605 < Confidence Interval (T89) (95%) (17.93) | 0 |
| Num KR605 > Max (T89) (21.7) | 1 |
| Num KR605 < Min (T89) (18.7) | 0 |

### Gene STT 97

The construction group 35S029 shows a maximum decrease of lignin of 8% compared to the wild type average, see Table 47 below.

TABLE 47

| | Lignin |
|---|---|
| Individual Name | Content (%) |
| 35s029-1B | 18.4 |
| 35s029-3A | 19.0 |
| 35s029-3B | 17.5 |
| 35s029-4B | 18.4 |
| 35s029-5A | 18.0 |
| 35s029-6A | 17.5 |
| 35s029-6B | 17 |
| T89-01 | 18.4 |
| T89-08 | 17.6 |
| T89-16 | 18.6 |
| T89-19 | 19.4 |
| T89-20 | 19.1 |
| T89-21 | 19.5 |
| T89-25 | 18.1 |
| T89-32 | 18.2 |
| T89-39 | 18.6 |
| T89-40 | 17.8 |

| | |
|---|---|
| Number(35s029) | 7 |
| Average(35s029) | 18.0 |
| Standard deviation(35s029) | 0.68 |
| Number(T89) | 10 |
| Average(T89) | 18.5 |
| Standard deviation(T89) | 0.64 |
| Max (T89) | 19.5 |
| Min (T89) | 17.6 |
| Confidence Interval (T89) (99%) | 18.53 +/− 1.45 |
| Ratio (Average(35s029)/Average(T89)) | 0.97 |
| T-test | 0.115 |
| Anova | 0.107 |
| Num35s029 > Confidence Interval (T89) (95%) (19.98) | 0 |
| Num35s029 < Confidence Interval (T89) (95%) (17.08) | 1 |
| Num 35s029 > Max (T89) (19.5) | 0 |
| Num 35s029 < Min (T89) (17.6) | 3 |

### Gene STT 187

The RNAi construction group KR489 shows a maximum increase of lignin of 8% compared to the wild type average, see Table 48 below.

TABLE 48

| Individual Name | Lignin Content (%) |
|---|---|
| KR489-3A-1 | 19.8 |
| KR489-3B-1 | 19.4 |
| KR489-3B-2 | 20.2 |
| KR489-4A-1 | 19.1 |
| KR489-4A-2 | 19.3 |
| KR489-4B-2 | 19.4 |
| T89-02 | 18.5 |
| T89-04 | 17.2 |
| T89-08 | 17.7 |
| T89-10 | 19.0 |
| T89-15 | 19.4 |
| T89-23 | 18.5 |
| T89-27 | 18.7 |
| T89-29 | 19.2 |
| T89-34 | 18.8 |
| T89-35 | 20 |

| | |
|---|---|
| Number(KR489) | 6 |
| Average(KR489) | 19.5 |
| Standard deviation(KR489) | 0.4 |
| Number(T89) | 10 |
| Average(T89) | 18.7 |
| Standard deviation(T89) | 0.81 |
| Max (T89) | 20.0 |
| Min (T89) | 17.2 |
| Confidence Interval (T89) (99%) | 18.7 +/− 1.83 |
| Ratio (Average(KR489)/Average(T89)) | 1.04 |
| T-test | 0.016 |
| Anova | 0.035 |
| NumKR489 > Confidence Interval (T89) (95%) (20.53) | 0 |
| NumKR489 < Confidence Interval (T89) (95%) (16.87) | 0 |
| Num KR489 > Max (T89) (20) | 1 |
| Num KR489 < Min (T89) (17.2) | 0 |

### Gene STT 311

The RNAi construction group KR599 shows a maximum decrease of lignin of 9% compared to the wild type average, see Table 49 below.

TABLE 49

| Individual Name | Lignin Content (%) |
|---|---|
| KR599-1A | 19.4 |
| KR599-1B | 17.8 |
| KR599-2B | 20.2 |
| KR599-3A | 20.5 |
| KR599-3B | 18.8 |
| T89-01 | 19.4 |
| T89-02 | 19.9 |
| T89-03 | 19.2 |
| T89-32 | 20.1 |
| T89-34 | 19.1 |
| T89-35 | 19.3 |

| | |
|---|---|
| Number(KR599) | 5 |
| Average(KR599) | 19.3 |
| Standard deviation(KR599) | 1.09 |
| Number(T89) | 6 |
| Average(T89) | 19.5 |
| Standard deviation(T89) | 0.4 |
| Max (T89) | 20.1 |
| Min (T89) | 19.1 |
| Confidence Interval (T89) (99%) | 19.5 +/− 1.03 |
| Ratio (Average(KR599)/Average(T89)) | 0.99 |
| T-test | 0.769 |
| Anova | 0.745 |
| NumKR599 > Confidence Interval (T89) (95%) (20.53) | 0 |
| NumKR599 < Confidence Interval (T89) (95%) (18.47) | 1 |
| Num KR599 > Max (T89) (20.1) | 2 |
| Num KR599 < Min (T89) (19.1) | 2 |

### Gene STT 315

The RNAi construction group KR603 shows a maximum increase of lignin of 11% compared to the wild type average, see Table 50 below.

TABLE 50

| Individual Name | Lignin Content (%) |
|---|---|
| KR603-1A | 18.9 |
| KR603-2A | 20.9 |
| KR603-2B | 19.9 |
| KR603-3B | 18.8 |
| KR603-4B | 20.1 |
| T89-14 | 18.6 |
| T89-17 | 19.3 |
| T89-26 | 18.2 |
| T89-27 | 19.4 |
| T89-30 | 19.0 |
| T89-34 | 18.0 |

| | |
|---|---|
| Number(KR603) | 5 |
| Average(KR603) | 19.7 |
| Standard deviation(KR603) | 0.88 |
| Number(T89) | 6 |
| Average(T89) | 18.8 |
| Standard deviation(T89) | 0.58 |
| Max (T89) | 19.4 |
| Min (T89) | 18.0 |
| Confidence Interval (T89) (99%) | 18.75 +/− 1.49 |
| Ratio (Average(KR603)/Average(T89)) | 1.05 |
| T-test | 0.074 |
| Anova | 0.055 |
| NumKR603 > Confidence Interval (T89) (95%) (20.24) | 1 |
| NumKR603 < Confidence Interval (T89) (95%) (17.26) | 0 |
| Num KR603 > Max (T89) (19.4) | 3 |
| Num KR603 < Min (T89) (18) | 0 |

### Gene STT 392

The RNAi construction group KR680 shows a maximum increase of lignin of 7% compared to the wild type average, see Table 51 below.

TABLE 51

| Individual Name | Lignin Content (%) |
|---|---|
| KR680-1A | 18.5 |
| KR680-1B | 19.4 |
| KR680-2A | 20.0 |
| KR680-3A | 19.1 |
| KR680-4A | 18.6 |
| T89-01 | 19.0 |
| T89-11 | 17.9 |
| T89-18 | 18.7 |
| T89-28 | 19.2 |
| T89-30 | 18.9 |
| T89-42 | 18.9 |

| | |
|---|---|
| Number(KR680) | 5 |
| Average(KR680) | 19.1 |
| Standard deviation(KR680) | 0.61 |
| Number(T89) | 6 |
| Average(T89) | 18.8 |
| Standard deviation(T89) | 0.45 |
| Max (T89) | 19.2 |
| Min (T89) | 17.9 |
| Confidence Interval (T89) (99%) | 18.77 +/− 1.16 |
| Ratio (Average(KR680)/Average(T89)) | 1.02 |
| T-test | 0.32 |
| Anova | 0.301 |
| NumKR680 > Confidence Interval (T89) (95%) (19.92) | 1 |
| NumKR680 < Confidence Interval (T89) (95%) (17.61) | 0 |
| Num KR680 > Max (T89) (19.2) | 2 |
| Num KR680 < Min (T89) (17.9) | 0 |

### Gene STT 398

The RNAi construction group KR686 shows a maximum decrease of lignin of 8% compared to the wild type average, see Table 52 below.

TABLE 52

| Individual Name | Lignin Content (%) |
|---|---|
| KR686-1A | 17.3 |
| KR686-2A | 17.6 |
| KR686-3A | 17.7 |
| KR686-3B | 17.3 |
| KR686-4A | 18.5 |
| T89-01 | 19.0 |
| T89-11 | 17.9 |
| T89-18 | 18.7 |
| T89-28 | 19.2 |
| T89-30 | 18.9 |
| T89-42 | 18.9 |

| | |
|---|---|
| Number(KR686) | 5 |
| Average(KR686) | 17.7 |
| Standard deviation(KR686) | 0.49 |
| Number(T89) | 6 |
| Average(T89) | 18.8 |
| Standard deviation(T89) | 0.45 |
| Max (T89) | 19.2 |
| Min (T89) | 17.9 |
| Confidence Interval (T89) (99%) | 18.77 +/− 1.16 |
| Ratio (Average(KR686)/Average(T89)) | 0.94 |
| T-test | 0.005 |
| Anova | 0.004 |
| NumKR686 > Confidence Interval (T89) (95%) (19.92) | 0 |
| NumKR686 < Confidence Interval (T89) (95%) (17.61) | 3 |
| Num KR686 > Max (T89) (19.2) | 0 |
| Num KR686 < Min (T89) (17.9) | 4 |

### Gene STT 442

The RNAi construction group KR730 shows a maximum increase of lignin of 7% compared to the wild type average, see Table 53 below.

TABLE 53

| Individual Name | Lignin Content (%) |
|---|---|
| KR730-1A | 18.4 |
| KR730-2A | 18.8 |
| KR730-2B | 18.6 |
| KR730-3B | 20.1 |
| KR730-4A | 18.9 |
| T89-05 | 18.6 |
| T89-08 | 18.8 |
| T89-22 | 19.1 |
| T89-30 | 19.3 |
| T89-33 | 18.3 |
| T89-41 | 19.1 |

| | |
|---|---|
| Number(KR730) | 5 |
| Average(KR730) | 19.0 |
| Standard deviation(KR730) | 0.67 |
| Number(T89) | 6 |
| Average(T89) | 18.9 |
| Standard deviation(T89) | 0.37 |
| Max (T89) | 19.3 |
| Min (T89) | 18.3 |
| Confidence Interval (T89) (99%) | 18.87 +/− 0.95 |
| Ratio (Average(KR730)/Average(T89)) | 1 |
| T-test | 0.789 |
| Anova | 0.775 |
| NumKR730 > Confidence Interval (T89) (95%) (19.82) | 1 |
| NumKR730 < Confidence Interval (T89) (95%) (17.92) | 0 |
| Num KR730 > Max (T89) (19.3) | 1 |
| Num KR730 < Min (T89) (18.3) | 0 |

### Gene STT 476

The RNAi construction group KR764 shows a maximum decrease of lignin of 8% compared to the wild type average, see Table 54 below.

TABLE 54

| Individual Name | Lignin Content (%) |
|---|---|
| KR764-2A | 18.4 |
| KR764-2B | 19.6 |
| KR764-3A | 18.7 |
| KR764-3B | 17.35 |
| KR764-4A | 19.6 |
| T89-05 | 18.6 |
| T89-08 | 18.8 |
| T89-22 | 19.1 |
| T89-30 | 19.3 |
| T89-33 | 18.3 |
| T89-41 | 19.1 |

| | |
|---|---|
| Number(KR764) | 5 |
| Average(KR764) | 18.7 |
| Standard deviation(KR764) | 0.94 |
| Number(T89) | 6 |
| Average(T89) | 18.9 |
| Standard deviation(T89) | 0.37 |
| Max (T89) | 19.3 |
| Min (T89) | 18.3 |
| Confidence Interval (T89) (99%) | 18.87 +/− 0.95 |
| Ratio (Average(KR764)/Average(T89)) | 0.99 |
| T-test | 0.772 |
| Anova | 0.749 |
| NumKR764 > Confidence Interval (T89) (95%) (19.82) | 0 |
| NumKR764 < Confidence Interval (T89) (95%) (17.92) | 1 |
| Num KR764 > Max (T89) (19.3) | 2 |
| Num KR764 < Min (T89) (18.3) | 1 |

### Gene STT 551

The construction group TF0003 shows a maximum increase of lignin of 7% compared to the wild type average, see Table 55 below.

TABLE 55

| Individual Name | Lignin Content (%) |
|---|---|
| TF0003-1A | 19.9 |
| TF0003-1B | 20.9 |
| TF0003-2A | 20.1 |
| TF0003-3A | 20.6 |
| TF0003-4A | 20.0 |
| T89-01 | 19.4 |
| T89-02 | 19.9 |
| T89-03 | 19.2 |
| T89-32 | 20.1 |
| T89-34 | 19.1 |
| T89-35 | 19.3 |

| | |
|---|---|
| Number(TF0003) | 5 |
| Average(TF0003) | 20.3 |
| Standard deviation(TF0003) | 0.43 |
| Number(T89) | 6 |
| Average(T89) | 19.5 |
| Standard deviation(T89) | 0.4 |
| Max (T89) | 20.1 |
| Min (T89) | 19.1 |
| Confidence Interval (T89) (99%) | 19.5 +/− 1.03 |
| Ratio (Average(TF0003)/Average(T89)) | 1.04 |
| T-test | 0.013 |
| Anova | 0.011 |
| NumTF0003 > Confidence Interval (T89) (95%) (20.53) | 2 |
| NumTF0003 < Confidence Interval (T89) (95%) (18.47) | 0 |
| Num TF0003 > Max (T89) (20.1) | 2 |
| Num TF0003 < Min (T89) (19.1) | 0 |

### Gene STT 577

The construction group TF0050 shows a maximum decrease of lignin of 8% compared to the wild type average, see Table 56 below.

TABLE 56

| Individual Name | Lignin Content (%) |
|---|---|
| TF0050-1A-1 | 17.7 |
| TF0050-1A-2 | 18.3 |
| TF0050-1AB | 17.5 |
| TF0050-2A-1 | 17.1 |
| TF0050-2AB | 16.9 |
| TF0050-2B-1 | 17.8 |
| TF0050-2B-2 | 18 |
| TF0050-2B-3 | 17.6 |
| T89-04 | 17.9 |
| T89-07 | 17.9 |
| T89-11 | 19.4 |
| T89-19 | 18.0 |
| T89-20 | 18.6 |
| T89-22 | 18.3 |
| T89-27 | 17.5 |
| T89-36 | 18.4 |
| T89-41 | 18.6 |
| T89-46 | 18.6 |

| | |
|---|---|
| Number(TF0050) | 8 |
| Average(TF0050) | 17.6 |
| Standard deviation(TF0050) | 0.45 |
| Number(T89) | 10 |
| Average(T89) | 18.3 |
| Standard deviation(T89) | 0.53 |
| Max (T89) | 19.4 |
| Min (T89) | 17.5 |
| Confidence Interval (T89) (99%) | 18.32 +/− 1.2 |
| Ratio (Average(TF0050)/Average(T89)) | 0.96 |
| T-test | 0.008 |
| Anova | 0.009 |
| NumTF0050 > Confidence Interval (T89) (95%) (19.52) | 0 |
| NumTF0050 < Confidence Interval (T89) (95%) (17.12) | 2 |
| Num TF0050 > Max (T89) (19.4) | 0 |
| Num TF0050 < Min (T89) (17.5) | 2 |

### Gene STT 584

The construction group TF0061 shows a maximum decrease of lignin of 8% compared to the wild type average, see Table 57 below.

TABLE 57

| Individual Name | Lignin Content (%) |
|---|---|
| TF0061-2A-1 | 20.1 |
| TF0061-2BA-1 | 20.0 |
| TF0061-2BB-1 | 17.8 |
| TF0061-4A-1 | 20.5 |
| TF0061-4A-2 | 19.9 |
| T89-27 | 19.1 |
| T89-3 | 20.0 |
| T89-36 | 19.1 |
| T89-39 | 19.7 |
| T89-45 | 19.3 |
| T89-6 | 18.7 |

| | |
|---|---|
| Number(TF0061) | 5 |
| Average(TF0061) | 19.7 |
| Standard deviation(TF0061) | 1.06 |
| Number(T89) | 6 |
| Average(T89) | 19.3 |
| Standard deviation(T89) | 0.47 |
| Max (T89) | 20.0 |
| Min (T89) | 18.7 |
| Confidence Interval (T89) (99%) | 19.32 +/− 1.21 |
| Ratio (Average(TF0061)/Average(T89)) | 1.02 |
| T-test | 0.531 |
| Anova | 0.491 |
| NumTF0061 > Confidence Interval (T89) (95%) (20.53) | 0 |
| NumTF0061 < Confidence Interval (T89) (95%) (18.11) | 1 |
| Num TF0061 > Max (T89) (20) | 2 |
| Num TF0061 < Min (T89) (18.7) | 1 |

### Gene STT 606

The construction group TF0094.2nd shows a maximum decrease of lignin of 8% compared to the wild type average, see Table 58 below.

TABLE 58

| Individual Name | Lignin Content (%) |
|---|---|
| TF0094.2nd-1A | 18.1 |
| TF0094.2nd-5A | 18.8 |
| TF0094.2nd-5B | 17.5 |
| TF0094.2nd-6A | 17.1 |
| TF0094.2nd-6B | 17.6 |
| TF0094.2nd-7A | 18.2 |
| TF0094.2nd-7B | 17.7 |
| T89-01 | 18.4 |
| T89-08 | 17.6 |
| T89-16 | 18.6 |
| T89-19 | 19.4 |
| T89-20 | 19.1 |
| T89-21 | 19.5 |
| T89-25 | 18.1 |
| T89-32 | 18.2 |
| T89-39 | 18.6 |
| T89-40 | 17.8 |

| | |
|---|---|
| Number(TF0094.2nd) | 7 |
| Average(TF0094.2nd) | 17.9 |
| Standard deviation(TF0094.2nd) | 0.56 |
| Number(T89) | 10 |
| Average(T89) | 18.5 |
| Standard deviation(T89) | 0.64 |
| Max (T89) | 19.5 |
| Min (T89) | 17.6 |
| Confidence Interval (T89) (99%) | 18.53 +/− 1.45 |
| Ratio (Average(TF0094.2nd)/Average(T89)) | 0.96 |
| T-test | 0.037 |
| Anova | 0.041 |
| NumTF0094.2nd > Confidence Interval (T89) (95%) | 0 |
| (19.98) | |
| NumTF0094.2nd < Confidence Interval (T89) (95%) | 0 |
| (17.08) | |
| Num TF0094.2nd > Max (T89) (19.5) | 0 |
| Num TF0094.2nd < Min (T89) (17.6) | 2 |

### Gene STT 633

The construction group TF0138 shows a maximum decrease of lignin of 8% compared to the wild type average, see Table 59 below.

TABLE 59

| Individual Name | Lignin Content (%) |
|---|---|
| TF0138-1A | 18.1 |
| TF0138-2A | 18.0 |
| TF0138-3B | 18.4 |
| TF0138-4A | 18.8 |
| TF0138-4B | 17.7 |
| TF0138-5A | 16.8 |
| TF0138-5B | 18.9 |
| T89-04 | 17.9 |
| T89-07 | 17.9 |
| T89-11 | 19.4 |
| T89-19 | 18.0 |
| T89-20 | 18.6 |
| T89-22 | 18.3 |
| T89-27 | 17.5 |
| T89-36 | 18.4 |
| T89-41 | 18.6 |
| T89-46 | 18.6 |

| | |
|---|---|
| Number(TF0138) | 7 |
| Average(TF0138) | 18.1 |
| Standard deviation(TF0138) | 0.72 |
| Number(T89) | 10 |
| Average(T89) | 18.3 |
| Standard deviation(T89) | 0.53 |
| Max (T89) | 19.4 |
| Min (T89) | 17.5 |
| Confidence Interval (T89) (99%) | 18.32 +/− 1.2 |
| Ratio (Average(TF0138)/Average(T89)) | 0.99 |
| T-test | 0.505 |
| Anova | 0.477 |
| NumTF0138 > Confidence Interval (T89) (95%) (19.52) | 0 |
| NumTF0138 < Confidence Interval (T89) (95%) (17.12) | 1 |
| Num TF0138 > Max (T89) (19.4) | 0 |
| Num TF0138 < Min (T89) (17.5) | 1 |

### Gene STT 658

The construction group TFSTT021 shows a maximum decrease of lignin of 7% compared to the wild type average, see Table 60 below.

TABLE 60

| Individual Name | Lignin Content (%) |
|---|---|
| TFSTT021-2A-2 | 18.0 |
| TFSTT021-2B | 20.2 |
| TFSTT021-3A-1 | 19.0 |
| TFSTT021-3A-2 | 18.2 |
| TFSTT021-3B-1 | 19.4 |
| T89-13 | 19.6 |
| T89-31 | 19.3 |
| T89-33 | 19.1 |
| T89-39 | 19.0 |
| T89-5 | 19.3 |

| | |
|---|---|
| Number(TFSTT021) | 5 |
| Average(TFSTT021) | 18.96 |
| Standard deviation(TFSTT021) | 0.9 |
| Number(T89) | 5 |
| Average(T89) | 19.26 |
| Standard deviation(T89) | 0.23 |
| Max (T89) | 19.6 |
| Min (T89) | 19 |
| Confidence Interval (T89) (99%) | 19.26 +/− 0.64 |
| Ratio (Average(TFSTT021)/Average(T89)) | 0.98 |
| T-test | 0.505 |
| Anova | 0.49 |
| NumTFSTT021 > Confidence Interval (T89) (95%) | 1 |
| (19.9) | |
| NumTFSTT021 < Confidence Interval (T89) (95%) | 2 |
| (18.62) | |
| Num TFSTT021 > Max (T89) (19.6) | 1 |
| Num TFSTT021 < Min (T89) (19) | 2 |

### Gene STT 167

The construction group LMP1-002 shows a maximum increase of lignin of 9% compared to the wild type average, see Table 61 below.

TABLE 61

| Individual Name | Lignin Content (%) |
|---|---|
| LMP1-002-1B | N/A |
| LMP1-002-3A | 19.7 |
| LMP1-002-3B | 17.9 |
| LMP1-002-5A | 17.6 |
| LMP1-002-5B | 18.7 |
| T89-01 | 17.5 |
| T89-03 | 18.6 |
| T89-12 | 17.4 |
| T89-15 | 18.3 |
| T89-19 | 18.0 |
| T89-23 | 18.3 |
| T89-28 | 18.2 |
| T89-29 | 18.8 |
| T89-30 | 18 |
| T89-31 | 18.1 |

| | |
|---|---|
| Number(LMP1-002) | 4 |
| Average(LMP1-002) | 18.475 |
| Standard deviation(LMP1-002) | 0.94 |
| Number(T89) | 10 |
| Average(T89) | 18.12 |
| Standard deviation(T89) | 0.43 |
| Max (T89) | 18.8 |
| Min (T89) | 17.4 |
| Confidence Interval (T89) (99%) | 18.12 +/− 0.97 |
| Ratio (Average(LMP1-002)/Average(T89)) | 1.02 |
| T-test | 0.513 |
| Anova | 0.338 |
| NumLMP1-002 > Confidence Interval (T89) (95%) | 1 |
| (19.09) | |
| NumLMP1-002 < Confidence Interval (T89) (95%) | 0 |
| (17.15) | |
| Num LMP1-002 > Max (T89) (18.8) | 1 |
| Num LMP1-002 < Min (T89) (17.4) | 0 |

### REFERENCES

Aspeborg et al., Plant Physiology (2005), 137(3), 983-997
Baucher, Critical Reviews in Biochemistry and Molecular Biology, 38:305-350, 2003
Blom Statistikteori med tillampningar ISBN 91-44-05592-7
Brady and Provart 2007. Journal of the Science of Food and Agriculture Vol 87, Pp 925-929
Breaking the Biological Barriers to Cellulosic Ethanol: A Joint Research Agenda. A Research Roadmap Resulting from the Biomass to Biofuels Workshop, Dec. 7-9, 2005, Rockville, Md.
Burke et al 2007. Current opinion in genetics and development vol 17, 525-532.
Bustin S A. Journal of Molecular Endocrinology, 2000. 25, 169-193.
Chen and Dixon. Nature Biotechnology Vol. 25 no 7: pp 759-761, 2007
Chern et al. 2001) Plant J. 27: 101 113
Falco et al. US2005034176
Fan and Dong (2002) Plant Cell 14: 1377 1389
Feng and Doolittle (1987) J. Mol. Evol. 25: 351 360
Flinn et al in. U.S. Pat. No. 6,451,604
Geladi P, Kowalski B R. Partial least-squares regression—a Tutorial. Analytica Chimica Acta 1986, 185:1-17.
Gilmour et al. (1998) Plant J. 16: 433 442.
Griffiths, P. R. and De Haseth, J. A. Fourier Transform Infrared Spectrometry. New York: Wiley, 1986
Henikoff, Till, and Comai, Plant Physiol. 2004 June; 135(2):630-6.
Heo et al., Plant and Cell Physiology (2005), 46(12), 2005-2018
Ichikawa et al. 1997 Nature 390 698-701;
Jaglo et al. (1998) Plant Physiol. 127: 910 917.
Kakimoto et al. 1996 Science 274: 982-985.
Karimi M, Inze D and Depicker A. Treands in plant Sciences. (2002) vol 7 nos pp 193-195
Kang et al., Cell. 2001 Jun. 1; 105(5):625-36
Klason-lignin analysis Tappi method T 222 om-88,
Kosugi and Ohashi, (2002) Plant J. 29: 45 59
Lee et al. (2002) Genome Res. 12: 493 502
Lichtenstein and Nellen (1997), Antisense Technology: A Practical Approach IRL Press at Oxford University, Oxford, England.
Mount (2001), in Bioinformatics: Sequence and Genome Analysis Cold Spring Harbor Laboratory Press, Cold Spring Harbor, N.Y., page 543.
Nilsson et al. (1992) Transgenic Research 1, pp 209-220
Ratcliffe et al. (2001) Plant Physiol. 126: 122 132.
Remm et al. (2001) J. Mol. Biol. 314: 1041 1052
Rognes T, 2001, Nucleic Acids Research, 29, 1647-1652, 1989
Sambrook et al., Molecular Cloning-A Laboratory Manual (3rd Ed.), Vol. 1-3, Cold Spring Harbor Laboratory, Cold Spring Harbor, N.Y., 2001.
Slade and Knauf, Transgenic Res. 2005 April; 14(2):109-15
Smith and Waterman (1981). Helene! DNA aligning
Thompson et al. (1994) Nucleic Acids Res. 22: 4673 4680.
Trygg J, Wold S. Orthogonal projections to latent structures (OPLS). Journal of Chemometrics 2002, 16:119-128.
Tuskan et al. Science 15 Sep. 2006: Vol. 313. no. 5793, pp. 1596-1604
Vanholme et al. Current Opinion in Plant Biology 2008, 11:1-8
Varshney et al 2005. Trends in Plant Science, Vol 10, 621-630
Vasil et al. 1984, Cell Culture and Somatic Cell Genetics of Plants, Vol I, II and III, Laboratory Procedures.
Wold S, Esbensen K, Geladi P. Principal component analysis. Chemometrics and Intelligent Laboratory Systems 1987, 2:37-52.
Zhou et al. Plant and Cell Physiology (2006), 47(9), 1229-1240
US 2002/0168707
US 2005/034176
U.S. Pat. No. 4,987,071
U.S. Pat. No. 5,107,065
U.S. Pat. No. 5,231,020
U.S. Pat. No. 5,543,508
U.S. Pat. No. 5,583,021
U.S. Pat. No. 6,451,604
U.S. Pat. No. 6,506,559
U.S. Pat. No. 7,402,428
WO 91/14772
WO 96/06166
WO 98/53057
WO 98/53083
WO 99/53050
WO 99/61631
WO2004/097024
WO2006/078431
WO2008/067840
WO2008/067841
WO2008/006033

1. A method of producing a plant having altered lignin properties compared to its wild type, comprising altering in the plant the level of a gene product of at least one gene comprising a nucleotide sequence selected from the group consisting of
a) a nucleotide sequence from SEQ ID NO 23-115, 157-195 or
b) a nucleotide sequence being at least 60% identical to a nucleotide sequence from SEQ ID NO 24-115, 157-195 or being at least 99.6% identical to the nucleotide sequence SEQ ID NO 23 or
c) a sub sequence or fragment of a nucleotide sequence of a) or b).

2. The method according to claim 1 for producing a plant having higher lignin content, comprising over expression of at least one of the genes with SEQ ID NO: 113, 114, 115, 178 and 179 and/or down regulating at least one of the genes with SEQ ID NO: 24-26, 30, 32, 46, 47, 53, 54, 58, 157, 159, 160 and 162 in the groups of a), b) or c).

3. The method according to claim 1 for producing a plant having lower lignin content, comprising over expression of at least one of the genes with SEQ ID NO: 176, 177 and 180-187 and/or down regulating at least one of the genes with SEQ ID NO: 23, 27-29, 31, 33-44, 48-52, 55-57, 158, 161 and 163 in the groups of a), b) or c).

4. The method according to any of claims 1-3, comprising
i) selecting plant species expressing at least one of the nucleotide sequences selected from the group consisting of
a) a nucleotide sequence from SEQ ID NO 23-115, 157-195 or
b) a nucleotide sequence being at least 60% identical to a nucleotide sequence from SEQ ID NO 23-115, 157-195 or
c) a sub sequence or fragment of a nucleotide sequence of a) or b),
ii) crossing a plant species selected in i) with the same or another plant species selected in i),
iii) selecting plants with modulated expression of at least one of the nucleotide sequences selected from the group consisting of
a) nucleotide sequence from SEQ ID NO 23-115, 157-195 or
b) a nucleotide sequence being at least 60% identical to a nucleotide sequence from SEQ ID NO 23-115, 157-195 or
c) a sub sequence or fragment of a nucleotide sequence of a) or b). compared to the plant species selected under i)
iv) optionally further crossing one or more times the plants obtained in iii) and selecting plants with modulated expression of at least one of the nucleotide sequences selected from the group consisting of
a) a nucleotide sequence from SEQ ID NO 23-115, 157-195 or
b) a nucleotide sequence being at least 60% identical to a nucleotide sequence from SEQ ID NO 23-115, 157-195 or
c) a sub sequence or fragment of a nucleotide sequence of a) or b) compared to any of the plant species used in i) and/or plants obtained in iii).

5. The method according to any of claims 1-3, the method steps comprising:
i) providing an expression vector comprising a nucleotide sequence selected from the group consisting of
a) a nucleotide sequence from SEQ ID NO 23-115, 157-195 or
b) a nucleotide sequence being at least 60% identical to a nucleotide sequence from SEQ ID NO 23-115, 157-195 or
c) a sub sequence or fragment of a nucleotide sequence of a) or b. and
d) at least one regulatory element operable linked to the polynucleotide sequence, wherein said at least one regulatory element controls expression of the polynucleotide sequence in a target plant;
ii) introducing the expression vector into at least one plant; and
iii) selecting at least one transgenic plant that has a modulated lignin quantity compared to its wild type.

6. The method according to any one of claims 1-5, wherein said modulated expression is effected by introducing a genetic modification preferably in the locus of a gene comprising SEQ ID NO 23-115, 157-195 encoding a polypeptide or a homologue of such polypeptide.

7. Method according to claim 6, wherein said modification is effected by one of: T-DNA activation, TILLING, homologous recombination, site-directed mutagenesis or directed breeding using one or more of SEQ ID NO 23 to 115, 157-195 or a fragment thereof as markers in any step of the process of producing the desirable plants.

8. The method according to any of claims 1-6, wherein the modulation is decreased yield in lignin.

9. The method according to any of claims 1-6, wherein the modulation is increased yield in lignin.

10. The method according to any of the preceding claims 1-3, 4, 6-8, comprising the step of providing a recombinant DNA construct comprising a nucleotide sequence selected from the group consisting of:
a) a nucleotide Sequence comprising a sequence selected from SEQ ID NO 23-115, 157-195 or b) a complementary nucleotide sequence of a nucleotide sequence of a) or
c) a sub sequence or fragment of a nucleotide sequence of a) or b) or
d) a nucleic acid sequence being at least 60% identical to any one of the sequences in a), b) and c); or
e) a nucleotide sequence which hybridizes under stringent conditions to a nucleotide sequence of a), b) or c).

11. The method according to any one of claims 1-3, 4, 6-9, wherein the nucleotide sequence encodes a polypeptide comprising a conservatively substituted variant of a polypeptide of (a).

12. The method according to any one of claims 1-3, 4, 6-10, wherein the nucleotide sequence comprises a silent substitution in a nucleotide sequence.

13. The method according to any one of claims 1-3, 4, 6-11, wherein the sub sequences or fragments have at least 65% Sequence identity to a conserved domain of a nucleotide sequence as described in claim 8 a).

14. The method according to any one of claims 9 to 12, wherein the recombinant DNA construct further comprises a constitutive, inducible, or tissue specific promoter operably linked to said nucleotide sequence.

15. The method according to any one of claims 1-13, wherein the recombinant DNA construct further comprises a strong constitutive promoter in front of a transcribed cassette comprising a nucleotide sequence as defined in claim 1 followed by a plant functional intron followed by the nucleotide sequence as defined in claim 1 in reverse orientation.

16. The method according to any one of claims 10 to 14, wherein the method comprises the further step of transforming regenerable cells of a plant with said recombinant DNA construct and regenerating a transgenic plant from said transformed cell.

17. A plant having a having altered lignin properties compared to its wild type, comprising a nucleotide capable of altering in the plant the level of a gene product of at least one gene wherein the at least one gene comprises a nucleotide sequence selected from the group consisting of
a) a nucleotide sequence from SEQ ID NO 23-115, 157-195 or
b) a nucleotide sequence being at least 60% identical to a nucleotide sequence from SEQ ID NO 23-115, 157-195 or
c) a sub sequence or fragment of a nucleotide sequence of a) or b).

18. The plant according to claim 17, having higher lignin content, comprising at least one over expressed gene with SEQ ID NO: 113, 114 and 115, 178 and 179 and/or at least one down regulated gene with SEQ ID NO: 24-26, 30, 32, 46, 47, 53, 54 and 58, 157, 159, 160 and 162 in the groups of a), b) or c).

19. The plant according to claim 17, having lower lignin content, comprising over expression of at least one of the genes with SEQ ID NO: 176, 177 and 180-187 and/or down regulating at least one down regulated gene with SEQ ID NO: 23, 27-29, 31, 33-44, 48-52, 55-57, 158, 161 and 163 in the groups of a), b) or c).

20. A transgenic plant comprising a recombinant polynucleotide (DNA construct) comprising a nucleotide sequence selected from the group consisting of:
a) a nucleotide sequence comprising a sequence selected from SEQ ID NO 23-115, 157-195 or
b) a complementary nucleotide sequence of a nucleotide sequence of a) or
c) a sub sequence or fragment of a nucleotide sequence of a) or b) or
d) a nucleic acid sequence being at least 60% identical to any one of the sequences in a), b) and c) or
e) a nucleotide sequence which hybridizes under stringent conditions to a nucleotide sequence of a), b) or c).

21. The transgenic plant according to claim 20, wherein the nucleotide sequence selected from SEQ ID NO 23-115, 157-195 encoding a polypeptide comprising a conservatively substituted variant of a polypeptide of a) or d).

22. The transgenic plant according to any one of claims 20 to 21, wherein nucleotide sequence comprising a silent substitution in a nucleotide sequence.

23. The transgenic plant according to any one of claims 20 to 22, wherein the sub-sequences or fragments have at least 60% sequence identity to a conserved domain of a nucleotide sequence of the claim 17.

24. The transgenic plant according to any one of claims 17 to 22, wherein the recombinant DNA construct further comprising a constitutive, inducible, or tissue specific promoter operably linked to said nucleotide sequence.

25. The transgenic plant to any one of claims 17 to 24, wherein the recombinant DNA construct further comprises a strong constitutive promoter in front of a transcribed cassette consisting comprising a nucleotide sequence as defined in claim 4 followed by a plant functional intron followed by the nucleotide sequence as defined in claim 17 in reverse orientation.

26. A plant cell or plant progeny of a plant, which may be transgenic according to any of claims 17 to 25.

27. Wood produced by a plant, which may be transgenic according to any of claims 17 to 25.

28. A DNA construct, such as a vector, comprising at least one sequence described in claims 10 to 13.

29. A plant cell, a plant progeny or a vegetable material comprising the DNA construct according to claim 28.

30. Use of a nucleotide sequence selected from the group consisting of
a) a nucleotide sequence from SEQ ID NO 23-115, 157-195 or
b) a nucleotide sequence being at least 60% identical to a nucleotide sequence from SEQ ID NO 23-115, 157-195 or
c) a sub sequence or fragment of a nucleotide sequence of a) or b)
for the identification of plants having altered lignin characteristics as compared to the wild-type.

31. Use of a nucleotide sequence selected from the group consisting of
a) a nucleotide sequence from SEQ ID NO 23-115, 157-195 or
b) a nucleotide sequence being at least 60% identical to a nucleotide sequence from SEQ ID NO 23-115, 157-195 or
c) a sub sequence or fragment of a nucleotide sequence of a) or b).
as candidate genes in marker assisted breeding.

32. Use of a nucleotide sequence selected from the group consisting of
a) at least one over expressed gene chosen from genes with SEQ ID NO: 113, 114, 115, 178 and 179 and/or at least one down regulated gene chosen from genes with SEQ ID NO: 24-26, 30, 32, 46, 47, 53, 54, 58, 157, 159, 160 and 162 or
b) a nucleotide sequence being at least 60% identical to a nucleotide sequence from SEQ ID NO 24-26, 30, 32, 46, 47, 53, 54, 58, 113-115, 157, 159, 160, 162, 178 and 179 or
c) a sub sequence or fragment of a nucleotide sequence of a) or b)
in a plant for increasing lignin levels for energy production.

33. Use of a nucleotide sequence selected from the group consisting of
a) at least one over expressed gene chosen from genes with SEQ ID NO: 176, 177 and 180-187 and/or one down regulated gene chosen from of the genes with SEQ ID NO 23, 27-29, 31, 33-44, 48-52, 55-57, 158, 161 and 163 or
b) a nucleotide sequence being at least 60% identical to a nucleotide sequence from SEQ ID NO, 27-29, 31, 33-44, 48-52, 55-57, 158, 161, 163 176, 177 and 180-187 or at least 99.6% identical to SEQ ID NO 23 or
c) a sub sequence or fragment of a nucleotide sequence of a) or b)
in a plant for lowering lignin levels improving saccharification for bio-refining and ethanol production and for lowering lignin levels in pulp and paper processing.


SEQUENCE LISTING (195 sequences)

SEQ ID NO: 1 - DNA, 42 bp, Artificial Sequence
  Primer Sequence
gggacaagtttgtacaaaaaagcaggctttttttttttttvn

SEQ ID NO: 2 - DNA, 34 bp, Artificial Sequence
  Primer Sequence
agaaagctgggtattgaatagccaaatatctctc

SEQ ID NO: 3 - DNA, 31 bp, Artificial Sequence
  Primer Sequence
agaaagctgggttccttaacttgtccgagag

SEQ ID NO: 4 - DNA, 34 bp, Artificial Sequence
  Primer Sequence
agaaagctgggttagagttcgagaggtcatcttc

SEQ ID NO: 5 - DNA, 32 bp, Artificial Sequence
  Primer Sequence
agaaagctgggtgcttagtggtctgggtttag

SEQ ID NO: 6 - DNA, 34 bp, Artificial Sequence
  Primer Sequence
agaaagctgggtgaaggtggtggttcaattatac

SEQ ID NO: 7 - DNA, 49 bp, Artificial Sequence
  Primer Sequence
ggggaccactttgtacaagaaagctgggtcgctgacaccaccactttcc

SEQ ID NO: 8 - DNA, 50 bp, Artificial Sequence
  Primer Sequence
ggggaccactttgtacaagaaagctgggtaacgtgatttcaaagggtcgc

SEQ ID NO: 9 - DNA, 54 bp, Artificial Sequence
  Primer Sequence
ggggaccactttgtacaagaaagctgggtgtaatacgactcactatagggcgaa

SEQ ID NO: 10 - DNA, 31 bp, Artificial Sequence
  Primer Sequence
aaaaagcaggctttgttgttgtttcgatctg

SEQ ID NO: 11 - DNA, 32 bp, Artificial Sequence
  Primer Sequence
aaaaagcaggctcgagaaagaagaacctagtg

SEQ ID NO: 12 - DNA, 33 bp, Artificial Sequence
  Primer Sequence
aaaaagcaggctatttgggtcattcttgctcac

SEQ ID NO: 13 - DNA, 33 bp, Artificial Sequence
  Primer Sequence
aaaaagcaggctaatcagttgcttctcattgtc

SEQ ID NO: 14 - DNA, 33 bp, Artificial Sequence
  Primer Sequence
aaaaagcaggctccatccctcatcactaaatcc

SEQ ID NO: 15 - DNA, 49 bp, Artificial Sequence
  Primer Sequence
ggggacaagtttgtacaaaaaagcaggctagaaagccaggggagaatgc

SEQ ID NO: 16 - DNA, 53 bp, Artificial Sequence
  Primer Sequence
ggggacaagtttgtacaaaaaagcaggctgctctgttgttactctggacatgc

SEQ ID NO: 17 - DNA, 54 bp, Artificial Sequence
  Primer Sequence
ggggacaagtttgtacaaaaaagcaggctattgattaaccatggagaaagcaca

SEQ ID NO: 18 - DNA, 54 bp, Artificial Sequence
  Primer Sequence
ggggacaagtttgtacaaaaaagcaggctaagctgatgatcaaatgaggacatc

SEQ ID NO: 19 - DNA, 54 bp, Artificial Sequence
  Primer Sequence
ggggacaagtttgtacaaaaaagcaggctattaatcatgtcaggtgatcaaagg

SEQ ID NO: 20 - DNA, 52 bp, Artificial Sequence
  Primer Sequence
ggggaccactttgtacaagaaagctgggtcctttttcccctacaaatcatgg

SEQ ID NO: 21 - DNA, 54 bp, Artificial Sequence
  Primer Sequence
ggggaccactttgtacaagaaagctgggtcaaggatgtatgcttggttctatga

SEQ ID NO: 22 - DNA, 54 bp, Artificial Sequence
  Primer Sequence
ggggaccactttgtacaagaaagctgggtaagaagccaaaggacttaagctgtc

SEQ ID NO: 23 - DNA, 1351 bp, Artificial Sequence
  Synthetic Construct
cgcacagagaggatttcaggaagcgctggggatgtgttggatgatgatccagtgggaaggttgaaggtctatgtttatgagcttccgagtaaatacaacaagaaactgctgcagaaggatcccagatgtctcacccatatgtttgctgcagaaatcttcatgcatagatttctcttatccagcccagttcgaacccttaacccagatgaagcagattggttttattcccctatataccccacttgtgatctcacacccatgggcttgccgttgcccttcaaatctcctaggatgatgagaagtgcgatacagctaatctcctccaattggccttactggaatcgtacggaaggggctgatcacttttttgttgtgccccatgactttggagcctgcttccactatcaggaagagaaagccgttgagcggggcattcttccgctactccagcgttctactttggttcaaacttttgggcaacgaaatcatgtgtgcttgaatgaggggtcaattacaattcctccttttgctcctcctcaaaagatgcaggcccaccagattccccctgacattccacggtccatttttgtctatttccgtggattgttttatgatgtaaataatgatccagaaggtggttattatgcaagaggagcaagggctgcagtttgggagaatttcaagaacaatccactcttcgacatctccactgaccatccaacaacatattatgaagacatgcagcgagctatattttgcttgtgcccgctcggttgggctccatggagccctagattagttgaagcagtggtatttggatgcattcctgtcatcatagcagatgatattgttttgccatttgccgatgctatcccatgggaggaaattggggtgtttgtagcagaggaagatgtccctaacctggacacaatcctaacatccataccaccagaggtgattttaaggaaacaaaggcttcttgcaaatccttctatgaaacgtgcaatgttattcccacaacctgcacaaccaggtgatgctttccaccaaatcctaaacgggctggctcgtaagttgccgcacgacaggagtgtttacttgaagtctggtcagaatattttgaattggacagcaggaccagttggggacctgaaaccttggtaagagaagtgtcatttcctcaacagacatggctgtggtgtatctactgtttcatctaatcgcaaggactgtaaacttctctttgaagttttgatttttgtatagattcttgaatttcacgtgtacatctgggacatagttcgatgctttaaataacttcgcatttttcttggtatcggcaaaaaaaaaa

SEQ ID NO: 24 - DNA, 813 bp, Artificial Sequence
  Synthetic Construct
tgcagactttgggaggtattctccaaatatagcaaatgttggcaaagatgtgattgcaccatacaaacatgtaattaaaagctatgccaacgactcctctaactttgacagccgcccgactttgctgtattttcaaggagccatatacaggaaagatggtggctttgctcggcaagaattattttatgctctaaaagatgaaaaagacgtgcattttcagttcggtagtgtacaaaaggatggagtgagcaaagcttctcaaggaatgcactcctccaaattctgccttaacatagcaggtgacacaccctcatcaaaccgcctctttgatgccattgccagccactgcgttccggtcatcatcagtgacgatattgagctcccttatgaagatgtcctcgactactctcagttctgcatatttgtccgcacttcagatgctgtcagagaaaagttcctcataaatcttgtcaggagtattaagaaggatgaatggactagaatgtggcagaggttgaaagaggtagaaaacttctttgagttccaatatccatcaaaggaaggagatgcagttcaaatgatatggcaggctgtagcaagaaaagtccctgcaattaggttgaaggtaaataagtttaggcgattttctcgttttgggacccctaaagatggagtgttgagaaaaataccatcacctagtaatttttggtgaaacattcagtgtttctttatagtgaactttgaggttctgtttatatataaaggtgagaaattcaatttattctcactgcagattttggcaaaaaaaaaaaa

SEQ ID NO: 25 - DNA, 807 bp, Artificial Sequence
  Synthetic Construct
attagcaatgaaagaacctatggcgaaaacagtccttgagaatggttctcagggacccaacgtggcagaatctgatattatgaaatttccacgggaccgaaggttgaatatagatgatgaccataacttctgtaatattccgtgtggccaaaatggtgatggaaatctcatgtctgctgctggaacatatgatgtatcacacttgaatggatttgcagtaggcagccagatgtctcttgcattgggattgcaaagtaacgacagtgattcatttcccacgtttgatggggcccatacgagaggtaacaccatatcagcttccccggttgggcataatgaggtggattatcactgtatggatacaggaaagcaacaagacaggattgccagttcccatcggttacatgattttgtggtttgagagaatccttctagctttacgagggagtttcatcaatcctgccaagagaaggattgatggagttttggataacataaagcagaagaaaacaagctcagtgatactggtttgttattcattcaagccccgtgctcttctcttggtttgaatagaagaaaaccaacacccttacagccacagacatagatgtaacataagggcatggtgcctgcttgtaatatacgtacatattacagggattgtagatatatcgatctgtgcatatacaataagatttttgtgatcttttgtactcttaagcaatttctacaagctctccctaattgtgacactgatgtatcatttcattcctaattctgaaataaatgtgatttgctaaaaaaaaa

SEQ ID NO: 26 - DNA, 849 bp, Artificial Sequence
  Synthetic Construct
agcagtcagagcttataactagctaattgattaaccatggagaaagcacacacaaaatctgctcttaagaagcttgttaaggctagctcacagtctgcatcgtggagcaatgctgctagaggaatggcaaaagatgatctcaaggatccgctttatgacaaatccaaggttgccccaaaacctcttgcaaaagaaaacactaagccccaggaattcaaactccacactggacaaagagctctcaaacgtgccatgttcaactattctgtggcaaccaagatatatatgaatgagcaacagaagaggcaaatagagaggatacaaaagatcatagaagaagaagaggttcgtatgatgaggaaggagatggttccaagagctcaattgatgccttactttgacagacctttctttccccaaagatcaagcaggccattgacagttcctagagagccaagtttccacatggtgaacagcaagtgttggagctgcatccctgaggatgaactttactactactttgaacatgctcatccccatgatcatgcctggaggcctgttaattaaataattgccatgatttgtaggggaaaaaggagagtggagtattctctctgtgctgctaatttcttcatgctgtccaagttagtcttgtataatttggacttttggagtgttgatagtgtttactgtttagatcatgtggtaggtcttgtagtgggtgtaagtagtcgctttttcatagccgaaagtccttttcaatgggggattttgcttaccaataaggcggatattgtacttcgttttgtactattaaaaagcaagtcaccttcttccaaaaaaaaaaaa

SEQ ID NO: 27 - DNA, 1139 bp, Artificial Sequence
  Synthetic Construct
ggaagacctactcaccgatgcaggcatggttctcactgcaccctctggctccggcaataattgaggtcaaggcattgcatcatttcgattggttcgcaaaggggaaggtgccagtcatggaagcaatggaaaaggatcagagggtgaggtcacagtttagagggggttcgtctgccattgtagcaaataacactgagaagcctcatgttatcgcagcaaaactacaaacacttagtcccaaatacaattcagtgatgaatcacatccgaattcatctacctgagctgttcccaagcctaaacaaggttgtgttcctagacgacgacatagtggtgcagtccgatctttcgcctctatgggacgtcgacatggatggaaaggttaacggtgcagtagaaacctgtcgaggagaagacaagtccgtcatgtcaaagaagttgaagagctatttgaacttctctcatcctttgatatcagaaaatttcaaacccaacgaatgtgcctgggcttatggaatgaacatatttgatttagaggcttggaggaaaaccacataagcacaacataccatcactgggttgaagagaacttgaaatcagacctgagcttgtggcagctaggaacactgcctccaggtctgatagcattccatggccacgtccacgttatcgatcccttttggcacatgttgggtcttggctaccaagaaaatacaagtttggcagatgctgagactgccggagtcatccatttcaacggtcgagcaaagccttggctggatatagcattccctcagctccgccccctgtgggcgaagtacatcaacttttccgacaagttcataaagggctgtcatattaggccatcttaacttgagttttgagatgaaaatgttggctgcgatcgtgattaatatatgtctatatataaggagaaaaaaagcagagaagagagagttcttcacaaatccaaggccttgaatccagagagtaaaatattcataaccaagcatttgtttgcttggtttttttagaattcatcctctgtgggcgaacaacccttccctattacttcaaatcttgattcaattttaatgagaaattaagcatgttatggattttcaacgggcaaaaaaaaaa

SEQ ID NO: 28 - DNA, 898 bp, Artificial Sequence
  Synthetic Construct
cgaaaaacgagatacacaagattaaccaaattgtagtagatcccagcttgcatcttcaaaagaggagccacctttattacgctgttgagactcgaacaacacctgatgctttcaagatatttggaggttcgccctggctgattcttacaagagctttcatggagtactgtgtccaaggatgggacaaccttccaagaaaactactaatgtacttcagcaacacggcatccccacttgaatcgtatttccactctgtcctctgcaactctcctgagtttcaaaacacaacagtaagcgatgatttaaggtacaatattctggaaactactacagatggggaatcaccttatgacaaaatgctaaatggtggagcagcatttgcaaggccatttaaagaagatgctgctgctctaaacatgatagatgagaacgtcttgaaccgtgaacccaacggattggtgcctggaaaatggtgcttagaccagggtatgaacaagagctcagaggcatcaaagcctccaggggaggatttgtgttcaacctggggtaacattaatgatgtcaagccaggatcttatggtatcaagcttgcatttttattgtctaagattgccagtgaagagaaattgacaactagtcaatgccttcaagctacaaaaatggggtcatcatagaaccaagcatacaacctagtgtggataatttttttcttttaatcgaaaactataggttaaatcaagcttcttagccatataaatcctttgctaaatgcactacaaatgcatacttgcttgttaaaacaaaagctagaagcacccagagatatattgtacatacattcacattttatcgaggatttctacaataaatttattgtttttcccccaaaaaaaaaaaa

SEQ ID NO: 29 - DNA, 1278 bp, Artificial Sequence
  Synthetic Construct
aatcactctttcaacttcaagctgagcgaattttgtgtgtccaaagactagcagcccaagctttaagtgaaggttctggaagctcagtttcaatacattggtacataatgaccagcccgtttactcatgattccacacgatttttttttgaaaatcacaagtactttggccttgaagcagatcaggttaccttcttccagcaaggcaccataccttgtgtttccaaggatggcagatttatcatggagactccatttagggtagctaaggctccgggtgggaatggaggagtatattcagcgctgaaatattcaaaattattagaggatatggcctcaagagggatcaagtacgtggactgctatggggttgacaacgcactggtacgtgtagctgatccagcttttttgggatatttcattgataaaggtgtagcagctgcagcaaaagttgttcgtaaggcatacccccaagaaaaggttggtgtttttgtaaggcaaggtaaaggtggacctcttaccgtggttgaatacagtgagctggatcagtcactggcttctgcagtcaatcaacaaactggacgccttcgtttttgttggagtaatgtgtgcttgcacatgttctctttggattttctaaaccaagtggcaaatggccttgagaaagacagcatttaccatctagctgagaaaagaattccttctattcatggtgatacgatgggattaaaactagagcaattcatattcgatgcattcccatatgctccttcaactgctctttttgaggtaccacgtgaagaagaatttgcacctgtaaaaaatgccaatgggtcaaattttgacactcctgagagtgctcggctgcttgttcttcgactgcatactcgctgggtggttgcagcaggtggcttcttaacacattcggtgcctttatatgcaactggtgtggaggtatcaccactttgctcctatgctggcgaaaatctagaagccatatgccggggaagaacatttcacgcaccttgtgagattacattctagttaaatttgtatccatatattcttgatttgtacggctcccattggaaaggggaaaggggttatccatcccacgcatgtactgttgtggtagattagcgagagatatgaatctgtaagagttccaggttttgttacatcctgggtgtgatgtcaagagtgatactatttgattaatgtatatcattcaagttattgttgagcaatgataatcctctcttcttcctaaaaaaaaaaa

SEQ ID NO: 30 - DNA, 491 bp, Artificial Sequence
  Synthetic Construct
gaaaagggttgcaattattacaccaagaatttccatggatcagttgaggctttccatgtagacaccgttgataccacaggtgctggtgatgcatttgttggtgctctcctttgcaagattgttgatgaccaatctgtgcttgaggacgagccaaggttgagagagatactaagatttgcaaatgcttgtggagccattacaaccaccaaaaagggagcaatccctgctcttcctactccggccgatgccctcaaactggtgaacgaaggaaaatgaactctttttttttaatccgcactgcatttttccctttccttttcagctcctggttctcaattcaatcgaagagctgttgcaatctcaaattttcgcctttttctctcgagtgtaatgatttgaaagacttgcgctttaaggtcttgccaaataaatttttttcttttgtgttagctttctccaaatattatatttgtttaaagcaaaaaaaaaaa

SEQ ID NO: 31 - DNA, 979 bp, Artificial Sequence
  Synthetic Construct
caaggaggtatattgagtggagagggcggtgatgaaaccaaagatattcttttattggacgtggctcccctcaccctgggtattgaaactgttggtggagtgatgaccaagttgattccaaggaacactgctattccaaccaagaaatctcaagttttcaccacttaccaggatcagcagactactgtctccattcaggtctttgaaggtgaaaggagtctcacaaaggattgcaggctgcttggtaaatttgatttgactagcattcctccagctccaaggggaacccctcaaattgaggtcacgtttgaggttgatgccaatggtatcctgaatgtcaaggctgaagacaagggcactggtaaatcagaaaagatcaccatcacgaatgacaagggtcgtttgagccaggaggaaatcgagcgcatggttcgtgaggcagaggagtttgccgaggaggacaagaaggtgaaggagaggattgatgctcgtaacagtttggagacctatgtatataacatgaaaaaccagattaacgacaaagataagcttgctgacaagctcgagtctgatgagaaggagaaaatagaaactgctaccaaggaggcccttgaatggctggatgacaaccagaatgccgagaaagaggattatgaagagaagctcaaggaggtggaagctgtttgcaacccaatcatcactgctgtttaccagagatctggtggagctgcaggtggtggatcagctgaagattccgaggacgactctcaagatgaactctgagaatctttctgcttccttctacccctcctggaaagcaagggcaagcgatgaggatgtaggctagatgaagtagaggatattgtaattttgttgatcctagaagctaaagggttaaaataacatatcccctttttttggttttttgaggttaaaaatacatggaatagtttgtttccaaaaaaaaaaaaaaaaaaaa

SEQ ID NO: 32 - DNA, 701 bp, Artificial Sequence
  Synthetic Construct
ggaatggagagctctaccgttacatttgtgataccaagggagcttttgtgcagcctgctttgtatgaggcttttggattgactgttgtggaggccatgacatgtggtttgccaacctttgctacttgcaatggcggtcctgctgagatcattgtgcatggaaaatccggattccatattgatccttaccatggagtacaggctgctgaactccttgttgacttctttgagaagtgcaaggctgatcccacttactgggacaaaatctcccagggaggcctgcagcgaatccaagagaagtatacctggaaaatttactctcaaaggctcctgactctcacaggagtttatggcttctggaagcacgtttccaaccttgatcaccgtgagagccgtcgctatctggaaatgttctatgcactgaaatatcgcaaattggctgattctgttcctttgactatcgagtaaatggaactggagaaatcgaggaaacatgggttggtttgagtcgggttccgggtccagaataatggtcatttcacgatagtgattggacaagaaaggctttgatcttctttttgtttttttgtttttcctttctgtgtgcatcttttttgttttcccttttcttttcatcatgcagcctcttcaatttcagtaaagattgtgtctgttgagctaaaaaaaaaaaaaaaaaaaaaa

SEQ ID NO: 33 - DNA, 1439 bp, Artificial Sequence
  Synthetic Construct
ctccaatcacgcgtccgctctacacaggtcgatccaaagcaattttctccattacaagagaagccaagaatttgtgacgaacttggtataaattagttttgttttgacaagagaaagacttgtttgtttaatttccatgtgtctgaggagggaacgaagaacaaccccagggtgcttttcattgttcggacacctcttagattctcagacaacttgaacacaaaatatttcccgagttccaggatcattttgaaggatacaaggaaagaggaataaaaagaaagaacaatggcattcacgggaaccctggataagtgcaaggcttgtgataagactgtttattttgttgatatgatgtctcttgaaggggtgccttatcataaatcctgcttcaaatgcagccattgcaaaggaactcttgtgatgagcaactactcatccatggatggagtcctctactgcaagactcattttgagcaacctttcaaggagggtggagatttcagcaggaattttcagaaagggaagcccgagaggactcatgacctgtctaggatccccagcaaactatctccagttgttctgtggaacccaggacaaatgctccacttgcggcaaaacagtgtaccctttggagaaggtttgttttcagctctaaacttaaataaatccattaggtcctaaagacctgtgtgctattcatacccctacatttatcagataaatgacgaacatattttaggtacatcttctctatggtagatggatctatctgagaaatgttttcaagtctctgttatttaatagccatgcctgtgataatctggaataagcacttaagtgctataaccattgatgacataggttaccatggagggagaatgttaccacaagacatgcttcaggtgtgctcatggtggatgtccactcacacactcatcttatgctgctcttgacggagtcctctactgcaaggtccacttcgcccagctcttcatggaaaagggaacctacagccatgtccttgcatcagctgcacataagagatcaaattcgacacctccggaactggccgggtcaaacccagaggaaggagctgctgcggaggaggaaaagtcagaagagcaatcttagacaaagcacatcatgttttcttgcattgctatcttttcaagttgccaggactgcttcgagttttcatgttgttgaatattattgccggattgtgttcaaagtttgtttttggatttgactatctatgttacaatgccttgctacaggatattgaattgcactagtttttacaaattcctcctcttgcaatttaatattcttggcaacaggatacaaatgaaaaacccaaatcctaggaaccagtatcaacattcattgatctcagctctttgctcagatggattgcgttttccaaaaaaaaaaaa

SEQ ID NO: 34 - DNA, 792 bp, Artificial Sequence
  Synthetic Construct
cttgttcgatccctgcactaatcctgattaatgcctgcattcccttttagattttgtgtttgattttgtcctgtaaggagctggatatgcgggatcatatggagagatttgtagttcttccattctccatcgcctgtgcttctcactccagtgttgatgtggcctccagtgaatcctccaagaaaccaagacccgaaaccaaatcacatgcatcaagaggacaagaaggggaggaaagctcttgtaaagaaaagacgaagaacagtacacttggtttcctgctggctcttccaaagccttgcatatccagtagcttgcacaaattgattagaggcatcaagactctctcccaagtatttgtgtacaaggaagaagacgaggagctaatggaaagagagatggaaatcggatatccaactgatgtgaagcatgtaacacacataggattggatggaactacgatgacaaatcctataaagggctgggattgtctgaaatctccagaaataattccattcccttcatttactttaaggcagttcgagcttgcaatggctgcacaagctcatggacctcttgttggggtcgatcattccaagcttgtttgattcattgatttttcttttcatttcctgatcttgtttctttgacactagatgactgatgtgatgaagattgatcaatgtttttgatggaggcactggttgcagtgatgtgtttttggtgtttgtgtgggaccttgacaatgtttttctgggtggccattgaaattgttcttgcaaaaaaaaaaaa

SEQ ID NO: 35 - DNA, 823 bp, Artificial Sequence
  Synthetic Construct
gctagctagctttgcagagttctgtcgattcaatggcgaccaaagtgtatattgtgtactattccatgtatggacatgtagaggaactggcagaagagattaagaaaggggcttcatctgttgaaggtgttgaggcccaattatggcaggttcctgagacactgccagaagaggtgcttggaaagatgagtgcaccaccaaagagtgatgtaccagtcattacacctagtgaactcgctgaagctgatggctttgtgcttggattcccgacaagatttggaatgatggctgcccaattcaaagcttttctggatgcaactggaagtctatggagaacacagcaacttgctggcaagcccgctggaatcttctttagcactggatcgcaaggtggtggccaagagaccacagcgttgactgctattacccaacttgttcaccatgggatgatatttgttcccattggctacacatttggtgctggcatgtttgagatggagaaggtgaaaggtggaagtccttacggcgcaggaactttcgctggggatgggtcgagacagccaaccgagcttgaattggagcaggctttccaccagggcaagtacattgctgccatcacaaagaagctcaaaggagctgcttaagtttactcaattacaagaatacggtctggtcttccattttttcaccctatagaatatgcatttgcaaagaataattcgtattccaccgtttattatatgcttttcatgtgtgtaacatgatagtgattattgtctcaatgatatcaaggttggttgtgttttcatttaaaaaaaaaaaa

SEQ ID NO: 36 - DNA, 1113 bp, Artificial Sequence
  Synthetic Construct
gcggacgcgtgggctcttttctttctttctccatcacccacgcgttttcttagtttcaattcttgtcaaaatagtcttgatcattttctctgcaaaagaaattaatcatgtcaggtgatcaaaggcctaaagattctgctgaaggctcttcgcgatctggaggcgatcaccaccagcttcaatcacctcctttgagccggtatgagtcgcagaaacggcgagactggaacactttcgggcagtacttgaagaatcagagacccccagtttcattatctcagtgtaattgcaatcatgtgcttgatttccttcggtatcttgatcagtttggcaagactaaggttcatcaccatgggtgcgtcttctttggacaacctgatcctcctgctccttgtacatgccctctaaggcaagcttgggggagccttgacgccctaatcggacgccttcgagcagcttttgaggagcatggaggatcagcggagactaacccttttggaaatggagctattcgggtttatttgcgtgaagtgaaagagtgtcaagctaaggcaagagggattccttacaagaagaagnagaagaagaagactcaaataggcccagagacgaagcaaagccttcgatgcagacagcttngtcctttggcttcttccgataaatggatcacaaaataggacaaatggaggtgaagttccagaaaggcaaatgttgggggcctagcctctgcctcagtacttaatatatagctagctagcttctctctttgcagatagtgttacatcacagcctacttctaaataatcttgtcctcgcaggttggtctaagaagctattcctagcttataggaattaaaactgtagtcgtaatgttcatcgacttcttaaatagtaagacgttagcactatatgtccagctctgttgtttctttgctgcagaagaaaactagtgtcttcagctaatggtgaaaatggcttttagcttctgcccaaatgtagctttgggcctgcctctcattacatgagctgtgctttctagtaatccttcaattgtccaaagtaccatgtttttcttcatgtttaatctctagttaatttcctaaaaaaaaaaaa

SEQ ID NO: 37 - DNA, 991 bp, Artificial Sequence
  Synthetic Construct
cgcaatccatcctatggtgctttatctgactatcagtttatgcgaccccgcgcacgtggcaagaaacttcttgaagagtgggctgcccccttgaaaagtgttgaggatatatacgagaaattcaaggtatactgccttgggaagttgaaaagtagtccttggtcagaactagatggccttcagccagagactaaaatcattaatgaacagctgggtaagatcaacttgaagggactcctgactatcaatagtcaaccagctgtcaatggggaaaaatctgattctccatctgttggttggggtgggccaggagggtatgtatatcagaaggcttatctagagtttttctgctccaaggacaagctgaatgctcttgtggagaagtgcaagtctttcccttttgtaacttacattgctgtgaacaaaggagggagttggatttctaatgttgccctgactgatgtgaatgctgtaacctggggagtcttcccagcaaaggagattatccaaccaactgttgtggatcccaccagcttcagtgtgtggaaggatgaggcatttgaaatctggtcaagaggatgggcttccttgtacccagagggtgacccatccagaaccctacttgaagaggtgaagagcagctactttttggtcagcttggtagataatgattacatccatggggatattttcgctgtcttcgccgatttatgatgcaaggggctctttgcaacccctgcatttgctaagagtatccaagtccccagaaaaatggtggtgctttaccctaagttgaatcccagctttaatcatcttatcggcatacattgtttttgaaattatttccgtattgaaatgtaataattcattgtgattttgctactcccctctccattatatgctgcaaatttgtatttggagtggcaactaattctaagctttaagaaaatatgattgcctctttttcttggtacttccgaaaaaaaaaaaa

SEQ ID NO: 38 - DNA, 1273 bp, Artificial Sequence
  Synthetic Construct
ctctgctttctctatctagaaaacatatctctctttttcgtccctcacgttcccttgcaatggcttccagtggagtgaacgacaagtcactgatcgtcagcttcggggagatgctgatagacttcgtaccaacggtctctggcgtctccctcgctgaagctccggggtttgtgaaggcaccgggtggtgctccggcgaacgtggcgatagcggtggcgaggctgggaggaaaggcggcatttgttggaaaacttggtgatgatgagtttggcaacatgctcgccggaattttgaaggaaaacggcgtgatcgctacagggataaattttgacacaggtgctaggactgctctcgcgtttgttactctacgcgctgatggagagcgtgagtttatgttttatagaaatccaagtgctgatatgctactaagaccagaagagttaaatcttgagttaattagatctgctaaggttttccactatggatcaataagtttgattgtggagccatgcagatcagcacatttacaggcaatgagggtggcgaaggatgcaggtgcattgctttcgtatgacccaaatctgaggctgccattgtggccatcagaagaggaggcgcgtgagcagatattgagcatctgggacgaggcagatgtgatcaaagtcagtgataatgagcttgagttcctcactggaagtgacaaagttgatgatgaaactgcatgtcactttggcgccctaactttaagttgctcttggtcacccttggtgaaaagggttgcaattattacaccaagaatttccatggatcagttgaggctttccatgtagacaccgttgataccacaggtgctggtgatgcatttgttggtgctctcctttgcaagattgttgatgaccaatctgtgcttgaggacgagccaaggttgagagagatactaagatttgcaaatgcttgtggagccattacaaccaccaaaaagggagcaatccctgctcttcctactccggccgatgccctcaaactggtgaacgaaggaaaatgaactctttttttttaatccgcactgcatttttccctttccttttcagctcctggttctcaattcaatcgaagagctgttgcaatctcaaattttcgcctttttctctcgagtgtaatgatttgaaagacttgcgctttaaggtcttgccaaataaatttttttcttttgtgttagctttctccaaatattatatttgtttaaagcaaaaatttctgcaaaaaaaaaaaa

SEQ ID NO: 39 - DNA, 803 bp, Artificial Sequence
  Synthetic Construct
cttcgtcatgggggagaaggagcccaagaaagctgaatctgagggagcctctctgcctacacaagttcaagaacatggacctgttaaggaagagaaagaagtccctttaaatgattctgctaatgagaagagctcggtcctagttactgaaaaggttgcagatccacctgctactgcaaaaaactccagggggtcaaatgacagagatgctgtgcttgcgagggttgaagcggagaaaagatgtgctctacttaaagcatgggaagaaaatgagaaagctaaagcggagaacaaggctcacaagaaactctctgccattggatcatgggagacaatcaagagagaatctgtggaggcaaaaataaagaagtatgaggaaaaagtggaaaagaagaaggctgaatatgcagagaaaatgaagagcaaagtagccgaactccacaaggcagccgaggagaagaaagcaatgattgaagcaaaaaaaggggaggaccgtctcaaggtagaggaaactgcagcgaaattccgagcaactggttatacaccaaggaagtgtctaagatgttttggcaaattctaatgaacaatagtggggtaaacggaagagaaaggctgctagaattggatttgcttgattttttggcttgttattttatgctgtgctcaaatattgttggttcagctgtaaatgtcttttgatatttgtaaaaaccaataaatgggagtgcatgtgttacattacccaagttctgacaagacccgagtttgaatttgatattgtcttttgtgcgaaaaaaaaaaaa

SEQ ID NO: 40 - DNA, 1116 bp, Artificial Sequence
  Synthetic Construct
caaacaagcactggcactggcaatggcaatgccacttcttcttcaactaccaccaccacttcttcttcttcaacatcttcaagttccatgagcagtaataacagtaatggcaacaatggttgtcagcagaaccaaagtaaccagcttggagagacaagatcaagtctttattattcaacaaatgcaatgtcttttgttaccaagtctttgttgcctactagaagaagactcaggcttgatcctcctaacaagctcttcttcccttatgaaccaggaaaacaggttaggagtgccattggcataaaaaacactagcaagtctcatgtagctttcaagttccaaacaactgcaccaaagagctgttacatgcgtcctcccggggctatacttgcacctggtgaaagtcttattgcaactgtattcaagtttgtggagcctccagagaacaacgagagactatttgatcagaagagcagggtaaagttcaagatcatgagcttgaaagtgaaaggagaaatggaatatgttcctgagatgtttgatgagcagaaggatcaagtagcagttgagcaaattttacgagttgtttttcttgatccagaatgccctagccctgcactggaaaagcttaaaagacaactggctgaggctgaagctgagctcgaagcacgcaagaagcctccagaagatgcaggcccccgagttgttggcgaaggacttgttatagatgagtggaaagagcggagggaaagatacctggcacgccagcaggttgaagttgattctgtgtgaagatcctgttttccttgtaagcacttgttttttcttgagctgaggtttccttcatgcaatgttgaatgctttgcctctcggtccgttggtggaggcggatgtgtgataagtccggtccagttaggggtgcagacataagatttggaaagcaggatttatcagcaaagcctgtaaaaaaaaggtaaaatcaaggcatttgtcatgtcatattaagctgtaaggcacttaattatgtacttgtgccctatttattgtagatatgggattttgattaacgatgaattagttaaatttgtgttgtgaaaaaaaaaaaaaaaaaaaaaa

SEQ ID NO: 41 - DNA, 868 bp, Artificial Sequence
  Synthetic Construct
gggccttatagacaaagatgtggtgaggactgatagagcactgtctttctatgatggggatgataatccaaatgtgaatatcttacgtgatattctgttgacgtactccttctataactttgatcttggttactgtcagggtatgagtgatttgctatccccaattttgtttgtgatggaggatgaatcagaatcgttttggtgttttgtggcactgatggaacgtcttggacccaattttaatcgtgatcaaaatggcatgcactctcagctttttgcattatccaagctggtggagttgcttgattgcccattacataactatttcaagcagaatgactgcttgaattacttcttttgtttccgctgggttctaatacaatttaaaagggaatttgaatacaagaaaacaatgcgattgtgggaagtgttgtggacccattatttgtctgagcatctgcaactgtatgtatgtgttgcaatcttgaagcgataccgcaacaaaataatgggggagcgcatggactttgacacactcttgaaatttatcaacgagctgagtggccatattgaccttgacgcaatncttagagatgcagaggctctgtgcatatgtgctggtgagaatggtgctgctcgcatcccaccaggaactccaccttcattgcctactgagaatgagaatgctttattatatgctcaagatgatgaagtactgtaatacagttcgtctcattggtacgatataattggctttatattttgttttcttcatgaaatagctgtgtataattgctgttttttcgaaataaaatgttaataatcaagcaatcaactccatttgctgacctccttgaaaaaaaaaaaa

SEQ ID NO: 42 - DNA, 752 bp, Artificial Sequence
  Synthetic Construct
gttcaataagataagatcatcaccacctccaaaattcaagttcttacaggatgcggatgagaaactacatagaaggaaattgatgcaagaagctggggagaaggtccaaaggcatgatgtttttgctcaggatcataccaaaattcctgctagttcaaattcccacaaagacgaggatgatgggccttttatcaccatcattattgacaggaacaaagaaagagagcttaacgaacaaaatcatcaactaccagactatcagtcaagcacttcacaggtacttcctcttgctactgcaccttcaacatcaaaatcagcagccaagaaaagttccttctttcattagatctccgtgatcgagtaaaacgagatagtttttttttttttttttcctatgtatttctattaaccaactttagtcttctttttaggagttctttttctttaatctttctatgggatggtgttagaaggttaaccttgccaaacccttttgcaatgtctgaaaaatcttcgggtgggtggggagttggacacactctctgcagtataatgttaaaagttggacaagtgcaggtgggggtcttgagggagtaatctgttgtattcactcttcatatatctctgagctgtctgatatctgtaatcattctggtgagtggtggtactactgatgtttttgtccaaaaggcactgtaagaattctttcagaccatgcacttctttatttattttttctaaaaaaaaaaaaa

SEQ ID NO: 43 - DNA, 939 bp, Artificial Sequence
  Synthetic Construct
tcgtacgcccacgcgtccgcacctaggcgcgcaccaaagagggttaaatcaacactgaagatcgataagttggacgatattcgcaatgaatggattgattctgatattctaatatttaattcagggcattggtggacaccgagtaaactctttgaaatgggctgttattttctggtaggcagatcgctgaagctaggaatgcctatcactgctgcctttgaaagggcactgcatacatgggcatcatggcttaacactaccataaatgcaaatagaacaagcgtgttcttccgcacttttgaatcttcccattggagtggccggaaccgcctttcttgcaaagtgactccgcagccttcatcaagaactggagggagggatcgtagcccaatatctgacaccataatcaaggttgtgaaggcaatggccgttcccgtaacagttttgcatgtgacacccatgggtgcattccggagtgatgctcatgttggcacttggagtgacaatccatctgtccctgattgcagccactggtgcttacctggtgtacctgatatgtggaacgagattctcttgtcaaatatgttgtccaggaattaagaggccattctttccataagctagaaaatgcaactgtgtagtgtgttccatattcttccctcttcaattgaccccattcaatttccaaaccttctactcaactactttcagtacatacgaagatcttctctgctgagaagttgtgtgcatttgtcacaaatattgtttgttgtgtagttcagggatccaaaaattacttgatttgtctgttctagttgcacttgagttcacatgtaatggtaatgatttctctccagcgttagctgaaatctggaagctcttctcttcagatgccaccgagctgttgtacaataaaagcgttattgcaaaaaaaaaaaa

SEQ ID NO: 44 - DNA, 556 bp, Artificial Sequence
  Synthetic Construct
ggggaagttttagaagctgtcataatgaaggatcgggccacgggtcgtgctcgaggatttggttttgttgttttcgcagaccctgctgttgctgagagagttgtgatggaaaaacaccttatagatggtagaaatgttgaggcaaagaaagcagttcctagagaggatcagaacaccctgaacaaaaacagtagcagcgttaatggttcacctggccctgcccgaacaaagaagatatttgtaggaggtttagcatctacagttacagagagtgactttaggaagtactttgatcagtttggagtgattacagatgtggtagttatgtatgatcacaacactcaaaggccaagaggttttggattcatcacctatgattcagaggaagcagtggatagagtattgcacaaaacctttcatgaactcaacggtaaaatggttgaggtcaagcgggctgtccccaaagagttatctccagggccaactaggaaccagttaggggggtttaattatagtcctagtagagtcagtagcttcctcaatggttacactcagg

SEQ ID NO: 45 - DNA, 580 bp, Artificial Sequence
  Synthetic Construct
tggtgctggagattttgatattggtctaggaggggtagggtatgggaggaacaataggacagctgttgcaccagcatcatctcatgctgcatctaatggtggttatgatggggcttatgcagacttttatgagaagggctcattatacggggataatacctggcaatcttcaccttcggaactggatgtgtctggctcatttggttttgggcttggaaatgcaacttctgatgttatgactaaaaattctgctggttatgttggtggttatagtgttgctaatagacaatcaaatagaggaattgctgcataggatgattatgatataggtgacggtgaagtgaatctcgtacatgtttcttgttcagatgatcaagtttcaactttgctcatcatatagagcagtcacaaggaaatctgtgtatggtttgagcttttgagatttttctaatataggttttctttcttggaattttattatgtaaaatcacattgtggggtatactcaacatgattgtatgatgcatctttggtgacagctatattaaaaaaggaaacaattttttagcaaaaaaaaaaa

SEQ ID NO: 46 - DNA, 1441 bp, Artificial Sequence
  Synthetic Construct
ctctttacttgtgcacgcttaaagaagagatcacacaatggcaaacccttctctctgtctcctcttcctcctctctctcctgaccccagccctcgtctcctcttccccggttcaggaccccgaacttgtagttcaagaagttcatagggccatcaatgcctctaggaggaagttgggatatctctcttgtggaaccggcaaccccattgatgactgctggagatgcgatcccaattgggagaagaaccgccagaggctagctgattgtgcgattgggtttggtaagaatgccattggtggaagaaatggtaagatttatgtggttacagaatctggtagtgatgatcccgtgaaccctaagccagggactctcaggcatgctgtcattcaagaagagccattgtggatcatttttgctcgtgacatgacaattcaattgaaggaagaactgatcatgaactcgttcaagactatcgatggtagaggtgcgagtgtccatattgctggtggtccatgcattactatacagtatgttaccaacattattatacatggactaaacatacacgattgcaagagaggagggaatgctatggtgagggactccccaaaccactttggatggaggactgtatcagatggtgatggtgtgccatctttggtggtctcacatctgggtggaccataattcattgtcaaattgtaacgatggactcgttgatgccatccatgggcctcagccatcaccatttcgaacaattacatgacccaccatgataaagtcatgcttctggggcatagtgattcctatactcaagacaagaacatgcaagtcaccatagccttcaatcactttggagagggtcttgtccagagaatgccaagatgcagacatggatatttccatgtggtcaacaatgactacacccattgggaaatgtacgccattggagggagtgctagcccnaccatcaacagccaaggcaacagatttgtagcacctgacatcaggttcagtaaagaggtaacaaaacatgaagatgcaccagagagtgaatggaagaattggaattggaggtctgaaggagatctactgttgaatggagcgtttttcgtagcatctggcgcaggcgcttcatcaagctatgctagggcatcaagcttgggtgcaagaccatcttcacttgtcggtccaatcacgatgggggcaggtgcacttaactgcaagaaggggggtcgttgctagctgattgtgaaagattacacaatattttgtgactagtaatttggactataattaattagctggctagaaaagtaattaagcagaagtggaattaaaaaatgaggagagtgaagaaaagtccgagcactagtttctcctggctttttcttccctccaaatttttattctttttacccgaaaaaaaaaaaaaaaaaa

SEQ ID NO: 47 - DNA, 650 bp, Artificial Sequence
  Synthetic Construct
cgagaccagtggccttttctatccaagaatcaagcatggataaaacagaacaagatcttgattcttgttctgttgtcaacaccaatgtatcatcaccagagttcttcaacgccttcgagtttatttcctcgatgtcatccggctttgatctgtctggtctttttgagactaagaggaaatcaagctcaatgttcacttcaaaattttcagcgagtgcgatcatggaaaaaattgaaggggttgcaagggggctgagttataaggtggcaaaagtcaaagatttcaaagtgacgttacaaggtccatgtgaggggagaaaggggaagctggcggtgacggcggaggtgttcgaggtggcaccggaggttgcggtggtggagttctccaagtcttccggagataccttggagtatactaggttctgcgaggaagatgttaggcctgcactaaaagacatcgtttggacatggcaaggagacaatgcttgtaatcaagataacaacaatagtcatgtagaagatcctgagattcaaatgttgtagaaaggctttgatcttaatttggtgagttttaggattttatataatctgtaaataagtttttgttatttaattaatgcatcaagttgagtgttcttgggaaaaaaaaaa

SEQ ID NO: 48 - DNA, 957 bp, Artificial Sequence
  Synthetic Construct
cccacgcgtccggtattttctgtggaggagaaacaaaaagcaatctcttgggtttttgttcttttaaagatttggtttgggttctctctttgaaagttatcaagaacagtaaaggggctcttgattgactgatgcaagtttaagattttctattctttttagagaaagaaagaagttttcttttgaaatgaatttgatgtgtgaggttttcttngctgaagaagaagagaaggttgaaggagagtgggaaaaggagagatagtatttaccaaagccaaggtagctagcagaagtagagaggcagatagcaataaactacaaaataaaatttcatctaaggggttttagtttgttctgcttctgctgggtaattggcaatgtctgctactataatcagcgaccctatggtggtctctgcaccggagacacaagcaacagcagcagccgcagctgcggcaacgacagcaacacaactcattgcacagatagaggttgagtccgtcagatgtgattgctgtggcctaatcgaagagtgcacgccagaatacattgaaagagtacgcgaaagatatcatggaaaatggatttgtgggttatgcacggaggctataaaatatgaaattgtaaggactgagaggcttattagcactgaagaagcaatgacaaagcacatgaacgtctgcaagaagtttgtctcctcagggccacctcctgatccaacaactcatttgatagccgccatgaggcaaatcctgagaagaagcttggattctccgagaggttcaaggtcaaccccaagtagtcctaacaaaacgaacggagcaatccgtgtcgctgcacttgctaggtccgaaagttgcttccctgctttgtctggttgatagataattctgagcaccgtggaagacggggtgattcataaaatggtgatgaagctattggaagcaaaaaaaaaaa

SEQ ID NO: 49 - DNA, 1020 bp, Artificial Sequence
  Synthetic Construct
gtcaagataaagtcccattctttttcctagccccaagtttgtttgaggggtgataaaaaatgaaccaagagatgaatggtgttgacactgagattgatcagaaccaccaagagaatgtgcaagagaaaatcgattatgtgtttaaggtggtggtgatcggtgactctgcagtgggcaagacgcaaattctttccaggtttaccaagaatgaattctgctttgattcaaagtctaccatcggtgtcgagttcccagactaggactgtcatcattaaagacaaggtcatcaaggctcagatctgggatactgctggccaagaaaggtaccgggcagtgacaagcgcatactatagaggggcactaggggccatgttagtctacgacattaccaagagaccaacgtttgatcatgtggctaggtgggtggaggagctccgagcccatgctgacagctcaattgtgatcatgctgatcggaaacaaggctgatcttgtggacctcagggcagttccaacagaagacgcggtggaatttgcagaggatcaaggcctctttttttctgagacatcagcccttagtggtgacaatgtggatggtgcatttttcaggctgctagaagaaatttacggtgtgatttgtaagaagtcattggaatgtggcaatggaaaaccccatgctgctgatgccataacgcttagaggttctaagattgatggcatatcagggacggatctggggattagtgagatgaagaaattatctgcttgctcgtgttgatttgatcatttctcttgtgaattgtgtactataagacttcaccactcccatgttcttaattgattctgtggctttctttggaaagtggtgatcggtcgtgtggtgagggtggcaagttttttcttttctgtgacctgtcaagattttagcagtattgtacttgtcttacagaacccatgaatttggtttttttatatgtattgatttggatggatggttttccttttcctctgaaaaaaaaaa

SEQ ID NO: 50 - DNA, 1160 bp, Artificial Sequence
  Synthetic Construct
atgaactagaaattcaaagcttccaacacatattttaatggcttacaatccttcttcaaggtcttgttcttatgattctttttgtttcctattcattacattcgttcttccatatttcttcctcacaaaagctcaagctgcaagccatggtagaacctcatgtggtaccattccaataaactacccttttggcatcgatgatggctgtggcagtccatattacaggcacatgcttctatgctccgattcgggcattctcgagcttcgaacgccttccgggagataccatgttcgtagcataagctactcagaccctcacatgatagtcaccgatccattcatgtggaagtgtcaagatggtcatcactttcgtgcaactagggcatttagccttgatgcaagcacacatttaacactctcctctcaaaatgactacctcttcttcaatngtagtgaagagaaagtgattgttgagccaaaacctatcttctgcgagaggtttcctgatcggtgcgactcgacatgtgatagtgctagttacctttgcaggcacttgnccaggatgtggtgctgcattaggaggangttcttgctgctcttacttcccaaaagcgaccgaatctttgaggctgatgctgaagtattgtgctagttacactagtatttattggagaattaatggtgcaaatgctcctgatgatcatgtacctgagtatggtattagagttgattttgacattccagtgactacagattgccttcaatgtcaagacatgaagaaaggaggtggaagatgtggatttgacacaaaatcacagaatttcttatgtctgtgcaatcagagatcaaatgtcacaacatattgcaatggtatatcacagcagcagtagccgtagcaaggcaggaataattgcagggactgtaactggagtttcagctgctggggccttaggaattggtgctggtctatggtattggaagaaagtgagagcctcagcaccagtaacgtgtggggttcaaagcaatgagaataggctcttttaagaacaaatcaacaaaagtgaatactaatcactttctcttttctaatttccctattttttgagttgcctttcgcactttaataagcaatttaatggatctgttttctttgttttaaa

SEQ ID NO: 51 - DNA, 896 bp, Artificial Sequence
  Synthetic Construct
cattatcccaaaaaacgagcaaggcagatacagcagccaaaaccatgtcacccggtcccgtcgttgaggctgaacccgccgccgccgtggaagatacacagaagaagaagcctcctcagcaggacgtggatgaacctgtggtggaggacgtgaaagaagatgagaaggaagaggacgacgacgatgatgatgaagacgacgatgatgaagatgatgacaaggatgatgataccccaggtgctaatgggagttccaagcagagcagaagtgaaaagaagagtcgcaaggcaatgttgaagcttggcatgaaacctgttactggtgttagcagagtcaccatcaagagaaccaaaaatatactgttttttatctcaaagcctgatgtcttcaagagccaaaattctgagacctatatcatatttggagaggcaaagatagaggatttgagctctcagctgcagacacaggctgctcagcagtttagggtgccagacatgtcatctatgctaccaaaatcagatgcttctactgcagctgctgctgcaccagcagatgaagaagaggaagaagtcgatgagactggggttgagcctagggacattgatcttgttatgacacaggctggagtttctaggagcaaggctgtcaaggctctccagacgaacaatggggacattgtcagtgctatcatggagcttactacataggttggctccctggttactctcctattttctgctcacaagttcttggaacaatttaatcatggtagtcacattggcttgccatctatgaggtcgctaattatccattgtttgtgtcaaatttgagattattacctattgcggtttttctttagtagcaagctcttattgtgctctttgcaaaaaaaaaaa

SEQ ID NO: 52 - DNA, 415 bp, Artificial Sequence
  Synthetic Construct
attgaatagccaaatatctctctctctggactagaatgtgaaggagcagttagcgaaggaaagattggagaatgccggagctggtgtaccaagagcagagctcgtcgagatcaggatttagagctcgagatgcaagtcctgactctgtaattttcactctggagtccaacttcagtctcttctcttctgcttctgctagcgtcgatcgctgctcttttgcttccgatgctcatgatcatgactctctcgcctctgaaatctctctggtaaaaccatttctctaacttccctgcatgcatgcgttttctaacccatggtttttgagtgagttttttgagagtgcaatgcagcatttggcagctggtcatgatcaacaagagaactcttcgagtggtccagatcgaaacaacaacaa

SEQ ID NO: 53 - DNA, 566 bp, Artificial Sequence
  Synthetic Construct
tccttaacttgtccgagagaggagcaatttttaacaccgggcttgcctcacatttttcattggaaaatgcatgcctgacaattccattggggcattttatcagaatgtttaaaatatttatattttgcgtcggctctatagcatatgcttataagcattgaaatatgattcaactgaagtattcgcacatgttttgctgtttacgttttatatgattgttgtgtttagttctccacaagcagcctggcagttattcgatccggtatcttgacgttatcttatcacatgctgtaaatcctatggctcaaactggcaattattcaaggcagttcttaaagagttcaaggttttctttcctgttgatcaacctttgtaaagcttatgaagcaattgtgcaaataattggtgctcgacagaaggcaatctcttagttcgctcacgacatttctagccgattttcgggaaaacattagtggactggagtcattgattcaatcaaggtgaaattggaaatcatttagaaaagcaattcctgttctttgacaacactaggttcttctttctcg

SEQ ID NO: 54 - DNA, 581 bp, Artificial Sequence
  Synthetic Construct
tagagttcgagaggtcatcttcttgttataaaatcctcaaaagcttgaggtgaaaaaataagcaagggagaggagagagctagctagctagtcaagttaagagtcagaaaacaagaatgtcttccaggaagaaaaagaaggcagctctttatgagaagttacgtgctgctaccaattctaatgctatgaacaaaacttcaatcatagtagacgcgtcaaaatatattggagagttgaagaacaaggtggataggctaaaaaaagaaatcggaacatcttcaaccccccaaaattcattacctgcgcaggtcacagtggaaaacctagaaaagggtttccttattaatgtattttcaggaaagaattgccctggattacttgtctccatacttgaagccttcgaggaactaggccttgatgtgctcgatgctagggtttcttgtgaagacaatttccaacttgaagcgattggtggagaccaaaaccaaggccatgatgctcaagtagtgaaacaagcagtgctgcaagctatccataactggaatgaaggcagctagctagtgagcaagaatgacccaaat

SEQ ID NO: 55 - DNA, 598 bp, Artificial Sequence
  Synthetic Construct
gcttagtggtctgggtttagtgagaaatgtgattggacactgtttgagtgggcgcggtggaggattcctcttcttcggggatgatctttatgattcatctcgtgtagcttggacaccaatgtcacccaattcgaaacactattcacctggattcgccgaattgacttttgatgggaaaactactgggttcaaaatcctgatcgtagcttttgacagcggggcctcttatacttaccttaattctcaggcgtatcaaggtctaatttctctgatgaagagagaaatacccgcgaagcatttgacagaagcgctggatgatcagacgcttccgatttgctggaaaggacggaaacctttcaaaagcgtacgtgatgtcaagaaatacttcaagacctttgcattgagctttaagaatggcggaaaatctaaaactcaacttgaattcccaccagaagcttatcttatagtatcatccaacggaaacgcttgcttgggaattctaaatggtacagaagtaggtctgaatgatctgaatgtcattggagacatatcaatgcaagacagagtggtgatttatgacaatgagaagcaactgatt

SEQ ID NO: 56 - DNA, 273 bp, Artificial Sequence
  Synthetic Construct
gaaggtggtggttcaattatacacatagttgaccacatggatttggagccttggagtgtgcctgaagtactacggccgctgtatgaatcctcaactgtacttgctcaaaagacaacaatggtggctctacgccagctgcggcagatagctcaggaagcttctcagtctaatgtgaccaactggggcagacgacctgcagctctacgagcactgagccagaggttgagcaggggttttaatgaggctctcaatggatttagtgatgagggatgg

SEQ ID NO: 57 - DNA, 274 bp, Artificial Sequence
  Synthetic Construct
cgctgacaccaccactttcctccttgacagcccctccttcctcaacttccttcaacatctcaacaccaatagccattgcgattgtgaagatgataatacaaacgccactattgactctatcatccccaccataaaaatcacttcttgcatgctagaaatggatccaatgcttgtgtgtgcagtgtgtaaagatcagttcttgattgatgttgaggccaagcagctgccctgcagtcacctgtaccatccaggttgcattctcccctggctttct

SEQ ID NO: 58 - DNA, 259 bp, Artificial Sequence
  Synthetic Construct
aacgtgatttcaaagggtcgcaaaggaactcaaatagagccccaagatcccacagaactagagttattgacagaggttcgaacgagtttccaaaagaaggggaggaggcttaaagacgttttgaggagtggaaactgcatactgaggaagttccaaaaacatcgagaggacgattctaatcaagttctttacttcttctctcaagtggatatgaagttagtagcaagggttctgagcatgtccagagtaacaacagagc

SEQ ID NO: 59 - DNA, 1464 bp, Populus trichocarpa
caccctgaagccccatttcggtttttagtcgaaatcttgggttgggggctggaaatatgaggacatgcttgtgggtttttgccgtggttcttgttttcggttttgttgatggaaaagaaattgaaaggttgcgcacggagaggatttcaggaagcgctggggatgtgttggatgatgatccagtgggaaggttgaaggtctatgtttatgagcttccgagtaaatacaacaagaaactgctgcagaaggaccccagatgtctcacccatatgtttgctgctgaaatcttcatgcatagatttctcttatccagcccagttcgaacccttaatccagatgaagcagattggttttattcccctatataccccacttgtgatctcacacccatgggcttgccgttgcccttcaaatctcctaggatgatgagaagtgcgatacagctaatctcctccaattggccttactggaatcgtacggaaggggctgatcacttttttgttgtgccccatgactttggagcctgcttccactatcaggaagagaaagccattgagcggggcattctcccgttactccagcgttctactttggttcaaacttttgggcaacgaaatcatgtgtgcttgaatgaggggtcaattacaattcctccttttgctcctcctcaaaagatgcaggcccaccagattccccctgacattccacggtccatttttgtctatttccgtggattgttttatgatgtaaataatgatccagaaggtggttattatgcaagaggagcaagggctgcagtttgggagaatttcaagaacaatccactcttcgacatctccactgaccatccaacaacatattatgaagacatgcagcgagctatattttgcttgtgcccgctcggttgggccccgtggagtcctagattagttgaagcagtggtatttggatgcattcctgtcatcatagcagatgatattgttttgccatttgccgatgctatcccatgggaggaaattggggtgtttgtagcagaggaagatgtccctaacctggacacaatcctaacatctataccaccagaagtgattttaaggaaacaaaggcttcttgcaaatccttctatgaaacgtgcaatgttattcccacaacctgcacaaccaggtgatgctttccaccaaatactaaacgggctggctcgtaagttgccgcacgacaggagtgtttacttgaagtctggtcaaaatattttgaattggacagcaggaccagttggggacctgaaaccttggtaagagaagtgtcatttcctcaacagacatggctgtggtgtatccactgtttcatctaatcgcaaggactgtaaacttctctttgaagttttgatttttgtatagattcttgaatttcacatgtacatctgggacatagttcgatgctttaaataacttcgcatttttcttg

SEQ ID NO: 60 - DNA, 1495 bp, Populus trichocarpa
attgcaatggctgagaagcactcctcatcatcccttggggttgtttctaggaaatctatgttttgcttgttttctttcgtgtctgccttgtttatactgtcatggttttttatgttgcgttccacggtcagtgccggatttattgaccttagtttgttacctcatcctaaacttaattctaaagatgatttaggagatgtgaaatgtaacagttttgataataattgcaatcaagttcttaaggttttcatgtatgatttgccttctgagtttcattttggactcttggattggaaacctcaagggggtagtgtttggcctgatcttagagctaaagtgcctgcatatcctggtgggctgaacttgcaacatagtattgaatattggcttacaatggatcttcttgcttcagagattcctggcatacctagagctggtagtgcagtaagagttcaaaattctagtgaagctgatgttatatttgtgccattcttttcttctataagctataaccgatattccaaggtgaatccacaccaaaagaagagcaagaacaaatcattggaagaaaagttggtcaagtttgtgacttcgcaaaaggaatggaagaggtctggggggcgagaccatatcattttggctcaccatccaaatagcatgttatatgcaaggatgaagctatggacggcaatgttcatacttgcagactttgggaggtattctccaaatatagcaaatgttggcaaagatgtgattgcaccatacaaacatgtaattaaaagctatgccaacgactcctctaactttgacagccgcccgactttgctgtattttcaaggagccatatacaggaaagatggtggctttgctcggcaagaattattttatgctctaaaagatgaaaaagacgtgcattttcagttcggtagtgtacaaaaggatggagtgagcaaagcttctcaaggaatgcactcctccaaattctgccttaacatagcaggtgacacgccctcatcaaaccgcctctttgatgccattgccagccactgcgttccggtcatcatcagtgatgatatcgagctcccttatgaggatgtccttgactactctcaattctgcatttttgtccgcacttcagatgctgtcagagaaaagttcctcataaaccttgtcaggagtattaagaaggatgaatggactagaatgtggcagaggttgaaagaggtagaaaacttctttgagttccaatatccatcaaaggaaggcgatgcagttcaaatgatatggcaggctgtagcaagaaaagtccctgcaattaggttgaaggtaaataagtttaggcgattttctcgttttgggacccatgaagatggagtgttaagaaaaataccatcacctagtaatttttggtgaaacattcagtgtttctttatactttgaggttctgtttatatataaaggtgagaaattcaatttattctcactacagattttg

SEQ ID NO: 61 - DNA, 2487 bp, Populus trichocarpa
atggctacctattacccaacttcaagaagtcagaataacaatttgcacgctttgcttacaggggaccagaaacttgcttcttattctgatctgccttccgatcttagtagcatgaaaagttatacgaaccacactccagctgctgggtcatactcggacatattgtatgggggttccttgtcttctcaaaatggtgctgaattctcatctagtggagctagaaatgagatagtattcattccacctacaagtgacacgatgaatttacaatctgttggtgggcagttgaatacagcagcaggtaacctggttggtgactctgttagtggagattcccaggctgttccgccaaggatgcaccttggcattccagactgtgagcagaatttccagtctcaaggattaccactgaggcttggcatgcaggtgcaatctgcagtttctatgccttcacttcagtatcagtatctaaaccaaaatttcccttcatctctgagttcacatctgcttgtgccagagaagtggactttaccctgtgaaggtgatgaaagcaatcaaagcaaggagttgagagaatttgaaggattgcctggatttgccggaagcagccataaccctatcaaaacagagtcttcacacaatcctcagtacatagttggcctcagagatatgcatgctgaaatgaatatgtatggattgtctggttatgctaacacactcttgaactccagatacctcaagtcagtgcaacacttgctcgatgaggtggttaatgtaaaaaaggctttgaagcagcctcaatccaacaaatgctccgatgacttcaaggagagtgataggaggccgagtagctgttctatgcttccctcatcaaatgtgaaaccaccggatcctgctgagtctactgccgactccactcctgagctatcgccagtagaacggcaggacttacttgacaaaaagactaagcttctgtccatgctggaagaggtagacagaaaatacaaacagtattaccatcaaatgcaaattgtggtgttatattttgacacggtggctggtcatggggcagctaaatcatatacagcactcgccctccaaacaatttctcgtcactttcgctgtttgcgcgatgcaatcagtggccaaatagaagttatcatgaaaaggcttggggagcaaggtacttcaccaaatggtcaaggaggtataccacgtctccgttatgtagatcatcagaccagacaacagagggctctccagcagcttggtgtgatgcgacatgcttggaggcctcaaagggggctccctgagagctctgtttcagtccttcgagcttggctctttgagcatttccttcatccctacccgagtgattcagagaaaatcatgttagcaaggcaggcaggcttgaccagaagccaggtcgcgaactggtttataaatgcgcgggtgcgtctttggaagcccatggtcgaggacatgtacaaagaagaatttggtgattcagagacaaactccaaatcttctttggacgaaacaaccaaagcccatggagataaatcaggcaatcacttgacatctgagaataggctacgtgagttgtatgagagtgtgacatctacagctgctgatatttcccagccagggcaagcccatgacataaaatctagtcatattcttgaattagaaatgaaagaacctatggcgaaaacagtccttgagaatggttctcagggacccaatgtggcagaatctgatattatgaaatttccacgggaccgaaggttgaatatagatgatgaccataacttctgtccacatgggaatattccgtgtggccaaaatggtgatggaaatcttatgtctgctgctgctacatatgatgtatcacacttgaatggatttgcagtaggcagccagatgtctcttgcattgggattgcaaagtaacgacagtgattcatttcccacgtttgatggggcccatatgagaggtaacactatatcagcttcctcggtggggcataatgaggtggattatcactgtatggatacaggaaagcaacaagacaggattgccaattcccatcggttacatgattttgtggtttaagagaatccttctagctttacgagggagtttcatcaatcctgccaagagaaggattgatggagttttggataacataaagcagaagaaaacaagctcagtgatattggtttgctattcattcaagccccgtgctcttctcttggtttgaatagaagaaacccaacacccttacagtcacagacatagatgtaacataagggcatggtgcctgcttgtaatatacgtacagattacagggattgtagatatatctgtgcatatacaataagatttttgtgatcttttgtactcttaaggaatttctacaagctctccctaattgtgacactgatgtatcatttcattcctaattctgaaataaatgtgatttgc

SEQ ID NO: 62 - DNA, 957 bp, Populus trichocarpa
attcaattcaattgtaccagcagcagcagcagtagtcagagcttataactagctaattgattaaccatggagaaagcacatacaaaatctgctcttaagaagcttgttaaggctagctcacagtctgcaccatggagcaatgctgctagaggaatggcaaaagatgatctcaaggatccgctttatgacaaatccaaggttgccccaaaaccttttgcaaaagaaaacactaagccccaagaattcaaactccacactggacaaagagctctcaaacgtgccatgttcaactattctgtggcaaccaagatatatatgaatgagcaacagaagaggcaaatagagaggatacaaaagatcatagaagaagaagaggttcgtacgatgaggaaggagatggttccaagagctcaattgatgccttactttgacagacctttctttccccaaagatcaagcaggccattgacagttcctagagagccaagtttccacatggtgaacagcaagtgttggagctgcatccctgaggatgaactttactactactttgaacatgctcatccccatgatcatgcctggaagcctgttaagtaaataaattgccatgatttgtaggggaaaaaggagagtggagtattctctctgtgatgttaatttcttcatgctgtccaagttagtcttgtataatttggagtgttgatagtgtttactgtttagatcatgtggtaggtcttgtagtgggtgtaagtagtcgctttttcatagccgaaagtccttttcaatggggggttttgcttaccaataaggcggatattgtacttcgttttgtactattaaaaagcaagtcaccctcttttctgtcttttagcagtatggtcgtgcatgcccaattaagcacttcattttattttgctggaaatgaattatcaaagcagactataattcttttagcca

SEQ ID NO: 63 - DNA, 2017 bp, Populus trichocarpa
acttgggtttctcgcatttctactcatcgccagttcttctttgggtgtgattgccaaataatgacatagccaaccaagagaaagaaaacgaggcacagctctatttcgggcttcttgtttcttggtttcgctgtgttgtgtgctcggtttaaaacaaaatgcagcttcatatatcgccgagtttgaggcatgttacagtttttcctggaaaaggtgtcagagagtttatcaaagtgagggttggagctagaagggtttcttatcgaatgctcttctattctctcttgttcttcacgtttcttctccggtttgtgttcgttttgtccacagttgactccattgatggagaaaccaagtgctctacactaggctgcttggggaaaagactagggccaaggatattggggagaaggcttgactcggctgtaccagaggttatgtttcaagtgctagaacaaccacttggcaatgatgaattaaagggaaggagtgatattcctcaaacattagaggaatttatggacgaagtcaagaacacaagattagacgcgaaaacttttgctctcaagctcagagaaatggtcactctactcgaacaaaggactagaaatgccaaaatccaagaatatctgtacaggcatgtagcatcaagtagcataccgaagcagcttcattgccttgccttgaggctagccagtgagcactccactaatgcagctgctcgacttcagctccccttgccggaactcgtccctgcgcttgttgacaatacttattttcactttgtcctggcttcagacaatgtgcttgctgctgctgttgttgccaattcacttgtccaaaatgcgttgcgcccccagaagtttgtgctgcacataataacagataggaaaacttattcacctatgcaagcatggttctcactgcaccctttggctccggcaataattgaggtcaaggcattgcatcatttcgattggttcgcaaaggggaaagtgccagtcatggaagcaatggaaaaggatcagagggtgaggtcacagtttagagggggttcatctgcaattgtagcaaataacaccgagaagcctcatattatcgcagcaaaactacaaacacttagtcccaaatacaattcagtgatgaatcacatccgaattcatctacctgagttgttcccaagcctaaacaaggttgtgttcctagacgatgacatagtggtgcagtccgatctttcgcctctctgggacattgacatgaatggaaaggttaacggggcagtagaaacctgtcgaggagaagacaagtttgtcatgtcaaagaagttgaagagctatttgaacttctctcatcccttgatatcagaaaatttcaagccgaacgaatgtgcctgggcttatggcatgaacatatttgatttagaggcttggaggaaaaccaacataagcacaacataccatcactgggttgaagagaacttgaaatcagacctgagcttgtggcagctaggaacactacctccaggtctgatagcattccatggccacgtccacgttatcgatcccttttggcacatgttgggtctaggctaccaagaaaatacaagtttggcagatgctgagactgccggcgtcatccatttcaatggtcgagcaaagccttggctagatatagcattccctcagctccgccccctgtgggctaagtacatcaacttttctgacaagttcataaagggctgtcatattaggccatcttaacttgaattttgagatgaaaatgttgactgcgatcgtgattaatatatgtctatatataaggagaaaaaagcagagaggagagagttcttcacaaatccaaggccttgaatccagaatgtaaaatattcataaccaggcatttgtctgcttggtttttttagaattcatcctctgtgggtgaacaacccttccttattacttcaaatcttgattcaattttaatgagaaattaagcatgttatggattttcaaccgtt

SEQ ID NO: 64 - DNA, 1496 bp, Populus trichocarpa
aagctgatgatcaaatgaggacatcaaaaatttcttcagggaattcaggctatcatgcatggattcttgcttttgcaatgagtctactgatactgattgccttgtcgaagtcatggttttatgatcacgcttctgctactgcaagtgaggatttgcaatacttttcagtgattgtcccttcaaaagggcgggcctatcctcctgttcttgcttattggatatgtggtactagtggagatgggaagagaatgttaaggctattaaaagcaatttatcatccaaggaaccaatatctattgcagcttgatgctgaatcctcagattatgaaagggcagagttagttgtttcagttcaatctgaaagtttgtttcaagcatacggtaatgttaatgttgttggaaaaggttatgctatcaacgaaatgggatcgtctgctcttgctgccatactcaacgctgctgcattgcttcttaagctgagtgcagactgggattggttcatcaatttaagtgtctcagactatcctctagtgagtcaagatgatctcctccatgccttcacttccttgccaagagatctcaacttcatcaactatactaatgacactgcgaaaaacgagatacacaagattaaccaaattgttgtagaccccagcttacatcttcagaagagtagccacctttattacgctgttgagactcgaacaacacctgatgctttcaagatatttggaggttcaccctggctgattcttacaagagctttcatggagtactgtgtccaaggatgggacaaccttccaagaaaactactaatgtacttcagcaacacggcatccccacttgaatcgtatttccactctgtcctctgcaactctcctgagtttcaaaacacaacagtaagcaatgatttaaggtacaatattctggaaactactacagatggggaatcaccttatgacaaaatgctaaatggtggagcagcatttgcaaggccatttaaagaagatgctgctgcactaaacatgatagatgagaatgtcttgaaccgtgaacccaatggattggtgcctggaaaatggtgcttagaccaaggtctgaacaagagctcagaggcatcaaagcctccaggggaggatttgtgttcaacttggggtaacattaatgatgtcaagccaggatcttatggtatcaagcttgcatttttattgtctaagattgccggtgaagagaaattgacaactagtcaatgccttcaagctacacaaatggggtcatcatagaaccaagcatacatccttgtgtggataatttgtttcttttaatcgaaaactataggttaaatcaagcttcttagccatatgaatcctttgctaaatgcactataaaggcgtacttgcttgttagaacaaaagctagaagcacccagtgatatattgtacatacattcacattttatcgaggatttctacaataaactcattgtttttccctta

SEQ ID NO: 65 - DNA, 1703 bp, Populus trichocarpa
gagagagagaatcttcgtattgaaatgagggaacctattgaaaccaacaacggatcacctccaccactacctccacaagcgttgctcgagaggcttaaagattatggccaggaagacgcttttgccctctgggacgagctctctactgaagagcgagaactccttgtcaaggacgtcgagagcttagatcttccaaggttggatcggataatccgatgttcgcttcgatctcaagggctaccggcggcggcaattgagccggttccggagaataccgtgtcgacggtagaagatagaacgatagaagaaagagagagatggtggaagatgggattgaaagcaatctctgatggcaaattggctgttgtgctattatctggtggccaggggacaaggctaggaagttcagatccaaaaggatgtttcaatatagcgcttccatctggcaaatcactctttcaacttcaagctgagcgaattttgtgtgtccaaagattagcagcccaagcttcaagtgaaggttctggaagctcagtttcgatacattggtacataatgaccagcccgtttactcatgattccacacgatttttttttgaaaatcacaagtacttcggccttgaagcagatcaggttaccttcttccagcaaggcaccataccttgtgtttccaaggatggcagatttatcatggagactccatttagggtagctaaggctccggatgggaatggaggagtatattcagcgctgaaatattcaaaattattagaggatatggcctcaagagggatcaagcacgtggactgctatggggttgacaacgcactggtacgtgtagctgatccagcttttttgggatatttcattgataaaggtgtagcagctgcagcaaaagttgttcgtaaggcatacccccaagaaaaggttggtgtttttgtaaggcaaggtaaaggtggacctcttaccgtggttgaatacagtgagctggatcagtcactggcttctgcagttaatcaacaaactggacgccttcgtttttgttggagtaatgtgtgcttgcacatgttctctttggattttctaaaccaagtggcaaatggccttgagaaagacagcatttaccatctagctgagaaaagaattccttctattcatggtgatacgatgggattaaaactagagcaattcatattcgatgcattcccatatgctccttcaacagctctttttgaggtaccacgtgaagaagaatttgcacctgtaaaaaatgccaatgggtcaaattttgacactcctgagagtgctcggctgcttgttcttcgactgcattctcgctgggtggttgcagcaggtggcttcttaacacattcggtgcctttatatgcaactggtgtggaggtatcaccactttgctcttatgctggcgaaaatctagaggccatatgccggggaagaacatttcacgcaccttgtgagattacattctagttaaatctgtatccaaatattcttgatttgtacggatcccattggaaaggggttatccatcccacgcatgtgttgtggtagattggcgagagataagagttccaggttttgttacatcctgggtgtgatgtcaagaatgatacaatttgattaacgtatatcattcaagttattgttgagcaatgataatcctctcttcttcttttttccaa

SEQ ID NO: 66 - DNA, 1312 bp, Populus trichocarpa
ctctctctctctccctctccctctccctgccattccctcctttgctttctctatccagaaaacacacaactctctttctcgtccctcacgttctcttgcaatggcttccaatggagtgaacgacaagtcactgatcgtcagcttcggggagatgctgatagacttcgtcccgacggtctctggcgtctccctcgctgaagctccggggtttgtgaaggcaccgggtggtgctccggcgaacgtggcgatagcggtggcgaggctgggaggaaaggcggcatttgttggaaaacttggtgatgatgagttcggcaacatgctcgccggaattttgaaggaaaacggcgtgatcgctactgggataaattttgacacaggtgctaggactgctctcgcgtttgttactctacgcgctgatggagagcgtgagtttatgttttatagaaatccaagtgctgatatgttactaaggccagaagagttaaatcttgagttaattagatctgctaaggttttccactatggatcaataagtttgattgtggagccatgcagatcagcacatttacaggcaatgagggtggcgaaggatgcaggtgcattgctttcgtatgacccaaatctgaggctgccattgtggccatcagcagaggaggcgcgtgagcagatattgagcatctgggacgaggcagatgtggtcaaagtcagtgataatgagcttgagttcctcactggaagtgacaaaattgatgatgaaactgctatgtcactctggcgtcctaactttaagttgctcttggtcactcttggtgaaaagggttgcaattattacaccaagaatttccatggatcagttgaggctttccatgtggacactgttgataccacaggtgctggtgattcatttgttggtgctctcctttgcaagattgttgacgaccactctgtgcttgaggatgagccaaggctgagagagatacttagatttgcaaatgcttgtggagccattacaaccaccaaaaagggagcaatccctgctctgcctactccggccgatgccctcaaactggtgaaagaaggaaaataaactatttttttgttaatccgcactgcattttatccctttccttttcagctcctggttctcaattcaatcgaagagctgttgcaatctcaaattttcgcctttttctcttgagtgtaatgatggaaaagacttgcgctttaaggtcttgccaaataaaatttttcttttaaacaaattgatcttgggcatcaagcagcaatcatcaaagtgttagctttctccaaa

SEQ ID NO: 67 - DNA, 2353 bp, Populus trichocarpa
gaaacactgaaacaagcagaactcaacatcacacgctctcgggaatattattcactctcgaaacaaccagaaactcctccattgtagattcagagagagagggaggcagacagacagagagtaggcgatggcgggcacgtggagagcacgtggagctgtggtatccgcgattctcttattcgggtgtttgtttgcgatttcgattgcgaaagaggaggcaacgaagctaggaacagtaattggaatagatcttggaacgacatattcatgtgttggtgtttacaagaacggtcatgtggaaatcatagcaaatgaccaaggaaaccgtattaccccatcgtgggtggcttttactgacagtgagaggttgatcggtgaagcagccaagaatctggcggctgtcaatcccgagaggaccatctttgatgtcaagagacttattggaagaaagtttgaggataaagaggtgcagaaggacatgaaacttgtgccttacaagattgtgaataaggatggaaagccttatattcaagttaagattaaggatggggagactaaggtcttcagtcctgaagagataagtgctatgattttgaccaagatgaaggaaacagctgaggcattccttggaaagaaaattaaggatgctgttgtcactgttcctgcatacttcaatgatgcccaaaggcaagctaccaaggatgctggtgtcatagctggactcaatgtggctagaattattaatgagccaactgccgctgctattgcctatggtttggataagaagggtggtgaaaagaacattcttgtctttgatcttggtggtggtacatttgatgtcagtgtcttgacaattgacaacggtgtctttgaggttctttctaccaatggtgacacacatttgggaggtgaggattttgaccagaggattatggagtacttcattaagttgatcaagaaaaagcatggaaaggatatcagcaaggacaacagggctcttggaaaactcaggagggaatgtgagcgtgccaagagagctcttagcagccagcaccaggtccgtgtagagattgaatcactccatgatggtatggatttctctgagccactgaccagagcccgttttgaagagttgaacaacgatttgttcagaaagaccatgggacctgtgaagaaggctatggacgatgctggtttggagaagaatcagattgatgagattgttcttgttggtggaagtaccagaattccaaaggttcaacaacttcttaaggattactttgatggaaaggagcctaacaagggtgtgaaccctgacgaggctgttgcttttggtgctgctgttcaaggaggtatattgagtggagagggcggtgatgaaaccaaagatattcttcttttggacgtggctcccctcaccctgggtattgaaactgttggtggagtgatgaccaagttgattccaaggaacactgttattccaaccaagaaatctcaagttttcaccacttaccaggatcagcagactactgtctccattcaggtctttgaaggtgaaaggagtctcacaaaggattgcaggctgcttggtaaatttgatttgactagcattcctccagctccaaggggaacccctcaaattgaggtcacatttgaggttgatgccaatggtatcctgaatgtcaaggccgaagacaagggcactggtaaatcggagaagatcaccatcactaatgacaggggtcgtttgagccaggaggaaatcgagcgcatggttcgtgaggcagaggaatttgccgaggaggacaagaaggtgaaggagaggattgatgctcgtaacagtttggagacctatgtatataacatgaaaaaccagattaacgacaaagataagcttgctgacaagctcgagtctgatgagaaggagaaaatagaaactgctaccaaggacgcccttgaatggctggacgacaaccagaatgccgagaaagaggactatgaagagaagctcaaggaggtggaagctgtttgcaacccaatcatcactgccgtgtaccagagatctgctggagccccaggtggtggatcagcagaagattccgaggacgactctcaagatgaactctgagaatctttctgcttccttctacccctgctggaaagcaagggcaagggatgaggatgtaggctagatgaagttttgctgatcctagaagctaaagggttaaaataacatatccccctttttttggttttttgaggttaaaaatacatggaatagtttgttttctctacatgtttaaaacaccttctacgagttatagaaggtaggtccgtgagattgagtagtgtt

SEQ ID NO: 68 - DNA, 2774 bp, Populus trichocarpa
ggagctatgtctgtacttactcgtgtccaaagcattcgtgaacgtttggatgagaccctcaagactcatcgcaacgagattgttgccttactgaccaggattgaaggcaagggaaaaggaattcttcaacaccaccagattattgctgagtttgaagcaatccctgaagagatcaggaagatattggcgggtggtgctttttctgaagttctcagatccacacaggaagcaattgttttgcctccatgggttgcccttgctgtgcgcccaaggcctggtgtctgggagtacgtcagagtgaatgtccaagcacttgttgttgaggagttgcgtgttgctgagtatcttcatttcaaggaggaacttgttgatggaggctccaatggcaacttcgtgcttgaattggactttgaaccattcagtgcatctttccctcgcccaactctttcgaagtacattggtaacggtgttgagttcctcaaccgccacctttcagctaagttgttccatgacaaggaaagcctgcaccccctgcttgcatttctcaaggtgcactgtcacaaggggaagaatatgatgctgaatgacagaattcataacctagactctctgcaatatgttctgaggaaggccgaggaatatctgtcttctctgaaacctgagactccatactctcaattcgaacacaagttccaggagatcggcttagagagaggatggggcaatactgctgagcgtgttcttcagatgattcaacttcttttggatcttcttgaggcaccagatccctgcactcttgaaactttccttggcagaattcctatggtcttcaatgttgtgatcatgtcccctcatggatacttcgcccaagacaatgttttggggtatcctgataccggaggccaggttgtttacattttggatcaagttcgtgccttggagagtgaaatgcttctgcgtatcaagcagcaaggacttgatatcaccccccgaattctcattattactcgactgctccctgatgcagtaggaaccacttgtggacaacgtctggagaaagtctatggatcggagcattgtgatattcttcgagttcccttcagagatgaaaagggaatggtccggaaatggatctctcgctttgaagtttggccatacctagaaacttacactgaggatgttgctgctgaaattgctaaggagttgcagggaaagcctgatctgatcattggaaattacagtgatggaaacgttgttgcctccttgctagcacacaaattaggtgttacagagtgcactattgcacatgctctagaaaaaacaaagtacccagattcagatatttactggaagaagtttgatgaaaagtaccacttttcatgccagtttacagctgatctttttgccatgaaccatacagatttcatcatcaccagcacattccaagagattgctggaagcaaggatactgttggacagtacgagagccacactgctttcactctccctggcctctacagagttgttcatggtatcgatgtatttgatcccaaattcaacattgtatcccctggtgctgacgagagcatatacttcccttacactgagaagaaacttaggttgacttctttccatgaagaaattgaagaacttctttacagctccgttgagaacgacgagcacttatgtgtgctaaaagaccgcaacaagccaattctattcaccatggcgaggctggacagagttaagaatttaactggtcttgtagaatggtatggaaagaataccaagctgcgcgaattagctaatcttgttgtagttggtggtgatagaagaaaggagtctaaagatatagaagagcaagctgagatgaagaaaatgtacagtcatatagagaaatacaaattgaatggccagttcagatggatttcttctcagatgaaccgcgtgaggaatggagagctctaccgttacatttgtgataccaagggagctttcgtgcagcctgctttgtatgaggcttttggattgactgttgttgaggccatgacatgtggtttgccaacctttgctacttgcaatggtggtcctgctgagatcattgtgcatggaaaatccggattccatattgatccttaccatggagtacaggctgctgaactccttgttgacttctttgagaagtgcaaggctgatcccagttactgggacaaaatctcccagggaggcctgcagcgaatccaagagaagtatacctggaaaatttactctcaaaggctcctgactctcacaggagtttatggcttctggaagcatgtttccaaccttgatcatcgtgagagccgtcgctatctggaaatgttctatgcactcaaatatcgcaaattggctgattctgttcctttgactatcgagtaaatggagctggagaaatcaaggaaacatgggttggtttgagtcgggttccgggtccagaataatggtgtcatttcacgatagtgattggacaagaaaggctttgatcttctttttgtttttttgtttttcctttctgtgtgtatcttttttgtctttccttttcttttcatcatgctgcctcttgaatttcagtaaagattgtgtcttttgagttcatcatcccctctatccatttcttgcaaattctctgtctcttgagattggaaatcatgggaaatgatccagcatcaattcatgtatttctcctcaatactcgagattggaagttgatggctattcttcatcattatataaag

SEQ ID NO: 69 - DNA, 1428 bp, Populus trichocarpa
ctccaatcacgtgtccgctttacacaggtcgatccaaagcaattttctccattgcaagagaagccaagaatttgtgacgaacttggtataaattagttttgttttgacaagagagagacttctttgtttaatttccatgtgtctgaggagggaacgaagaacaaccccagggtgcttttcattgttcggacacctcttagattctcagacaacttgaacacaaaaatatttcccgagttccaggatcattttgaaggatacaaggaaagaggaataaaaagaaagaacaatggcattcacgggaaccctggataagtgcaaggcttgtgataagactgtttattttgttgatatgatgtctcttgaaggggtgccttatcataaatcctgcttcaaatgcagccactgcaaaggaactcttgtgatgagcaactactcatccatggatggagtcctctactgcaagactcattttgagcaacttttcaaggagggtggagatttcagcaagaattttcagaaagggaagcccgagaggactcatgacctgtctaggatccccagcaagctatcttcagtgttctgtggaacccaagacaaatgctccgcttgcggcaaaacagtgtacccattggagaaggtttgttttcagctctgaacttaaataaatccattaggtcctaaagacctgtgtgctatttatacccctacatttatcagataaatgaccaacatattcttaggtacatcttatctattatagatggatctatctgagaaatgttttcaagtctctgttatttaataggcatgcctatgataatctggaataagtacagatgtgctataaccattgatgacataggttaccatggagggagaatgttaccacaagacatgcttcaggtgtgctcatggtggatgtccactcacacattcatcttatgctgctcttgatggagtcctctactgcaaggtccacttcgctcagctcttcatggaaaagggaacctacagccatgtccttgcatcagctgcacacaagagatcaaattccacacctcctgaactggccgggtcaaacccagaggaaggagctgctgtggaggaggaaaagccagaagagcaatcttaaacaaagcacatcatgttttcttgcattgctatcttttcaagttgccaggactgctttgagttttcatgttgttgaatattattgccggattgtgttcaaagtttgtttttggatttgactatctgtgttacagatgccttgctacaggatattgaattgcaatagtttttacaaattcctcctcttgcaatttaatactcttggcaacaggatacaaatgaaaaacccaaatcctaggaaccagtatcaacatttattgatctcagctctttgctcagatggcttgcgttttc

SEQ ID NO: 70 - DNA, 776 bp, Populus trichocarpa
cttgttcaatcccggcactactcctgaatgcctgcattcccttttagattttgtgtttgattttgtcctgtaaggaggatatgcgggatcatatggaaagatttgtagttcttccattctccatcgcctgtgcctctcactccagtgttgatgtggcctccagtgaatcctgcaagaaaccaaaacccgaaaccaaatcacatgcaacaagaggacaagaaggggaggaaagctcttgtaaagaaaagacgaagaacaatacatttggtttcctgctggctcttccaaagccttgcatatccagtagcattcacaaattgattaggggcattaagactctctcccaagtatttgtgtacaaagaagaagacgaggagctaatggaaagagagatggaaatcggatatccaactgatgtgaagcatgtaacacacataggattggatggaactactatgacaaatcctattaagggctgggaatgcctgaaatctccagaaataattccattcccttcatttactttaaggcagttcgagcttgcaatggctgcacaagctcatggacctcttgttggggtcgatcattccaagcttgtttgattcattgatttttcttttcatttcctgatcttgtttctttgacactagatgactgatgtgatgaagattgatcaatgtttttgatggaggcactggttgcagtgatgtgtttatggtgtgtgtgtgggaccttgacaatgtttttctgggtggccattgaaattgttcttgcaa

SEQ ID NO: 71 - DNA, 819 bp, Populus trichocarpa
gagtgtaagctagctagctttgcagagttctgccgattcaatggcgaccaaagtgtatattgtgtactattccatgtatggacatgtagagaaactggcagaagagattaagaaaggggcttcatctgttgaaggtgttgaggcccaattatggcaggttcctgagacactgccagaagaggtgcttggaaagatgagtgcaccaccaaagagtgatgtaccaatcattacacctggtgaactcgctgaagctgatggatttgtgtttggattcccgacaagatttggaatgatggctgcccaattcaaagcttttctggatgcaactggaggtctatggaaaacacagcaacttgctggcaagcccgctggaatcttctttagcactggatcgcaaggtggtggccaagagaccacagcgttgactgctattacccaacttgttcaccatgggatgatatttgttcccattggctacacatttggtgctggcatgtttgagatggagaaggtgaaaggtggaagtccttacggcgcaggaactttcgctggggatgggtcaagacagccaaccgagcttgaattggagcaggctttccaccagggcaagtacattgctgccatcacaaagaagctcaaaggagctgcttaagtttactcaattacaagaatacggtctggtcttccattttttcaccctatagaatatgcatttgcaaagaataattcttattctactgtttattatatgcttttcatgtgtgtaacacgatagtgattattgtctcaatgatatcaaggttggttgtgttttcattt

SEQ ID NO: 72 - DNA, 1246 bp, Populus trichocarpa
aaggaaaagaaatcaaaattgttgctaattggacgagaggaagaaaaggagaacaaaagcaccttcatagggcccatttcatgtctcctcatgagacctaagcttccccccccactgcacaatcacactattcttctttcatctcttttctttccttctttctccatcacccacccgttttcttaatttcaattcttgtcaaaatagtcttgatcattttctctgcaaaagaaattaatcatgtcaggtgatcaaaggcctaaagattctgctgaaggctcttcgcgatctggaggcgatcaccaccagcttcaatcagctcctttgagccggtatgagtcgcagaaacggcgagactggaacactttcgggcagtacttgaagaatcagagacccccagtttcattatctcagtgtaattgcaatcatgtgcttgatttccttcggtatcttgatcagtttggcaagactaaggttcatctacatgggtgcgtcttctttggacaacctgatcctcctgctccttgtacatgccctctaaggcaagcttgggggagccttgatgccctaatcggacgccttcgagcagcttttgaggagcatggaggatcagcggagactaacccttttggaaatggagctattcgggtttatttgcgtgaagtgaaagagtgtcaagctaaggcaagagggattccttacaagaagaagaagaagaagaagactcaaataaggccaagagacgaagcaaagccttcgatgcagacagcttaagtcctttggcttcttccgataaatggatcacaaaataggacaaatggagatgaagttccagaaaggcaaatgttgggggtctagcctctgcttcagtacttattatatagctagctagcttctctctttgcagatagtgttacatcacagcctacttctaaataatcttgtcctgtcaggttggtctaagaagctattcctagcttataggaattaaaactgtagtcgtaatgtttctactacatcctcgacttcttaattagtaagacgttaacactatatgtccagctctgttgtttctttgctgcagaagaaaactagtgtcttcagctaatggtgaaaatggcttttagcttctgcctaaatgtagctttgggcttgcctctcattagatgagctgtacttactagtaatccttcaattgtccaaagtaccaagtttttcttcatgtttaatcttcagttaatttcct

SEQ ID NO: 73 - DNA, 2176 bp, Populus trichocarpa
acaactacaactgctacttgtaggtgtgggtttgctcttaactctttcattctatctgcgatcctctctccctcccccttggcacttgtcaagcagcagagaatgaaggttattgataagataagggcggcagcagcagcaggagattcaaatggaggagatagcagcagcaataataaggttgttttctccttcgaattctttcctccaaagaccgatgatggagtggacaacctgttcgagagaatggacaggatggtctctcacaatccttccttctgtgacatcacttggggtgctggtggctccaccgcagatctgaccttagacatcgccaacaagatgcagaacatcatctgtgtcgagactatgatgcatctcacctgcaccaacatgcccgttgagaagatcgatcacgccctcgaaaccatcaaatccaatggaatccagaatgttcttgctcttcgtggtgatcctcctcacggtcaggataaattcgtccagatccaaggcggcctcgcttgcgctctcgacctcgtcaaacatatcagatccaaatatggtgattacttcggcattactgtcgctggctatccagaggcacatcctgatgtgattggaagtgatggatttgctacgcttgaggattaccataaagatcttgcttatttgaagcagaaggtggatgctggggctgatctaattgttactcaactcttttatgatactgatattttcttgaaatttgtgaatgactgccgccaaatcggaatcaattgtcccattgtccctggtattatgcctattaataactacaagggcttcatacgtatggctggtttttgcaaaaccaaggtaccagccgaggttactgcggcattggagcctatcaaggataatgaagaagccgtcagagcctatgggattcaccttggaactgaaatgtgcaagaagattttagctcatggaatcaagacgttgcatctttatactctgaacatggagaaatctgctttagctatattaatgaatcttggtttgattgaagagtccaagattacaaggccgttaccatggcgccccccgacaaatgttttccgtgtaaaagaagatgtccgtccgatattttgggctaatcgtccaaagagctacctaacaaggacaataggttgggaccagtaccctcatggccgatggggtgattctcgcaatccatcctatggtgctttatctgactatcagtttatgcgaccccgggcacgtggcaagaaacttcttgaagagtgggctgcccccttgaaaagtgttgaggatatatacgagaaattcaaggtatactgccttgggaagttgaaaagtagtccttggtcagaactagatggccttcagccagagactaaaatcattaatgaacagctgggtaagatcaacttgaagggattcctgactatcaatagtcaaccagctgtcaatggggaaaaatctgattctccatctgttggttggggtgggccaggagggtatgtatatcagaaggcttatctagaatttttctgctccaaggacaagctgaatgctcttgttgacaagtgcaagtctttcccttttgtaacttacattgctgtgaacaaaggagggagttggatctctaacgttgccctgactgatgtgaatgctgtaacctggggagtcttcccaggaaaggagattatccaaccaactgttgtggatcccactagcttcagtgtgtggaaggatgaggcatttgaaatctggtcaagaggatgggcttccttgtacccagagggtgacccatccagaacgctacttgaagaggtgcagagcagctactttttggtcagcttggtagataatgattacatccatggggacatttttgctgtcttcgcggatttatgatgctaggggccctttgcagcccctgcactttctaagagtctatccaagtccccaaaaaatggtgctttatcctaagttgaatcccagctttaatcttcttatggcatacattgtttttgaaattatttccgtattgaaatgtaataataatccattgtgattttgctactcccctctccattatatgctgcaaatttgtatttggagtggcaactaattgtaagcttaaagaaaatatgattgcctctttttcttg

SEQ ID NO: 74 - DNA, 1304 bp, Populus trichocarpa
ctctctctctctccctctccctctccctgccattccctcctttgctttctctatccagaaaacacacaactctctttctcgtccctcacgttctcttgcaatggcttccaatggagtgaacgacaagtcactgatcgtcagcttcggggagatgctgatagacttcgtcccgacggtctctggcgtctccctcgctgaagctccggggtttgtgaaggcaccgggtggtgctccggcgaacgtggcgatagcggtggcgaggctgggaggaaaggcggcatttgttggaaaacttggtgatgatgagttcggcaacatgctcgccggaattttgaaggaaaacggcgtgatcgctactgggataaattttgacacaggtgctaggactgctctcgcgtttgttactctacgcgctgatggagagcgtgagtttatgttttatagaaatccaagtgctgatatgttactaaggccagaagagttaaatcttgagttaattagatctgctaaggttttccactatggatcaataagtttgattgtggagccatgcagatcagcacatttacaggcaatgagggtggcgaaggatgcaggtgcattgctttcgtatgacccaaatctgaggctgccattgtggccatcagcagaggaggcgcgtgagcagatattgagcatctgggacgaggcagatgtggtcaaagtcagtgataatgagcttgagttcctcactggaagtgacaaaattgatgatgaaactgctatgtcactctggcgtcctaactttaagttgctcttggtcactcttggtgaaaagggttgcaattattacaccaagaatttccatggatcagttgaggctttccatgtggacactgttgataccacaggtgctggtgattcatttgttggtgctctcctttgcaagattgttgacgaccactctgtgcttgaggatgagccaaggctgagagagatacttagatttgcaaatgcttgtggagccattacaaccaccaaaaagggagcaatccctgctctgcctactccggccgatgccctcaaactggtgaaagaaggaaaataaactatttttttgttaatccgcactgcattttatccctttccttttcagctcctggttctcaattcaatcgaagagctgttgcaatctcaaattttcgcctttttctcttgagtgtaatgatggaaaagacttgcgctttaaggtcttgccaaataaaatttttcttttgtgttagctttctccaaatattatatttgtttaaagctaaaatttctgc

SEQ ID NO: 75 - DNA, 853 bp, Populus trichocarpa
gaagtatttggtgacaattgtcttcgtacatgggtttccgggtcttggtggcgaaagtaaaagacttcgttatgggggagaaggagcccaggaaagctgaatctgagggagcttctctgcctacacaagctgaagaacatggacctgttaaagaagagaaagaagcccctttaaatgattatgctaatgagaagagctcggtcctggttactgaaaaggttgcagatccacctgctactgcaaaaaactcaagggggccaaatgacagagatgctgtgcttgcgagggttgaagcggagaaaagatgtgctctaattaaagcatgggaagaaaatgagaaagctaaagcggagaacaggctcacaagaaactctctgccattggatcatgggagacaatcaagagagaatctgtggaggcaaaaataaagaagtatgaggaaaaagtggaaaagaagaaggctgaatatgcagagaaaatgaagaacaaagtagccgaactccacaaggcagccgaggagaagaaagcaatgattgaagcaaaaaaaggtgaggaccgtctcgaggtagaggaaactgcagcgaaattccgagcaactgggtatacaccaaggaagtgtctaagatgttttggcaaattctaatgaacaacagtggggtaaacgggagagaaaaggctgctagaattggatttgcttgattttttggcttgttagtttatgctgtgctcaaatattgttggttcagctgtaaatgtcttttgatatttgtaaaaaacagtaaatgggagtgcatgcgttacattacccaagttctgacaagacccgagtttgaatttgatattttcttttgtg

SEQ ID NO: 76 - DNA, 849 bp, Populus trichocarpa
caaacaagcactggcactggcactggcaatggcaatgccacttcttcttcaactaacaccaccacttcttcttcttcaacatcttcaagttccatgagcagtaataacagtaatggcaacaatggttgtcagcagaaccaaagtaaccagcttggagaggcaagatcaagtctttattattcaacaaatgcaatgtcttttgttaccaagtctttgttgcctactagaagaagactcaggctcgatcctcctaacaagctcttcttcccttatgaaccaggaaaacaggttaggagtgccattggcataaaaaacactagcaagtctcatgtagctttcaagttccaaacaactgcaccaaagagctgttacatgcgtcctcccggggctatacttgcacctggtgaaagtcttattgcaactgtattcaagtttgtggagcctccagagaacaatgagagactactggatcagaagagcagggtaaagttcaagatcatgagcttgaaagtgaaaggagaaatggaatatgttcctgagatgtttgatgagcagaaggatcaagtagcagttgagcaaattttacgagttgtttttcttgatccagaatgccctagccctgcactggaaaagcttaaaagacaactggctgaggctgaagctgagctcgaagcacgcaagaagcctccagaagatgcaggcccccgagttgttggcgaaggacttgttatagatgagtggaaagagcggagggaaagatacctggcacgccagcaggttgaagttgattctgtgtgaagatcctgtttccttgcaagcacttgttttttcttgagctgaggtttcctt

SEQ ID NO: 77 - DNA, 1598 bp, Populus trichocarpa
atgggttttaaccgggttttgcagaaaaccttgtcctctttagagttgcctgtttatattgcaagtggaccatctgcatccgtttcagatggtggtgaatcttcatcttgtgaactccaagagaggattggtgactctatccgtgatgaaattcctaggccttctcaaaatcctgggaggcagaagcataagagtcatgatcctgctcgagatcttacgatccatgtattggaaaaattctctcttgtgaccaaatttgctcgggacacaagttcacaactatttcgagaaagtaatagcaatggctatggtgctgtagaaaggaaaagttctagctactctctccctgatgtcccccataagccatccatggatgcagagatagctcttgaggaaggtcctgttccctcagatcctctggagttcgataaaatgacactcgtatggggaaaaccacgacagccacccttggggtcagaagagtgggctactttcttggattctgaagggcgagtcatggattcaaaagccttaaaaaagagaatattctatggtggagttgagcacacaacacgcagagaggtctggccatttttgctgggatatcatgcttatgattcaacgtatgcggagagggaatacctcaagtcttccaaaaagtcagagtatgagacagtaagacaacaatggcagagtatctctactgaacaagcaaagagatttacaaaatttagggaaaggaagggccttatagacaaagatgtggtgaggactgatagagcgctgtctttctatgatggggatgataatccaaatgtgaatatcttacgtgatattctgttgacgtactccttctataactttgatcttggttactgtcagggtatgagtgacttactatccccaattttgtttgtgatggaggatgaatcagaatcgttttggtgttttgtggcactgatggaacgtcttggacccaattttaatcgcgatcaaaatggcatgcactctcagctttttgcattatccaagctggtggagttgcttgattgcccattacataactatttcaagcagaatgactgcttgaattacttcttttgtttccgctgggttctaatacaatttaaaagggaatttgaatacaagaaaacaatgcgattgtgggaggtgttgtggacccattacttgtctgagcatctgcacctgtatgtatgtgttgcaatcttgaagcgataccgcaagaagataatgggggagcacatggactttgacacgctcttgaaatttatcaatgagctgagtggccatattgaccttgacgcaatacttagagatgcagaggctctgtgcatatgtgctggtgagaatggtgctgctcacatcccaccaggaactccaccttcattgcctactgagaatgagaatgctttattatatgctcaagatgatgaagtactgtaatacagttcttctcattggtacgatataagcggctttattttttgttttcttcatcaaatagctgtgtataattgctgttttttcgaaataaaatgttaataatcaagcaatcaactccatttgctgtcctcctt

SEQ ID NO: 78 - DNA, 1222 bp, Populus trichocarpa
agagattggagatgattacagcagcccagcgagagagctcttctttatgttctgtttaagaaagccatcttctttaaggcacaatcaagaactatgttcatcagtgagaataacagatgcacttgtccaacacgagcaagaatcccagctcaatattaatacaagcaaagatatattgctccggccctggagtgatgataatgtggagacagagctcatgaggctgcatagtctctcaggtccaccaagatttctctttacaattgttgaagaaacaaaggaggacttagagtctgaagatggcaggtctagaggtgatcctaagagtgcgaaaggatcaagaagtagaagcttaagtgatttgcttcgtactgtggagactccatatctaacccctctttcttctccgccatttttcacacctcctcttactcctagctataatcagattggattcaatcatctctttgaatcgtcaaaagatgcagagttcaataagataagatcatcaccacctccaaaattcaagttcttacaggatgcggatgagaaactacatagaaggaaattgatgcaagaagcaggggagaaggtccaaaggcatgatgtttttgctcaggatcataccaaaatacctgctagttcaaattcacacaaagatgaggatgatgggccttttatcaccatcattattgacaggaacaaagaaagagagcttaatgaacaaaatcatcaactaccagactaccagtcaagcacttcccaggtacttcctcttgctacttcaccttcaacatcaaaatcagcagccaagaaaagttccttctttcattagatctccgtgatcaagtaaaacgagatagtttttttttttccccatgtatttctattaaccaactttagtcttctttttaggagttctttttctttaatatttttatgggatcgtgttagaaggttaaccttgccaaacccttttgcaatgtctgaaaaatcttcgggtgggtgggggagttggacacactctctgcagtataatgttaaaagttggacaagtgcaagtgggggtcttgagggagtgatctgttgtcttcactcttcatgaatctctgagctgtctgatatctgtaatcattctggtgagtggtggtactactgatgtttttgtccaaaaggcactgtaagaattctttcaagaccattcacttctttatttattttt

SEQ ID NO: 79 - DNA, 1418 bp, Populus trichocarpa
atggcatatgtatatgtgattctgtttccaagatttcagccagttattaataagacgtatgagactccccatttcaatagttctgtaagtgagtgcaattattttgagggaaactggatacaagatgagagttaccctttatatgatgcatctcagtgtcctttcgctgagaatggatttaattgcttggctaatgggagaagagacggaggttatacaaaatggaggtggaagcctaaaaattgtgaaattccgaggtttaatgcgcgtgaaattcttgaaaagatgcgtgggaagcgaattgtttttgttggtgattcattgagtagaacacagtgggagtctttgatatgtatgctcatgacaggtgtggaggacaagagaagtgtttatgaaatcaatgggaataaaatcaccaagcagattaggtttttaggtgtacggtttagttcttttgacttgagaattgatttctatagatcagtttttctggtgcagcctggtccggcacctaggcgcgcgccaaagagggttaaatcaacactgaagatcgataagttggacgatattcgcaatgaatggattgattctgatattctaatatttaattcagggcattggtggacaccgagtaaactttttgaaatgggctgttattttctggtaggcggatcgctgaagctaggaatgcctatcactgctgccttcgaaagggcactgcatacatgggcatcatggcttaatactaccataaatgcaaatagaacaagcgtgttcttccgcacttttgaatcttcccattggagtggccggaaccgcctttcttgcaaagtgactcggcggccttcatcaagaactggagggagggatcgtagcctgatatctgacatcataatcaaggttgtgaaggcaatggcagttcccgtaacagttttgcatgtgacacccatgggtgcattccggagtgatgctcatgttggcacttggagtgataatccatctgtgcctgattgcagccactggtgcttacctggtgtacctgatatgtggaacgagattctcttgtcaaatatgttgtccaggaattaataggccattctttcccataagctagaaaatgcaactgtgtagtgtgttccatattcttccctcttcaattgaccccattcaatttccaaaccttctactcaactactttcaatacatacgaagattttctctgctgagaagttgtgtgcatttgtcacaaatattgtttgttgtgtagttcagggatccaaaaattacttgatttgtcttttctagttgcacttgagttcacatgtaatggtaatgatttctttccagcgttagctgaaatctggaagctcttctcttcagatgccaccgagctgttgtacaataaaagcgttattgct

SEQ ID NO: 80 - DNA, 2038 bp, Populus trichocarpa
aggtacccatttttttgttgttggaaacaaacccattttctctctctgtctttgtgtctttctttgataaattttaatcatcaacaacaacgacaacagtaacttgcaaagagagaaagagggaggagagagagcaaaaaagaaagggtctctctcaccctctcttattgttatgtctcctcgcagatcttagattctttcagtttcctatctgggttcttctttttgtttgtcaggattcctggattgcttttgattgatatttgattgctcaaaggttgtttctacttttggattaaatccaagagtgaccttttgttgtaaatatccaagctttgctttgtatttttgacaagaatggaaatggagcttggaaagttgttcattggtgggatttcatgggacacgaacgaagaccgtcttaaggagtatttccgggcttttggggaagttttagaagctgtcataatgaaggatcgggccacgggtcgtgctcgaggatttggttttgttgttttcgcagaccctgctgttgctgagagagttgtgatggaaaagcaccttatagatggtagaaatgttgaggcaaagaaggcagttcctagagaggatcagaacaccctgaacaaaaacagtagcagcgttaatggttcacctggccctgcccgaacaaagaagatatttgtaggaggtttagcatctacagttacagagagtgactttaggaagtactttgatcagtttggggtgattacagatgtggtggtcatgtatgatcacaacactcaaaggccaagaggttttggattcatcacctacgattcagaggaagcagtggatagagtattgcacaaaacctttcatgaactcaacggtaaaatggttgaggtcaagcgggctgtccccaaagagttatctccaggaccagctaggaaccagttaggggggtttaactatagtcctagtagagtcagtagcttcctcaatggttacactcagggatacaatccaagttttgttggagggtatggcgttagaatggatggcaggtttagtcctgttactgctgggcggagtaacttttctccatttggtagtggttatgggatgggattgaattttgagcaggtgttgaacccagtttatgggggaaattcaaacatcagttctaatgctggctatggacgagtaagtccttcatatagtggaaatgcaagcaggtacagcaaccccattggatttagtggaggcaatggaggaagtagttctgtcttaaatccaattgcccactctttatggggaaatggaagtattaatcatgcttctaacaccacaaactccagttcttttatgagttctggaactgggagctcaggaatgggttcttttagcagtatgggagcactttggggttcctctgctaattctgaacaaggtggaggagttcgcgcagtcaatagcagtaatcttagctttggtggtggagattttgatattggtctagaaggggtagggtatggaaggaacagtaggacaggtgttgcaccagcatcatctcatgctgcatctaatggtggttatgatggggcttatgcagacttttatgagaagggctcattatacggggataatacctggcaatcttcaccttcggaactggatgtgtctggctcatttggttttgggcttggaaatgcaacttctgatgttatgactaaaaattctgctggttatgttggtggctatagtgttgctaatagacaatcaaatagaggaattgctgcataggatgattatgatataggtgacggtgaagtgaatttcgtacatgtttcttgtccagatgatcatgtttcaactttgctcatcacaaggaaatctgtgtatggtttgagcttttgagatttttctaatataggttttctttcttggaattttattaagatgtaaaatcacattgtggggtatactcaacatgattgtatgatgcatctttggtgagagctatattaaaaaaggaaacaattttttagcatta

SEQ ID NO: 81 - DNA, 1611 bp, Populus trichocarpa
acactcttccctctctttacttgtgcacgcttaaagaagagatcacacaatggcaaacccttctctctgtctcctcctcctcctctctctcctgaccccagccctcgtctcctcttccccggttcaggacccagaatttgtagtacaagaagtacatagggccatcaatgcctctaggaggaagttgggatatctctcttgtggaaccggcaaccccattgatgactgctggagatgcgatcccaattgggagaagaaccgccagaggctagctgattgtgcaattgggtttggtaagaatgccattggtggaagaaatggtaagatttatgtggttacagaatctggtaatgatgatcccgtgaaccctaagccagggactctcaggcatgctgtcattcaagaagagcctttgtggatcatttttgctcgtgacatgacaattcaattgaaggaagaactgatcatgaactcgttcaagactatcgatggtagaggtgcaagtgtccatattgccggtggcccatgcattactatacagtatgtgaccaacattattatacatggactaaacatacacgattgcaagagaggagggaatgctatggtgagggactccccaaaccactttggatggaggactgtatcagatggtgatggtgtgtccatctttggtggtgctcacatctgggtggaccataattcattgtcaaattgtaacgatggactcgttgatgccatccatgggtcctcagccatcaccatttcgaacaattacatgacccaccatgataaagtcatgcttctggggcatagtgattcctacactcaagacaagaacatgcaagtcaccatagccttcaatcactttggagaaggtcttgtccagagaatgccaagatgtagacatggatatttccatgtggtcaacaatgactacacccattgggaaatgtacgccattggagggagtgctagcccaaccatcaacagccaaggcaacagatttgtagcacctgacatcaggttcagtaaagaggtaacaaaacatgaagatgcaccagagagtgaatggaagaattggaattggaggtctgaaggagatctactattgaatggagcatttttcgtagcatctggcgcaggcgcttcatcaagctatgctagggcatcaagcttgggtgcaagaccatcttcacttgtcggtccaatcacgatgggggcaggtgcacttaactgcaggaaggggggtcgttgctagctgattgtgaaagattaaacaatattttgtgacgagtaatttggactataattaattagctggctagaaaagtaattaagcagaagggaattaaaaaatgaggagggtgaagaaaagtccaagcactagtttcccctggctttttcttccctccaaatttttattctttttacccattgttatctgctcttatctttcctcccccttctccttttgtagtcaaaccttgaagattacaacctcagcctgttactctttgaacctaaggcattctccttgagaattcttggtgggcatagagaaaacaagtgctcaagtgttgatatagtatatatcaattatctattcat

SEQ ID NO: 82 - DNA, 1798 bp, Populus trichocarpa
atataaaaataatactaggtgctctccaatggctgcaaaaccatcaacagcgttccttttctcttctatattttaaagaaaaaacaccttctttcaacaaccttttgatggattccatcagaattagcatctcttcaatcctttcttggattcccttagaacatgttctttgatagccaaatcaccaaagttgaacaaaaagagaaaatcttgttgagttcttgagctaaacgagaggaggaggagaattgtttttcgtttttcaaatatggaggagaggcatgttatttttgggaagtatgaaatgggtaggcttttagggaagggaacttttgctaaagtttactatgggaaacacttggtgacaggagagagtgtggcaatcaaagtcataagcaaagatcaagtcaagaaagaagggatgatggagcaaatccagagagaaatctcagtcatgcgtcttgttcgtcatcccaacattgtagagctcaaggaagtcatggctaccaagacaaaaatcttcttcatcatggagtatgttcgaggaggagagttgtttgccaaagtagccaaaggaaggctgaaagaagaagctgctcgaaaatatttccagcaactaatcagcgcaattgattattgtcatagcagaggtgtttatcatagagatttgaagccagagaatatgttgcttgatgaagatgaaaacttgaaaatctctgattttggcttgtcagccttgcctgaacaatttcggcaagatgggcttttgcatactcagtgtggaactcctgcttatgttgccccagaagtcttgagaaagaaaggctatgatggatcaaaagctgatatatggtcatgtggggtgattctttatgtgcttcttgcaggattcttgccatttcaagatgagaatgttatgaagatgtacaggaaagtcttcaaggctgaatatcagtttccgccttggttttcaacagattctaagaggttgatttcaagacttcttgttgctgatcctgaaaagagaatcacaattcctgccataatgagaaatcattggtttcttaaagggttttcaagaccagtggccttttctatccaagaatcaagcatggatcaaacaggacaagaacaagatcttgattcttgttctgttgtcaacaccaaggtatcatcaccagaattcttcaatgcattcgagtttatttcctcaatgtcatccggctttgatctgtctagtctttttgagactaagaggaaatcaggctctatgttcacttcaaaattttcagcgagtgctatcatggaaaaaattgaaggggttgcaaaagggctgagttataaggtggcaaaagtcaaagatttcaaagtgacgttacaaggtccatgcgagggtagaaaggggaagctggcagtgacggcggaggtgttcgaggtggcaccggaggttgcggtggtggagttctccaagtcttccggagataccttggagtatactaagttctgtgaggaagatgttaggcctgcactaaaagacatcgtttggacatggcaaggagacaatgtttgtaataaagataacaacaatagtcatgtagaagatcgtgaaattcaaatgttgtagaaattaaggctttgatcttaatttggtgagttaattttaggattttatataatctgtaaataagttttcgttatttaattaatgcatcaagttgagtgtttttgttaagctagcacagtgtgtgtctatatatatatatatatatatatattattaagcttgaattcaagatta

SEQ ID NO: 83 - DNA, 1178 bp, Populus trichocarpa
gtattttctgtggaggagaaacaaaaagcaatctcttgggttttaaagatttggtttgggttctctctttgaaagttatcaagaacagtaaaggggctcttgattgactgatgcaagtttaagattttctattctttttagagaaagaaggaagttttcttttgaaatgaatttgatgtgtgaggttttctttgctgaagaagaagagaaggttgaagaaggagagtgggaaaaggagagatagtatttaccaaagccaaggtagctagcagaagtagagaggcagatagctataaacaacatacaaaataaaatttcatctaaggggttttagtttgttctgcttctgctgggtaattggcaatgtctactactataatcagtgaccctatggtggtctcagcaccagagacacaagcaacagcagcagctgcagctgcagcaacgacagcaacacaactcattgcacagatagaggttgagtcagtcaaatgtgattgctgtggcctaatcgaagagtgcacaccagcatacattgaaagagtacgcgaaagatatcatggaaaatggatttgtgggttatgtgcggaggctataaaatatgagattgtaaggactgagaggcttattagcactgaagaagcaatgacaaagcacatgaacttctgcaagaagtttgtttcctcagggccacctcctgatccaacaactcatttgatagccgccatgagacaaatcctgagaagaagcttggattctccgagaggtttaaggtcaaccccaagtagtccaaacaaaacgaacggagcaattcgtgccgctgcacttgctaggtccgaaagttgcttccctgctctgtctggttgatatataatactgagcatcgtggaagacggggtgattcataaaatggtgatgaagctattggaaaaaaaaaagacagaaaaaaacacattccattttcatgcaaataagaaagatcaaaggattgaagttaggtgcctaatctagctacttatatagttgttaaatacagaacttcctgtcacaagaaaaatcacggaactcccttttttcctgaagctttatatttcagctgtacgtacttgtgtaatctgtctttttaaaaccatggcttccttcctctttttaccaaattatatttctcaattttgata

SEQ ID NO: 84 - DNA, 1010 bp, Populus trichocarpa
gtcaagataaagtcccattctttttccttgccccaagtttgtttgaggggttataaaaaatgaaccaagagatgaatggtgttgatacagagattgatcagaaccaccaagagaatgtgcaagagaaaatcgattatgtgtttaaggtggtggtgatcggtgactctgcagtaggcaagacgcaaattctttccaggtttaccaagaatgaattctgctttgattcaaagtctaccatcggtgtcgagttccagactaggactgtcatcattaaagacaaggtcatcaaggctcagatctgggatactgctggccaagaaaggtaccgggcagtgacaagcgcatactatagaggggcattaggggctatgttagtctacgacattaccaagagaccaacgtttgatcatgtggctaggtgggtggaggagctccgagcccatgctgacaactcaattgtgatcatgctgattggaaacaaggctgatcttgtggacctcagggcagttccaacagaagacgcggtggaatttgcagaggagcaaggcctatttttttctgagacatcagcccttagtggtgacaatgtggacggtgcatttttcaggctgctagaagaaatttacggtgtgatttgtaagaagtcattggaatgtggcaatggaaatccccatgctgctgatgccataacgcttagaggttctaagattgatggcatatcagggactgatctggagattagtgagatgaagaaattatctgcttgctcgtgttgatttgatcatttttctagtgaattgtgtactataagactttaccactcgatgttcttaattgattctgtggctttctttggaaagtggtgatcgtttcggtagtgtggtaagggtggcaagttttttcttctctgtgacctgtcaagattttagcagtattgtacttgtcttacagaaaccatgaatttggtttttttatatgtattgatttggatggatggttttccttttcct

SEQ ID NO: 85 - DNA, 1159 bp, Populus trichocarpa
atgaactagaaattcaaagcttccatcacatattttaatggcttacaatccttcttcaaggtcctgtttttatgattctttttgtttcctattcattacattcgttcttccatttttcttcctcacaaaagctcaagctgcaagccattgtagaacctcatgtggtaccattccaataaactacccttttggcatcgatgatggctgtggcagtccatattacaggcacatgcttttatgctccgattcgggcattctcgagcttcgaacgccttccgggagataccaagttcgtagcataagctactcagaccctcacatgatagtcaccgatccattcatgtggaagtgtaaagatggtcatcactttcgtgcaactagggcatttagccttgatacaagcacacatttaacactctcctctcaaaatgactacctcttcttcaattgcagtgaagagaaagtgattgttgagccaaaacctatcttctgcgagaggtttcctgatcggtgcgactcgacatgtgatagtgctagttacctttgcaggcacttgccaggatgtggtgctgcattaggaggacgttcttgctgctcttacttcccaaaagcgaccgaatctttgaggctgatgctgaagtattgtgctagttacactagtatttattggagaattaatggtgcaaatgctcctgatgatcatgtacctgagtatggtattagagttgattttgacattccagtgactacagattgccttcaatgtcaagacatgaagaaaggaggtggaagatgtggatttgacacacaatcacagaatttcctatgtctgtgcaatcagagatcaaatgtcacaacatattgcaatggtatatcacagcagcagtagccatagcaaggcaggaataattgcagggactgtaactggagtttcagctgctggggcattaggaattggtgctggtctatggtattggaagaaagtgagagcctcagcaccagtaacatgtggggttcaaagcaatgagaataggctcttttaagaacaaatcaacaaaagtgaataccaatcactttctcttttctaatttccctattttttgagttgcctttcgcactttaataagcaacttaatggatctgttttctttgttttaaa

SEQ ID NO: 86 - DNA, 884 bp, Populus trichocarpa
cattatcccaaaaaacgagtaaggcatatacagcagccaaaaccatgtcacccggtcccgtcgttgaggctgaacccgccgccgccggtggaagatacacagcagaagaagcctcctcacaggacgtggatgaacctgtggtggaggacgtgaaagaagatgagaaggaagaggacgatgatgatgatgatgaggacgatgatgatgaagatgatgacaaggatgatgataccccaggtgctaatgggagttccaagcagagcagaagtgaaaagaagagtcgcaaggcaatgttgaagcttggcatgaaacctgttactggtgttagcagggtcaccatcaagagaaccaaaaatatactgttttttatctcaaagcctgatgtcttcaagagcccaaattctgagacctatatcatatttggagaggcaaagatagaggatttgagctctcagctgcagacacaggctgctcagcagtttagggtgccagacatgtcatctatgttaccaaaaccagatgcttctactgcagctgctgctgcaccagcagatgaagaagaggaagaagtcgatgagacaggggttgagcctagggacattgatcttgttatgacacaggctggagtttctaggagcaaggctgtcaaggctctccagacgaataatggggacattgtcagtgctatcatggagcttactacttaggatggctccctggttactctcttattttatgctgacaagttctcggaacaatttaatcatggtagtcaaattggcttgccatctatgaggtcgctaattatccattgtttgtgtcaaatctgagatttttacctattgcggtttttctttagtagcaggctcttattgtgctctttg

SEQ ID NO: 87 - DNA, 2371 bp, Populus trichocarpa
atgccattgaatagccaaatctctctctctcttagaatgtgaaggagcagttagcgaaggaaaaattggagaatgccggagctggtgtaccaagagcagagctcgtcgagatcaggatttagagctcgagatgcaagtcctgactctgtaattttcactctagagtccaacttcagtctcttctcttctgcttctgctagcgtcgatcgctgctcttttgcttccgatgctcatgatcatgactctctcgcctctgaaatctctctggtaaaaccatttctctaacttccctgcatgcatgcgttttctaacccatggtttttgagtgagttttttgagagtgcaatgcagcatttggcagctggtcatgatcaacaagagaactcttcgagtggtccagatcgaaacaacaacaagcagaagcagcacactcacacccgtctctccagaaaagcagaaaaagttaaagctgttaaaaaagaagacaacagaattagttctgtagaagacgatattcatctcctagattctgcaagaagctccttctctctcgctctcaaagagtgtcaggagaggagaccgagatctgaagcgatcacaaagaaaccagacaggcggaggcctgcgtcactagatcttaacaatgtggtcacatcttcttcgccgagattgggtaacatgaagaagagtatagcttgttcatctcgaaaatctggtacatttccgagtcctgggaccccaaattaccttaatcagtatcattctagtgttggaatgcagaagggttggagttcagagagagtgccgttgccaaataatagtaataggcggcaagtgatgaatactactggagctgctgttttgccttataataataatggaaggacattgccgtccaaatgggaggatgctgagaggtggatatttagtcctgtttcgggagatggggttgttaggagttcgattcagcctgcacagaggaggcctaagtcaaagagtggaccacttggaccaccgggagttgcgtattattccttgtattcgcctgggatgcaggtgttcgatggagggaatgcggggaattttgttgctggttctccgttttcagctggtgttattgctgctgatggattgggtattaggtcacatgggagccatggtgtgtcatttcccatgcggacagagccctgtatggctcgttcggttagtgtgcatggttgttctgagatggtagctcagtcttcgttgccatcacaagatgaaaagcttgatggtgtcaaggatgctgcaactgatatctcccgtgttgtttcaagaagggacatggccacccaaatgagccctgtaggtagtaatcattcatctcccaccaggaagccatcattctccacctccaccccctctgtcctacctattgtggaactgcagagcgtaccttcctctagatctgagaccagggatgtacaggtggatgagagggttactgtcacaaggtggtctaagaaacacagagcccgcaaccatggaaaaagttcacaagttgttgatgactggagaaagaaagctgctgatactctttcatcaggttgggatgtttcagaggctggaaagagcatttcaaaggtcaaaagagaggaagctaaaatcacagcatgggagaacctgcagaaggcaaaagctgaggcagaaataaggaaacttgaggttctttttccccacgcgttttcgtcgtacaattttgactaacttgaaggattaattcacttaatgatcagccttggtgcagcaaagaagatattttctctaatatttcctttctgctcacttgaaagctttgctagtacttaattataagatactggtcttaactgactagattcattgcagatgaagcttgagaaaaagagatcatcatctatggatagaattatgaataagctgagatcagctcaaaagagagcccaggaaatgaggagctcggtgctagcaaaccaggcacatcaagtttccacgaactctcacaaagttatatcattccgcagaacccgtcagaagggttccctgagtggttgtttcacatgccatgctttctgatatctcatccagccacatgttggtacacttcgatagatataaaacctggtcaaattgcaggaaaaactgcagcagaatatggaagcgtataggagggaaaccaccattgacactattttggtgtcaatggagttgagttggggcgtgaagaacaagtactactgttaagatttcagacgggacacaagaccctggatggtatcaaaatctccaggaaatttgtctactaaaccttgtatgcatgtatgtatgtatgtatatcaacaattattattat

SEQ ID NO: 88 - DNA, 2514 bp, Populus trichocarpa
tttttttttaaacctcctcctcggatattatatacttcgttttctctcttctctgccattaatcatacctcacaccgatccagattttgtttgctcataaacagagggagtgggagagacccccgtttaacttttctttttttccttttgaagtaatacgacatctccctctcctcctcctcctcctcctcctcctctatttgttcttgatttcgattatctgatcggacggtcacggtcttatttgtattcacctgcaatatcgtacggccagggatctctcttctcttcttactgagcaagttgcatctcataagcactggcgatcatggagctgaatggtcaaacaagagtgagaagaaaggaccattttgctcatacaaacggtgatttagcattaccaagtgttggtgatgtagatccctggactgcatgggcatataagcctcgaactatttcgttgttacttattggtgcttgctttctaatatgggcaagtggagccctagatccagagagctgcacatctggtgatgttgttacatctgtgaaaaggggtatatgggcaatgactgcagtttttcttggttattgcttgctacaggccccttcaacggttctaataaggccacatccagcaatttggcgcttagttcatggattggccattgtttatcttgttgccctcacatttctgctttttcagaagcgtgatgatgcgcggcaatttatgaagtttctccatcctgaccttggaattgaactacctgaaagatcatatggtgctgattgtcgcatttatgtgcctgaaaatcctacaagcaagtttaagaacgttttggacactctttttgatgaatttgttctagcacatatctttggatggtggggcaaggctatattaatccgtaatcagccacttctatgggtattgtcaattggttttgagctgatggagtttaccttccgccacatgctaccaaacttcaatgaatgctggtgggacagtatcattcttgatattttgatatgcaattggtttggcatttgggctggaatgcatacagtcgggtattttgatggaaaaacatacgagtgggttggtataagtcgccaacctaatattatgagcaaggtcaaacgaacattggaacaatttacacctgcacagtgggacaaagatgaatggcatcccttgcttggtccatggcgatttatccaagttcttagtctctgtattgtcttcctgactgtagagctcaacacattctttttgaagttttgtctatgggttcctcctcggaaccctgtaatagtgtacaggttgatcttgtggtggctaattgccatacctacaacacgtgaatacaattcatatctccaagaccgaaagcctgtgaaaaaggtaggcgctttttgttggctttcccttgctatttgcatcgtggaacttctcatttgcatcaagtttggacatggtctttatcccaaaccaatgcctgcatggttggtcatcttctggacatctgttggagttagccttgtaatattcctgattatgtggtcatggaaaagtttgggaagaaagagacgatgaatttgctagcatggtacactattcttttatttcttcatgttcttatgggtatacatcttgtaaatgccctatagcttcatggaattgtaaatgcttattctcttaggttagtggatcacagttttagagtattcaattcttttatgataggtagttggaaaatgcctctaatggtaggttcatgtgttgctaatttgcagcaatcatcaaattggtaattttcccttttaacttgtctgagagaggagcaatttttaacaccgggcttgcctcacatttttcattggaaaatgcatgcctgacaattccattggggcattttatcagaatgtttaaaatatttatattttgcgtcggctctatagcatatgcttataagcattgaaatatgattcaactgaagtattcgcacatgttttgctgtttacgtttatatgattgttgtgtttagttcgccacaagcagcctggcagttattcgatccggtatcttgacgttatcttatcacctgctgtaaatcctatggctcaaactggcaattattcaaggcagttcttgaaagagttcaaggttttctttcctgttgatcgacctttgtaaagcctatgaagcaattgtgcaaataattggtgctcgacagaaggcaatctctagtttgctcacgacaattctagccgattttcgggaaaacatttgtggactggagtcattgattcaatcaaggtgaaattggaaatcatttagaaaagcaattcctgttctttgacaacactaggttcttctttctcgatccgtgcccatgtcactgtgtgtatatatagaaatagaaaaagtgtatctctcttaatcatgcaggattttgttttttgcagtcaatatgcccatgacttataatctcagtcttcttcgaaaacact

SEQ ID NO: 89 - DNA, 852 bp, Populus trichocarpa
acgaacaagcaatgatagagttcgagaggtcatcttcttgttataaaatcctcaaaagctcgaggtgaaaaaataagcaagggagaggagagagctagctagctagtcaagttaagaggcagaaaacaagaatgtcttccaggaagaaaaagaaggcagctctttatgagaagttacgtgctgctaccaattctaatgctatgaacaaaacctcaatcatagtagacgcgtcaaaatatattggagagttgaagaacaaggtggataggctaaaaaaagaaatcggaacatcttcaaccccccaaaattcattacctgcgcaggtcacagtggaaaacctagaaaagggtttccttattaatgtattttcaggaaagaattgccctggattacttgtctccatacttgaagccttcgaggaactaggccttgatgtgcttgatgctagggtttcttgtgaagacaatttccaacttgaagcgattggtggagaccaaaaccaaggccatgatgctcaagtagtgaaacaggcagtgctgcaagctatccataactggaatgaaggcagctagctagtgagcaagaatgacccaaatattttcttcccctatcttcctgtacgctactgagaaattgggaatttgtatattgaatttcaagcacactaggcagtcctaattccaagttagtagtcagtcaaaatgtgcagcaatgggattctggtaattaattttcagtttttaattaagcaacttttaccttcgatgatctatacatatatttgagcataaaatgcttaattaaaatccgtatgattggtttcagctaattaaggagctctttggcaagatc

SEQ ID NO: 90 - DNA, 1320 bp, Populus trichocarpa
agagttccatcatccattgtcttaccacttcatgggaatgtctatcctactgggttctataatgtgaccctcaacattggccaaccatcaaaaccttactttctcgatgtagatactggaagtgatcttacatggctccaatgtgatgttccccgtgcccaatgcacagaggcaccgcatccatattacaaacccagtaacaatctagtggcttgtaaggaccccatttgtcaatccttgcacaccggtggtgaccaaagatgtgaaaacccaggacagtgtgactatgaggttgagtatgctgatggtggatcatcccttggcgtccttgtcaaggatgcctttaatctcaactttacaagtgaaaagcgccagagtcctcttctggcccttgggttgtgtggatatgatcagcttcctggtgggacttatcatcccatagacggagtgcttggtcttggccggggaaaacctagcattgtatcacagcttagtggtctgggtttagtgagaaatgtgattggacactgtttgagtgggcgcggtggaggattcctcttcttcggggatgatctttatgattcatctcgtgtagcttggacaccaatgtcacccaatgcgaaacactattcacctggatttgctgaattgacttttgatgggaaaactactgggttcaaaaacctgatcgtagcttttgatagtggggcctcttatacttaccttaattctcaggtgtatcaaggtctaatttctctgataaagagagaattatccacgaagcctttgagagaagcactggatgatcagacgcttccgatttgctggaaaggacggaaacctttcaaaagcgtacgtgatgtcaagaaatacttcaagacctttgcattgagctttgcgaatgacggaaaatctaaaactcagcttgaattcccaccagaagcttatcttatagtatcatctaagggaaacgcctgcttgggagttctaaatggtacagaagtaggtctgaatgatctgaatgtcattggagacatatcaatgcaagacagagtggtgatttatgacaatgagaagcaactgattggatgggcacctcgaaattgcgacaggattcctaaatccagaagcattatcatttgatgacaggtgttctaaacgttaaaaaaaacttgtcctgtacagaaagctgctaagctgtaacattgtattcttccaccattacagcagattccttgtaaagtaatactatgtatatgtggtctggtgaatcagaccccacaattatacttgatgggttatgaaaatttaataaaataa

SEQ ID NO: 91 - DNA, 2811 bp, Populus trichocarpa
atggaaatgtcctgcaaggatggtaagcagcctatcatggacaatggcaagtatgtccggtacacgcctgagcaggtcgaagccctggaaaggctctatcatgattgtcccaaacccagctccattcgccgccagcagctcattagggagtgtccaattctctccaatattgagcccaaacaaatcaaagtttggttccaaaatagaagatgtcgagagaaacagaggaaagaagcgtctcgcctccaggcggtgaataggaagctgagtgcaatgaacaagcttctgatggaagagaatgataggttgcagaagcaggtgtcgcagctggtgtatgagaatggctactttcgccaacatacccataacacaccgcttgcaaccaaagatacaagctgtgaatcagtggtgacaagcggtcaacaccacctgacacctcagcatccgccaagggatgctagtcctgcagggcttttgtccattgcagaagagactttaacagagtttctttcgaaggctactggaactgctgtagagtgggtccaaatgcctggaatgaagcctggtccggattccagtggaatcgttgctatttctcatggttgtgctggtgtgggagcacgagcttgtggcctagtgggtcttgaacctacaagggttgctgaaatcctcaaggatcggccatcatggtttcgtgattgccgagctgtggatgtgcttaatgtgctgcccaccgcaaatggtggaaccattgagctgctttatatgcagctctatgcgccaactacgttggcacctggtcgtgacttctggttgttgcgttatacttctgttttagaagatggaagccttgtggtttgtgagagatctctgaaaaatactcaaaatggcccgagcatgccaccagtgcagcattttgtgagagcagaaatgctcccaagtgggtatctggtacggccttgtgaaggtggtggttcaatcatacacatagttgaccacatggatttggagccttggagtgtgcctgaagtactacggccgctgtatgaatcctcaactgtacttgctcaaaagacaacaatggtggctctacgccagctgcggcagatagctcaggaagcttctcagtccaatgtgaccaactggggcagacgacctgcagctctacgagcactgagccagaggttgagcaggggttttaatgaggctctcaatggatttagtgatgagggatggtcaatgatcggaaatgatggcatggatgatgttactatcctcgtgaactcatctcctgacaagttgatgggttcaaatctttccttcactaatgggtttccagctgtcagcagtgctgtcctgtgtgctaaagcatcgatgcttttacagaatgtccctcctgcaatccttctcagattcttgcgagagcacaggtcagaatgggcagataacaatattgatgcctatgcagctgcagcagttaaagttggtccttttagcctacaaggttctcgagttggaagtttcgggggtcaagttatacttccgctggctcacactattgaacatgaagagttcctggaggtcataaaactggaaggtgttggccattctcctgaagatccaataatgcccagagatgtgtttcttttacaactctgctgtggaatggacgagaatgctgttggaacatgtgctgaacttatatttgctcccattgatgctacttttgctgatgatgcaccgcttttaccttctggttttcgcatcattccccttgattctgggaaggaagcctccagtccaaatcgtaccttagatcttgcggctgctcttgaagtcggaccagctggaaacagagcatccagtgatcattctgctaattctggttgcacaagatctgtaatgacaatcgcatttgaatttgcgtttgagagccacatgcaagaacatgtagcatcaatgactcggcaatatatccgcagcattatatcttcagttcagagggtggcattagcactatctcctcatcttggttcacaggctggtcttcggtcaccactgggtactcctgaagcacagacacttgctcgttggatctgccagagctacaggagctatttgggtgtggagctactcaaatccaatggcgaaggaagtgaatctattctgaaaaccttgtggcatcattcagatgctattatgtgctgctcactgaaggcgttgcccatctttacttttgcaaaccaagctggacttgacatgcttgagacaaccttagttgcactgcaagacataactttggaaaagatatttgatgatcatggaagaaaaactctctgctcagaattctcacagattatgcaacagggttttacttgtcttcaaggtggtatctgtttgtcaagcatgggcagaccagtttcatatgaaagagctgtggcctggaaagtgttgaatgaagaagaaaatgcgcattgcatctgctttatgtttataaactggtcttttgtctgagacttgaaaccagaatctagtggagtggctgatttaagagtgagaactctatatctgtcttattttgctggcttgtatttcttttatgtataacatgttgcaaccgtggagcacctagttcatttgctatgttataaatttgtgtggggttcattatagtagagattatggacgagacgatatagttgccggggatcattttgtttctgactggtttgttcttaagtcctcacctgggtctatgttgattattgcaaccgaaatctgtcaatcctggtttttcctgtgttgcttaca

SEQ ID NO: 92 - DNA, 886 bp, Populus trichocarpa
gttcagggaatcagcctagccaagaagagattacaacgccagaagctaagtcacactgatcagagcaacaaaaaaagtacaccaacaatgtcttccaccccaacaaatgaatggcaaacctactggtgtcatgaatgtgacctcagcatccaccttctcaccaccaccactcctctttgtcctcactgccaccatgactttcttgaactcatggaccccattcccacctccaccgccgctgacaccaccactttcctccttgacagcccctccttcctcaacttccttcaacatctcaacaccaatagccattgcgattgtgaagatgataatataaacgccactattgactctatcatccccaccataaaaatcacttcttgcatgctagaaatggatccaatgcttgtgtgtgcagtgtgtaaagatcagttcttgattgatgttgaggccaagcagctgccctgcagtcacttgtaccatccaggctgcattctcccctggctttctaaccacaactcttgtcctctctgtcgcttccagttacaaacgccagtagtcagagaggagaatttggaaaattggtctcctgatcatcctcatcatgatgctaatcatgctcatgtcggggttttgtccacctctcttcctccacacttttgaatttgaatgcaggtaaataaacgcttctggggattttgggtttttggtttggaattgatcgagtgaagtgtaaatcaacagtgaacgattgtgttacacaaagaacttgtgatggttgtgcatctccttctccccttcagactttcactatggatattgtaaagtaataccttgtttgggattggattttggccagaaatacagcgtggttgaattgattttt

SEQ ID NO: 93 - DNA, 887 bp, Populus trichocarpa
gacccccttcaatcgattgcaaggcatgaagcgtcagctccagcattgacatcccttcttccacagaaattcctgctaagcaagcgcagaaagtccagttctgacccgatgatgaactttgttagggaatatcataatgatttggaagttgtgtatgttgggcaactctgcctttcatgggaaatccttcattggcagtatgaaaaggcactcgagctatgggattccgacccttatgggatgcgacagtacaatgaagttgctggtgaatttcaacagtttcaagtgatattgcaaagatttatagagaatgaaccttttgaaggtccacgggtgaaaaattacattaagaatcgatacgtgttgcgtaatctccttcaagttccggtaataaaagaggacagtatgaaggataaaaaggcaagacgaaaaggaagggatgatggttctattacaagtgatatgctagtagagataatggaagaatcaataaggattttttggcgatttgttcgatctgataaagatgcacaaaacgtgatttcaaagggtcgcaaaggaactcaaatagagccccaagatcccactgaactagagttattgacagaggtgcgaacgaatttccaaaagaaggagaggaggcttaaagacgtcttgagaagtggaaactgcatactgaagaagttccaaaaacatcgagaggacaattctaatcaagttctttacttcttctctcaagtggatatgaagttagtagcgagggttctgagcatgtccagagtaacaacagaccagctactatggtgtcacaataaattaagcaagataaattttgttagccggaagatacacgtagagccatcatttttgctttttccatcatgatg

SEQ ID NO: 94 - DNA, 1471 bp, Populus tremula x tremuloides
ccccatttcggtttttagtcgaaatcttgggttgggggctggaaatatgaggacatgcttgtgggtttttgctttggttcttgttttcggttttgttgatggaaaaaaaattgaaaggttgcgcacagagaggatttcaggaagcgctggggatgtgttggatgatgatccagtgggaaggttgaaggtctatgtttatgagcttccgagtaaatacaacaagaaactgctgcagaaggaccccagatgtctcacccatatgtttgctgcagaaatcttcatgcatagatttctcttatccagcccagttcgaacccttaatccagatgaagcagattggttttattcccctatataccccacttgtgatctcacacccatgggcttgccgttgcccttcaaatctcctaggatgatgagaagtgcgatacagctaatctcctccaattggccttactggaatcgtacggaaggggctgatcacttttttgttgtgccccatgactttggagcctgcttccactatcaggaagagaaagccgttgagcggggcattcttccgctactccagcgttctactttggttcaaacttttgggcaacgaaatcatgtgtgcttgaatgaggggtcaattacaattcctccttttgctcctcctcaaaagatgcaggcccaccagatcccccctgacattccacggtccatttttgtctatttccgtggattgttttatgatgtaaataatgatccagaaggtggttattatgcaagaggagcaagggctgcagtttgggagaatttcaagaacaatccactcttcgacatctccactgaccatccaacaacatattatgaagacatgcagcgagctatattttgcttgtgcccactcggttgggctccatggagccctagattagttgaagcagtggtatttggatgcattcctgtcatcatagcagatgatattgttttgccatttgccgatgctatcccatgggaggaaattggggtgtttgtagcagaggaagatgtccctaacctggacacaatcctaacatccataccaccagaagtgattttaaggaaacaaaggcttcttgcaaatccttctatgaaacgtgcaatgttattcccacaacctgcacaaccaggtgatgctttccaccaaatcctaaacgggctggctcgtaagttgccgcacgacaggagtgtttacttgaagtctggtcagaatattttgaattggacagcaggaccagttggggacctgaaaccttggtaagagaagtgtcatttcctcaacagacatggctgtggtgtatctactgtttcatctaatcgcaaggactgtaaacttctctttgaagttttgatttttgtatagattcttgaatttcacgtgtacatctgggacatagttcgatgctttaaataacttcgcatttttcttggtaaaaaaaaaaaaaaa

SEQ ID NO: 95 - DNA, 1351 bp, Populus tremula x tremuloides
cagcttcattgccttgccctgaggctagccagtgagcactccaccaatgcagctgctcgactgcagctccccttgccggaactcgtccctgcgctcgttgacaatacttattttcactttgtcctggcttccgacaatgtgctcgccgctgctgttgttgccaattcacttgtccaaaatgcgctgcgcccccagaagtttgtgttacacataataacagataggaagacctactcaccgatgcaggcatggttctcactgcaccctctggctccggcaataattgaggtcaaggcattgcatcatttcgattggttcgcaaaggggaaggtgccagtcatggaagcaatggaaaaggatcagagggtgaggtcacagtttagagggggttcgtctgccattgtagcaaataacactgagaagcctcatgttatcgcagcaaaactacaaacacttagtcccaaatacaattcagtgatgaaccacatccgaattcatctacctgagctgttcccaagcctaaacaaggttgtgttcctagacgatgacatagtggtgcagtccgatctttcgccgctctgggacatcgacatggatgggaaggttaacggggcagtagaaacctgtcgaggagaagacaagttcgtcatgtcaaagaagttgaagagctatttgaacttctctcatcctttgatatcagaaaatttcaaacccaacgaatgtgcctgggcttatggaatgaacatatttgatttagaggcttggaggaaaaccaacataagcacaacataccatcactgggttgaagagaacttgaaatcagacctgagcttgtggcagctaggaacactgcctccaggtctgatcgcattccatggccacgtccacgttatcgatcccttttggcacatgttgggtcttggctaccaagaaaatacaagtttggcagatgctgagactgccggagtcatccatttcaacggtcgagcaaagccttggctagatatagcattccctcagctccgccccctgtgggcgaagtacatcaacttttccgacaagttcataaagggctgtcatattaggccatcttaacttgagttttgagatgaaaatgttggctgcgatcgtgattaatatatgtctatatataaggagaaaaaaagcagagaagagagagttcttcacaaatccaaggccttgaatccagagagtaaaatattcataaccaagcatttgtttgcttggtttttttagaattcatcctctgtgggcgaacaacccttccctattacttcaaatcttgattcaattttaatgagaaattaagcatgttatggattttcaacgg

SEQ ID NO: 96 - DNA, 1507 bp, Populus tremula x tremuloides
  misc_feature (597)..(597): n is a, c, g, or t
aagctgatgatcaaatgaggacatcaaaaatttcttcagggaattcaggctaccatgtatggattcttgcttttgcaatgagtctactgatactgattgccttgtcgaagtcatggttttatgatcacgcttctgctgctgcaagtgaggatttgcagtacttttcagtgatcgtcccttcaaaagggcgggactatcctcctgttcttgcttattggatatgtggtaccagtggagatgggaagagaatgttaaggctattaaaagcaatttatcatccaaggaaccaatatctattgcaacttgatgcggaatcctcagattatgaaagggcggagttggttgtttcagttcaatctgaaagtttgtttcaagcattcggtaatgttaatgttgttggaaaaggttttgctatcaacgaaatgggatcctctgctcttgctgccatactcaacgctgctgcgttgcttcttaagctgagtacagatggggactggttcatcaatttaagtgtctcagactatcctctagtgagtcaagatgatctcctccatgccttcacttccttgccaagagatctcaacttcatcaactatnctaatgacactgcgaaaaacgagatacacaagattaaccaaattgtagtagatcccagcttgcatcttcaaaagaggagccacctttattacgctgttgagactcgaacaacacctgatgctttcaagatatttggaggttcgccctggctgattcttacaagagctttcatggagtactgtgtccaaggatgggacaaccttccaagaaaactactaatgtacttcagcaacacggcatccccacttgaatcgtatttccactctgtcctctgcaactctcctgagtttcaaaacacaacagtaagcgatgatttaaggtacaatattctggaaactactacagatggggaatcaccttatgacaaaatgctaaatggtggagcagcatttgcaaggccatttaaagaagatgctgctgctctaaacatgatagatgagaacgtcttgaaccgtgaacccaacggattggtgcctggaaaatggtgcttagaccagggtatgaacaagagctcagaggcatcaaagcctccaggggaggatttgtgttcaacctggggtaacattaatgatgtcaagccaggatcttatggtatcaagcttgcatttttattgtctaagattgccagtgaagagaaattgacaactagtcaatgccttcaagctacaaaaatggggtcatcatagaaccaagcatacatccttgtgtggataatttttttcttttaatcgaaaactataggttaaatcaagcttcttagccatataaatcctttgctaaatgcactacaaatgcatacttgcttgttaaaacaaaagctagaagcacccagagatatattgtacatacattcacattttatcgaggatttctacaataaatttattgtttttcccccaaaaaaaaaaaa

SEQ ID NO: 97 - DNA, 1903 bp, Populus tremula x tremuloides
ttttggggtatcctgataccggaggccaggttgtttacattttagatcaagttcgtgccttggagaatgaaatgcttctgcgtatcaagcagcaaggacttgatatcatcccccgaattctcattattactcgactgctccctgatgcagtaggaaccacttgtggacaacgtctggagaaagtctatggatcggagcattgtgatattcttcgagttcccttcagagatgaaaagggaatggtccggaaatggatctctcgctttgaagtttggccatacctagaaacttacactgaggatgttgctgctgaaattgctaaggagttgcagggaaagcctgatctgatcattggaaattacagtgatggaaacgttgttgcctccttgctagcacacaaattaggtgttacagagtgcactattgcacatgctctagaaaaaacaaagtacccagattcagatatttactggaagaagtttgatgaaaagtaccacttttcatgccagtttacagctgatctttttgccatgaaccatacagatttcatcatcaccagcacattccaagagattgctggaagcaaggatactgttggacagtacgagagccacacagctttcactctccctggcctctacagagttgttcatggtatcgatgtatttgatcccaaattcaacattgtatcccctggtgctgacgagagcatatacttcccttacactgaggagaaacttaggctgacttctttccatgaagaaattgaagaacttctttacagccccgttgagaacgacgagcacttatgtgtgctaaaagaccgcaacaagccaattctattcaccatggcgaggctggacagagttaagaatttaactggtcttgtagaatggtatggaaagaataccaagctgcgcgagttagctaatcttgttgtagttggtggtgatagaagaaaggagtctaaagatatagaagagcaagctgagatgaagaagatgtacagtcatatagagaaatacaacttgaatggccagttcagatggatttcttctcagatgaaccgcgtgaggaatggagagctctaccgttacatttgtgataccaagggagcttttgtgcagcctgctttgtatgaggcttttggattgactgttgtggaggccatgacatgtggtttgccaacctttgctacttgcaatggcggtcctgctgagatcattgtgcatggaaaatccggattccatattgatccttaccatggagtacaggctgctgaactccttgttgacttctttgagaagtgcaaggctgatcccacttactgggacaaaatctcccagggaggcctgcagcgaatccaagagaagtatacctggaaaatttactctcaaaggctcctgactctcacaggagtttatggcttctggaagcacgtttccaaccttgatcaccgtgagagccgtcgctatctggaaatgttctatgcactgaaatatcgcaaattggctgattctgttcctttgactatcgagtaaatggaactggagaaatcgaggaaacatgggttggtttgagtcgggttccgggtccagaataatggtcatttcacgatagtgattggacaagaaaggctttgatcttctttttgtttttttgtttttcctttctgtgtgcatcttttttgttttcccttttcttttcatcatgcagcctcttcaatttcagtaaagattgtgtctgttgagttcatcatcccttatctgatctctatccatttcttgcaaattctctgtctcttgagttggaaatcatgggaattgatccaacatcaattcctgtatttctcctcagtactcgagattggaagttgatggctattcttcatcattatataaag

SEQ ID NO: 98 - DNA, 2198 bp, Populus tremula x tremuloides
gacaactacaactgctacttgtcggtgtgggtttgctcttaactctttcattctatctgcgatcctctctccctcccccttggcacttgtcaagcagcagagaatgaaggttattgataagataagggcggcagcagcagcaggagattcaaatggaggagatagcagcagcaataataaggttgttttctccttcgaattctttcctccaaagaccgatgatggagtggacaacctgttcgagagaatggacaggatggtctctcacaatccttccttctgtgacatcacttggggtgctggtggctccaccgcagatctgaccttagacatcgccaacaagatgcagaacatcatctgtgtcgagactatgatgcatctcacctgcaccaacatgcccgttgagaagatcgatcacgccctcgaaaccatcaaatccaatggaatccagaatgttcttgctcttcgtggtgatcctcctcacggtcaggataaattcgtccagatccaaggcggcctcgcttgcgctctcgacctcgtcaaacatatcagatccaaatatggtgattacttcggcattactgtcgctggctatccagaggcacatcctgatgtgattggaagtgatggatttgctacgcctgaggattaccataaagatcttgcttatttgaagcagaaggtggatgctggggctgatctaattgttactcaactgttttatgatactgatattttcttgaaatttgtgaatgactgccgccaaatcggaatcacttgtcccattgtccctggtattatgcccattaataactacaagggcttcatacgtatgactggtttttgcaaaaccaaggtacccgccgaggttactgcagcattggagcctatcaaggataatgaagaagccgtcagagcctatgggattcaccttggaactgaaatgtgcaagaagattttagctcatggaatcaagacattgcatctctatactctgaacatggagaaatccgctttagctatattaatgaatcttggtttgattgaagagtccaagattacaaggccgttaccatggcgccccccgacaaatgttttccgtgtaaaagaagatgtccgtccgatattttgggctaatcgtccaaagagctacctaacaaggacaataggttgggaccagtaccctcatggccgatggggtgattctcgcaatccatcctatggtgctttatctgactatcagtttatgcgaccccgcgcacgtggcaagaaacttcttgaagagtgggctgcccccttgaaaagtgttgaggatatatacgagaaattcaaggtatactgccttgggaagttgaaaagtagtccttggtcagaactagatggccttcagccagagactaaaatcattaatgaacagctgggtaagatcaacttgaagggactcctgactatcaatagtcaaccagctgtcaatggggaaaaatctgattctccatctgttggttggggtgggccaggagggtatgtatatcagaaggcttatctagagtttttctgctccaaggacaagctgaatgctcttgtggagaagtgcaagtctttcccttttgtaacttacattgctgtgaacaaaggagggagttggatttctaatgttgccctgactgatgtgaatgctgtaacctggggagtcttcccagcaaaggagattatccaaccaactgttgtggatcccaccagcttcagtgtgtggaaggatgaggcatttgaaatctggtcaagaggatgggcttccttgtacccagagggtgacccatccagaaccctacttgaagaggtgaagagcagctactttttggtcagcttggtagataatgattacatccatggggatattttcgctgtcttcgccgatttatgatgcaaggggctctttgcaacccctgcatttgctaagagtatccaagtccccagaaaaatggtggtgctttaccctaagttgaatcccagctttaatcatcttatcggcatacattgtttttgaaattatttccgtattgaaatgtaataattcattgtgattttgctactcccctctccattatatgctgcaaatttgtatttggagtggcaactaattctaagctttaagaaaatatgattgcctctttttcttggtacttccgaaaaaaaaaaaa

SEQ ID NO: 99 - DNA, 1548 bp, Populus tremula x tremuloides
  misc_feature (1266)..(1266): n is a, c, g, or t
gtggaccatctgcatccgtttcagatggtggtgaatcttcatcttttgaactccaagagaggattggtgactctatccgtgatgaaattcctaggccttctcaaaatcctggtaggcagaagcataagagtcatgatcctgctcgagatcttacgatccatgtattggaaaaattctctcttgtgaccaaatttgctcgggacacaagttcacaactatttcgagaaagtaatagcaatggctatggtgctgtagaaaggaaaagttctagctactctctccctgatgtcccccataagccatccatggatgcagagatggctcccgaggaaggtcctgttccctcagatcctctggagttcgataaaatgacactcgtatggggaaaaccacgacagccacccttggggtcatgaagagtgggctactttcttggattctgaagggcgagtcatggattcaaaagccttaaaaaagagaatattctatggtggagttgagcacacaacacgcagagaggtctggccatttttgctgggatatcatgcttatgattcaacgtatgcggagagggaatacctcaagtcttccaaaaagtcagagtatgagacagtaagacaacaatggcagagtatctctactgaacaagcaaagagatttacaaaatttagggaaaggaagggccttatagacaaagatgtggtgaggactgatagagcactgtctttctatgatggggatgataatccaaatgtgaatatcttacgtgatattctgttgacgtactccttctataactttgatcttggttactgtcagggtatgagtgatttgctatccccaattttgtttgtgatggaggatgaatcagaatcgttttggtgttttgtggcactgatggaacgtcttggacccaattttaatcgtgatcaaaatggcatgcactctcagctttttgcattatccaagctggtggagttgcttgattgcccattacataactatttcaagcagaatgactgcttgaattacttcttttgtttccgctgggttctaatacaatttaaaagggaatttgaatacaagaaaacaatgcgattgtgggaagtgttgtggacccattatttgtctgagcatctgcaactgtatgtatgtgttgcaatcttgaagcgataccgcaacaaaataatgggggagcgcatggactttgacacactcttgaaatttatcaacgagctgagtggccatattgaccttgacgcaatncttagagatgcagaggctctgtgcatatgtgctggtgagaatggtgctgctcgcatcccaccaggaactccaccttcattgcctactgagaatgagaatgctttattatatgctcaagatgatgaagtactgtaatacagttcgtctcattggtacgatataattggctttatattttgttttcttcatgaaatagctgtgtataattgctgttttttcgaaataaaatgttaataatcaagcaatcaactccatttgctgacctccttgaaaaaaaaaaaa

SEQ ID NO: 100 - DNA, 648 bp, Populus tremula x tremuloides
  misc_feature (579)..(579): n is a, c, g, or t
gacatcctgtgtgctccaagaatataaaaataatactaggtgctctctaatggctgcaaaaccaacaacagcgttccttttctcttctatattttaaagaaaaaacaccttctttcaacaaccctttgatggattccatcagaattagcatctcttcaatcctttcttggattcccttaaaacatgttctttgatagccaaatcaccaaagttgaacagaaaaggaaaatcttgttgagttcttgagctaaacgagaggaggaggagaattgtttttcgtttttcagatatggaggagaggcatgttatttttgggaagtatgaaatggggaggcttttagggaagggaacttttgctaaagtttactatgggaaacacttggtgacaggagagagtgtggcaatcaaagtcataagcaaagatcaagtcaagaaagaagggatgatggagcaaatccagagagaaatctcagtcatgcgtcttgttcgtcatcccaacattgtagagctcaaggaagtcatggctaccaagactaaaatcttcttcatcatggagtatgtccgaggaggagagttgtntgccaaagtagccaaaggaaggctgaaagaagaagctgctcgaaaatatttccagcaactaatcagcgc

SEQ ID NO: 101 - DNA, 1030 bp, Populus tremula x tremuloides
tgatgaagatgagcaacttgaaaatctgctgattttggcttgtcacccttgccggaacagtctccggcaacgacgggcttttgtactactcagtgcggcaactcctgcttatgttgccccggaagtcttgagaaagaaaggctatgatggcatcaaaagctgatatatggtcatgtggagtgattctttatgttcttcttgcagggttcttgccatttcaagacgagaatgttatgaagatgtacaggaaagttttcaaggctgaatatcagttccccccttggtttcaacagattccaagaggttgatttcaagacttcttgttgctgatcctgaaaagagaatcacaattcctgccataatgagaaatcattggtttcttaaagggttttcgagaccagtggccttttctatccaagaatcaagcatggataaaacagatcaagatcttgattcttgttctgttgtcaacaccaatgtatcatcaccagagttcttcaacgccttcgagtttatttcctcgatgtcatccggctttgatctgtctggtctttttgagactaagaggaaatcaagctcaatgttcacttcaaaattttcagcgagtgcgatcatggaaaaaattgaaggggttgcaagggggctgagttataaggtggcaaaagtcaaagatttcaaagtgacgttacaaggtccatgtgaggggagaaaggggaagctggcggtgacggcggaggtgttcgaggtggcaccggaggttgcggtggtggagttctccaagtcttccggagataccttggagtatactaggttctgcgaggaagatgttaggcctgcactaaaagacatcgtttggacatggcaaggagacaatgtttgtaataaagataacaacaatagtcatgtagaagatcctgagattcaaatgttgtagaaaggctttgatcttaatttggtgagttttaggattttatataatctgtaaataagtttttgttatttaattaatgtatcaagttgagtgttcttg

SEQ ID NO: 102 - DNA, 1215 bp, Populus tremula x tremuloides
cccacgcgtccggtattttctgtggaggagaaacaaaaagcaatctcttgggtttttgttcttttaaagatttggtttgggttctctctttgaaagttatcaagaacagtaaaggggctcttgattgactgatgcaagtttaagattttctattctttttagagaaagaaagaagttttcttttgaaatgaatttgatgtgtgaggttttcttcgctgaagaagaagagaaggttgaaggagagtgggaaaaggagagatagtatttaccaaagccaaggtagctagcagaagtagagaggcagatagcaataaactacaaaataaaatttcatctaaggggttttagtttgttctgcttctgctgggtaattggcaatgtctgctactataatcagcgaccctatggtggtctctgcaccggagacacaagcaacagcagcagccgcagctgcggcaacgacagcaacacaactcattgcacagatagaggttgagtccgtcagatgtgattgctgtggcctaatcgaagagtgcacgccagaatacattgaaagagtacgcgaaagatatcatggaaaatggatttgtgggttatgcacggaggctataaaatatgaaattgtaaggactgagaggcttattagcactgaagaagcaatgacaaagcacatgaacgtctgcaagaagtttgtctcctcagggccacctcctgatccaacaactcatttgatagccgccatgaggcaaatcctgagaagaagcttggattctccgagaggttcaaggtcaaccccaagtagtcctaacaaaacgaacggagcaatccgtgtcgctgcacttgctaggtccgaaagttgcttccctgctttgtctggttgatagataattctgagcaccgtggaagacggggtgattcataaaatggtgatgaagctattggaagaaaataaaaaaaagagagaaaaaaacacattccattttcatgcaaataagaaagatcaaaggattgaagttaggtgcctaatctagccacttatatagttgtttaatacagaacttcctgtcagaagaaaaatcatggaactcccttttttcctgaagctttatatttcagctgtacgtacttgtgtaatctgtctttttgaaaccatggtttccttcctctttttaccaagctgtatctctcaattttgataaaaaaaaaaaaaaaaaa

SEQ ID NO: 103 - DNA, 484 bp, Populus tremula x tremuloides
ggatgccattgaatagccaaatatctctctctctggactagaatgtgaaggagcagttagcgaaggaaagattggagaatgccggagctggtgtaccaagagcagagctcgtcgagatcaggatttagagctcgagatgcaagtcctgactctgtaattttcactctggagtccaacttcagtctcttctcttctgcttctgctagcgtcgatcgctgctcttttgcttccgatgctcatgatcatgactctctcgcctctgaaatctctctggtaaaaccatttctctaacttccctgcatgcatgcgttttctaacccatggtttttgagtgagttttttgagagtgcaatgcagcatttggcagctggtcatgatcaacaagagaactcttcgagtggtccagatcgaaacaacaacaagcagcagcagcagcagcagcacactcacacccgtctctccagaaaagcagaaaaagttaaag

SEQ ID NO: 104 - DNA, 812 bp, Populus tremula x tremuloides
tgatactcttccatcgggttgggatgtttcagaggctggaaagagcatttcaaaggtcaaaagagaggaagccaaaatcacagcatgggagaacctgcagaaggcaaaagctgaggcagaaataaggaaacttgaggttctttttccccacgcgtttcgtcgtacaattttgacacttaaacgattaattcacttaatgatcagccttggtgcagcaaagaagatattctatctaatatttcctttctgcccacttgaaagcttagctaatacttaactataagatactggtcttaactgactcgattcatttcagatgaagcttgagaaaaagagatcgtcatctatggatagaattatgaataagctgagatcagctcaaaagagagcccaggaaatgaggagctcggtgctagcaaaccaggcacatcaagtttccacgaactctcacaaagttttatcattccgcagaacccgtcagaagggttccctgagtggttgtttcacatgccatgctttctgatatctcatccagccacatgttggtacactttgatagatataatacctggtcaaattgcaggaaaaactgcagcagaatgtggaagcgtataggagggaaaccaccattgacactattttggtgtcaatggagttgagttggggcgtgaagaacaagtactactgttaagatttcagacgggacacaaaccctggatggtatcaaaatctccaggaaatttgactgctaaaccttgtatgtatgcatgtatgtatgtatgtatatcaacgattattattattattattat

SEQ ID NO: 105 - DNA, 2550 bp, Populus tremula x tremuloides
  misc_feature (127)..(127): n is a, c, g, or t
gccttccgttttttttttaaacctcctcctcagatattatatacttcgttttctctcttctctgccattaatcatacttcacaccgatccaggttttgtttgctcctaaacagagagtgggagaganccgtttaacttttctttttttccttttgaagtaatactacatctccctctcctcctcctcctccacctcctcctctatttgttcttgatttcgattatctgatcggacggtcacggtcttatttgtattcgcctgcaatatcgtacggcctgggatctctcttctcttactgagcaagttgcatctcataagcactggcgatcatggagctgaatggtcaaacaagagtgagaagaaaggaccattttgctcatacaaacggtgatttagcattagcaagtgttggtgatgtagatccctggactgcatgggcatataagcctcgaactatttcgctgttacttattggtgcttgctttctaatatgggcaagtggagccctagatccagagagctgtacatctggtgatgttgttacatctgtgaaaaggggtatatgggcaatgactgcagtttttcttggttattgcttgctacaagccccttcaacggttctaataaggccacatccagcaatttggcgcttagttcatggattggccattgtttatcttgttgccctcacatttctgctttttcagaagcgtgatgatgcgcggcaatttatgaagtttctccatcctgaccttggaattgaactacctgaaagatcatatggtgctgattgtcgcatttatgtgcctgaaaatcctacaagcaagtttaagaacgttttggacactctttttgatgaatttgttctagcacatatctttggatggtggggcaaggctatattaatccgtaatcagccacttctatgggtattgtcaattggttttgagctgatggagtttaccttccgccacatgctaccaaacttcaatgaatgctggtgggacagtatcattcttgatattttgatatgcaattggtttggcatttgggctggaatgcatacagtcgggtattttgatggaaaaacatacgagtgggttggtataagtcgccaacctaatattatgagcaaggtcaaacgaacattggaacaatttacacctgcacagtgggacaaagatgaatggcatcccttgcttggtccatggcgatttatccaagttcttagtctctgtattgtcttcctgactgtagagctcaacacattctttttgaagttttgtctatgggttcctcctcggaaccctgtaatagtgtacaggctgatcttgtggtggctaattgccatacctacaacacgtgaatacaattcatatctccaagaccgaaagcctgtgaaaaaggttggtgctttttgttggctttcccttgctatttgcattgtggaacttctcatttgcatcaagtttggacatggtctttatcccaaaccaatgcctgcatggttggtcatcttctggacatctgttggagttagccttgtaatattcctgattatgtggtcatggaaaagtttgggaagaaagagacgatgattttgctagcatggtacactattcttttattccttcatgttcttatgggtatacatcttgtaaatgccctatagcttcatggaattgtaaatgcttattctcttaggttagtggatcacagttttagagtattcaattcttttatgataggtagttggaaaatgcttctaatggtaggttcatgtgttgctaatttgcagcagtcatcaaaatggtaattttccttaacttgtccgagagaggagcaatttttaacaccgggcttgcctcacatttttcattggaaaatgcatgcctgacaattccattggggcattttatcagaatgtttaaaatatttatattttgcgtcggctctatagcatatgcttataagcattgaaatatgattcaactgaagtattcgcacatgttttgctgtttacgttttatatgattgttgtgtttagttctccacaagcagcctggcagttattcgatccggtatcttgacgttatcttatcacatgctgtaaatcctatggctcaaactggcaattattcaaggcagttcttaaagagttcaaggttttctttcctgttgatcaacctttgtaaagcttatgaagcaattgtgcaaataattggtgctcgacagaaggcaatctcttagttcgctcacgacatttctagccgattttcgggaaaacattagtggactggagtcattgattcaatcaaggtgaaattggaaatcatttagaaaagcaattcctgttctttgacaacactaggttcttctttctcgatccgtgcccatgtcactgtgtgtgtatatatagaaacataaaaagtgtatctctctcaatcatgcaggattttgtattttgcaatcaatacgcccatgacgtatactctcagtcttcttcgaaaacactcttaccaaaaaaaaaaaaaaaaaaaaaaaaaaaa

SEQ ID NO: 106 - DNA, 787 bp, Populus tremula x tremuloides
ggacgaacaagcaatgatagagttcgagaggtcatcttcttgttataaaatcctcaaaagcttgaggtgaaaaaataagcaagggagaggagagagctagctagctagtcaagttaagagtcagaaaacaagaatgtcttccaggaagaaaaagaaggcagctctttatgagaagttacgtgctgctaccaattctaatgctatgaacaaaacttcaatcatagtagacgcgtcaaaatatattggagagttgaagaacaaggtggataggctaaaaaaagaaatcggaacatcttcaaccccccaaaattcattacctgcgcaggtcacagtggaaaacctagaaaagggtttccttattaatgtattttcaggaaagaattgccctggattacttgtctccatacttgaagccttcgaggaactaggccttgatgtgctcgatgctagggtttcttgtgaagacaatttccaacttgaagcgattggtggagaccaaaaccaaggccatgatgctcaagtagtgaaacaagcagtgctgcaagctatccataactggaatgaaggcagctagctagtgagcaagaatgacccaaatattttcttcccctatcttcctgtacgctactgagaaattgggaatttgtatattgaatttcaagcacactaggcagtcctaattccaagttagtagtcagtcaaaaagtgcagcaatgggattctggtaattaattttcagtttttgattaattaagcaacttttaccttcgatgatgtaaaaaaaaaa

SEQ ID NO: 107 - DNA, 880 bp, Populus tremula x tremuloides
gatgggaactggcatatggcggggaaaactagcattgtatcacagcttagtggtctgggtttagtgagaaatgtgattggacactgtttgagtgggcgcggtggaggattcctcttcttcggggatgatctttatgattcatctcgtgtagcttggacaccaatgtcacccaattcgaaacactattcacctggattcgccgaattgacttttgatgggaaaactactgggttcaaaatcctgatcgtagcttttgacagcggggcctcttatacttaccttaattctcaggcgtatcaaggtctaatttctctgatgaagagagaaatacccgcgaagcatttgacagaagcgctggatgatcagacgcttccgatttgctggaaaggacggaaacctttcaaaagcgtacgtgatgtcaagaaatacttcaagacctttgcattgagctttaagaatggcggaaaatctaaaactcaacttgaattcccaccagaagcttatcttatagtatcatccaacggaaacgcttgcttgggaattctaaatggtacagaagtaggtctgaatgatctgaatgtcattggagacatatcaatgcaagacagagtggtgatttatgacaatgagaagcaactgattggatgggcacctggaaattgcgacaggcttcctaaatccagaagcattatcatttgatgacaggtgttctaaacgttaaaaaaaacaattgtcctgtacagaaagctgctaagctgtaacattgtattcttccgccattacagcagattccttgtagagtaaaactatgtatatgtggtctggtgaatcagaccccacaattatacttgattggttatgaaaatttaataaaataacg

SEQ ID NO: 108 - DNA, 486 bp, Populus tremula x tremuloides
agatcactgaaaaatactcagaatggcccgagcatgccaccagtgcagcattttgtgagagcagaaatgctcccaagtgggtatctggtacggccttgtgaaggtggtggttcaattatacacatagttgaccacatggatttggagccttggagtgtgcctgaagtactacggccgctgtatgaatcctcaactgtacttgctcaaaagacaacaatggtggctctacgccagctgcggcagatagctcaggaagcttctcagtctaatgtgaccaactggggcagacgaccagcagctctacgagcactgagccagaggttgagcacggggttttaatgaggctctcaatggatttagtgatgagggatggtcaatgatcggaaatgatggcatggatgatgttactatcctcgtgaactcatctcctgacaagttgatgggttcaaatctttccttcactaatgggtatccagctgtcagcag

SEQ ID NO: 109 - DNA, 854 bp, Populus tremula x tremuloides
ggccacatgcaagagcatgtagcatcaatggctcggcaatatatccgcagcattatatcttcagttcagagggtggcattagcactatctccttctcatcttggttcacaggctggtcttcggtcaccactgggtactcctgaagcacagacacttgctcgttggatctgccagagctacaggagctatttgggtgtggagctactcaaatccaatggcgaaggaagtgaatctattctgaaaaccttgtggcatcattcagatgccattatgtgctgctcactgaaggtgttgcccgtctttacttttgcaaaccaagctggacttgacatgcttgagacaacattagttgcactgcaagacataactttggaaaagatatttgatgatcatggaagaaaaactctctgctcagaattcccacagatcatgcaacagggttttacttgtcttcaaggtggtatctgtttgtcaagcatgggcagaccagtttcatatgaaagagctgtggcctggaaagtgttgaatgaagaagaaaatgcgcattgcttctgctttatgtttataaactggtcttttgtctgagacttgaaaccagaatctagtggagtggctgatttaagagtgagaactctatacctgtcttattttgctggcttgtatttcttctatgtataacatgttacaaccgtggagcacccagttcatttgctatgttataaatttgtgtggggttcattatcgtagagattgtggacgagacgatctagttgccggggatcattttgtttctgactggtttgttctgaagtcctcacctgggtctatgttgattattgcaacccaaatctgtc

SEQ ID NO: 110 - DNA, 607 bp, Populus tremula x tremuloides
ggaatcagcctagccaagtagagattacaacgccaaaagctaggtcacactgatcagagcaacaacaaaagtacaccaacaatgtcttccaccccaacaaatgaatggcaaacctactggtgtcatgaatgtgacctcagcatccaccttctcaccaccaccactcctctttgccctcactgccaccatgactttcttgaactcatggaccccattcccacctccaccaccgctgacaccaccactttcctccttgacagcccctccttcctcaacttccttcaacatctcaacaccaatagccattgcgattgtgaagcatgataatacaaacgccactattgactctatcatccccaccataaaaatcacttcttgcatgctagaaatggatccaatgcttgtgtgtgcagtgtgtaaagatcagttcttgattgatagttgaggccaagcagcatgccctgcagtcacctgtaccatccaggttgcattctcccctggctttctaaccacaactcttgtccctctctgtcgcttccagttacaaacgccagtatgtcagagaggagaattacgaaacttgggtctcctgatcatcctcatcatg

SEQ ID NO: 111 - DNA, 259 bp, Populus tremula x tremuloides
gggttttgaccaccttttttcctccacacttttgaatttgaatgcaggtaaataaatgcttttggggattttgggtttttggtttggaattgatcgagtgaagtgtaaatcaacagtgaacgattgtgttacacaaagaacttgtgagggttgggcatccccttccccccttcagatttccactatggatattgtaaagtaaccccttgtttgggattggattttggccaaaaaaacagggtggttgaattgatttttt

SEQ ID NO: 112 - DNA, 475 bp, Populus tremula x tremuloides
gcacgggtgaaaagttacattaagaatcgatatgtgttgcgcaatctccttcaagttccggtaataaaagaggacagtatgaaggataaaaaggcaagacaaaaaggaagggatgatgattcaattacaagtgatatgctagtagagataatggaagaatcaataaggattttttggcgatttgttcgatctgataaagatgcacaaaacgtgatttcaaagggtcgcaaaggaactcaaatagagccccaagatcccacagaactagagttattgacagaggttcgaacgagtttccaaaagaaggagaggaggcttaaagacgttttgaggagtggaaactgcatactgaggaagttccaaaaacatcgagaggacgattctaatcaagttctttacttcttctctcaagtggatatgaagttagtagcaagggttctgagcatgtccagagtaacaacagagcagctactat

SEQ ID NO: 113 - DNA, 572 bp, Artificial Sequence
  Synthetic Construct
attgattaaccatggagaaagcacacacaaaatctgctcttaagaagcttgttaaggctagctcacagtctgcatcntggagcaatgctgctagaggaatggcgaaagatgatctcaaggatccgctttatgacaaatccaaggttgccccaaaaccccttgcaaaagaaaacacgaagccccaagaattcaaactccacactggacaaagagctctcaaacgtgccatgttcaactattctgtggcaaccaagatatatatgaatgagcaacagaagaggcaaatagagaggatacaaaagatcatagaagaagaagaggttcgtatgatgaggaaggagatggttccaagagctcaattgatgccttactttgacagacctttctttccccaaagatcaagcaggccattgacagttcctagagagccaagtttccacatggtgaacagcaagtgttggagctgcatccctgaggatgaactttactactactttgaacatgctcatccccatgaccatgcctggaagcctgttaagtaaataattgccatgatttgtaggggaaaaagg

SEQ ID NO: 114 - DNA, 1302 bp, Artificial Sequence
  Synthetic Construct
aagctgatgatcaaatgaggacatcaaaaatttcttcagggaattcaggctaccatgtatggattcttgcttttgcaatgagtctactgatactgattgccttgtcgaagtcatggttttatgatcacgcttctgctgctgcaagtgaggatttgcagtacttttcagtgatcgtcccttcaaaagggcgggactatcctcctgttcttgcttattggatatgtggtaccagtggagatgggaagagaatgttaaggctattaaaagcaatttatcatccaaggaaccaatatctattgcaacttgatgcggaatcctcagattatgaaagggcggagttggttgtttcagttcaatctgaaagtttgtttcaagcattcggtaatgttaatgttgttggaaaaggttttgctatcaacgaaatgggatcctctgctcttgctgccatactcaacgctgctgcgttgcttcttaagctgagtacagatggggactggttcatcaatttaagtgtctcagactatcctctagtgagtcaagatgatctcctccatgccttcacttccttgccaagagatctcaacttcatcaactatnctaatgacactgcgaaaaacgagatacacaagattaaccaaattgtagtagatcccagcttgcatcttcaaaagaggagccacctttattacgctgttgagactcgaacaacacctgatgctttcaagatatttggaggttcgccctggctgattcttacaagagctttcatggagtactgtgtccaaggatgggacaaccttccaagaaaactactaatgtacttcagcaacacggcatccccacttgaatcgtatttccactctgtcctctgcaactctcctgagtttcaaaacacaacagtaagcgatgatttaaggtacaatattctggaaactactacagatggggaatcaccttatgacaaaatgctaaatggtggagcagcatttgcaaggccatttaaagaagatgctgctgctctaaacatgatagatgagaacgtcttgaaccgtgaacccaacggattggtgcctggaaaatggtgcttagaccagggtatgaacaagagctcagaggcatcaaagcctccaggggaggatttgtgttcaacctggggtaacattaatgatgtcaagccaggatcttatggtatcaagcttgcatttttattgtctaagattgccagtgaagagaaattgacaactagtcaatgccttcaagctacaaaaatggggtcatcatagaaccaagcatacatccttg

SEQ ID NO: 115 - DNA, 556 bp, Artificial Sequence
  Synthetic Construct
attaatcatgtcaggtgatcaaaggcctaaagattctgctgagggctcttcgcgatctggcggcgaccactaccagcctcaaccggctcctttgagcaggtatgagtcgcagaaacggcgagactggaacacttttgggcagtacttgaagaatcagagacccccagtttcactatctcagagtaattgcaaccacgtgcttgatttccttcggtatctcgatcagtttggcaagactaaggttcatctacatggctgtgtcttttttggacaacctaatcctcctgctccttgtacctgccctctaaggcaagcttgggggagcctcgatgccctaatcggacggcttcgagcagcttatgaggagcacggaggatctgcagagaccaacccctttggaaatggagctattcgggtttatttgcgtgaagtgaaagagtgtcaagctaaggcaagggggattccatacaagaagaaaacgaagaagaagactcaaataagggcaagaaatgaagcgaagcctcctatgcagtcagcttaagtcctttggcttctt

SEQ ID NO: 116 - DNA, 4470 bp, Artificial Sequence
  Cloning vector pDONR201
ctttcctgcgttatcccctgattctgtggataaccgtattaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatctcgggccccaaataatgattttattttgactgatagtgacctgttcgttgcaacaaattgatgagcaatgcttttttataatgccaagtttgtacaaaaaagcagaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtgggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 117 - DNA, 13599 bp, Artificial Sequence
  Cloning vector pK7GWIWG2(I)
ctagagggcccgacgtcgcatgcctgcaggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcggactctagcatggccgcgggatatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatatcactagtgcggccgcctgcaggtcgaccatatggtcgacctgcaggcggccgcactagtgatgctgttatgttcagtgtcaagctgacctgcaaacacgttaaatgctaagaagttagaatatatgagacacgttaactggtatatgaataagctgtaaataaccgagtataaactcattaactaatatcacctctagagtataatataatcaaattcgacaatttgactttcaagagtaggctaatgtaaaatctttatatatttctacaatgttcaaagaaacagttgcatctaaacccctatggccatcaaattcaatgaacgctaagcttaatatgactctcaataaagtctcataccaacaagtgccaccttattcaaccatcaagaaaaaagccaaaatttatgctactctaaggaaaacttcactaaagaagacgatttagagtgttttaccaagaatttctgtcatcttactaaacaactaaagatcggtgtgatacaaaacctaatctcattaaagtttatgctaaaataagcataattttacccactaagcgtgaccagataaacataactcagcacaccagagcatatatattggtggctcaaatcatagaaacttacagtgaagacacagaaagccgtaagaagaggcaagagtatgaaaccttacctcatcatttccatgaggttgcttctgatcccgcgggatatcaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgtgatatcactagtgcggccgcctgcaggtcgactagaatagtaaattgtaatgttgtttgttgtttgttttgttgtggtaattgttgtaaaaatacggatcgtcctgcagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatggcctttgattcagtaggaactactttcttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttgatgagacctgccgcgtaggcctctctaaccatctgtgggtcagcattctttctgaaattgaagaggctaatcttctcattatcggtggtgaacatggtatcgtcaccttctccgtcgaactttcttcctagatcgtagagatagagaaagtcgtccatggtgatctccggggcaaaggagatcagcttggctctagtcgaccatatgggagagctcaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctagctagagtccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtatgataattcgagggtacccggggatcct

SEQ ID NO: 118 - DNA, 11135 bp, Artificial Sequence
  Cloning vector pKSGW7
ctcccatatggtcgactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgacctgcaggcggccgcactagtgatatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatatcccgcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcgacgtcgggcccaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctagctagagtccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtatgataattcgagct

SEQ ID NO: 119 - DNA, 20 bp, Artificial Sequence
  Construct specific primer
gggtaagatcaacttgaagg

SEQ ID NO: 120 - DNA, 20 bp, Artificial Sequence
  Construct gene specific primer
acttctccacaagagcattc

SEQ ID NO: 121 - DNA, 22 bp, Artificial Sequence
  Construct-specific forward primer
gtctatggatcggagcattgtg

SEQ ID NO: 122 - DNA, 20 bp, Artificial Sequence
  Construct-specific reverse primer
cagcagcaacatcctcagtg

SEQ ID NO: 123 - DNA, 22 bp, Artificial Sequence
  PCR primer
cactatgtcctctacttccttg

SEQ ID NO: 124 - DNA, 19 bp, Artificial Sequence
  PCR primer
atggtcttgggtcttcctg

SEQ ID NO: 125 - DNA, 20 bp, Artificial Sequence
  PCR primer
cactggtacgtgtagctgat

SEQ ID NO: 126 - DNA, 20 bp, Artificial Sequence
  PCR primer
gtaagaggtccacctttacc

SEQ ID NO: 127 - DNA, 52 bp, Artificial Sequence
  Forward primer
ggggaccactttgtacaagaaagctgggttccgagaaagaaaatggtgaccg

SEQ ID NO: 128 - DNA, 53 bp, Artificial Sequence
  Forward primer
ggggaccactttgtacaagaaagctgggtaaccaaccctgtacaaataccacg

SEQ ID NO: 129 - DNA, 52 bp, Artificial Sequence
  Forward Primer
ggggaccactttgtacaagaaagctgggtgctgcagaacagatgcttactcc

SEQ ID NO: 130 - DNA, 52 bp, Artificial Sequence
  Forward primer
ggggaccactttgtacaagaaagctgggtgaagaattagcaatgggcaaacc

SEQ ID NO: 131 - DNA, 52 bp, Artificial Sequence
  Forward primer
ggggaccactttgtacaagaaagctgggtattcacatgggaaaggtagtggg

SEQ ID NO: 132 - DNA, 53 bp, Artificial Sequence
  Forward primer
ggggaccactttgtacaagaaagctgggttttctctaggtcccagacagttgg

SEQ ID NO: 133 - DNA, 52 bp, Artificial Sequence
  Forward primer
ggggaccactttgtacaagaaagctgggttaagtggtccacgaaacagttgg

SEQ ID NO: 134 - DNA, 52 bp, Artificial Sequence
  Reverse primer
ggggacaagtttgtacaaaaaagcaggcttctttgcctaactttgggacctc

SEQ ID NO: 135 - DNA, 50 bp, Artificial Sequence
  Reverse primer
ggggacaagtttgtacaaaaaagcaggctaatcggaaccgtctgtcttgg

SEQ ID NO: 136 - DNA, 50 bp, Artificial Sequence
  Reverse primer
ggggacaagtttgtacaaaaaagcaggctttacaagatgctccgaatggg

SEQ ID NO: 137 - DNA, 53 bp, Artificial Sequence
  Reverse primer
ggggacaagtttgtacaaaaaagcaggctcagaaaacagataatcaacgagcg

SEQ ID NO: 138 - DNA, 51 bp, Artificial Sequence
  Reverse primer
ggggacaagtttgtacaaaaaagcaggctgaagcacaacggaagactgagg

SEQ ID NO: 139 - DNA, 54 bp, Artificial Sequence
  Reverse primer
ggggacaagtttgtacaaaaaagcaggctttgataaggtttgtctaatctcccg

SEQ ID NO: 140 - DNA, 54 bp, Artificial Sequence
  Reverse primer
ggggacaagtttgtacaaaaaagcaggctcgcttttgttcttattatagctggc

SEQ ID NO: 141 - DNA, 54 bp, Artificial Sequence
  Forward primer
ggggacaagtttgtacaaaaaagcaggctgagttcttgagctaaacgagaggag

SEQ ID NO: 142 - DNA, 23 bp, Artificial Sequence
  Forward primer
cggtcttatttgtattcgcctgc

SEQ ID NO: 143 - DNA, 19 bp, Artificial Sequence
  Forward primer
atgcaaccaggcttggatg

SEQ ID NO: 144 - DNA, 27 bp, Artificial Sequence
  Forward primer
atggtggagaagatcatcaatatgaac

SEQ ID NO: 145 - DNA, 20 bp, Artificial Sequence
  Forward primer
atggagtttgatgggctggc

SEQ ID NO: 146 - DNA, 23 bp, Artificial Sequence
  Forward primer
atgttgatggctttaaaatcccg

SEQ ID NO: 147 - DNA, 23 bp, Artificial Sequence
  Forward primer
atggacaacaatacctctcgctc

SEQ ID NO: 148 - DNA, 25 bp, Artificial Sequence
  Forward primer
atggaaacaaagtgtccagtagatg

SEQ ID NO: 149 - DNA, 55 bp, Artificial Sequence
  Reverse primer
ggggaccactttgtacaagaaagctgggttaactcaccaaattaagatcaaagcc

SEQ ID NO: 150 - DNA, 26 bp, Artificial Sequence
  Reverse primer
gcaaattagcaacacatgaacctacc

SEQ ID NO: 151 - DNA, 22 bp, Artificial Sequence
  Reverse primer
taaatgcagtaaatgatggagc

SEQ ID NO: 152 - DNA, 28 bp, Artificial Sequence
  Reverse primer
attacacaaatattcacactagaaaggg

SEQ ID NO: 153 - DNA, 22 bp, Artificial Sequence
  Reverse primer
caggggatatctagcttagggc

SEQ ID NO: 154 - DNA, 22 bp, Artificial Sequence
  Reverse primer
gaagttaacgccaagcagatgc

SEQ ID NO: 155 - DNA, 27 bp, Artificial Sequence
  Reverse primer
gacaaacttatggacccgttaataatc

SEQ ID NO: 156 - DNA, 24 bp, Artificial Sequence
  Reverse primer
ctactttttgttggggtcttgctc

SEQ ID NO: 157 - DNA, 336 bp, Artificial Sequence
  Synthetic Construct
tccgagaaagaaaatggtgaccgttgatgaaattcggaaggcgcagagggcagagggccctgccaccatcttggccatcggaacatcaacccctccgaactgtgttgatcaaagcacctatcctgactattattttcgtatcacaaacagcgagcataaggtggagcttaaagaaaaattcaagcgaatgtgcgagaaatccatgatcaagaagagatacatgcacttgacggaggagatcttgaaagaaaatcctagtgtatgcgaatacatggcaccttcattggatgctaggcaagacatggtggtggtggaggtcccaaagttaggcaaaga

SEQ ID NO: 158 - DNA, 322 bp, Artificial Sequence
  Construct seqeunce
aaccaaccctgtacaaataccacgggttccaacacagtaccaggcactgagccgcagttccccaattacccgagccttctaaccaaatgccatttgaatgtctccaacacaagggatggagctgtgatgcctgcttgtgttcccaacctggatccaagtcttgtgctcactaacttcacacggactacatactccgataaccttaatgatactaagtttacaggagctgggattggttctgatggtaactggatagttgttgttctgaccacaagcacacccgagggaagttttgtgacttccaagacagacggttccgatt

SEQ ID NO: 159 - DNA, 349 bp, Artificial Sequence
  Synthetic Construct
gctgcagaacagatgcttactccagcttctccagctctcccattctctctatgattgccctcttgcttgtttcttgaagctccccagcgagaatgaattcatcaagtatcaaataaaccttgtgaaagttaaacaccaaatctagctcacacacattgctgaagaaatgatccaatatctccacaaataaatgaatgcactccaaataagccaattcgttatccgttatgtcgacacacagcgaaaaaaacaatccagcataccgcctgtgtttttgaaaggcaatcttggaatttttttttcctttgttttttttccctgtggtctccccattcggagcatcttgtaa

SEQ ID NO: 160 - DNA, 326 bp, Artificial Sequence
  Synthetic Construct
gaagaattagcaatgggcaaaccagatggtaaacgagcttctcctggaagtcaggtcagtgtgcactatattggtaagctgaagaacggaaaagtatttgactctaatgtcggaagggctccctttaaattccgcctaggtgttggacaagtcataaaaggatgggatgttggagttaatggtatgcgagttggggacaaaagaagactcacagttccaccatcgatgggttacggagatcggggtgctggcgggaagatacctccaaactcatggcttgtgtttgatgttgagttggtgaacgctcgttgattatctgttttctg

SEQ ID NO: 161 - DNA, 296 bp, Artificial Sequence
  Synthetic Construct
attcacatgggaaaggtagtgggtcccttatcagtggggcgatgggagggttaagtttggaagaagggagtaactacgagaaagagaaagttgctactgataaggagagtcacagctaccatgattatcgtcgcgaatattgaaggaatagagagactgtgtgcgtgtgtgtgtgaaggtatatttgagctttttatactcgcattgtggctttgactgatggatatacttttgtatgtacatgcaaaaaattatcataactgctagatgctgccctcagtcttccgttgtgcttc

SEQ ID NO: 162 - DNA, 248 bp, Artificial Sequence
  Synthetic Construct
tttctctaggtcccagacagttgggaaccaacagcatcaattctgcaaatcaaggaatatcttacataaaggaaggaagaaaaattcaggggcaaaagatgtttcttcgacctgacctctcttctcatcttcgagatgaagcagcttcagttgtctcatcattttctgggccattatctccctgaattccaattcggacatttgaatcgaaaatcacatgtcacgggagattagacaaaccttatcaa

SEQ ID NO: 163 - DNA, 234 bp, Artificial Sequence
  Constsruct sequence
taagtggtccacgaaacagttggtgatttgcactccaactaggttatgctctttaattggttggatttcttttgacaggcaaacttgcctgttttagtgtgctctataataacagtcttgaaaaataagtgtacttttaagacaacaatgcatatggtatttcatcaattaagtgaagtttctgtatttgaatggaaagctgatgagaggccagctataataagaacaaaagcg

SEQ ID NO: 164 - DNA, 1176 bp, Populus trichocarpa
atggtgaccgttgatgaaattcggaaggcgcagagggcagagggccctgccaccatcttggccattggaacatcaacccctccgaattgtgttgatcaaagcacctatcctgactattattttcgtatcaccaacagcgagcataaggtggagcttaaagaaaagttcaagcgaatgtgcgagaaatccatgatcaagaagaggtacatgcacttgactgaggagatcttgaaagaaaatcctagtgtgtgcgaatacatggcaccttcattggatgctaggcaggacatggtggtggtcgagataccaaagttgggcaaagaagcagctgccaaggccatcaaggaatggggccagcccaagtccaaaataacccatttggtcttttgcacaaccagtggtgttgacatgcccggggctgactatcaactcactaagctcttgggtcttcgctcatcagtaaaacgtttcatgatgtaccagcaaggttgttttgctggtgggacggtgctccgcctagctaaagaccttgctgaaaacaacaaaggtgctcgcgtgctagtcgtgtgctcagaaataacagcagtgacatttcgtggccctagtgacacccacctcgatagtcttgtgggtcaagcgttgtttggtgacggcgcagctgctatcataataggctcggatcctgtgctgggggtcgagaagcctttgtttgagctggtgtctgccgcccagactattctacccgatagtgagggggctatagatggacatcttcgcgaagttgggcttacatttcaccttctcaaggatgtgcccgggcttatttcaaaaaacgtcgaaaagagtctaaccgaggctttcaagcccttgggcatctcggattggaactcccttttctggattgcgcaccctggtgggcccgcaattctggaccaggtggaggctaagttggagcttaaacctgaaaaactgcgagccactcgacaagtactggctgactatggtaacatgtccagtgcatgtgtgctatttattttggacgagatgaggaagaaatctgccaaggatgggcttaaatctactggagaagggttggaatggggcgtccttttcgggttcgggcctgggcttactgttgagacagtggtgctccacagtctgcctgccacaatttag

SEQ ID NO: 165 - DNA, 618 bp, Populus trichocarpa
atggcttcttcaagattttctcttctcttcccattctttgttttcttcattattctctgcttgatcagtcaccctgtcatttgtgacggtgatcaggaggatgcccttcttcaagggattaacaactacaggacatccttcaatttgactaccctcacaaaaaatgataacgcagagtgccttgctgaggagatagctgaccaatttaagaaccaaccttgtacaaataccacgggttccaacacagtaccaggcactgagccgcagttccccaattacccgagccttctagccaaatgccatttgaatgtctccaacacaagggatggagctgtgatgcctgcttgtgttccccacctggatccaagtcttgtgctcactaacttcacacggactccatactccgataaccttaatgatactaagtttacaggagctgggattggttctgatggtaactggatcgttgttgttctgaccacaagcacacctgagggaagttatgtgacttccaagacagacggttcggattacaatgcagccaatttgaccgccaagaatactggtctaatctatcacttgctgtttttgctgataggttctctcttcttgttgtga

SEQ ID NO: 166 - DNA, 429 bp, Populus trichocarpa
atgatccgatttatactcctgcaaaacagacaaggcaagactcgtctcgctaaatactatgttcctcttgaggattccgagaagcacaaagtcgaatacgaggttcaccgattggttgttaatagagatgctaaattcacgaattttgttgagtttaggacacacaaggtgatatacaggcggtatgctggattgtttttttcgctgtgtgtcgacataacggataacgaattggcttatttggagtgcattcatttatttgtggagatattggatcatttcttcagcaatgtgtgtgagctagatttggtgtttaactttcacaaggtttatttgatacttgatgaattcattctcgctggggagcttcaagaaacaagcaagagggcaatcatagagagaatgggagagctggagaagctggagtaa

SEQ ID NO: 167 - DNA, 1503 bp, Populus tricocharpa
atgagcttctggggtattgaagtgaaaccaggcaagcctcatccttatcactctgatgatgtgcaaggaaagcttcgggtaactcaggcaactttgggccttggttcgtcaaaagaaaggagcatactccagtgttcagttggacacaggagtccaatttttgtatgttcactgttgccagggaaagctgaaagttgctctctgaatcttgaatttacggatgaattagtagcgttctcagttattggaccacaaagcatccatctttgtggttactttgattctgtagaaggagatcacctccgagatgaatatgaatatgattcgggagaggatattgttgatacggagtcggatgagtccagtgaatatgattatgatgatgaatatgatgacgacgatgatgatcttcaaatgtattccccttcgcctgttcccaaaagtggagttgtaattgaggagataactgaagatgacaaacctacaaaagaaaatggcaagtctaaacggctaaagaagaagaataatcaatcaagtgacaaggaagaccaaaacaactctcaacaacaaattgttcttaagagggatagaggtatttctgttctggaaagtgaagatgaagatggttttccaatttcttcctctgcaaaaagcaaagatacagttcaggagcagcaagcagaactagatggacaaaaagataaggaaacaacccaagagactaagaaaaagatggcaagtgaggataatgatgacactagaaaaaagagaaaagttaaaagcgttgatcaagatggtcaaccagaaaggaaaacaaagaaaaagaagaagcagagagaacgaggtacagaggcaaaggttgatgaaatggatgacaaggaagagatcaacaatgcctctagagatgaaatcgagtccaagcaagtgaaaaagcaggattctaccaaccgaaacaaacatgaacagagggaccttgacactgatgcagatagtatgcccggtgaagattcatcggataagaaaaagaagaagaataagaagaagaaaaagacccaggatagtggagcaacaactaatgatcaagctgtttcagctgcgggaggtgaagccaagtcttcattggagtctgaggataagcaatctacagccaagtcctcacaagttagaaccttttcaaatggattggttattgaagaattagcaatgggcaaaccagatggtaaacgagcttctcctggaagtcaggtcagtgtgcactatattggtaagctgaagaacggaaaaatatttgactctaatgtcgggagggctccctttaaattccgcctaggtgttggacaagtcataaaaggatgggatgttggagttaatggtatgcgagttggggacaaaagaagactcacaattccaccatcgatgggttacggagatcggggtgctggcgggaagatacctccaaactcatggcttgtgtttgatgttgagttggtgaacgctcgttga

SEQ ID NO: 168 - DNA, 927 bp, Populus trichocarpa
atggcttcctcgaagtctcacgatctcgagatcacaataatctcagctaaacacctcaaaaacgttaattggcgaaacggcgatctcaaaccgtacgcaaccttctatctcgacaactcggaccgccgactcgcaactcacgctgacgactcactctcaactcgcccagtctggaacgaacgatttactctccccataactcgccagatctacgattcagtcctcaccctcgagatcttccactccaagccatcggaaaccccccaacctctcgtaggcactgtgaaattcccactgagtaatctcatggtctcggatgagtcgctgtcctgcgaagtcctcacgctcgagctcctacgtccctctggtcgtccacagggtaaagtccgggtgaaactagaggtgaaagaacggcctttgcctccgccagtgcaggattaccatactgcccctaattatagtcattattacaaccctgcccctgccccccctcctcctcctccaccacctcctgcacgtgactacagggaatactctccatctccatacggctacgccgatccgtacggttactaccctgcctattatcctccacaaccaccccgacctctctacaatcgagcatccaattacagcttgccaggtgggccttctgctccagttgatctatctgcccaatcatcaccatcgccctacgatcacaagccgccgccgccgccgccgggattgttccaaaagacgtccaattatggtgtgccaagtggaccgtcggctcccgtcgattattcacatgggaaaggtagtgggtcccttatcagtggggcaatgggagggttaagtttggaagaagggagtaactacgagaaagagaaagttgctactgataaggagagtcacagctaccatgattatcgtcgcgaatattga

SEQ ID NO: 169 - DNA, 1716 bp, Populus trichocarpa
atgtggatatctacttcaggaattgcaggcaagactgcctcttggaaatcaatgatattaatttatcaatcaataacagcattgcttttttcttcacgatctctttcccttcctcttaatatgaaacagatgatatgggaagaagttggggaagatcagtttgaacgagagaaggtcctgctggatttagaacaagagtgcttagaggtttataggagaaaagttgacaatgcaaacatatcaagagctcgcctgcatcaagagctggcggaatctgaagctgaattcacccatcttcttctgtcccttggtgaacgatcactcccaggacggccagaaagaaagtcaggaacactgaaggagcagctagatgcgatcaccccagctctacgggagatgcgtttgagaaaagaagagagggtgaatcaatttagatctgtgcaaggtcaaattcaaaaaatttctgcagaaattgcaggtcaatcagtgtacgatgacccgataacaaatgtcaaggtaaatgagaatgatctttcattaaagaaacttgaggaataccaaattgagctacaaaggctgcacaatgagaagaatgacaggctccagctagtggacacatatattgacacaatccatgatttgtcctcaacattaggaatggaatcctccatgatcataacaaaggtgcatccaactttgaatgaattgtgtggaatatcaaaaaatataagtgacagtattctggataaactcaacagcactgtggaatctctcaaagaagaaaaacaaaagcggcttgagaagcttcatcagcttggaaaggcactgaaaaacttgtggaatctaatggacacgccctataaagattgtcactcattttccaatgtcacggacttattatttctctcatcagatgaagtatcaggtcctggaagccttgctctaaatataatccagcaggctaaaaaaggggagattgaccatgccgatctcctcatgagcttggatgagcagatatcaagagcaaaagaggaagcttctagcaggaaggctataatggaaaaggttgaaaagtggatattagcacgtgatgaagagcgctggttagaagaatacagcatggacgagaatcgatattcagttcgcagaggtgctcataagaatctgcaacgtgcagaacgtgcccgggtaatagtcaacaagatcccagttctagtggcatcgctcgtagcaaagactgagagttgggaagaggaaagaaacaaagtattcctgtatgatggagtacccctgctggcaatgctggaagagtataacatatcgaggcaggagatggaagaggaaaagcaaagacaaagggctagttcatccaacactctacaggcaaaggaaaagaaggtcccaagccatgtagaagttgagcaagaaaatttgatcgggtcaagctcaaggccaagcaccagcagtcgacgtctttcaaacaagagcttgaatggaggatttagcaatgcaacgcctttaaacagaaggctttctcttggtcccagacagctgggaaccaacagcatcaattctgcaaatcaaggaatatcttacataaaggaaggaagaaaaattcaggggcaaaagatgtttcttcgacctgacctctcttctcatcttcgagatgaagcagcttcagttgtctcatcattttctgggccattatctccctga

SEQ ID NO: 170 - DNA, 2763 bp, Populus trichocarpa
atgcaagcagcctccggaactaagcgtggttctagtggttccccggtgattgataagcaagggagggcagtggccttgaacgctgggggcagtgtatcatcttcatcagccttctatttacatttagagagagttgttagggcattggcatttctccagaagtccaaagatgcctgtaaaaacaaatgggaagcagtttctatacctcgtggcacccttcaggtaacgtttctccataaaggatttgatgagacacgccggcttggtcttccgaatgaaacagagcagatggtgcggcaagcctctgcaccaggtgaaactggaatgttggttgttgactctgtggtgccatgtgggccagctgataggcaattggagtcaggggatgtgcttgtgcgtgtgaatggggaagtgactactcagtttttaaaattggaggcattgcttgatgacagtgttgacaaaaagatcgaattacagattgaaaggggtggcacatcattaaccgttgatatagtggttcaggatttgcacttgataactcctgactgcttcttagaagtaagtggtgcagtgatacaccgtttgtcttatcaacaggccagaaacttctgcttccaatgtggtcttgtatatgtttcggaccccggatacatgctatctagggctggcattcctcgtcatgccatcattaagaagtttgccgatgatgagatatcacaacttgaagatgtaatctctgttctgtctaagctatgtaagggtgatagagtgccattggagtatataagctacagggatcgccatcgaagaaagtctaccctggtcacaattgatcgccatgaatggtacgatgctccgaagatatacacacgggatgatagttctggtttatggatggcaaggcctgctattcaaccgaccaccctgcagttgtcaccttgtagtagcaatgtcactcaaggtctaaacagccaagcatcttcattgaatagtgaatcaactcctgctgaaggtacggatcaagctaacaaccaggagttgacacatgatattttaagaacagaagccggttatgaacatatttctgaggaggtccattccagggaagagtgtgatgttaaaactaataagcaacaggtacaggggaacttgtcctctgatgaaattgcagttgctgaccattcctcgcttgaaattggagaaatgaaattggagactccaggtaccactgaaattacagtttcgaatggctatgaaggtgcaatagcagcagcaactaatgcttcatttgcggaatgtgtggtggagcccactcttgtaacattggaggtcaatgtgccaccatcatgtttgcttgatggcatccattcagtgcatgcttctgggactggtgttgttgtacatcattctcaagacatgggattggttgctattgacaagaacaccgttgaaacatctgcatgtgatgtgatgctgtcatttgctgcttttcctatcgagattccaggagaggttgtctttcttcatcctgtttacaattatgctcttgttggttatgatccctctgctctgggagctgttggtgcttctatggttcgtgctgctgagttacttcctgagcctgcattatgtcgtggagatccaatatatctgattgggttaagtaaaaatcaacgagcaaagtctaggaaatcaattgtgacaaacccttatgttacattaaattttggctatgctgatcgtccacggtatagagcaataaatatggaagtgattgagcttgacactgattttggtaatgcatttactggtgtgctatgtaatgagcatggaaaggttcaggctatatgggggagcttttcaaataagccaaaatctagtcacactacatcaaaggatcatcaatttgttcgaggtgtcccaatctacatgattagccaagtacttgacaaaatcatatctggtgcaaatggggcttctattctcataaacggtatcaaaaggtcaatgccacttgttaggactttagaggtggaactttgctctagattgctttcaaaggcccggagttttggtcttggtgatgaatggatccaaagacttgtaaagaaagacccaatgagacgacaagttttgcgtgttaaaggttgtttggctggatctaacactgagaatctattaaaacaaggcgatatgttgttggcaatcaacaaagagccagttacttgcttccaagatgtagaaaatgcatgtcaagcattagagaactgtgttgacagtgatgggaagcttaaaattaccatttgtcggcagggaggtgaagttgatctacttgttggaacagacattagggatggtaatggtacaacacgagcagtaaattggtgtggctgtcttgttcaggatcctcatccagcagttcgtgctcttggatttcttcctggtgaaggtcatggtgtctatgcagcaatgtgctgccgtggaagtcctgcagatagatatgctctaggtgctcttagatggattgttcgagtcaatggaaaacccactcctgacttggatgcttttgtaaatgtgacaaagggattacgatatgatgagttcgttcgtgtaaagaccatcaatctggatgggaagccacgagtgctaacgttgaagcaggacttgcactactggcctacatgggagttgaggtttgatccaaacaccgctaggtggcggagagagacaataaaggctttagattgcaatattgaataa

SEQ ID NO: 171 - DNA, 651 bp, Populus tremula x Populus tremuloides
agaaaacaacagtagcttcaattagcagcagcagcaatggcttcttcaagattttctcttctcttcccattctttgttttcttcattattctctgcttgatcagtcaccctgtcatttgtgacggtgatgaggaggatgctcttcttctagggattaacaagtataggacatccttcaatttgagtaccctcacaaaaaatgataacgcagagtgccttgcagaggagatagctgaccaatttaagaaccaaccctgtacaaataccacgggttccaacacagtaccaggcactgagccgcagttccccaattacccgagccttctaaccaaatgccatttgaatgtctccaacacaagggatggagctgtgatgcctgcttgtgttcccaacctggatccaagtcttgtgctcactaacttcacacggactacatactccgataaccttaatgatactaagtttacaggagctgggattggttctgatggtaactggatcgttgttgttctgaccacaagcacacctgagggaagttttgtgacttccaagacagacggttccgattccaatgcagccaatttgaccgccaagaaaactggtctaatctatcacttactgttttgctgataggttctctcttcatgttgt

SEQ ID NO: 172 - DNA, 826 bp, Populus tremula x Populus tremuloides
cccctcggaggtcgaccccacgcgtgccgctaatgcagataatatacccggtgaagattcatcggataagaaaaagaagaagaagaagaaaaagaaaaagacccaggatagtggagcaacaactaatgatcaagctgtttcagctgtgggaggtgaagccaagtcttcattggagtctgaggataagcaatctacagccaagtcctcacaagttagaaccttttcaaatggattggttattgaagaattagcaatgggcaaaccagatggtaaacgagcttctcctggaagtcaggtcagtgtgcactatattggtaagctgaagaacggaaaagtatttgactctaatgtcggaagggctccctttaaattccgcctaggtgttggacaagtcataaaaggatgggatgttggagttaatggtatgcgagttggggacaaaagaagactcacagttccaccatcgatgggttacggagatcggggtgctggcgggaagatacctccaaactcatggcttgtgtttgatgttgagttggtgaacgctcgttgattatctgttttctgtgctccttttctgcttttgaactcaatggctgcttttacagacagtgggtatagatggatctcgaaaattggtgttttaactgtaggcttgttttacaatactgcaagttcacccccatcgcataacttaagtgtcaagatggatggtgtgaataaaattttggttaattcaattgtagcaagactagtgtgctctgcaaaaattttggttaaaactagctgttctgtttcagcagaatgtaaaaaaaaaaaaaaaaaa

SEQ ID NO: 173 - DNA, 731 bp, Populus tremula x Populus tremuloides
gggaatacgctccatctccatacggctacgccgatccgtacggttactaccctgcctattatcctccacaaccaccccgacctctctacaatcgagcatccaattacagcttgccaggtgggccttctgctccagttgatctatctgcccaatcatcaccatcgccctacgatcacaagccgccgccgccgccgcgggattgttccaaaagacgtccaattatggtgtgccaagtggaccgtcggctcccgtcgattattcacatgggaaaggtagtgggtcccttatcagtggggcgatgggagggttaagtttggaagaagggagtaactacgagaaagagaaagttgctactgataaggagagtcacagctaccatgattatcgtcgcgaatattgaaggaatagagagactgtgtgcgtgtgtgtgtgaaggtatatttgagctttttatactcgcattgtggctttgactgatggatatacttttgtatgtacatgcaaaaaattatcataactgctagatgctgccctcagtcttccgttgtgcttcaattatttcttatctagtgtgtctgtttctcttacttctgtaatggatgtttggttctatacttgcttctttgtttcaagtcttaattcgagtttgctagcttatacacttgggtactccattgattgttaattgtagtataaatagtgaatgattagagaatgatgggggaacttaa

SEQ ID NO: 174 - DNA, 822 bp, Populus tremula x Populus tremuloides
aaaaaaaaaaaaaaaaaactcgagactagatcgcttgtaacaaagactgagagttgggaagaggaaagaaacaaagtattcctgtatgatggggtacccctgctggcaatgctggaagagtacaacatatcgaggcaggagatggaagaggaaaagcaaagccaatggggtagttcatccaacactttacaggcaaaggaaaagaaggtcccaagccatgtagtagttgagcaagaaaatttgatcgggtcaaggccaagcaccagcagtcgacgtctttcaaacaagagcttgaatggaggatttagcaatgcaacgcctttaaacagaaggctttctctaggtcccagacagttgggaaccaacagcatcaattctgcaaatcaaggaatatcttacataaaggaaggaagaaaaattcaggggcaaaagatgtttcttcgacctgacctctcttctcatcttcgagatgaagcagcttcagttgtctcatcattttctgggccattatctccctgaattccaattcggacatttgaatcgaaaatcacatgtcacgggagattagacaaaccttatcaaggaccataatagtaactgttatttctgcaaaacaataatttacctatttgcatcaatttgataagaaataacttgtgtatctatgccttgtaccaacacccgtatttagaacagaaccccaaagaactgtcattgcaatgaaaaataattattcaggtttaaaactatgtagagctatgttttgtaatcaagaagctttgattttcctattctattgaaattatgaaacatgataatttc

SEQ ID NO: 175 - DNA, 829 bp, Populus tremula x Populus tremuloides
ctgtgttgacagtgatgggaagcttaaaattaccatttgtcggcagggaggtgaagttgatctacttcttggaacagacattagggatggtaatggtacaacacgagcaataaattggtgcggctgtcttgttcaggatcctcatccagcagttcgtgctcttggatttcttcctggtgaaggtcatggtgtctatgcagcaatgtgctgtcgaggaagtcccgcagatagatatgctctaggtgctcttagatggattgttcaagtcaatggaaaacccactcctgacttggatgcttttgtaaatgtgacgaagggattacgatatgatgagttcgttcgtgtaaagaccatcaatctggatgggaagccacgagtgctaacgttgaagcaggacttgcactactggcctacatgggagttgaggtttgatccaaacaccgctaggtggcggagagagacaataaaggctttagattgcaataatgaataaacagattgttctgatctcacttcgtgttctttcctggaatggttattgcacgttcaacactgcttcaagagcgtaagtggtccacgaaacagttggtgatttgcactccaactaggttatgccctttaattggttggatttcttttgacaggcaaacttgcctgttttagtgtgctctataataacggtcttgaaaaataagtgtacttttaagacaacaatgcatatggtatttcatcaattaagtgaagtttctgtatttgaatggaaagctgatgagaggccagctataataagaacaaaagcgaatgctttattttgtgaatgtaaaaaaaa

SEQ ID NO: 176 - DNA, 487 bp, Artificial Sequence
  Synthetic Construct
gagttcttgagctaaacgagaggaggaggagaattgtttttcgtttttcagatatggaggagaggcatgttatttttgggaagtatgaaatggggaggcttttagggaagggaacttttgctaaagttttctatgggaaacacttggtgacaggggagagtgtggcaatcaaagtcataagcaaagatcaagtcaagaaagaagggatgatggagcaaatccagagagaaatctcagtcatgcgtcttgttcgtcatcccaacattgtagagctcaaggaagtcatggctaccaagactaagatcttcttcatcatggagtatgttcgaggaggagagttgtttgccaaagtagccaaaggaaggctgaaagaagaagctgctcgaaaatatttccagcaactaatcagcgcaattgattattgccatagcagaggtgtttaccatagagatttgaagccagagaatttgttgcttgatgaagatga

SEQ ID NO: 177 - DNA, 452 bp, Artificial Sequence
  Synthetic Construct
taactcaccaaattaagatcaaagcctttctacaacatttgaatctcaggatcttctacatgactattgttgttatcttgattacaaacattgtctccttgccatgtccaaacgatgtcttttagtgcaggcctaacatcttcctcgcagaacctagtatactccaaggtatctccggaagacttggagaactccaccaccgcaacctccggtgccacctcgaacacctccgccgtcaccgccagcttcccctttctcccctcacatggaccttgtaacgtcactttgaaatctttgacttttgccaccttataactcagcccccttgcaaccccttcaattttttccatgatcgcactcgctgaaaattttgaagtgaacatagagcttgatttcctcttagtctcaaaaagaccagacagatcaaagccggatgacatcgaggaaataaa

SEQ ID NO: 178 - DNA, 1560 bp, Artificial Sequence
  Synthetic Construct
cggtcttatttgtattcgcctgcaatatcgtacggcctgggatctctcttctcttcttactgagcaagttgcatctcataagcactggcgatcatggagctgaatggtcaaacaagagtgagaagaaaggaccattttgctcatacaaacggtgatttagcattagcaagtgttggtgatgtagatccctggactgcatgggcatataagcctcgaactatttcgctgttacttattggtgcttgctttctaatatgggcaagtggagccctagatccagagagctgtacatctggtgatgttgttacatctgtgaaaaggggtatatgggcaatgactgcagtttttcttggttattgcttgctacaagccccttcaacggttctaataaggccacatccagcaatttggcgcttagttcatggattggccattgtttatcttgttgccctcacatttctgctttttcagaagcgtgatgatgcgcggcaatttatgaagtttctccatcctgaccttggaattgaactacctgaaagatcatatggtgctgattgtcgcatttatgtgcctgaaaatcctacaagcaagtttaagaacgttttggacactctttttgatgaatttgttctagcacatatctttggatggtggggcaaggctatattaatccgtaatcagccacttctatgggtattgtcaattggttttgagctgatggagtttaccttccgccacatgctaccaaacttcaatgaatgctggtgggacagtatcattcttgatattttgatatgcaattggtttggcatttgggctggaatgcatacagtcgggtattttgatggaaaaacatacgagtgggttggtataagtcgccaacctaatattatgagcaaggtcaaacgaacattggaacaatttacacctgcacagtgggacaaagatgaatggcatcccttgcttggtccatggcgatttatccaagttcttagtctctgtattgtcttcctgactgtagagctcaacacattctttttgaagttttgtctatgggttcctcctcggaaccctgtaatagtgtacaggctgatcttgtggtggctaattgccatacctacaacacgtgaatacaattcatatctccaagaccgaaagcctgtgaaaaaggttggtgctttttgttggctttcccttgctatttgcattgtggaacttctcatttgcatcaagtttggacatggtctttatcccaaaccaatgcctgcatggttggtcatcttctggacatctgttggagttagccttgtaatattcctgattatgtggtcatggaaaagtttgggaagaaagagacgatgattttgctagcatggtacactattcttttattccttcatgttcttatgggtatacatcttgtaaatgccctatagcttcatggaattgtaaatgcttattctcttaggttagtggatcacagttttagagtattcaattcttttatgataggtagttggaaaatgcttctaatggtaggttcatgtgttgctaatttgc

SEQ ID NO: 179 - DNA, 561 bp, Artificial Sequence
  Synthetic Construct
atgcaaccaggcttggatgagatcacaatgacggggtggaagaatctgaagcaacggctgtcattcaagggcctgggtagttgctgcgggagcacaagctggatttccagaagtaccacccaaaccatgccctttatcgatatagaggaagaagaagaggaagagaccaccatgcaaaaccaagctcaaagaggaggaggaggaggagcagcagcagcgccaggtgctgggatgaatctggcaatggcattagctgctgagcgcgatttaggggattcaaatgtcaagacattgatgagtttgatcgaagaaacggacggtgttgattggaggaagaagaataacagtaataacaaaagcaggagggacaaggaacaggaacagaagcaggaagaagagaaggattgggtatgctgcgtgtgcatggagagaaataaaggagcagcttttattccatgtggacacaccttttgtagggtttgttcaagagaaatgtgggttaatcgagggtgctgtcctgtctgcaaccgttccattctcgacatccttgatatcttctag

SEQ ID NO: 180 - DNA, 369 bp, Artificial Sequence
  Synthetic Construct
atggtggagaagatcatcaatatgaacagtcaagatcatctgaggagtactaattataaagatgatgatgatgaagaagaagaagaagttcaacttccagggtttcgatttcacccaacagatgaagaacttgttgggttctatcttcgtagaatggttgacaagaagcctctcagaattgaactcatcaaacaagtcgagatctacaaatatgatccatgggatctaccaattaagtcaagctgtgtgggagataaggaagggtacttcttttgcaaacgaggaagaaagtataggaatagcataagacctaacagggtgacaggttctggattttggaaagcaaccggcattgataagcctgtgtat

SEQ ID NO: 181 - DNA, 410 bp, Artificial Sequence
  Synthetic Construct
attacacaaatattcacactagaaagggatcaaaagcacaatctacaactgatgtaagctcatcccagtccccatgcgtaagaatctgatttccaaatgggctggaaatgttcaaagatgaagccatagatggtggttgagctacgtagctcaactcgtccacatgcagtggtttcctctccttgacatgatgaactggaggcttattatcgtaatgttgaatgagtggagcaccaaaattgacgtagctttcttgattgtaattagattccacttgacagcatgtcttggagctcgcgtcgattggtgcttgttgaggtgtagttgataattgcttcaaatctggtgtgtattgtctgcatgacacatttcttttgaaaattctacacagtgtccatacctcagcttct

SEQ ID NO: 182 - DNA, 787 bp, Artificial Sequence
  Synthetic Construct
atggagtttgatgggctcccaggttttcgattccacccaacagaagaagaactcttagatttctaccttacaaagactgtcttaggtcaagacacagctgatatcattggtttcctcaacatatacaatcacgaaccttgggacttgccaggattatcgaagataggggagagggagtggtatttccttgtgcacagggagagcgttcttgggagacccaggaggacaaccgagaaaggttactggaaggcaactggttcggacaggcccatccgatgcttgatggatccgaaaaggttgttgggtcatagaaaaactatggttttctacagaggaagagctccacgagggagcaaaacggattgggtcatgaatgagtacagattgcctagcaactgctatttatcgaaggagatcgtgctttgcaaagtatatagaaaggcaacatcattgaaggtcttggagcaaagggcagccatagaagaaattgttagagcacctaacgtcacatctttatctcctccaatgcgagggaatttctctttttatgaccaccaaaagagcttcaacaagttcttgttagaacaaaataatgttgttcacaaagtagagggagtgacagtaatagaagaagaagaagacaacaaaatcggttacccatcccaaataatgggagttggacccatcaaagagcactactccgagctcctgtcaccaaagttagtcctggattggaccatggattcactttggcctaactaaggagccctaagctagatatcccctg

SEQ ID NO: 183 - DNA, 276 bp, Artificial Sequence
  Synthetic Construct
atgttgatggctttaaaatcccgggttaattctgctgaaaagacaccttctgtaagggagttgtacagcaaggaacatgtagatgcaacccagtccttatatgacttgtcaccttttttcaagcttggtttcatggctgccaatcttgccatcattgaggccacaagagagcaaagccgagagatgaatagctgcagcaatggcttccatgttgttgattttgatatcggtcaaggagggcaatacatgaaccttctacacgcgctttctgggctt

SEQ ID NO: 184 - DNA, 364 bp, Artificial Sequence
  Synthetic Construct
gaagttaacgccaagcagatgcgacagtgagagtttttcccttccaaccaaaacataccccaccgttctcttctttaacagtaaatcctgggttaactcggtttgttaaactcaatctcgctttcattgactcagcaacgatttgactcatcggcttcaactcgaatcctgccattcccatccgggcccgccatttcccaaagacctcgcatctttcaatccggtccctgccttcgcaagcaaccgagtttactatcgtccgacccagtcccccctcgacccttgctcgctttgagctatcccctttccattgttgagtcgattgagtccgaatagcgcctccgtaatatgaacatgattcgtt

SEQ ID NO: 185 - DNA, 1681 bp, Artificial Sequence
  Synthetic Construct
atggacaacaatacctctcgctctgaggtttccgactgcggcgatccaacgaggccggagtctggcggcggcggtgctaggtacaagttgatgtcgccggccaagcttccgatctcgaggtcggcttgcatcacgatccctcctggactcagtccgacctcgtttcttgagtctccagttcttctctctaatgttaaggcagagccttcccccactactggtacgtttactaagcctcgaacagttcttggctctcttagctccactccatattctgctacaactgtttcatccacagcgtgtggagaaagaaaatccgacagctttgagtttagaccatatgctaggtcaaacatggtttctgcagatattaaccatcagaggagcacacaatgtgctcaagttcaaagccagtgccattcacaatcatttgcctcaccacctttggtaaaaggtgagatggaagtatccacaaatgagttgagtctgtcagcaccccttcacatggttacttcagttgctagtgcacctgctgaagttgattcagatgaactaaaccaaacagggctctctggcagtgggcttcaggcatcacagtctgatcatagagtaggaagtgcgccttcaatgtcatctgatgatggatataactggagaaaatatggacagaaacatgttaaaggaagtgagttccctcgcagctattacaaatgtacgcatcctaactgtgaagtgaaaaagttatttgagcgctcccatgatggacaaataacagagattatctacaagggtacacatgatcatcctaaaccacaacccagccgccgatatgcctctggttctgttttgtccatgcaggaagatagatttgacaagtcttcatctctgcctaaccaagatgacaagtcacctggtgcttatggacaggtgcctcatgctattgagccaaatggtgcccttgaactgtctactggagcaaatgatgatactggagaaggtgctgaagacgatgatgatcccttctcgaaaagaaggaggttggatgctggaggttttgatgttactccagtagtcaaacctatccgggaaccacgtgttgtcgtgcaaactctgagcgaggttgatatactggatgatgggtacaggtggcgcaaatatggccagaaagtggtgagaggaaatcctaatccaaggagttactacaaatgcacaaatgctggatgcccagttagaaagcacgttgagagggcatcacatgatccaaaagcagttataaccacatatgaggggaaacacaatcatgatgtacctacagctaggacaaacagtcatgacatggcgggaccatcagctgtgaatggaccctcaaggatcagaccagatgaaaatgagaccataagccttgatcttggggtcgggatcagttctacaactgaaaaccagtccggcgatcagcaacaagctttccgtgcggaacttatcaaacatgaaaaccaagcaagtggttctagtttcagagtagttcatgcaaccccaattacggcttactacggtgttttaaatggtggcatgaatcagtatggatctaggcaaattccaggtgaaagccgtagcattgaaattccaccttatccatatccgcagaacatgggaagattattaacgggtccataagtttgtc

SEQ ID NO: 186 - DNA, 312 bp, Artificial Sequence
  Synthetic Construct
atggaaacaaagtgtccagtagatgcaaggtgcgcagctgaaatatctgccatgctcacccctccttctcctcttcaactccaggagtattttgaggagattatatctgaaaggaaatgccatggcattgaagtcaaacgagatggaaacttgagcaaaggggtttatgcaactatggaacttaaagaaggagaacttatactgaaggacaagatacttgtgggtttacagcatgttccaaataagcttgactgtctggtgtgcagctattgtttccaatttattgagtcagttgaatatcagattgggagg

SEQ ID NO: 187 - DNA, 235 bp, Artificial Sequence
  Synthetic Construct
ctactttttgttggggtcttgctctaagcaagcgttgcacctgcatttgaatccataatctgctagcaatgcctgtctgtcttcaaatggaaggtcctcgtctatgtatgaaacggtaacctcttctccctttagaattggtttcagggtgatgattgctgcctggccatctctgtcctcatctcttttgaaggcgtgtgcatttgggcagcaggagtgattcatgcagctctgt

SEQ ID NO: 188 - DNA, 1356 bp, Populus trichocarpa
atggaggagaggcatgttatttttgggaagtatgaaatgggtaggcttttagggaagggaacttttgctaaagtttactatgggaaacacttggtgacaggagagagtgtggcaatcaaagtcataagcaaagatcaagtcaagaaagaagggatgatggagcaaatccagagagaaatctcagtcatgcgtcttgttcgtcatcccaacattgtagagctcaaggaagtcatggctaccaagacaaaaatcttcttcatcatggagtatgttcgaggaggagagttgtttgccaaagtagccaaaggaaggctgaaagaagaagctgctcgaaaatatttccagcaactaatcagcgcaattgattattgtcatagcagaggtgtttatcatagagatttgaagccagagaatatgttgcttgatgaagatgaaaacttgaaaatctctgattttggcttgtcagccttgcctgaacaatttcggcaagatgggcttttgcatactcagtgtggaactcctgcttatgttgccccagaagtcttgagaaagaaaggctatgatggatcaaaagctgatatatggtcatgtggggtgattctttatgtgcttcttgcaggattcttgccatttcaagatgagaatgttatgaagatgtacaggaaagtcttcaaggctgaatatcagtttccgccttggttttcaacagattctaagaggttgatttcaagacttcttgttgctgatcctgaaaagagaatcacaattcctgccataatgagaaatcattggtttcttaaagggttttcaagaccagtggccttttctatccaagaatcaagcatggatcaaacaggacaagaacaagatcttgattcttgttctgttgtcaacaccaaggtatcatcaccagaattcttcaatgcattcgagtttatttcctcaatgtcatccggctttgatctgtctagtctttttgagactaagaggaaatcaggctctatgttcacttcaaaattttcagcgagtgctatcatggaaaaaattgaaggggttgcaaaagggctgagttataaggtggcaaaagtcaaagatttcaaagtgacgttacaaggtccatgcgagggtagaaaggggaagctggcagtgacggcggaggtgttcgaggtggcaccggaggttgcggtggtggagttctccaagtcttccggagataccttggagtatactaagttctgtgaggaagatgttaggcctgcactaaaagacatcgtttggacatggcaaggagacaatgtttgtaataaagataacaacaatagtcatgtagaagatcgtgaaattcaaatgttgtag

SEQ ID NO: 189 - DNA, 1272 bp, Populus trichocarpa
atggagctgaatggtcaaacaagagtgagaagaaaggaccattttgctcatacaaacggtgatttagcattaccaagtgttggtgatgtagatccctggactgcatgggcatataagcctcgaactatttcgttgttacttattggtgcttgctttctaatatgggcaagtggagccctagatccagagagctgcacatctggtgatgttgttacatctgtgaaaaggggtatatgggcaatgactgcagtttttcttggttattgcttgctacaggccccttcaacggttctaataaggccacatccagcaatttggcgcttagttcatggattggccattgtttatcttgttgccctcacatttctgctttttcagaagcgtgatgatgcgcggcaatttatgaagtttctccatcctgaccttggaattgcagaactacctgaaagatcatatggtgctgattgtcgcatttatgtgcctgaaaatcctacaagcaagtttaagaacgttttggacactctttttgatgaatttgttctagcacatatctttggatggtggggcaaggctatattaatccgtaatcagccacttctatgggtattgtcaattggttttgagctgatggagtttaccttccgccacatgctaccaaacttcaatgaatgctggtgggacagtatcattcttgatattttgatatgcaattggtttggcatttgggctggaatgcatacagtcgggtattttgatggaaaaacatacgagtgggttggtataagtcgccaacctaatattatgagcaaggtcaaacgaacattggaacaatttacacctgcacagtgggacaaagatgaatggcatcccttgcttggtccatggcgatttatccaagttcttagtctctgtattgtcttcctgactgtagagctcaacacattctttttgaagttttgtctatgggttcctcctcggaaccctgtaatagtgtacaggttgatcttgtggtggctaattgccatacctacaacacgtgaatacaattcatatctccaagaccgaaagcctgtgaaaaaggtaggcgctttttgttggctttcccttgctatttgcatcgtggaacttctcatttgcatcaagtttggacatggtctttatcccaaaccaatgcctgcatggttggtcatcttctggacatctgttggagttagccttgtaatattcctgattatgtggtcatggaaaagtttgggaagaaagagacgatga

SEQ ID NO: 190 - DNA, 564 bp, Populus trichocarpa
atgcaaccaggcttggatgagatcacaatgacggcgtggaagaatctaaagcaacggctctcattcaagggcctgggtagttgctgcgggagcacaagctggagttccagaagtgccaccccaaccatgccctttatcgatatagagcaagaagaagaggaagagcccatcatgcaaaaccaagctcaaagaggaggaggagctgcagcagcagcagcagcgccaggtgctgggatgaatctggcaatggcattagctgctgagcgcaatttaggggattcaaatgtcaagacattgatgagtttgatcgaagaaacggacggtgttgattggaggaagaagaataacagtaatgataaaagtaggagggacaaggaacaggaacagaagcaggaagaagagaaggattgggtatgctgcgtttgcatggagagaaataaaggcgcagcttttattccatgtggacacaccttttgtagggtttgttcaagagaaatgtgggttaatcgagggtgctgtcctatctgcaaccgttccattctcgacatccttgatatcttctag

SEQ ID NO: 191 - DNA, 927 bp, Populus trichocarpa
atggtggagaagatcatcaatatgaacagtcaagatcatctgaggagtactaattataaagatgatgatgatgaagaagaagttcaacttccagggtttcgatttcacccaacagatgaagaacttgttgggttctatcttcgtagaatggttgacaagaagcctctcagaattgaactcatcaaacaagtcgagatctacaaatatgacccttgggatctaccaaagtcaagctgtgtgggagataaggaagggtacttcttttgcaaacgaggaagaaagtataggaatagcataagacctaacagggtgacagggtctggattttggaaagcaactggcattgataagccagtgttttcacttggaggagaaggccgtgacagcattggactcaagaaaacacttgtttactaccgcggaagtgcaggaaaaggtacaaaaaccgattggatgatgcacgagtttcgccttcccacgaaagataacagcacctccacggccactgtcaaagctaaaatctctgaccaagaagctgaggtatggacactgtgtagaattttcaaaagaaatgtgtcatgcagaaaatacacaccagatttgaagcaattatcaactacacctcaacaaccaccaatcgacacaagctccaagttatgctgtcaagtggaatctaattacactcaagaaagctacgtcaattttggtgctccactcattcaacattacgataataagcctccagttcatcatgtcaaggaaaggaaaccactgcatgtggatcagttgagctacgtagctcaaccaccatctatggcttcatctttaaacatttccagcccatatggaaatcagattcttacgcatggggactgggatgagcttacatcagttgtagattgtgcttttgatccctttcttgtgtga

SEQ ID NO: 192 - DNA, 762 bp, Populus trichocarpa
atggagtttgatgggctcccaggttttcgattccacccaacagaagaagaactcttagatttctaccttacaaagactgtcttaggtcaagacacagctgatatcattggtttcctcaacatatacaatcacgaaccttgggacttgccaggattatcgaagataggggagagggagtggtatttccttgtgcacagggagagcgttcttgggagacccaggaggacaaccgagaaaggttactggaaggcaactggttcggacaggcccatccgatgcttgatggatccgaaaaggttgttgggtcatagaaaaactatggttttctacagaggaagagctccacgagggagcaaaacggattgggtcatgaatgagtacagattgcctagcaactgctatttatcgaaggagattgtgctttgcaaagtatatagaaaggcaacatcattgaaggtcttggagcaaagggcagccatagaagaaattgttagagcacctaacgtcacatctttatctcctccaatgcaagggaatttctctttttatgacccccaaaagagcttcaacaagttcttgttagaacaaaataatgttgttcacaaagtagagggagtgacagtaatagaagaagaagaagacaacaaaattggttacccatcccaaataatgggagttggacccatcaaagagcactactccgagctcctgtcaccaaagttagtcctggattggaccatggattcactttggcctaactaa

SEQ ID NO: 193 - DNA, 1005 bp, Populus trichocarpa
atgttgatggctttaaaatcccggcttaattcttctgaaaacacaacttctgtaatggagctgtatagcaaggaacatgtggatgcaacccagttgttatacgacctgtctccttgtttcaaacttggtttcatggctgccaatcttgccatcattgacgctacaagagagcaagaacaagaggcgaacacttccagcaatggcttccatgttgttgattttgatattggtcatggagggcaatacaagaaccttctacacgcgctttctgggcttcagaattcgaagccagccattgttaagatcacagccgttgctgctgatagcaatggcgtagaggaggagaggctgaggctagttggtgaaacgcttacccaactcgctcgtcgagttggtctgaatttgtgtttcaacgttgtgagctgcaaactcagtgagttgactcgggagtccctcgggtgcgagccggacgaggcattggctgtcaattttgcgttcaagctgtatagaatgccggacgagagcgtgtcctccaccgagaatcccagggatgagcttttgaggcgcgtgaaggggctggcgccgcgcgtggtgactgtagtggagcaagaaatgaataccaacacggccccattcatggcgcgcgtgaacgaatcctgttcatattacggagcgctattcgactcgattgagtcaacggttaaaggggataactcggagcgagccaaggtcgaggagggactggggcggaggatggtaaactcggttgcttgtgaagggagggaccgtgttgaaagatgcgaggtgtttggaaaatggcgggcccggatgggcatggcagggttcgagttgaagcctctgagtcacaacatagccgagtcgatgaaaacgagattgagtttagcaaacagagttaacccaggatttagtgttaaagaagagaatggtggggtatgtttcggttggatgggaaaaactctcacagtcgcatctgcttggcgttaa

SEQ ID NO: 194 - DNA, 1674 bp, Populus trichocarpa
atggacaacaatacctctcgctctgaggtttccgactgcggcgatccaacgaggccggagtccggcggcggcggtgctaggtacaagctgatgtcgccggccaagcttccgatctcgaggtcggcttgcatcacgatccctcctggactcagtccgacctcgtttcttgagtctccagttcttctctctaatgttaaggcagagccttcccccactactggtacgtttactaagcctcgaacagctcttggctctcttagctccactccatattctgctacaactgtttcatccacagcgtgcggagaaagaaaatccgactactttgagtttagaccatatgctaggtcaaacatggtttctgcagatataaaccatcagaggagcacacaatgtgctcaagtccaaagccagtgccattcacaatcatttgcctcaccacctttggtaaaaggtgagatggaagtatccacaaatgagttgagtctgtcagcatcccttcacatggttacttcagttgctagtgcacctgctgaagttgattcagatgaactaaaccaaacggggctctctagcagtgggcttcaggcatcacagtctgatcatagagcaggaactgcgccttcaatgtcatctgatgatggatataactggagaaaatatggacagaaacatgttaagggaagtgagttccctcgaagctattacaaatgtacacatcctaactgtgaagtgaaaaagttatttgagcgctcccatgatggacaaataacagagattatctacaagggtacacatgatcatcctaaaccacaacccagccgccgatatgcctctggttctgttttgtccatgcaggaagatagatttgacaagtcttcatctctgcctaaccaaggtgacaagtcacctggtgcttatggacaggtgcctcatgctatcgagccaaatggtgcccttgaactgtctactggagcaaatgatgatactggagaaggtgccgaagacgatgatgatcccttctcaaaaagaaggaggttggatgctggaggttttgatgttactccagtggtcaaacctatccgggaaccacgtgttgtcgtgcaaactctgagtgaggttgatatactggatgatgggtacaggtggcgcaaatatggccagaaagtggtgagaggaaatcctaatccaaggagttactacaaatgcacaaatgctggatgcccagttagaaagcacgttgagagggcatcgcatgatccaaaagcagttataaccacatatgaggggaaacacaatcatgatgtacctacagctaggacaaacagtcatgacatggcgggaccatcagctgtgaatggaccctcaaggattagaccagatgaaaacgagacaattagccttgatcttggggtcgggatcagttctacaactgaaaaccagtccagcgatcagcaacaagctttgcatgctgaacttatcaaacacgagaaccaagcaagtggttctagtttcagagtagttcatgcaaccccaattacggcttactatggtgttttaaatggtggcatgaatcagtatggatctaggcaaattccaggtgaaagccgtagcattgaaattccaccttatccatatccgcagaacatgggaagattattaacgggtccataa

SEQ ID NO: 195 - DNA, 1371 bp, Populus trichocarpa
atggaaacaaagtgtccagtagatgcaaagtgcgcagctgaaatatctgccatgctcacccctccttctcctcttcaactccaggagtatttcgaggagattatatctgaaaggaaatgccatggcattgaagtcaaacaagatggaaacttgagcaaaggggtttatgcaactatggaacttaaagaaggagaacttatactgaaggacaagatacttgtgggtttacagcatgttccaaataagcttgactgtctggtgtgtggctattgtttccaatttattgagtcagttgaatatcagattgggaggaaactttatttgcaaagtctaggcgtgcccagctgcaatggatgtgatgagggggaatgtagttccagcagctcatacaataaagcttgtttgcctgaaggagttatagaagcattaatgaatggtgaattggtattgccttactctgacaaatttcccttgccttcaactgttccttgtcctggggggtgtcaagaagcttattattgcagcaaatcttgtgcgcagactgattgggaatcttcacattctttactttgcaccggggagaggtcagaatcattatctatagaggcactttcaaaatttatacagcatgctactgaaacaaatgatattttcctcctagcagccaagacaatttctttcaccattttaaggtataggaagctgaaagcagctaatgcagatcgttctgaactttccttacttttggaggcatggaagccaatatcaatgggatacaagagaaggtggtgggagtgcatttcatttccagatgatgttgatcgttctgatgacactgcatttaggatgcaaatacagcagcttgcattcaagtcactgcagcttctcaaggctgccatatttgatgaggaatgcgagccattattctcccttgaaatctatgggaatattattggcatgtttgagctgaataatcttgatctggtcgtagcatctccagttgaggattattttctgtacattgatgatcttccagatcctgaaaaggaaaaggccgaaaaaattgcacggcaacttctagatgctcttggagatgactattcaatttgttgccaaggtactgcgttctatcctttacagagctgcatgaatcactcctgctgcccaaatgcacacgccttcaaaagagatgaggacagagatggccaggcagcaatcatcaccctgaaaccaattcgaaagggagaagaggttaccgtttcatacatagacgaggaccttccatttgaagacagacaggcattgctagcagattatggattcaaatgcaggtgcaacgcttgcttagagcaagaccccaacaaaaagtag